SEQUENCE LISTING (109 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 11 - protein, 7 aa, synthetic construct
SNSAAWN

SEQ ID NO: 12 - protein, 18 aa, synthetic construct
KTYYRFKWYSDYAVSVKG

SEQ ID NO: 13 - protein, 14 aa, synthetic construct
ESTTYDLLAGPFDY

SEQ ID NO: 14 - protein, 16 aa, synthetic construct
SSQTVLYSSNNKKYLA

SEQ ID NO: 15 - protein, 7 aa, synthetic construct
WASTRES

SEQ ID NO: 16 - protein, 9 aa, synthetic construct
QQYYSTPFT

SEQ ID NO: 17 - protein, 23 aa, synthetic construct
DIVMTQSPDSLAVSLGERATINC

SEQ ID NO: 18 - protein, 15 aa, synthetic construct
WYQQKPGQPPNLLIY

SEQ ID NO: 19 - protein, 32 aa, synthetic construct
GVPDRFSGSGSGTDFTLTISSLQAEDVAVYYC

SEQ ID NO: 20 - protein, 10 aa, synthetic construct
FGPGTKVEIK

SEQ ID NO: 21 - protein, 30 aa, synthetic construct
  VARIANT 1: note = E or Q
XVQLQQSGPGLVKPSQTLSLTCAISGDSVS

SEQ ID NO: 22 - protein, 14 aa, synthetic construct
WIRQSPSRGLEWLG

SEQ ID NO: 23 - protein, 32 aa, synthetic construct
RITINPDTSKNQFSLQLNSVTPEDTAVFYCTR

SEQ ID NO: 24 - protein, 11 aa, synthetic construct
WGQGTLVTVSS

SEQ ID NO: 25 - protein, 30 aa, synthetic construct
EVQLQQSGPGLVKPSQTLSLTCAISGDSVS

SEQ ID NO: 26 - protein, 30 aa, synthetic construct
QVQLQQSGPGLVKPSQTLSLTCAISGDSVS

SEQ ID NO: 27 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 28 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 29 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 7 aa, synthetic construct
GFSFSSY

SEQ ID NO: 36 - skipped

SEQ ID NO: 37 - protein, 9 aa, synthetic construct
TGRVYFALD

SEQ ID NO: 38 - protein, 11 aa, synthetic construct
SESVDTSDNSF

SEQ ID NO: 39 - skipped

SEQ ID NO: 40 - protein, 6 aa, synthetic construct
NYDVPW

SEQ ID NO: 41 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 42 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 43 - protein, 5 aa, synthetic construct
DYTMN

SEQ ID NO: 44 - protein, 17 aa, synthetic construct
VISWDGGGTYYTDSVKG

SEQ ID NO: 45 - protein, 12 aa, synthetic construct
GLTDTTLYGSDY

SEQ ID NO: 46 - protein, 11 aa, synthetic construct
RASQSISSYLN

SEQ ID NO: 47 - protein, 7 aa, synthetic construct
AASTLQS

SEQ ID NO: 48 - protein, 9 aa, synthetic construct
QQTYSSPLT

SEQ ID NO: 49 - protein, 121 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSS

SEQ ID NO: 50 - protein, 107 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIK

SEQ ID NO: 51 - protein, 443 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 451 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 56 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 57 - protein, 422 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 58 - protein, 5 aa, synthetic construct
GGGGS

SEQ ID NO: 59 - protein, 10 aa, synthetic construct
GGGGSGGGGS

SEQ ID NO: 60 - protein, 10 aa, synthetic construct
SGGGGSGGGG

SEQ ID NO: 61 - protein, 14 aa, synthetic construct
GGGGSGGGGSGGGG

SEQ ID NO: 62 - protein, 10 aa, synthetic construct
GSPGSSSSGS

SEQ ID NO: 63 - protein, 15 aa, synthetic construct
GGGGSGGGGSGGGGS

SEQ ID NO: 64 - protein, 20 aa, synthetic construct
GGGGSGGGGSGGGGSGGGGS

SEQ ID NO: 65 - protein, 8 aa, synthetic construct
GSGSGSGS

SEQ ID NO: 66 - protein, 8 aa, synthetic construct
GSGSGNGS

SEQ ID NO: 67 - protein, 8 aa, synthetic construct
GGSGSGSG

SEQ ID NO: 68 - protein, 6 aa, synthetic construct
GGSGSG

SEQ ID NO: 69 - protein, 4 aa, synthetic construct
GGSG

SEQ ID NO: 70 - protein, 8 aa, synthetic construct
GGSGNGSG

SEQ ID NO: 71 - protein, 8 aa, synthetic construct
GGNGSGSG

SEQ ID NO: 72 - protein, 6 aa, synthetic construct
GGNGSG

SEQ ID NO: 73 - protein, 461 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 74 - protein, 10 aa, synthetic construct
GYTFTSYNMH

SEQ ID NO: 75 - protein, 17 aa, synthetic construct
AIYPGNGDTSYNQKFKG

SEQ ID NO: 76 - protein, 13 aa, synthetic construct
VVYYSNSYWYFDV

SEQ ID NO: 77 - protein, 10 aa, synthetic construct
RASSSVSYMH

SEQ ID NO: 78 - protein, 7 aa, synthetic construct
APSNLAS

SEQ ID NO: 79 - protein, 9 aa, synthetic construct
QQWSFNPPT

SEQ ID NO: 80 - protein, 122 aa, synthetic construct [sequence omitted]

SEQ ID NO: 81 - protein, 106 aa, synthetic construct [sequence omitted]

SEQ ID NO: 82 - protein, 25 aa, synthetic construct [sequence omitted]

SEQ ID NO: 83 - protein, 14 aa, synthetic construct
WVRQAPGKGLEWVG

SEQ ID NO: 84 - protein, 32 aa, synthetic construct
RFTISVDKSKNTLYLQMNSLRAEDTAVYYCAR

SEQ ID NO: 85 - protein, 11 aa, synthetic construct
WGQGTLVTVSS

SEQ ID NO: 86 - protein, 23 aa, synthetic construct
DIQMTQSPSSLSASVGDRVTITC

SEQ ID NO: 87 - protein, 15 aa, synthetic construct
WYQQKPGKAPKPLIY

SEQ ID NO: 88 - protein, 32 aa, synthetic construct
GVPSRFSGSGSGTDFTLTISSLQPEDFATYYC

SEQ ID NO: 89 - protein, 10 aa, synthetic construct
FGQGTKVEIK

SEQ ID NO: 90 - protein, 5 aa, synthetic construct
NYYIH

SEQ ID NO: 91 - protein, 17 aa, synthetic construct
WIYPGDGNTKYNEKFKG

SEQ ID NO: 92 - protein, 10 aa, synthetic construct
DSYSNYYFDY

SEQ ID NO: 93 - protein, 17 aa, synthetic construct
KSSQSLLNSRTRKNYLA

SEQ ID NO: 94 - protein, 7 aa, synthetic construct
WASTRES

SEQ ID NO: 95 - protein, 8 aa, synthetic construct
TQSFILRT

SEQ ID NO: 96 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 97 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 98 - protein, 30 aa, synthetic construct
EVQLVQSGAEVKKPGASVKVSCKASGYTFT

SEQ ID NO: 99 - protein, 14 aa, synthetic construct
WVRQAPGQGLEWIG

SEQ ID NO: 100 - protein, 32 aa, synthetic construct
RATLTADTSTSTAYLELSSLRSEDTAVYYCAR

SEQ ID NO: 101 - protein, 11 aa, synthetic construct
WGQGTLVTVSS

SEQ ID NO: 102 - protein, 23 aa, synthetic construct
DIVMTQSPDSLAVSLGERATINC

SEQ ID NO: 103 - protein, 15 aa, synthetic construct
WYQQKPGQPPKLLIY

SEQ ID NO: 104 - protein, 32 aa, synthetic construct; identical to SEQ ID NO: 19

SEQ ID NO: 105 - protein, 10 aa, synthetic construct
FGQGTKVEIK

SEQ ID NO: 106 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 109 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0287182 A1
(43) Pub. Date: Aug. 29, 2024

(54) METHODS AND COMPOSITIONS FOR TREATING CANCER

(71) Applicants: Genentech, Inc., South San Francisco, CA (US); Hoffmann-La Roche Inc., Little Falls, NJ (US)

(72) Inventors: Christoph MARKERT, Penzberg (DE); Raymond D. MENG, Mountain View, CA (US); Merlind MUECKE, Basel (CH); Shiraj SEN, Denver, CO (US); Volker TEICHGRAEBER, Basel (CH); Volker Andreas WIEBKING, Redwood City, CA (US); Edward Namserk CHA, San Carlos, CA (US); Christopher Roland COTTER, Portland, OR (US); Michelle Yuri DORAL, San Francisco, CA (US)

(21) Appl. No.: 18/402,031

(22) Filed: Jan. 2, 2024

Related U.S. Application Data
(63) Continuation of application No. PCT/US2022/073365, filed on Jul. 1, 2022.
(60) Provisional application No. 63/286,525, filed on Dec. 6, 2021.
(60) Provisional application No. 63/226,714, filed on Jul. 28, 2021.
(60) Provisional application No. 63/218,194, filed on Jul. 2, 2021.

(51) Int. Cl. C07K 16/28; A61K 39/00; A61P 35/04

(52) U.S. Cl. CPC C07K 16/2818; A61P 35/04; C07K 16/2803; A61K 2039/507; A61K 2039/545; C07K 2317/76

(57) ABSTRACT

This invention relates to methods and compositions for use in treating cancer in a subject. For example, the invention relates to methods and compositions for use in treating esophageal cancer or colorectal cancer (CRC) (e.g., metastatic CRC (e.g., microsatellite instability (MSI) high (MSI-H) metastatic CRC)) in a subject by administering to the subject an anti-T-cell immunoreceptor with Ig and ITIM domains (TIGIT) antagonist antibody (e.g., tiragolumab) and a PD-1 axis binding antagonist (e.g., atezolizumab); methods and compositions for use in treating metastatic CRC (e.g., MSI-H metastatic CRC) in a subject by administering to the subject an anti-TIGIT antagonist antibody (e.g., tiragolumab), a PD-1 axis binding antagonist (e.g., atezolizumab), and an anti-VEGF antibody (e.g., bevacizumab); methods and compositions for use in treating melanoma in a subject by administering to the subject a bispecific antibody targeting programmed cell death protein 1 (PD-1) and lymphocyte activation gene-3 (LAG3), optionally with an anti-TIGIT antagonist antibody (e.g., tiragolumab); and methods and compositions for use in treating a CD20-positive cell proliferative disorder (e.g., non-Hodgkin's lymphoma (NHL); e.g., relapsed or refractory NHL) in a subject by administering to the subject a bispecific antibody targeting CD20 and CD3 (mosunetuzumab) and an anti-TIGIT antagonist antibody (e.g., tiragolumab), optionally with a PD-1 axis binding antagonist (e.g., atezolizumab).

### SEQUENCE LISTING

The instant application contains a Sequence Listing which has been submitted electronically in XML format and is hereby incorporated by reference in its entirety. Said XML copy, created on Nov. 29, 2023, is named 50474-257005_Sequence_Listing_11_29_23.xml, and is 102,293 bytes in size.

### FIELD OF THE INVENTION

This invention relates to methods and compositions for use in treating cancer in a subject. For example, the invention relates to methods and compositions for use in treating esophageal cancer or colorectal cancer (CRC) (e.g., metastatic CRC (e.g., microsatellite instability (MSI) high (MSI-H) metastatic CRC)) in a subject by administering to the subject an anti-T-cell immunoreceptor with Ig and ITIM domains (TIGIT) antagonist antibody (e.g., tiragolumab) and a PD-1 axis binding antagonist (e.g., atezolizumab); methods and compositions for use in treating metastatic CRC (e.g., MSI-H metastatic CRC) in a subject by administering to the subject an anti-TIGIT antagonist antibody (e.g., tiragolumab), a PD-1 axis binding antagonist (e.g., atezolizumab), and an anti-VEGF antibody (e.g., bevacizumab); methods and compositions for use in treating melanoma in a subject by administering to the subject a bispecific antibody targeting programmed cell death protein 1 (PD-1) and lymphocyte activation gene-3 (LAG3), optionally with an anti-TIGIT antagonist antibody (e.g., tiragolumab); and methods and compositions for use in treating a CD20-positive cell proliferative disorder (e.g., non-Hodgkin's lymphoma (NHL); e.g., relapsed or refractory NHL) in a subject by administering to the subject a bispecific antibody targeting CD20 and CD3 (mosunetuzumab) and an anti-TIGIT antagonist antibody (e.g., tiragolumab), optionally with a PD-1 axis binding antagonist (e.g., atezolizumab).

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

There is a particularly pressing need for therapeutic approaches for treatment of cancers that are common and difficult to treat.

Esophageal cancer is the seventh most commonly diagnosed cancer worldwide and the sixth most common cause of cancer-related death. Most patients with esophageal cancer are diagnosed with advanced disease, where the disease is frequently recurrent. Treatment options for patients with advanced or metastatic esophageal cancer are limited and patients have a poor prognosis with minimal overall survival benefit from current treatments. Hence, there remains a significant need for novel therapeutic approaches in this population.

Melanoma is a malignant tumor of melanocytes. This potentially deadly form of skin cancer is one of the fastest-growing malignancies. More than 300,000 people worldwide are currently diagnosed with melanoma each year, and 57,000 people die of the disease. Most people with advanced melanoma have a poor prognosis. Patients with lymph-node involvement (Stage III) have a high risk of local and distant relapse after surgery, and the 5-year survival rate is 32%-93% in this patient group. Few patients have metastatic disease (Stage IV) at presentation, but some develop metastases after their initial definitive treatment. Immunotherapy and targeted therapies have improved the outcomes of those patients, and the 5-year survival rate is around 50%. Melanoma continues to be a serious health issue, with a high medical need and a steadily increasing incidence over the past 30 years. Hence, there remains a significant need for novel therapeutic approaches in this population.

B cell proliferative disorders are a leading cause of cancer-related deaths. For example, non-Hodgkin's lymphoma (NHL) advances quickly and is fatal if untreated. In the United States, B-cell lymphomas constitute approximately 80%-85% of all cases of NHL. Diffuse large B-cell lymphoma (DLBCL) is the most common type of NHL accounting for approximately 30%-40% of all NHL diagnosis, followed by follicular lymphoma (FL; 20%-25% of all NHL diagnosis) and mantle cell lymphoma (MCL; 6%-10% of all NHL diagnosis). B-cell chronic lymphocytic leukemia (CLL) is the most common leukemia in adults, with approximately 15,000 new cases per year in the United States (American Cancer Society 2015). Hence, there remains a significant need for novel therapies in this population.

Colorectal cancer (CRC) is a cancer that develops from the colon or rectum. CRC tumor cells with mismatch-repair (MMR) deficiency have an impaired ability to regulate the length of microsatellites during DNA replication, which is known as microsatellite instability (MSI). CRC tumors can be further characterized as MSI-high (MSI-H). A large proportion of patients with MSI-H metastatic CRC do not benefit from the current approved immunotherapies, and some patients who initially respond to therapy later develop secondary resistance to therapy. Therefore, there is a significant need for novel, effective therapies for patients with MSI-H metastatic CRC.

Thus, there is an unmet need in the field for the development of efficacious immunotherapies and methods of dosing the same for the treatment of cancer, including esophageal cancer, melanoma, B cell proliferative disorders, and CRC.

### SUMMARY OF THE INVENTION

The present invention involves methods of achieving a clinical response in a subject having a cancer (e.g., a metastatic esophageal cancer, a relapsed and/or refractory non-Hodgkin's lymphoma (NHL), or a melanoma) comprising administering to the subject a dosing regimen comprising one or more dosing cycles of a treatment regimen that includes an anti-TIGIT antagonist antibody (e.g., tiragolumab) and a PD-1 axis binding antagonist (e.g., atezolizumab); a treatment regimen that includes a bispecific antibody targeting PD-1 and LAG3; a treatment regimen that includes mosunetuzumab and an anti-TIGIT antagonist antibody (e.g., tiragolumab), or a treatment regimen that includes a bispecific antibody targeting PD-1 and LAG3 and an anti-TIGIT antagonist antibody (e.g., tiragolumab) in an amount effective to achieve the clinical response.

In one aspect, the disclosure provides a method for treating a subject having a melanoma, the method comprising administering to the subject one or more dosing cycles of an anti-TIGIT antagonist antibody and a bispecific antibody targeting PD-1 and LAG3 comprising a first antigen-binding domain that specifically binds to programmed cell death protein 1 (PD-1) and a second antigen-binding domain that specifically binds to lymphocyte activation gene 3 (LAG3).

In another aspect, the disclosure provides an anti-TIGIT antagonist antibody for use in combination with a bispecific antibody targeting PD-1 and LAG3, the bispecific antibody comprising a first antigen-binding domain that specifically binds to programmed cell death protein 1 (PD-1) and a second antigen-binding domain that specifically binds to lymphocyte activation gene 3 (LAG3), in treating a subject having a melanoma, wherein the anti-TIGIT antagonist antibody and the bispecific antibody targeting PD-1 and LAG3 are formulated for administration to the subject in a dosing regimen comprising one or more dosing cycles.

In another aspect, the disclosure provides a bispecific antibody targeting PD-1 and LAG3 for use in combination with an anti-TIGIT antagonist antibody, the bispecific antibody comprising a first antigen-binding domain that specifically binds to PD-1 and a second antigen-binding domain that specifically binds to LAG3, in treating a subject having a melanoma, wherein the bispecific antibody targeting PD-1 and LAG3 and the anti-TIGIT antagonist antibody are formulated for administration to the subject in a dosing regimen comprising one or more dosing cycles.

In another aspect, the disclosure features a method for treating a subject having a melanoma, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the one or more dosing cycles are administered as a neoadjuvant therapy.

In another aspect, the disclosure provides a bispecific antibody targeting PD-1 and LAG3 comprising a first antigen-binding domain that specifically binds to PD-1 and a second antigen-binding domain that specifically binds to LAG3 for use in treating a subject having a melanoma, wherein the bispecific antibody targeting PD-1 and LAG3 is formulated for administration to the subject as a neoadjuvant therapy in a dosing regimen comprising one or more dosing cycles.

In another aspect, the disclosure features a method for treating a subject having a melanoma, the method comprising administering to the subject one or more dosing cycles of an anti-TIGIT antagonist antibody and a PD-1 axis binding antagonist, wherein the one or more dosing cycles are administered as a neoadjuvant therapy.

In another aspect, the disclosure provides an anti-TIGIT antagonist antibody for use in combination with a PD-1 axis binding antagonist in treating a subject having a melanoma, wherein the anti-TIGIT antagonist antibody and the PD-1 axis binding antagonist are formulated for administration to the subject as a neoadjuvant therapy in a dosing regimen comprising one or more dosing cycles.

In another aspect, the disclosure provides a PD-1 axis binding antagonist for use in combination with an anti-TIGIT antagonist antibody in treating a subject having a melanoma, wherein the PD-1 axis binding antagonist and the anti-TIGIT antagonist antibody are formulated for administration to the subject as a neoadjuvant therapy in a dosing regimen comprising one or more dosing cycles.

In another aspect, the disclosure provides a method of achieving a clinical response in a subject having a metastatic esophageal cancer comprising administering to the subject a dosing regimen comprising one or more dosing cycles of tiragolumab and atezolizumab in an amount effective to achieve the clinical response.

In another aspect, the disclosure provides tiragolumab for use in combination with atezolizumab in achieving a clinical response in a subject having a metastatic esophageal cancer, wherein the tiragolumab and atezolizumab are formulated for administration to the subject in a dosing regimen comprising one or more dosing cycles.

In another aspect, the disclosure provides atezolizumab for use in combination with tiragolumab in achieving a clinical response in a subject having a metastatic esophageal cancer, wherein the tiragolumab and atezolizumab are formulated for administration to the subject in a dosing regimen comprising one or more dosing cycles.

In another aspect, the invention features a method of treating a subject having a relapsed or refractory (R/R) non-Hodgkin's lymphoma (NHL), the method comprising administering to the subject tiragolumab and mosunetuzumab.

In another aspect, the invention features tiragolumab for use in combination with mosunetuzumab in treating a subject having a R/R NHL, wherein the tiragolumab and mosunetuzumab are formulated for administration to the subject.

In another aspect, the invention features mosunetuzumab for use in combination with tiragolumab in treating a subject having a R/R NHL, wherein the tiragolumab and mosunetuzumab are formulated for administration to the subject.

In another aspect, the disclosure features a method of treating a subject having a metastatic colorectal cancer (CRC) comprising administering to the subject tiragolumab and atezolizumab, wherein the metastatic CRC is a microsatellite instability-high (MSI-H) CRC.

In another aspect, the disclosure features tiragolumab for use in combination with atezolizumab in treating a subject having a metastatic CRC, wherein the tiragolumab and atezolizumab are formulated for administration to the subject, and wherein the metastatic CRC is a MSI-H CRC.

In another aspect, the disclosure features atezolizumab for use in combination with tiragolumab in treating a subject having a metastatic CRC, wherein the tiragolumab and atezolizumab are formulated for administration to the subject, and wherein the metastatic CRC is a MSI-H CRC.

In another aspect, the disclosure features a method of treating a subject having a metastatic CRC comprising administering to the subject tiragolumab, atezolizumab, and bevacizumab, wherein the metastatic CRC is a MSI-H CRC.

In another aspect, the disclosure features tiragolumab for use in combination with atezolizumab and bevacizumab in treating a subject having a metastatic CRC, wherein the tiragolumab, atezolizumab, and bevacizumab are formulated for administration to the subject, and wherein the metastatic CRC is a MSI-H CRC.

In another aspect, the disclosure features atezolizumab for use in combination with tiragolumab and bevacizumab in treating a subject having a metastatic CRC, wherein the tiragolumab, atezolizumab, and bevacizumab are formulated for administration to the subject, and wherein the metastatic CRC is a MSI-H CRC.

In another aspect, the disclosure features bevacizumab for use in combination with atezolizumab and tiragolumab in treating a subject having a metastatic CRC, wherein the tiragolumab, atezolizumab, and bevacizumab are formulated for administration to the subject, and wherein the metastatic CRC is a MSI-H CRC.

In another aspect, the disclosure features a method of treating a subject having a metastatic CRC, the method comprising administering to the subject a dosing regimen comprising one or more 21-day dosing cycles of tiragolumab at a dose of about 600 mg on Day 1 of each dosing cycle and atezolizumab at a dose of about 1200 mg on Day 1 of each dosing cycle, wherein the metastatic CRC is a MSI-H CRC.

In another aspect, the disclosure features a method of treating a subject having a metastatic CRC, the method comprising administering to the subject a dosing regimen comprising one or more 21-day dosing cycles of tiragolumab at a dose of about 600 mg on Day 1 of each dosing cycle, atezolizumab at a dose of about 1200 mg on Day 1 of each dosing cycle, and bevacizumab at a dose of about 15 mg/kg on Day 1 of each dosing cycle, wherein the metastatic CRC is a MSI-H CRC.

### BRIEF DESCRIPTION OF THE DRAWINGS

FIG. 1 is a bar graph showing the best percent change in the sum of longest diameters (SLD) for patients with metastatic esophageal cancer who were treated with tiragolumab and atezolizumab. Each patient is represented by a bar. Adenocarcinoma (light gray) or squamous cell carcinoma (dark gray) esophageal histopathological subtype is shown for each patient. The number of prior lines of systemic therapy and the PD-L1 expression status of tumor cells (TC) or immune cells (IC) (<5% or ≥5%) are shown under the x-axis for each patient. Reference lines indicating a 20% increase in the best change in the SLD and a 30% decrease in the best change in the SLD are shown. PD-L1 expression was measured using the SP142 IHC assay. NTL=non-target lesion; PD=progression of disease.

FIG. 2 is a graph showing the percent change in the SLD over time (study days) for patients with metastatic esophageal cancer who were treated with tiragolumab and atezolizumab. Adenocarcinoma (light gray) or squamous cell carcinoma (dark gray) esophageal histopathological subtype is shown for each patient. Reference lines indicating a 20% increase in the SLD and a 30% decrease in the SLD are shown.

FIG. 3 is a series of computerized/computed tomography (CT) scan images showing transverse sections of a metastatic esophageal adenocarcinoma showing durable partial response in a patient who received multiple prior lines of therapy. The images were captured on the dates shown.

FIG. 4 is a flow chart showing the study design of the phase Ib/II clinical trial in patients with melanoma. Atezo=atezolizumab; CIT=cancer immunotherapy; CLND=completion lymph node dissection; Ipi=ipilimumab; Nivo=nivolumab; R=randomization; Tira=tiragolumab.

FIG. 5 is a schematic diagram of the study schema showing an overview of the study schedule and activities in Cohort 1 of the phase Ib/II clinical trial in patients with melanoma. CLND=completion lymph node dissection; Comp.=completion; CT=computed tomography; Discon.=discontinuation; M=month; R=randomization; Q3M=every 3 months; SFU=survival follow-up; Tx=treatment; W=week.

FIG. 6 is a schematic diagram of the study schema showing an overview of the study schedule and activities in Cohorts A-F of the phase Ib/II clinical trial in patients with relapsed or refractory B-cell non-Hodgkin's lymphoma. DLBCL=diffuse large B cell lymphoma; FL=follicular lymphoma; Gr=Grade; HGBL=high grade B cell lymphoma; IV=intravenous administration; R/R=relapsed or refractory; SC=subcutaneous administration; trFL=transformed follicular lymphoma. ^{a}For Cohort A, see FIG. 7A for detailed dosing schedule and dosing information. ^{b}For Cohort B, see FIG. 7B for detailed dosing schedule and dosing information. ^{c/d}For Cohorts E and F, see FIG. 7C for detailed dosing schedule and dosing information. Mosunetuzumab and tiragolumab dosing for Cohorts C, D, E, and F will follow either mosunetuzumab and tiragolumab dosing of Cohort A or Cohort B based on safety results of Cohorts A and B.

FIG. 7A is a schematic diagram of the dosing schedule and dosing levels of Cohort A of the phase Ib/II clinical trial in patients with relapsed or refractory B-cell non-Hodgkin's lymphoma. D=day; DLT=dose limiting toxicity; IV=intravenous administration; SC=subcutaneous administration. ^{a}Participants hospitalized for 72 hours following end of administration of the last study drug.

FIG. 7B is a schematic diagram of the dosing schedule and dosing levels of Cohort B of the phase Ib/II clinical trial in patients with relapsed or refractory B-cell non-Hodgkin's lymphoma. D=day; DLT=dose limiting toxicity; IV=intravenous administration; SC=subcutaneous administration. ^{a}Participants hospitalized for 72 hours following end of administration of the last study drug.

FIG. 7C is a schematic diagram of the dosing schedule and dosing levels of Cohorts E and F of the phase Ib/II clinical trial in patients with relapsed or refractory B-cell non-Hodgkin's lymphoma. D=day; DLT=dose limiting toxicity; IV=intravenous administration; SC=subcutaneous administration. ^{a}Participants hospitalized for 72 hours following end of administration of the last study drug.

FIG. 8A is a schematic diagram of the study design of the atezolizumab+tiragolumab+bevacizumab treatment arm for patients in the microsatellite instability-high (MSI-H) metastatic colorectal cancer (CRC) cohort in the INTRINSIC (identifying and targeting subpopulations in CRC) phase Ib/II study. Atezo=atezolizumab; Bev=bevacizumab; C=cycle; IHC=immunohistochemistry; MSI-H=microsatellite instability high; NGS=next generation sequencing; Q3W=every 3 weeks; Q6W=every 6 weeks; Q12W=every 12 weeks; Tira=tiragolumab.

FIG. 8B is a schematic diagram of the study design of the atezolizumab+tiragolumab treatment arm for patients in the MSI-H metastatic CRC cohort in the INTRINSIC (identifying and targeting subpopulations in CRC) phase Ib/II study. Atezo=atezolizumab; Bev=bevacizumab; C=cycle; IHC=immunohistochemistry; MSI-H=microsatellite instability high; NGS=next generation sequencing; Q3W=every 3 weeks; Q6W=every 6 weeks; Q12W=every 12 weeks; Tira=tiragolumab.

### DETAILED DESCRIPTION OF THE INVENTION

The present invention provides therapeutic methods and compositions for treatment of cancer, for example, esophageal cancer, melanoma, CD20-positive cell proliferative disorders, and colorectal cancer. The invention is based, at least in part, on the discovery that immunotherapies including an anti-TIGIT antibody (e.g., an anti-TIGIT antagonist antibody, such as tiragolumab) in combination with a PD-1 axis binding antagonist (e.g., an anti-programmed death ligand-1 (PD-L1) antibody (e.g., atezolizumab) or an anti-programmed death-1 (PD-1) antibody) can be useful in the treatment of cancer. Compositions, uses, and kits involving such combinations and/or dosing regimens are also provided herein.

### I. Definitions

The following abbreviations are used herein:

| | |
|---|---|
| ASCT | autologous stem cell therapy |
| CAR | chimeric antigen receptor |
| CAS | Chemical Abstracts Service |
| CDR | complementarity determining region |
| CR | complete response |
| CRC | colorectal cancer |
| DLBCL | diffuse large B cell lymphoma |
| DNA | deoxyribonucleic acid |
| DCR | disease control rate |
| DOR | duration of response |
| Fab | fragment antigen-binding |
| Fc | fragment crystallizable |
| FFPE | formalin-fixed and paraffin-embedded |
| FL | follicular lymphoma |
| FR | framework |
| HGBL | high grade B cell lymphoma |
| HVR | hypervariable region |
| IHC | immunohistochemistry |
| IV | intravenous |
| MSI | microsatellite instability |
| MSI-H | microsatellite instability-high |
| NHL | non-Hodgkin's lymphoma |
| ORR | overall response rate/objective |
| | response rate |
| OS | overall survival |
| PD-1 | programmed death 1 |
| PD-L1 | programmed death ligand 1 |
| PD-L2 | programmed death ligand 2 |
| PFS | progression-free survival |
| PR | partial response |
| Q2W | once every 2 weeks |
| Q3W | once every 3 weeks |
| Q4W | once every 4 weeks |
| RNA | ribonucleic acid |
| R/R | relapsed or refractory |
| SC | subcutaneous |
| SLD | sum of the longest diameters |
| trFL | transformed follicular lymphoma |

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

As used herein, “achieving a clinical response” refers to achieving one or more indicators of therapeutic efficacy for a disease (e.g., a cancer, e.g., an esophageal cancer) in a patient or population of patients during or following treatment with one or more agents intended to treat the disease (e.g., during or following a dosing regimen comprising one or more agents, e.g., during or following a dosing regimen comprising one or more dosing cycles of tiragolumab and atezolizumab), wherein the improvement is attributed to the treatment. The indicator of therapeutic efficacy may be, e.g., progression-free survival (PFS) (e.g., a duration of PFS that is at or above a target duration of PFS); overall survival (OS) (e.g., a duration of OS that is at or above a target duration of OS); a partial response (PR); a complete response (CR); a reduction in the sum of longest diameters (SLD) of one or more target lesions; a pathologic response rate (pRR) in a population of patients that is at or above a target pRR; an overall response rate (ORR) in a population of patients that is at or above a target ORR; a duration of response (DOR) in a patient that is at or above a target DOR; a median DOR in a population of patients that is at or above a target DOR; or a disease control rate (DCR) in a population of patients that is at or above a target DCR. In some instances, the indicator of therapeutic efficacy is an improvement relative to a comparator population (e.g., a comparator arm of a study), e.g., a duration of PFS that is at or above the duration of PFS in a comparator arm; a duration of OS that is at or above the duration of OS in a comparator arm; a higher proportion of patients achieving PR or CR relative to a comparator arm; a greater reduction in the SLD of one or more target lesions relative to the reduction in SLD of tumors in a comparator arm; a pRR in a population of patients that is at or above the pRR in a comparator arm; an ORR in a population of patients that is at or above the ORR in a comparator arm; a DOR in a patient that is at or above a comparator DOR; a median DOR in a population of patients that is at or above the DOR in a comparator arm; or a DCR in a population of patients that is at or above the DCR in a comparator arm.

The term “comparator” or “comparator arm” as used herein refers to a reference (e.g., a reference population of patients) used as a basis of comparison for a treatment or treatment arm in a study, e.g., a clinical trial. For example, a comparator arm may be a control arm in a clinical trial. The comparator arm may include a population of patients who have received a control treatment, such as one or more previously approved treatments or marketed products.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

As used herein, “tiragolumab” is a fully human IgG1/kappa MAb-derived in Open Monoclonal Technology (OMT) rats that binds TIGIT and comprises the heavy chain sequence of SEQ ID NO: 33 and the light chain sequence of SEQ ID NO: 34. Tiragolumab comprises two N-linked glycosylation sites (N306) in the Fc domain. Tiragolumab is also described in WHO Drug Information (International Nonproprietary Names for Pharmaceutical Substances), Proposed INN: List 117, Vol. 31, No. 2, published Jul. 7, 2017 (see page 343).

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one aspect, the extent of binding of an anti-TIGIT antagonist antibody to an unrelated, non-TIGIT protein is less than about 10% of the binding of the antibody to TIGIT as measured, e.g., by a radioimmunoassay (RIA). In certain aspects, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-TIGIT antagonist antibody binds to an epitope of TIGIT that is conserved among TIGIT from different species or an epitope on TIGIT that allows for cross-species reactivity. In some aspects, the anti-TIGIT binding antibody has intact Fc-mediated effector function (e.g., tiragolumab, vibostolimab, etigilimab, EOS084448, or TJ-T6). In some aspects, the anti-TIGIT binding antibody has enhanced Fc-mediated effector function (e.g., SGN-TGT). In other aspects, the anti-TIGIT binding antibody lacks Fc-mediated effector function (e.g., domvanalimab, BMS-986207, ASP8374, or COM902). In some aspects, the anti-TIGIT binding antibody is an IgG1 class antibody (e.g., tiragolumab, vibostolimab, domvanalimab, BMS-986207, etigilimab, BGB-A1217, SGN-TGT, EOS084448 (EOS-448), TJ-T6, or AB308). In other aspects, the anti-TIGIT binding antibody is an IgG4 class antibody (e.g., ASP8374 or COM902). In one aspect, the anti-TIGIT antagonist antibody is tiragolumab.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-88, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1) (SEQ ID NO: 32). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1 LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is esophageal cancer. The cancer may be locally advanced or metastatic. In some instances, the cancer is locally advanced. In other instances, the cancer is metastatic. In some instances, the cancer may be unresectable (e.g., unresectable locally advanced or metastatic cancer). Examples of cancer include, but are not limited to, carcinoma, lymphoma, blastoma, sarcoma, and leukemia or lymphoid malignancies. More particular examples of cancers include, but are not limited to, esophageal cancer (e.g., squamous cell carcinoma (e.g., esophageal squamous-cell carcinoma (ESCC)), adenocarcinoma (e.g., esophageal adenocarcinoma (EAC)), or esophageal cancers having neuroendocrine histopathology (e.g., esophageal neuroendocrine carcinoma (ENEC)). Additional examples include metastatic esophageal cancer (e.g., metastatic ESCC, metastatic EAC, or metastatic ENEC). In one instance, the cancer is a colorectal cancer (CRC). As used herein, the term “colorectal cancer,” “CRC,” “colon cancer,” or “bowel cancer” refers to a cancer that develops from the large intestine, e.g., the colon or rectum (e.g., colorectal adenomacarcinoma). In some instances, the CRC is metastatic. In some instances, the CRC is microsatellite instability (MSI)-high (MSI-H) (e.g, MSI-H metastatic CRC). Other examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be relapsed or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, α heavy chain disease, γ heavy chain disease, μ heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a relapsed and/or refractory FL or transformed FL), diffuse large B cell lymphoma (DLBCL) (e.g., a relapsed or refractory DLBCL or a Richter's transformation), MCL, high grade B-cell lymphoma, or PMLBCL). In another embodiment, the B cell proliferative disorder is a leukemia, such as chronic lymphocytic leukemia (CLL). In one embodiment, the B-cell proliferative disorder is a relapsed and/or refractory FL.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

As used herein, “microsatellite instability status” or “MSI status” refers to a characterization of microsatellite stability in a tumor tissue of a patient. The tumor tissue of a patient may be characterized as “microsatellite instability high” (“MSI-H”) or “microsatellite stable” (“MSS”). MSI status may be assessed, for example, by using a PCR-based approach such as the MSI Analysis System (Promega, Madison, WI), which is comprised of five pseudomonomorphic mononucleotide repeats (BAT-25, BAT-26, NR-21, NR-24, and MONO-27) to detect MSI and two pentanucleotide loci (PentaC and PendaD) to confirm identity between normal and tumor samples. The size in bases for each microsatellite locus can be determined, e.g., by gel electrophoresis, and a tumor may be designated MSI-H if two or more mononucleotide loci vary in length compared to the germline DNA. See, e.g., Le et al. NEJM372:2509-2520, 2015. MSI status may also be assessed, for example, by using next-generation sequencing (e.g., the blood-based FOUNDATIONONE® Liquid CDx NGS assay), immunohistochemistry (IHC), or a combination thereof. In other embodiments, a patient may have a low level of microsatellite instability (e.g., MSS).

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

“Refractory disease” is defined as a disease, particularly a CD20-positive cell proliferative disorder (e.g., a B cell proliferative disorder, e.g., a non-Hodgkin's lymphoma (NHL), e.g., a diffuse large B cell lymphoma (DLBCL), a high grade B cell lymphoma (HGBL), a follicular lymphoma (FL), e.g., a transformed FL (trFL) and FL Grade 1, 2, 3a, or 3b), for which no complete remission occurs to at least a first-line therapy. In one embodiment, refractory CD20-positive cell proliferative disorder (e.g., refractory NHL) is defined as no response to, or relapse within 6 months of, prior therapy. In one embodiment, refractory NHL is characterized by one or more of the following: progressive disease (PD) as best response to first-line therapy; stable disease (SD) as best response after at least one first-line therapy (e.g., at least one containing an anti CD20-directed therapy, e.g., including an anti-CD20 antibody, e.g., an anti-CD20 monoclonal antibody, e.g., rituximab or obinutuzumab); partial response (PR) as best response; and biopsy-proven residual disease or disease progression after the partial response. “Relapsed disease” is defined as a disease, particularly a CD20-positive cell proliferative disorder (e.g., a B cell proliferative disorder, e.g., a non-Hodgkin's lymphoma (NHL), e.g., a diffuse large B cell lymphoma (DLBCL), a high grade B cell lymphoma (HGBL), a follicular lymphoma (FL), e.g., a transformed FL (trFL) and FL Grade 1, 2, 3a, or 3b), for which complete remission occurs to first-line therapy followed by disease (e.g., CD20-positive cell proliferative disorder, e.g., NHL) recurrence. In one embodiment, NHL relapse is proven by biopsy. In one embodiment, patients have relapsed after, or failed to respond to, at least one prior systemic treatment regimen (e.g., at least one containing an anti CD20-directed therapy, e.g., including an anti-CD20 antibody, e.g., an anti-CD20 monoclonal antibody, e.g., rituximab or obinutuzumab). In one embodiment, patients have relapsed after, or failed to respond to, at least two prior systemic treatment regimens (e.g., at least one containing an anti CD20-directed therapy, e.g., including an anti-CD20 antibody, e.g., an anti-CD20 monoclonal antibody, e.g., rituximab or obinutuzumab).

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and an anti-TIGIT antagonist antibody, e.g., atezolizumab and tiragolumab). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment.

Herein, an “effective amount” refers to the amount of a therapeutic agent (e.g., a PD-1 axis binding antagonist (e.g., atezolizumab) or a combination of therapeutic agents (e.g., a PD-1 axis antagonist and an anti-TIGIT antagonist antibody, e.g., atezolizumab and tiragolumab)), that achieves a therapeutic result. In some examples, the effective amount of a therapeutic agent or a combination of therapeutic agents is the amount of the agent or of the combination of agents that achieves a clinical endpoint of improved pathologic response rate (PRR), improved overall response rate (ORR), improved disease control rate (DCR), a complete response (CR), a pathological complete response (pCR), a partial response (PR), improved survival (e.g., disease-free survival (DFS), and/or progression-free survival (PFS) and/or overall survival (OS)), and/or improved duration of response (DOR).

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used here, “progressive disease” and “PD” refers to at least a 20% increase in the SLD of target lesions, taking as reference the smallest sum on study (nadir), including baseline. The appearance of one or more new lesions may also be considered PD.

As used herein, “stable disease” and “SD” refers to neither sufficient shrinkage to qualify for PR nor sufficient increase to qualify for PD, taking as reference the smallest sum.

As used herein, “disease control rate” and “DCR” refer to the percentage of patients with advanced or metastatic cancer who have achieved CR, PR, and stable disease (SD). For example, DCR may be defined as the proportion of patients with SD for ≥12 weeks or a CR or PR, as determined by the investigator according to RECIST v1.1.

As used herein, “overall response rate,” “objective response rate,” and “ORR” refer interchangeably to the sum of CR rate and PR rate. For example, objective response may be defined as a CR or PR per Response Evaluation Criteria in Solid Tumors (RECIST) v.1.1, as determined by investigator assessment and confirmed by repeat assessment ≥4 weeks after initial documentation. In another example, ORR may be defined as the proportion of patients with CR or PR on two consecutive occasions 24 weeks apart, as determined by the investigator according to RECIST v1.1.

As used herein, “pathologic response rate” and “pRR” refer interchangeably to the proportion of patients with pathologic complete response (pCR, e.g., a complete absence of viable tumor in the treated tumor bed), pathologic near complete response (pnCR, e.g., <10% of of the treated tumor bed is occupied by viable tumor cells), and pathologic partial response (pPR, e.g., <50% of the treated tumor bed is occupied by viable tumor cells), e.g., at the time of surgery.

As used herein, “progression-free survival” and “PFS” refer to the length of time during and after treatment during which the cancer does not get worse. PFS may include the amount of time patients have experienced a CR or a PR, as well as the amount of time patients have experienced stable disease. For example, PFS may be defined as the time from the first study treatment to the first occurrence of progression or death from any cause, whichever occurs first, per RECIST v.1.1 as determined by the investigator. In another example, PFS may be defined as the time from study enrollment to the first occurrence of progression or death from any cause, whichever occurs first, per RECIST v.1.1 as determined by the investigator.

As used herein, “overall survival” and “OS” refer to the length of time from either the date of diagnosis or the start of treatment for a disease (e.g., cancer) that the patient is still alive. For example, OS may be defined as the time from first study treatment to death from any cause.

As used herein, the term “duration of response” and “DOR” refer to a length of time from documentation of a tumor response until disease progression or death from any cause, whichever occurs first. For example, DOR may be defined as the time from the first occurrence of a documented objective response to the time of the first documented disease progression or death from any cause, whichever occurs first, per RECIST v1.1 as determined by the investigator.

As used herein, the term “chemotherapeutic agent” refers to a compound useful in the treatment of cancer, such as esophageal cancer. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “patient” or “subject” refers to a human patient or subject. For example, the patient or subject may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

A “naked antibody” refers to an antibody that is not conjugated to a heterologous moiety (e.g., a cytotoxic moiety) or radiolabel. The naked antibody may be present in a pharmaceutical composition.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “monospecific” antibody as used herein denotes an antibody that has one or more binding sites each of which bind to the same epitope of the same antigen. The term “bispecific” antibody as used herein means that the antibody is able to specifically bind to at least two distinct antigens, for example two binding sites each formed by a pair of an antibody heavy chain variable domain (VH) and an antibody light chain variable domain (VL) binding to different antigens or to different epitopes on the same antigen. Such a bispecific antibody is an 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigen.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an immunohistochemical (IHC) assay, e.g., an IHC assay staining for PD-L1 using the antibody SP142, SP263, 22C3, or 28-8. Accordingly, a PD-L1-positive tumor cell fraction may be calculated using the PD-L1 IHC SP142 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively.

As used herein, the “Ventana SP142 IHC assay” is conducted according to the Ventana PD-L1 (SP142) Assay package insert (Tucson, AZ: Ventana Medical Systems, Inc.), which is incorporated herein by reference in its entirety.

As used herein, the “Ventana SP263 IHC assay” is conducted according to the Ventana PD-L1 (SP263) Assay package insert (Tucson, AZ: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, CA: Dako, Agilent Pathology Solutions), which is incorporated herein by reference in its entirety.

As used herein, the “pharmDx 28-8 IHC assay” is conducted according to the PD-L1 IHC 28-8 pharmDx package insert (Carpinteria, CA: Dako, Agilent Pathology Solutions), which is incorporated herein by reference in its entirety.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and an anti-TIGIT antagonist antibody (e.g., tiragolumab). As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v4.0 or v5.0 (NIH CTCAE). In some aspects, the AE is a low-grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high-grade AE, e.g., a Grade 3, Grade 4, or Grade 5 AE. In some instances, the AE is a Grade 3 or a Grade 4 AE. Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “treatment-related AE” refers to an AE that is judged by an investigator to have occurred as a result of a treatment, e.g., a PD-1 axis binding antagonist therapy (e.g., atezolizumab therapy) and/or an anti-TIGIT antagonist antibody therapy (e.g., tiragolumab therapy).

The term “valent” as used within the current application denotes the presence of a specified number of binding domains in an antigen binding molecule. As such, the terms “bivalent,” “tetravalent,” and “hexavalent” denote the presence of two binding domains, four binding domains, and six binding domains, respectively, in an antigen binding molecule. The bispecific antibodies according to the invention are at least “bivalent” and may be “trivalent” or “multivalent” (e.g., “tetravalent” or “hexavalent”). In a particular aspect, the antibodies of the present invention have two or more binding sites and are bispecific. That is, the antibodies may be bispecific even in cases where there are more than two binding sites (i.e., that the antibody is trivalent or multivalent).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g., scFv); multispecific antibodies formed from antibody fragments and single domain antibodies. For a review of certain antibody fragments, see Hudson et al., Nat Med 9, 129-134 (2003). For a review of scFv fragments, see e.g., Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding domains that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g., U.S. Pat. No. 6,248,516 B1). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies. Antibody fragments can be made by various techniques, including but not limited to proteolytic digestion of an intact antibody as well as production by recombinant host cells (e.g., E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e., the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab (VLVH). On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab (CLCH1).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding domain which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g., described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g., U.S. Pat. No. 7,166,697B1). Evibodies are around the same size as the isolated variable region of an antibody (e.g., a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33-residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

The term “a bispecific antibody comprising a first antigen-binding domain that specifically binds to PD-1 and a second antigen-binding domain that specifically binds to LAG3,” “a bispecific antibody that specifically binds PD-1 and LAG3,” “bispecific antigen binding molecule specific for PD-1 and LAG3” or an “anti-PD-1/anti-LAG3 antibody” are used interchangeably herein and refer to a bispecific antibody that is capable of binding PD-1 and LAG3 with sufficient affinity such that the antibody is useful as a diagnostic and/or therapeutic agent in targeting PD-1 and LAG3.

The term “PD-1,” also known as Programmed cell death protein 1, is a type I membrane protein of 288 amino acids that was first described in 1992 (Ishida et al., EMBO J., 11 (1992), 3887-3895). PD-1 is a member of the extended CD28/CTLA-4 family of T cell regulators and has two ligands, PD-L1 (B7-H1, CD274) and PD-L2 (B7-DC, CD273). The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals. This is consistent with binding of SHP-1 and SHP-2 phosphatases to the cytoplasmic tail of PD-1 upon ligand binding. While PD-1 is not expressed on naïve T cells, it is upregulated following T cell receptor (TCR)-mediated activation and is observed on both activated and exhausted T cells (Agata et al., Int. Immunology 8 (1996), 765-772). These exhausted T-cells have a dysfunctional phenotype and are unable to respond appropriately. Although PD-1 has a relatively wide expression pattern its most important role is likely as a coinhibitory receptor on T cells (Chinai et al, Trends in Pharmacological Sciences 36 (2015), 587-595). Current therapeutic approaches thus focus on blocking the interaction of PD-1 with its ligands to enhance T cell response. The terms “Programmed Death 1,” “Programmed Cell Death 1,” “Protein PD-1,” “PD-1,” PD1,” “PDCD1,” “hPD-1” and “hPD-1” can be used interchangeably, and include variants, isoforms, species homologs of human PD-1, and analogs having at least one common epitope with PD-1. The amino acid sequence of human PD-1 is shown in UniProt (www.uniprot.org) accession no. Q15116 (SEQ ID NO: 55).

The terms “LAG3” or “Lag-3” or “Lymphocyte activation gene-3” or “CD223” as used herein refer to any native LAG3 from any vertebrate source, including mammals such as primates (e.g., humans) and rodents (e.g., mice and rats), unless otherwise indicated. The term encompasses “full-length,” unprocessed LAG3 as well as any form of LAG3 resulting from processing in the cell. The term also encompasses naturally occurring variants of LAG3, e.g., splice variants or allelic variants. In one preferred embodiment the term “LAG3” refers to human LAG3. The amino acid sequence of an exemplary processed (without signal sequences) LAG3 is shown in SEQ ID NO: 56. The amino acid sequence of an exemplary Extracellular Domain (ECD) LAG3 is shown in SEQ ID NO: 57.

The terms “anti-LAG3 antibody” and “an antibody that binds to LAG3” refer to an antibody that is capable of binding LAG3 with sufficient affinity such that the antibody is useful as a diagnostic and/or therapeutic agent in targeting LAG3. In one aspect, the extent of binding of an anti-LAG3 antibody to an unrelated, non-LAG3 protein is less than about 10% of the binding of the antibody to LAG3 as measured, e.g., by a radioimmunoassay (RIA). In certain embodiments, an antibody that binds to LAG3 has a dissociation constant (KD) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-LAG3 antibody binds to an epitope of LAG3 that is conserved among LAG3 from different species. In one preferred embodiment, an “anti-LAG3 antibody,” “an antibody that specifically binds to human LAG3,” and “an antibody that binds to human LAG3” refers to an antibody specifically binding to the human LAG3 antigen or its Extracellular Domain (ECD) with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/1 to 1.0×10⁻¹³ mol/l. In this context the binding affinity is determined with a standard binding assay, such as surface plasmon resonance technique (BIAcore®, GE-Healthcare Uppsala, Sweden) e.g., using the LAG3 extracellular domain. The term “anti-LAG3 antibody” also encompasses bispecific antibodies that are capable of binding LAG3 and a second antigen.

The “knob-into-hole” technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 58) GGGGSGGGGS (SEQ ID NO: 59), SGGGGSGGGG (SEQ ID NO: 60) and GGGGSGGGGSGGGG (SEQ ID NO: 61), but also include the sequences GSPGSSSSGS (SEQ ID NO: 62), (G4S)₃ (SEQ ID NO: 63), (G4S)₄ (SEQ ID NO: 64), GSGSGSGS (SEQ ID NO: 65), GSGSGNGS (SEQ ID NO: 66), GGSGSGSG (SEQ ID NO: 67), GGSGSG (SEQ ID NO: 68), GGSG (SEQ ID NO: 69), GGSGNGSG (SEQ ID NO: 70), GGNGSGSG (SEQ ID NO: 71) and GGNGSG (SEQ ID NO: 72). Peptide linkers of particular interest are (G₄S) (SEQ ID NO: 58), (G₄S)₂ or GGGGSGGGGS (SEQ ID NO: 59), (G₄S)₃ (SEQ ID NO: 63) and (G₄S)₄ (SEQ ID NO: 65), more particularly (G₄S)₂ or GGGGSGGGGS (SEQ ID NO: 59).

By “fused to” or “connected to” is meant that the components (e.g., an antigen-binding domain and a Fc domain) are linked by peptide bonds, either directly or via one or more peptide linkers.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (KD) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species. In some embodiments, the anti-CD20 antibody is a monoclonal antibody. In some embodiments, the anti-CD20 antibody or anti-CD20 monoclonal antibody is rituximab. In some embodiments, the anti-CD20 antibody or anti-CD20 monoclonal antibody is obinutuzumab.

As used herein, the term “rituximab” or “RITUXAN®” refers to an anti-CD20 antibody (e.g., anti-CD20 monoclonal antibody) having the Proposed International Nonproprietary Names for Pharmaceutical Substances (Proposed INN) List 77 (WHO Drug Information, Vol. 11, No. 2, 1997, p. 99), or the CAS Registry Number 174722-31-7.

As used herein, the term “obinutuzumab” or “GAZYVA®” refers to an anti-CD20 antibody (e.g., anti-CD20 monoclonal antibody) having the Proposed International Nonproprietary Names for Pharmaceutical Substances (Proposed INN) List 99 (WHO Drug Information, Vol. 22, No. 2, 2008, p. 396), Proposed International Nonproprietary Names for Pharmaceutical Substances (Proposed INN) List 108 (WHO Drug Information, Vol. 26, No. 4, 2012, p. 453), or the CAS Registry Number 949142-50-1.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

The terms “anti-CD20/anti-CD3 bispecific antibody,” “bispecific anti-CD20/anti-CD3 antibody,” and “antibody that binds to CD20 and CD3,” refer to mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 303), or the CAS Registry Number 1905409-39-3.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Such antagonists are also referred to herein as “VEGFR inhibitors.” Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF biological activities” specifically includes VEGF-mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a KD value of between 100 nM and 1 pM. Antibody affinities may be determined, e.g., by a surface plasmon resonance based assay (such as the BIAcore® assay as described in PCT Application Publication No. WO2005/012359); enzyme-linked immunoabsorbent assay (ELISA); and competition assays (e.g. radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including, but not limited to, the antibody known as bevacizumab (BV; AVASTIN®).

The anti-VEGF antibody “bevacizumab (BV),” also known as “rhuMAb VEGF” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference.

### II. Therapeutic Methods and Compositions for Esophageal Cancer

### A. Methods of Achieving a Clinical Response

In one aspect, provided herein is a method of achieving a clinical response in a subject having a metastatic esophageal cancer comprising administering to the subject tiragolumab and atezolizumab in an amount effective to achieve the clinical response. In some embodiments, the tiragolumab and atezolizumab are administered to the subject in a dosing regimen that comprises one or more dosing cycles. In some embodiments, the metastatic esophageal cancer is a squamous cell carcinoma, an adenocarcinoma, or an esophageal cancer having neuroendocrine histopathology.

In some aspects, the clinical response is maintained for at least 1 month, at least 2 months, at least 3 months, at least 4 months, at least 5 months, at least 6 months, at least 7 months, at least 8 months, at least 9 months, at least 10 months, at least 11 months, at least 1 year, at least 1 year and 1 month, at least 1 year and 2 months, at least 1 year and 3 months, at least 1 year and 4 months, at least 1 year and 5 months, at least 1 year and 6 months, at least 1 year and 7 months, at least 1 year and 8 months, at least 1 year and 9 months, at least 1 year and 10 months, at least 1 year and 11 months, at least 2 years, at least 2 years and 1 month, at least 2 years and 2 months, at least 2 years and 3 months, at least 2 years and 4 months, at least 2 years and 5 months, at least 2 years and 6 months, at least 2 years and 7 months, at least 2 years and 8 months, at least 2 years and 9 months, at least 2 years and 10 months, at least two years and 11 months, at least 3 years, at least 3.5 years, at least 4 years, at least 4.5 years, at least 5 years, at least 5.5 years, at least 6 years, at least 6.5 years, at least 7 years, at least 7.5 years, at least 8 years, at least 8.5 years, at least 9 years, at least 9.5 years, at least 10 years, or more than 10 years (e.g., the clinical response is maintained for 1-2 months, 2-4 months, 4-6 months, 6-8 months, 8-10 months, 10-12 months, 1 year to 1.5 years, 1.5 years to 2 years, 2 years to 2.5 years, 2.5 years to 3 years, 3 years to 3.5 years, 3.5 years to 4 years, 4 years to 4.5 years, 4.5 years-5 years, 5 years to 6 years, 6 years to 7 years, 7 years to 8 years, 8 years to 9 years, or 9 years to 10 years). For example, in some aspects, the clinical response is maintained for 1 month to 10 years, 6 months to 5 years, 1 year to 4 years, 1 year to 3 years, or 1 year to 2 years.

In some aspects, the clinical response is maintained for at least 1 year. In some aspects, the clinical response is maintained for at least 2 years.

i. Clinical Responses

In some aspects, the clinical response is progression-free survival (PFS). PFS refers to the length of time during and after treatment during which a subject's cancer (e.g., a metastatic esophageal cancer) does not get worse. PFS may include the amount of time subjects have experienced a complete response (CR) a partial response (PR), or stable disease.

In some aspects, the clinical response is a partial response (PR). PR refers to at least a 30% decrease in the sum of the longest diameters (SLD) of target lesions, taking as reference the baseline SLD prior to treatment.

In some aspects, the clinical response is a compete response (CR). CR refers to disappearance of all target lesions.

In some aspects, the clinical response is a reduction in the sum of longest diameters (SLD) of one or more target lesions (e.g., metastatic esophageal cancer tumors). In some aspects, the SLD is decreased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or the SLD is decreased by 100% (e.g., target lesions disappear) during or following administration of the one or more dosing cycles of tiragolumab and atezolizumab (e.g., is decreased relative to a measurement taken before administration of the one or more dosing cycles of tiragolumab and atezolizumab). In some aspects, the SLD is decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or the SLD is decreased by about 100% (e.g., target lesions disappear) during or following administration of the one or more dosing cycles of tiragolumab and atezolizumab. In some aspects, the SLD is decreased by 1%-5%, 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100% during or following administration of the one or more dosing cycles of tiragolumab and atezolizumab. In some instances, the SLD is decreased relative to a measurement taken before administration of the tiragolumab and atezolizumab. In some instances, the clinical response may be a reduction in the SLD of one or more target lesions relative to the SLD in a comparator arm.

ii. Prior Therapy

In some embodiments, the subject has not been previously treated with an anti-cancer therapy (e.g., a cancer immunotherapy and/or a chemotherapeutic agent) for the cancer (e.g., esophageal cancer, e.g., metastatic esophageal cancer). In some embodiments, the subject has received prior treatment with an anti-cancer therapy (e.g., a cancer immunotherapy and/or a chemotherapeutic agent) for the cancer (e.g., esophageal cancer, e.g., metastatic esophageal cancer). In some instances, the subject has received at least one line of prior therapy. In some instances, the subject has received two or more prior anti-cancer therapies for the cancer (e.g., esophageal cancer). In some instances, the subject has received three or more prior anti-cancer therapies for the cancer (e.g., esophageal cancer). In some instances, the subject has received two lines of prior therapy. In some instances, the subject has received three lines of prior therapy. In some instances, the subject has received four lines of prior therapy. In some instances, the subject has received more than four lines of prior therapy. In some instances, the subject experienced disease progression during or following treatment with the prior anti-cancer therapy. In some instances, the prior therapy is chemotherapy, surgery, and/or radiotherapy.

In some instances, the subject has not received prior systemic therapy (e.g., prior systemic therapy with curative intent, e.g., chemotherapy) within at least the month prior to the administration with the PD-1 axis binding antagonist and the anti-TIGIT antagonist antibody (e.g., within the two months prior, three months prior, four months prior, six months prior, one year prior, two years prior, three years prior, four years prior, five years prior, or ten years prior to the administration with the PD-1 axis binding antagonist and the anti-TIGIT antagonist antibody). In some instances, the subject is chemotherapy naïve. In some instances, the subject has not received prior immunotherapy.

iii. Lack of Treatment-Related Adverse Events

In some embodiments, the subject does not experience a treatment-related adverse event (AE) (e.g., a Grade 1, Grade 2, Grade 3, or Grade 4 treatment-related adverse event) during or following the one or more dosing cycles of tiragolumab and atezolizumab. In some embodiments, the subject experiences a treatment-related Grade 1 or Grade 2 adverse event during or following the one or more dosing cycles of tiragolumab and atezolizumab. In some embodiments, the subject does not experience a treatment-related Grade 3 or Grade 4 adverse event during or following the one or more dosing cycles of tiragolumab and atezolizumab. Treatment-related adverse events include, e.g., tiragolumab-related adverse events and/or atezolizumab-related adverse events. Adverse events are graded according to the National Cancer Institute Common Terminology Criteria for Adverse Events (NCI CTCAE), Version 4.0.

Causality of adverse events (e.g., determination of whether an adverse event is treatment-related) may be based on the following guidance:
Temporal relationship of event onset to the initiation of study drug.
Course of the event, considering especially the effects of dose reduction, discontinuation of study drug, or reintroduction of study drug (as applicable).
Known association of the event with the study drug or with similar treatments.
Known association of the event with the disease under study.
Presence of risk factors in the subject or use of concomitant medications known to increase the occurrence of the event.
Presence of non-treatment-related factors that are known to be associated with the occurrence of the event.
   In general, an adverse event may be attributed to the study drug (e.g., tiragolumab and/or atezolizumab) if there is a plausible temporal relationship between the onset of the adverse event and administration of the study drug, and the adverse event cannot be readily explained by the subject's clinical state, intercurrent illness, or concomitant therapies; and/or the adverse event follows a known pattern of response to the study drug; and/or the adverse event abates or resolves upon discontinuation of the study drug or dose reduction and, if applicable, reappears upon re-challenge.

An adverse event may be identified as non-treatment-related if evidence exists that the adverse event has an etiology other than the study drug (e.g., preexisting medical condition, underlying disease, intercurrent illness, or concomitant medication); and/or the adverse event has no plausible temporal relationship to administration of the study drug (e.g., cancer diagnosed 2 days after first dose of study drug).

Several potential risks exist for tiragolumab based on the mechanism of action, known effect of similar checkpoint inhibitors, and nonclinical data. As an antagonist of TIGIT, tiragolumab is anticipated to enhance T-cell and NK cell proliferation, survival, and function. Therefore, tiragolumab may increase the risk of autoimmune inflammation (also described as immune-mediated adverse events). In addition, due to the intact Fc-effector function of tiragolumab, lymphopenia via antibody-dependent cellular cytotoxicity (ADCC) is a theoretical risk. Particular adverse events associated with itagolumab include infusion-related reactions (IRRs), immune-mediated adverse events, and lymphopenia.

Atezolizumab has been associated with risks such as the following: IRRs and immune-mediated hepatitis, pneumonitis, colitis, pancreatitis, diabetes mellitus, hypothyroidism, hyperthyroidism, adrenal insufficiency, hypophysitis, Guillain-Barré syndrome, myasthenic syndrome or myasthenia gravis, meningoencephalitis, myocarditis, myositis and nephritis. Immune-mediated adverse reactions may involve any organ system and may lead to hemophagocytic lymphohistiocytosis (HLH) and macrophage activation syndrome (MAS).

In any of the preceding examples, each dosing cycle may have any suitable length, e.g., about 7 days, about 14 days, about 21 days, about 28 days, or longer. In some instances, each dosing cycle is about 21 days. In some instances, tiragolumab is administered every three weeks (e.g., on Day 1 of each 21-day dosing cycle) and atezolizumab is administered every three weeks (e.g., on Day 1 of each 21-day dosing cycle).

The subject is preferably a human.

The present invention includes methods and uses involving administration of an effective amount of an anti-TIGIT antagonist antibody (e.g., an anti-TIGIT antagonist antibody as disclosed herein, e.g., tiragolumab) to a subject in need thereof every three weeks (e.g., on Day 1 of each 21-day dosing cycle). In some instances, the anti-TIGIT antagonist antibody (e.g., an anti-TIGIT antagonist antibody as disclosed herein, e.g., tiragolumab) is administered every three weeks (e.g., on Day 1 of each 21-day dosing cycle) and the PD-1 axis binding antagonist (e.g., an anti-PD-L1 antagonist antibody, e.g., atezolizumab, or an anti-PD-1 antagonist antibody, such as e.g., pembrolizumab) is administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle). In certain instances, the present invention includes methods and uses involving administration of an effective amount of an anti-TIGIT antagonist antibody (e.g., an anti-TIGIT antagonist antibody as disclosed herein, e.g., tiragolumab) to a subject in need thereof every three weeks (e.g., on Day 1 of each 21-day dosing cycle). In some instances, administration of the effective amount of the anti-TIGIT antagonist antibody (e.g., an anti-TIGIT antagonist antibody as disclosed herein, e.g., tiragolumab) results in a CR or a PR. In some instances, administration of the effective amount of the anti-TIGIT antagonist antibody (e.g., an anti-TIGIT antagonist antibody as disclosed herein, e.g., tiragolumab) results in an increase in PFS of the subject compared to a reference. In some instances, administration of the effective amount of the anti-TIGIT antagonist antibody (e.g., an anti-TIGIT antagonist antibody as disclosed herein, e.g., tiragolumab) extends OS of the subject. In some instances, the present invention includes a method of treating a subject having a cancer, the method comprising administering to the subject a dosing regimen comprising one or more dosing cycles of an anti-TIGIT antagonist antibody at a dose of about 500 mg to about 700 mg every three weeks, a PD-1 axis binding antagonist at a dose of about 900 mg to about 1500 mg every three weeks, a platinum-based chemotherapeutic agent every three weeks, and a non-platinum-based chemotherapeutic agent every three weeks. In some instances, the method comprises administering to the subject a dosing regimen comprising one or more dosing cycles of an anti-TIGIT antagonist antibody at a dose of 500 mg to 700 mg every three weeks, a PD-1 axis binding antagonist at a dose of 900 mg to 1500 mg every three weeks, a platinum-based chemotherapeutic agent every three weeks, and a non-platinum-based chemotherapeutic agent every three weeks.

In certain instances, the PD-1 axis binding antagonist and the anti-TIGIT antagonist antibody are administered without a chemotherapeutic agent (e.g., without any chemotherapeutic agent, e.g., the entire dosing regimen is devoid of administration of a chemotherapeutic agent to the subject).

iv. Responses to Treatment

In some embodiments of any of the methods described herein, the response to the treatment (e.g., atezolizumab and tiragolumab) of a subject or population of subjects having a cancer (e.g., an esophageal cancer (e.g., metastatic esophageal cancer)) can be characterized by one or more measures. In some embodiments, the treatment results in an increase in PFS or DOR in the subject. In some embodiments, the treatment results in an increase in the ORR or DCR in the population of subjects. In some embodiments, the treatment results in a CR or a PR in the subject.

In some instances, the treatment results in an increase in PFS of the subject, e.g., as compared to a PFS in a population (e.g., a comparator arm) treated with the PD-1 axis binding antagonist without the anti-TIGIT antagonist antibody or treated with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in PFS of the subject, e.g., as compared to a PFS in a population (e.g., a comparator arm) treated with the PD-1 axis binding antagonist without the anti-TIGIT antagonist antibody or treated with the anti-TIGIT antagonist antibody without the PD-1 axis binding antagonist.

In some instances, the treatment extends OS of the subject, e.g., as compared to an OS in a population (e.g., a comparator arm) treated with the PD-1 axis binding antagonist without the anti-TIGIT antagonist antibody or treated with the anti-TIGIT antagonist antibody without the PD-1 axis binding antagonist. For example, in embodiments in which no chemotherapeutic agent is administered (e.g., only an anti-TIGIT antagonist antibody (e.g., tiragolumab) in combination with a PD-1 axis binding antagonist (e.g., atezolizumab) is administered), the treatment may result in an increase in OS of the subject, e.g., as compared to an OS in a population (e.g., a comparator arm) treated with the PD-1 axis binding antagonist without the anti-TIGIT antagonist antibody or treated with the anti-TIGIT antagonist antibody without the PD-1 axis binding antagonist.

Progression-free survival of the subject can be measured according to RECIST v1.1 criteria, as described in Eisenhauer et al., Eur. J. Cancer. 2009, 45:228-47. In some embodiments, PFS is measured as the period of time from the start of treatment to the first occurrence of disease progression as determined by RECIST v1.1 criteria. In some embodiments, PFS is measured as the time from the start of treatment to the time of death.

In some embodiments, a treatment described herein results in PFS of the subject of at least about 1 month (e.g., 1 month, 1.5 months, 2 months, 2.5 months, 3.0 months, 3.5 months, 4.0 months, 4.5 months, 5.0 months, 5.5 months, 6.0 months, 6.5 months, 7.0 months, 7.5 months, 8.0 months, 8.5 months, 9.0 months, 9.5 months, 10 months, 10.5 months, 11 months, 11.5 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months).

In some embodiments, treatment results in a median PFS of the population of subjects of about 1.2 months to about 5.6 months (e.g., 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.7, 4.8, 4.9, 5.0, 5.1, 5.2, 5.3, 5.4, 5.5, or 5.6 months, e.g., 1.2-1.4, 1.4-1.6, 1.6-1.8, 1.8-2.0, 2.0-2.2, 2.2-2.4, 2.4-2.6, 2.6-2.8, 2.8-3.0, 3.0-3.2, 3.2-3.4, 3.4-3.6, 3.6-3.8, 3.8-4.0, 4.0-4.2, 4.2-4.4, 4.4-4.6, 4.6-4.8, 4.8-5.0, 5.0-5.2, 5.2-5.4, or 5.4-5.6 months).

In some embodiments, a treatment described herein results in a DOR of the subject of at least about 7 months (e.g., 7 months, 7.5 months, 8.0 months, 8.5 months, 9.0 months, 9.5 months, 10 months, 10.5 months, 11 months, 11.5 months, 12 months, 12.5 months, 13 months, 13.5 months, 14 months, 14.5 months, 15 months, 15.5 months, 16 months, 16.5 months, 17 months, 17.5 months, 18 months, 18.5 months, 19 months, 19.5 months, 20 months, 20.5 months, 21 months, 21.5 months, 22 months, 23 months, 24 months, 25 months, 26 months, 27 months, 28 months, 29 months, 30 months, 31 months, 32 months, 33 months, 34 months, 35 months, or 36 months).

In some embodiments, treatment results in a median DOR of the population of subjects of about 7 months to about 25 months (e.g., 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 23.0, 24.0, or 25.0 months, e.g., 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, 16.0-16.5, 16.5-17.0, 17.0-17.5, 17.5-18.0, 18.0-18.5, 18.5-19.0, 19.0-19.5, 19.5-20.0, 20.0-20.5, 20.5-21.0, 21.0-21.5, 21.5-22.0, 22.0-22.5, 22.5-23.0, 23.0-23.5, 23.5-24.0, 24.0-24.5, or 24.5-25.0 months).

In some embodiments of any of the methods described herein, a population of subjects' response to the treatment (e.g., atezolizumab and tiragolumab) can be characterized by one or more measures. In some embodiments, the treatment of the population of subjects results in an increased ORR or DCR.

In some instances, the treatment results in an increased ORR in the population of subjects, e.g., as compared to an ORR in a population (e.g., a comparator arm) treated with the PD-1 axis binding antagonist without the anti-TIGIT antagonist antibody or treated with the anti-TIGIT antagonist antibody without the PD-1 axis binding antagonist. For example, the treatment may result in an increase in ORR of the population of subjects, e.g., as compared to an ORR in a population (e.g., a comparator arm) treated with the PD-1 axis binding antagonist without the anti-TIGIT antagonist antibody or treated with the anti-TIGIT antagonist antibody without the PD-1 axis binding antagonist.

In some instances, the treatment results in an increased DCR in the population of subjects, e.g., as compared to a DCR in a population (e.g., a comparator arm) treated with the PD-1 axis binding antagonist without the anti-TIGIT antagonist antibody or treated with the anti-TIGIT antagonist antibody without the PD-1 axis binding antagonist. For example, the treatment may result in an increase in DCR of the population of subjects, e.g., as compared to a DCR in a population (e.g., a comparator arm) treated with the PD-1 axis binding antagonist without the anti-TIGIT antagonist antibody or treated with the anti-TIGIT antagonist antibody without the PD-1 axis binding antagonist.

In some embodiments, a population of subjects treated as described herein has an ORR of at least about 28% (e.g., 28.0%, 28.1%, 28.2%, 28.3%, 28.4%, 28.5%, 28.6%, 28.7%, 28.8%, 28.9%, 29.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35%, 35.5%, 40.0%, 40.5%, 41.0%, 41.5%, 42.0%, 42.5%, 43.0%, 43.5%, 44.0%, 45.0%, 46.0%, 47.0%, 48.0%, 49.0%, 50.0%, or 55.0%). In some embodiments, a population of subjects treated as described herein has an ORR of between about 25% to about 55% (e.g., 25.0% to 25.5%, 25.5% to 26.0%, 26.0% to 26.5%, 26.5% to 27.0%, 27.0% to 27.5%, 27.5% to 28.0%, 28.0% to 28.5%, 28.5% to 29.0%, 29.0% to 29.5%, 29.5% to 30.0%, 30.0% to 30.5%, 30.5% to 31.0%, 31.0% to 31.5%, 31.5% to 32.0%, 32.0% to 32.5%, 32.5% to 33.0%, 33.0% to 33.5%, 33.5% to 34.0%, 34.0% to 34.5%, 34.5% to 35.0%, 35.0% to 36.0%, 36.0% to 37.0%, 37.0% to 38.0%, 38.0% to 39.0%, 39.0% to 40.0%, 40.0% to 41.0%, 41.0% to 42.0%, 42.0% to 43.0%, 43.0% to 44.0%, 44.0% to 45.0%, 45.0% to 46.0%, 46.0% to 47.0%, 47.0% to 48.0%, 48.0% to 49.0%, 49.0% to 50.0%, 50.0% to 51.0%, 51.0% to 52.0%, 52.0% to 53.0%, 53.0% to 54.0%, or 54.0% to 55.0%).

In some embodiments, a population of subjects treated as described herein has a DCR of at least about 50% (e.g., 50.0%, 50.1%, 50.2%, 50.3%, 50.4%, 50.5%, 50.6%, 50.7%, 50.8%, 50.9%, 51.0%, 51.1%, 51.2%, 51.3%, 51.4%, 51.5%, 51.6%, 51.7%, 51.8%, 51.9%, 52.0%, 52.1%, 52.2%, 52.3%, 52.4%, 52.5%, 52.6%, 52.7%, 52.8%, 52.9%, 53.0%, 53.1%, 53.2%, 53.3%, 53.4%, 53.5%, 53.6%, 53.7%, 53.8%, 53.9%, 54.0%, 54.1%, 54.2%, 54.3%, 54.4%, 54.5%, 54.6%, 54.7%, 54.8%, 54.9%, 55.0%, 55.1%, 55.2%, 55.3%, 55.4%, 55.5%, 55.6%, 55.7%, 55.8%, 55.9%, 56.0%, 56.1%, 56.2%, 56.3%, 56.4%, 56.5%, 56.6%, 56.7%, 56.8%, 56.9%, 57%, 57.5%, 58.0%, 58.5%, 59.0%, 59.5%, 60.0%, 60.5%, 61.0%, 61.5%, 62.0%, 63.0%, 64.0%, 65.0%, 66.0%, 67.0%, 68.0%, or 73.0%). In some embodiments, a population of subjects treated as described herein has a DCR of between about 45% to about 55% (e.g., 45.0% to 45.2%, 45.2% to 45.4%, 45.4% to 45.6%, 45.6% to 45.8%, 45.8% to 46.0%, 46.0% to 46.2%, 46.2% to 46.4%, 46.4% to 46.6%, 46.6% to 46.8%, 46.8% to 47.0%, 47.0% to 47.2%, 47.2% to 47.4%, 47.4% to 47.6%, 47.6% to 47.8%, 47.8% to 48.0%, 48.0% to 48.2%, 48.2% to 48.4%, 48.4% to 48.6%, 48.6% to 48.8%, 48.8% to 49.0%, 49.0% to 49.2%, 49.2% to 49.4%, 49.4% to 49.6%, 49.6% to 49.8%, 49.8% to 50.0%, 50.0% to 50.2%, 50.2% to 50.4%, 50.4% to 50.6%, 50.6% to 50.8%, 50.8% to 51.0%, 51.0% to 51.2%, 51.2% to 51.4%, 51.4% to 51.6%, 51.6% to 51.8%, 51.8% to 52.0%, 52.0% to 52.2%, 52.2% to 52.4%, 52.4% to 52.6%, 52.6% to 52.8%, 52.8% to 53.0%, 53.0% to 53.2%, 53.2% to 53.4%, 53.4% to 53.6%, 53.6% to 53.8%, 53.8% to 54.0%, 54.0% to 54.2%, 54.2% to 54.4%, 54.4% to 54.6%, 54.6% to 54.8%, or 54.8% to 55.0%).

### III. Therapeutic Methods and Compositions for Melanoma

### A. Methods Comprising an Anti-TIGIT Antagonist Antibody and a Bispecific Antibody Targeting PD-1 and LAG3

In one aspect, the disclosure provides a method for treating a subject having a melanoma, the method comprising administering to the subject an anti-TIGIT antagonist antibody and a bispecific antibody targeting PD-1 and LAG3 comprising a first antigen-binding domain that specifically binds to programmed cell death protein 1 (PD-1) and a second antigen-binding domain that specifically binds to lymphocyte activation gene 3 (LAG3). In some embodiments, the anti-TIGIT antagonist antibody and the bispecific antibody are administered to the subject in a dosing regimen that comprises one or more dosing cycles.

In some aspects, the method comprises administering to the subject (a) the anti-TIGIT antagonist antibody at a fixed dose of about 600 mg (e.g., a fixed dose of 600 mg) every three weeks; and (b) the bispecific antibody at a fixed dose of about 2100 mg (e.g., a fixed dose of 2100 mg) every three weeks.

In some aspects, the method comprises administering to the subject (a) the anti-TIGIT antagonist antibody at a fixed dose of about 600 mg (e.g., a fixed dose of 600 mg) every three weeks; and (b) the bispecific antibody at a fixed dose of about 600 mg (e.g., a fixed dose of 600 mg) every three weeks.

In some aspects, the length of each of the one or more dosing cycles is 21 days. In some aspects, the method comprises administering to the subject the anti-TIGIT antagonist antibody and the bispecific antibody on about Day 1 (e.g., on Day 1) of each of the one or more dosing cycles.

In some aspects, the method comprises administering to the subject the bispecific antibody before the anti-TIGIT antagonist antibody. In other aspects, the method comprises administering to the subject the anti-TIGIT antagonist antibody before the bispecific antibody.

In some aspects, the method comprises administering to the subject the bispecific antibody and the anti-TIGIT antagonist antibody intravenously.

i. Neoadjuvant Therapy

In some aspects, the one or more dosing cycles are administered as a neoadjuvant therapy.

In some aspects, the anti-TIGIT antagonist antibody and the bispecific antibody targeting PD-1 and LAG3 are administered as a neoadjuvant therapy.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

ii. Treatment of Stage IV Melanoma

In some aspects, the melanoma is a Stage IV melanoma.

In some aspects, (a) the subject has received no more than two prior lines of systemic treatment; or (b) the melanoma is a BRAF-mutant melanoma and the subject has received no more than three prior lines of systemic treatment.

In some aspects, the treating results in an increase in overall response rate (ORR) as compared to a reference ORR. In some aspects, the reference ORR is an ORR of a population of subjects who have received (a) a treatment comprising a bispecific antibody targeting PD-1 and LAG3 and not comprising an anti-TIGIT antagonist antibody; and/or (b) a treatment comprising an anti-TIGIT antagonist antibody and not comprising a bispecific antibody targeting PD-1 and LAG3.

In some aspects, the treating results in an increase in progression-free survival (PFS) as compared to a reference PFS; an increase in duration of response (DOR) as compared to a reference DOR; an increase in OS as compared to a reference OS; an increase in disease control rate (DCR, e.g., stable disease for 12 or more weeks, a complete response (CR), or a partial response (PR)) as compared to a reference DCR. In some aspects, the reference PFS, OS, DOR, or DCR is one of a population of subjects who have received a control therapy. In some aspects, the control therapy is a therapy comprising an anti-TIGIT antagonist antibody and not comprising a bispecific antibody targeting PD-1 and LAG3; a therapy comprising a bispecific antibody targeting PD-1 and LAG3 and not comprising an anti-TIGIT antagonist antibody; or a therapy comprising ipilimumab and nivolumab.

In some aspects, the subject is a human.

### B. Methods Comprising a Bispecific Antibody Targeting PD-1 and LAG3

In another aspect, the disclosure features a method for treating a subject having a melanoma, the method comprising administering to the subject a bispecific antibody targeting PD-1 and LAG3 comprising a first antigen-binding domain that specifically binds to PD-1 and a second antigen-binding domain that specifically binds to LAG3. In some embodiments, the bispecific antibody is administered to the subject in a dosing regimen that comprises one or more dosing cycles. In some embodiments, the one or more dosing cycles are administered as a neoadjuvant therapy.

In some aspects, the method comprises administering to the subject the bispecific antibody at a fixed dose about 2100 mg (e.g., a fixed dose of 2100 mg) every three weeks.

In some aspects, the method comprises administering to the subject the bispecific antibody at a fixed dose about 600 mg (e.g., a fixed dose of 600 mg) every three weeks.

In some aspects, the length of each of the one or more dosing cycles is 21 days. In some aspects, the method comprises administering to the subject the bispecific antibody on about Day 1 (e.g., on Day 1) of each of the one or more dosing cycles.

In some aspects, the method comprises administering to the subject the bispecific antibody intravenously.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising ipilimumab and nivolumab.

In some aspects, the subject is a human.

### C. Methods Comprising an Anti-TIGIT Antagonist Antibody and a PD-1 Axis Binding Antagonist

In another aspect, the disclosure features a method for treating a subject having a melanoma, the method comprising administering to the subject one or more dosing cycles of an anti-TIGIT antagonist antibody and a PD-1 axis binding antagonist, wherein the one or more dosing cycles are administered as a neoadjuvant therapy. In another aspect, the disclosure features a method for treating a subject having a melanoma, the method comprising administering to the subject an anti-TIGIT antagonist antibody and a PD-1 axis binding antagonist, wherein the anti-TIGIT antagonist antibody and the PD-1 axis binding antagonist are administered as a neoadjuvant therapy.

In some aspects, the method comprises administering to the subject (a) the anti-TIGIT antagonist antibody at a fixed dose of about 600 mg (e.g., a fixed dose of 600 mg) every three weeks; and (b) the PD-1 axis binding antagonist at a fixed dose of about 1200 mg (e.g., a fixed dose of 1200 mg) every three weeks.

In some aspects, the length of each of the one or more dosing cycles is 21 days.

In some aspects, the method comprises administering to the subject the anti-TIGIT antagonist antibody and the PD-1 axis binding antagonist on about Day 1 (e.g., on Day 1) each of the one or more dosing cycles.

In some aspects, the method comprises administering to the subject the PD-1 axis binding antagonist before the anti-TIGIT antagonist antibody. In other aspects, the method comprises administering to the subject the anti-TIGIT antagonist antibody before the PD-1 axis binding antagonist.

In some aspects, the method comprises administering to the subject the PD-1 axis binding antagonist and the anti-TIGIT antagonist antibody intravenously.

In some aspects, the melanoma is a Stage III melanoma with measurable lymph node metastases.

In some aspects, the subject has not had in-transit metastases within six months prior to the initiation of treatment.

In some aspects, the subject has not previously been treated with a cancer immunotherapy.

In some aspects, the melanoma is not a mucosal melanoma or a uveal melanoma.

In some aspects, a first dosing cycle is initiated prior to a surgery.

In some aspects, at least one dosing cycle or (e.g., one, two, three, four, or more than four dosing cycles) or at least two dosing cycles (e.g., two, three, four, or more than four dosing cycles) are completed prior to the surgery. In some aspects, two dosing cycles are completed prior to the surgery.

In some aspects, the surgery is performed within about one week after the last dosing cycle.

In some aspects, the surgery is a completion lymph node dissection (CLND).

In some aspects, the treating results in an increase in pathologic response rate (pRR) as compared to a reference pRR. In some aspects, the reference pRR is a pRR of a population of subjects who have received a control therapy. In some aspects, the control therapy is a therapy comprising an anti-TIGIT antagonist antibody and not comprising a PD-1 axis binding antagonist; a therapy comprising a PD-1 axis binding antagonist and not comprising an anti-TIGIT antagonist antibody; or a therapy comprising ipilimumab and nivolumab.

In some aspects, the treating results in an increase in event-free survival (EFS) as compared to a reference EFS; an increase in recurrence-free survival (RFS) as compared to a reference RFS; an increase in overall survival (OS) as compared to a reference OS; and/or an increase in overall response rate (ORR) as compared to a reference ORR. In some aspects, the reference EFS, RFS, OS, or ORR is one of a population of subjects who have received a control therapy. In some aspects, the control therapy is a therapy comprising an anti-TIGIT antagonist antibody and not comprising a PD-1 axis binding antagonist; a therapy comprising a PD-1 axis binding antagonist and not comprising an anti-TIGIT antagonist antibody; or a therapy comprising ipilimumab and nivolumab.

In some aspects, the subject is a human.

### D. Agents for Use in Methods of Treating Melanoma

i. Bispecific Antibodies Targeting PD-1 and LAG3

Further examples of bispecific antibodies targeting PD-1 and LAG3, and dosing regimens for the same, are provided in Section XI, below.

ii. Anti-TIGIT Antagonist Antibodies

Exemplary anti-TIGIT antagonist antibodies, and dosing regimens for the same, are provided in Sections VI and IX, below.

iii. PD-1 Axis Binding Antagonists

Exemplary PD-1 axis binding antagonists, and dosing regimens for the same, are provided in Sections VI and X, below.

### IV. Therapeutic Methods and Compositions for CD20-Positive Cell Proliferative Disorders

Provided herein are methods of treating a subject having a CD20-positive cell proliferative disorder (e.g., a B cell proliferative disorder, e.g., an NHL (e.g., an aggressive NHL or a relapsed or refractory (R/R) NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)) comprising administering to the subject tiragolumab and mosunetuzumab. In some embodiments, subject has relapsed after, or is refractory to, at least two prior therapies.

### A. Therapeutic Methods for Dosing of Tiragolumab and Mosunetuzumab

The invention provides methods for treating a subject having a relapsed or refractory non-Hodgkin's lymphoma (NHL) (e.g., a B cell proliferative disorder, e.g., an NHL (e.g., an aggressive NHL or an R/R NHL; e.g., an R/R DLBCL, an R/R FL (e.g., an R/R Grade 3b FL), or an R/R high grade B cell lymphoma (HGBL) or an R/R transformed FL (trFL))) that includes administering to the subject tiragolumab and mosunetuzumab.

In some aspects, the R/R NHL is a R/R follicular lymphoma (FL), R/R diffuse large B cell lymphoma (DLBCL), or R/R high grade B cell lymphoma (HGBL).

In some aspects, the R/R FL is a R/R transformed FL (trFL) or a R/R Grade 3b FL.

In some aspects, the subject has relapsed after, or is refractory to, at least two (e.g., at least three, at least four, at least five, at least six, at least seven, at least eight, at least nine, or at least ten; e.g., two, three, four, five, six, seven, eight, nine, ten, or more) prior therapies (e.g., prior systemic therapies).

In some aspects, the subject has relapsed after, or is refractory to, at least one (e.g., at least two, at least three, at least four, at least five, at least six, at least seven, at least eight, at least nine, or at least ten; e.g., one, two, three, four, five, six, seven, eight, nine, ten, or more) prior therapy comprising an anti-CD20 monoclonal antibody.

In some aspects, the anti-CD20 monoclonal antibody is rituximab or obinutuzumab. In some aspects, the anti-CD20 monoclonal antibody is rituximab. In some aspects, the anti-CD20 monoclonal antibody is obinutuzumab.

In some aspects, the subject has relapsed after, or is refractory to, at least one (e.g., at least two, at least three, at least four, at least five, at least six, at least seven, at least eight, at least nine, or at least ten; e.g., one, two, three, four, five, six, seven, eight, nine, ten, or more) prior therapy comprising anthracycline.

In some aspects, the anthracycline is daunomycin or doxorubicin. In some aspects, the anthracycline is daunomycin. In some aspects, the anthracycline is doxorubicin.

In some aspects, the subject has relapsed after, or is refractory to, at least one (e.g., at least two, at least three, at least four, at least five, at least six, at least seven, at least eight, at least nine, or at least ten; e.g., one, two, three, four, five, six, seven, eight, nine, ten, or more) prior therapy comprising an alkylating agent.

In some aspects, the alkylating agent is bendamustine, carboplatin, cisplatin, or cyclophosphamide. In some aspects, the alkylating agent is bendamustine, carboplatin, cisplatin, or cyclophosphamide. In some aspects, the alkylating agent is bendamustine. In some aspects, the alkylating agent is carboplatin. In some aspects, the alkylating agent is cisplatin. In some aspects, the alkylating agent is cyclophosphamide.

In some aspects, the subject is ineligible for autologous stem cell therapy (ASCT) or chimeric antigen receptor (CAR) T-cell therapy. In some aspects, the subject is ineligible for autologous stem cell therapy (ASCT). In some aspects, the subject is ineligible for chimeric antigen receptor (CAR) T-cell therapy.

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is between 1 to 10 mg (e.g., between 1 to 9 mg, between 1 to 8 mg, between 1 to 7 mg, between 1 to 6 mg, between 2 to 9 mg, between 3 to 9 mg, between 4 to 9 mg, between 3 to 7 mg, between 4 to 6 mg, between 4.5 to 5.5 mg, between 1 to 5 mg, between 5 to 10 mg, between 2.5 to 5 mg, or between 5 to 7.5 mg; e.g., about 1 mg, about 2 mg, about 3 mg, about 3.5 about 4 mg, about 4.5 mg, about 4.8 mg, about 4.9 mg, about 5 mg, about 5.1 mg, about 5.2 mg, about 5.3 mg, about 5.5 mg, about 6 mg, about 6.5 mg, about 7 mg, about 8 mg, about 9 mg, or about 10 mg), the C1D2 of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg), and the C1D3 of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg); and (b) the second dosing cycle comprises a single dose (C2D1) of mosunetuzumab, wherein the C2D1 of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg).

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is about 5 mg, the C1D2 of mosunetuzumab is about 45 mg, and the C1D3 of mosunetuzumab is about 45 mg; and (b) the second dosing cycle comprises a single dose (C2D1) of mosunetuzumab, wherein the C2D1 of mosunetuzumab is about 45 mg. In some aspects, the C1D1 is of mosunetuzumab 5 mg, the C1D2 of mosunetuzumab is 45 mg, the C1D3 of mosunetuzumab is 45 mg, and the C2D1 of mosunetuzumab is 45 mg.

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is between 1 to 10 mg (e.g., between 1 to 9 mg, between 1 to 8 mg, between 1 to 7 mg, between 1 to 6 mg, between 2 to 9 mg, between 3 to 9 mg, between 4 to 9 mg, between 3 to 7 mg, between 4 to 6 mg, between 4.5 to 5.5 mg, between 1 to 5 mg, between 5 to 10 mg, between 2.5 to 5 mg, or between 5 to 7.5 mg; e.g., about 1 mg, about 2 mg, about 3 mg, about 3.5 about 4 mg, about 4.5 mg, about 4.8 mg, about 4.9 mg, about 5 mg, about 5.1 mg, about 5.2 mg, about 5.3 mg, about 5.5 mg, about 6 mg, about 6.5 mg, about 7 mg, about 8 mg, about 9 mg, or about 10 mg), the C1D2 of mosunetuzumab is between 10 and 20 mg (e.g., between 11 to 19 mg, between 11 to 18 mg, between 11 to 17 mg, between 11 to 16 mg, between 12 to 19 mg, between 13 to 19 mg, between 14 to 19 mg, between 13 to 17 mg, between 14 to 16 mg, between 14.5 to 15.5 mg, between 11 to 15 mg, between 15 to 20 mg, between 12.5 to 15 mg, or between 15 to 17.5 mg; e.g., about 11 mg, about 12 mg, about 13 mg, about 13.5 about 14 mg, about 14.5 mg, about 14.8 mg, about 14.9 mg, about 15 mg, about 15.1 mg, about 15.2 mg, about 15.3 mg, about 15.5 mg, about 16 mg, about 16.5 mg, about 17 mg, about 18 mg, about 19 mg, or about 20 mg), and the C1D3 of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg); and (b) the second dosing cycle comprises a single dose (C2D1) of mosunetuzumab, wherein the C2D1 of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg).

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is about 5 mg, the C1D2 of mosunetuzumab is about 15 mg, and the C1D3 of mosunetuzumab is about 45 mg; and (b) the second dosing cycle comprises a single dose (C2D1) of mosunetuzumab, wherein the C2D1 of mosunetuzumab is about 45 mg. In some aspects, the C1D1 is of mosunetuzumab 5 mg, the C1D2 of mosunetuzumab is 15 mg, the C1D3 of mosunetuzumab is 45 mg, and the C2D1 of mosunetuzumab is 45 mg.

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is between 0.5 mg and 2 mg (e.g., between 0.5 mg and 1 mg, between 1 mg and 2 mg, between 1 mg and 1.5 mg, between 0.75 mg and 1.25 mg, between 0.8 mg and 1.2 mg, or between 0.9 mg and 2.1 mg; e.g, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg), the C1D2 of mosunetuzumab is about 2 mg, and the C1D3 of mosunetuzumab is between 25 mg and 35 mg (e.g., between 26 mg and 35 mg, between 27 mg and 35 mg, between 28 mg and 35 mg, between 29 mg and 35 mg, between 26 mg and 34 mg, between 26 mg and 33 mg, between 26 mg and 32 mg, between 26 mg and 31 mg, between 27.5 mg and 32.5 mg, between 28 mg and 32 mg, between 29 mg and 31 mg, between 25 mg and 30 mg, or between 30 mg and 35 mg; e.g., about 25 mg, about 26 mg, about 27 mg, about 28 mg, about 28.5 mg, about 29 mg, about 29.5 mg, about 29.8 mg, about 29.9 mg, about 30 mg, about 30.1 mg, about 30.2 mg, about 30.5 mg, about 31 mg, about 31.5 mg, about 32 mg, about 33 mg, about 34 mg, or about 35 mg); and (b) the second dosing cycle comprises a single dose (C2D1) of mosunetuzumab, wherein the C2D1 of mosunetuzumab is between 25 mg and 35 mg (e.g., between 26 mg and 35 mg, between 27 mg and 35 mg, between 28 mg and 35 mg, between 29 mg and 35 mg, between 26 mg and 34 mg, between 26 mg and 33 mg, between 26 mg and 32 mg, between 26 mg and 31 mg, between 27.5 mg and 32.5 mg, between 28 mg and 32 mg, between 29 mg and 31 mg, between 25 mg and 30 mg, or between 30 mg and 35 mg; e.g., about 25 mg, about 26 mg, about 27 mg, about 28 mg, about 28.5 mg, about 29 mg, about 29.5 mg, about 29.8 mg, about 29.9 mg, about 30 mg, about 30.1 mg, about 30.2 mg, about 30.5 mg, about 31 mg, about 31.5 mg, about 32 mg, about 33 mg, about 34 mg, or about 35 mg).

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is about 1 mg, the C1D2 of mosunetuzumab is about 2 mg, and the C1D3 of mosunetuzumab is about 30 mg; and (b) the second dosing cycle comprises a single dose (C2D1) of mosunetuzumab, wherein the C2D1 of mosunetuzumab is about 30 mg. In some aspects, the C1D1 is of mosunetuzumab 1 mg, the C1D2 of mosunetuzumab is 2 mg, the C1D3 of mosunetuzumab is 30 mg, and the C2D1 of mosunetuzumab is 30 mg.

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is between 0.5 mg and 2 mg (e.g., between 0.5 mg and 1 mg, between 1 mg and 2 mg, between 1 mg and 1.5 mg, between 0.75 mg and 1.25 mg, between 0.8 mg and 1.2 mg, or between 0.9 mg and 2.1 mg; e.g, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg), the C1D2 of mosunetuzumab is about 2 mg, and the C1D3 of mosunetuzumab is between 55 mg and 65 mg (e.g., between 56 mg and 65 mg, between 57 mg and 65 mg, between 58 mg and 65 mg, between 59 mg and 65 mg, between 56 mg and 64 mg, between 56 mg and 63 mg, between 56 mg and 62 mg, between 56 mg and 61 mg, between 57.5 mg and 62.5 mg, between 58 mg and 62 mg, between 59 mg and 61 mg, between 55 mg and 60 mg, or between 60 mg and 65 mg; e.g., about 55 mg, about 56 mg, about 57 mg, about 58 mg, about 58.5 mg, about 59 mg, about 59.5 mg, about 59.8 mg, about 59.9 mg, about 60 mg, about 60.1 mg, about 60.2 mg, about 60.5 mg, about 61 mg, about 61.5 mg, about 62 mg, about 63 mg, about 64 mg, or about 65 mg); and (b) the second dosing cycle comprises a single dose (C2D1) of mosunetuzumab, wherein the C2D1 of mosunetuzumab is about 60 mg (e.g., between 56 mg and 65 mg, between 57 mg and 65 mg, between 58 mg and 65 mg, between 59 mg and 65 mg, between 56 mg and 64 mg, between 56 mg and 63 mg, between 56 mg and 62 mg, between 56 mg and 61 mg, between 57.5 mg and 62.5 mg, between 58 mg and 62 mg, between 59 mg and 61 mg, between 55 mg and 60 mg, or between 60 mg and 65 mg; e.g., about 55 mg, about 56 mg, about 57 mg, about 58 mg, about 58.5 mg, about 59 mg, about 59.5 mg, about 59.8 mg, about 59.9 mg, about 60 mg, about 60.1 mg, about 60.2 mg, about 60.5 mg, about 61 mg, about 61.5 mg, about 62 mg, about 63 mg, about 64 mg, or about 65 mg).

In some aspects, the tiragolumab and mosunetuzumab are administered to the subject in a dosing regimen that comprises at least a first dosing cycle and a second dosing cycle, wherein: (a) the first dosing cycle comprises a first dose (C1D1) of mosunetuzumab, a second dose (C1D2) of mosunetuzumab, and a third dose (C1D3) of mosunetuzumab, wherein the C1D1 of mosunetuzumab is about 1 mg, the C1D2 of mosunetuzumab is about 2 mg, and the C1D3 of mosunetuzumab is about 60 mg; and (b) the second dosing cycle comprises a single dose (C2D1) of mosunetuzumab, wherein the C2D1 of mosunetuzumab is about 60 mg. In some aspects, the C1D1 is of mosunetuzumab 1 mg, the C1D2 of mosunetuzumab is 2 mg, the C1D3 of mosunetuzumab is 60 mg, and the C2D1 of mosunetuzumab is 60 mg.

In some aspects, the first and second dosing cycles are 21-day dosing cycles (±1 day). In some aspects, the first and second dosing cycles are 28-day dosing cycles (±1 day). In some aspects, the first dosing cycle is a 21-day dosing cycle (±1 day) and the second dosing cycle is a 28-day dosing cycle (±1 day).

In some aspects, the C1D1, the C1D2, and the C1D3 of mosunetuzumab are administered on or about Days 1, 8 (±1 day), and 15 (±1 day), respectively, of the first dosing cycle. In some aspects, the C1D1, the C1D2, and the C1D3 of mosunetuzumab are administered on Days 1, 8, and 15, respectively, of the first dosing cycle.

In some aspects, the C2D1 of mosunetuzumab is administered on Day 1 of the second dosing cycle.

In some aspects, the first dosing cycle comprises a single dose (C1D1) of tiragolumab.

In some aspects, the C1D1 of tiragolumab is administered on Day 1 of the first dosing cycle.

In some aspects, the C1D1 of tiragolumab is between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg). In some aspects, the C1D1 of tiragolumab is about 600 mg. In some aspects, the C1D1 of tiragolumab is 600 mg.

In some aspects, the C1D1 of tiragolumab is administered after administration of the C1D1 of mosunetuzumab. In some aspects, the C1D1 of tiragolumab is administered simultaneously with the administration of the C1D1 of mosunetuzumab.

In some aspects, tiragolumab is not administered to the subject during the first dosing cycle

In some aspects, the second dosing cycle comprises a single dose (C2D1) of tiragolumab.

In some aspects, the C2D1 of tiragolumab is administered on Day 1 of the second dosing cycle.

In some aspects, the C2D1 of tiragolumab is between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg). In some aspects, the C2D1 of tiragolumab is about 600 mg. In some aspects, the C2D1 of tiragolumab is 600 mg.

In some aspects, the C2D1 of tiragolumab is administered after administration of the C2D1 of mosunetuzumab. In some aspects, the C2D1 of tiragolumab is administered simultaneously with the administration of the C2D1 of mosunetuzumab.

In some aspects, the dosing regimen additionally comprises administering to the subject atezolizumab.

In some aspects, the second dosing cycle comprises a single dose (C2D1) of atezolizumab.

In some aspects, the C2D1 of atezolizumab is administered on Day 1 of the second dosing cycle.

In some aspects, the C2D1 of atezolizumab is between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200). In some aspects, the C2D1 of atezolizumab is about 1200 mg. In some aspects, the C2D1 of atezolizumab is 1200 mg.

In some aspects, the C2D1 of atezolizumab is administered after administration of the C2D1 of tiragolumab.

In some aspects, atezolizumab is not administered to the subject during the first dosing cycle.

In some aspects, the dosing regimen further comprises one or more (e.g., one, two, three, four, five, six, seven, eight, nine, ten, eleven, twelve, thirteen, fourteen, fifteen, or more) additional dosing cycles.

In some aspects, the dosing regimen further comprises six to fifteen (e.g., six, seven, eight nine, ten, eleven, twelve, thirteen, fourteen, or fifteen) additional dosing cycles.

In some aspects, the dosing regimen further comprises six additional dosing cycles.

In some aspects, the dosing regimen further comprises fifteen additional dosing cycles.

In some aspects, each additional dosing cycle is a 21-day dosing cycle (±1 day). In some aspects, each additional dosing cycle is a 28-day dosing cycle (±1 day).

In some aspects, each additional dosing cycle comprises administration of an additional dose of mosunetuzumab.

In some aspects, each additional dose of mosunetuzumab is between 40 and 50 mg (e.g., between 41 to 49 mg, between 41 to 48 mg, between 41 to 47 mg, between 41 to 46 mg, between 42 to 49 mg, between 43 to 49 mg, between 44 to 49 mg, between 43 to 47 mg, between 44 to 46 mg, between 44.5 to 45.5 mg, between 41 to 45 mg, between 45 to 50 mg, between 42.5 to 45 mg, or between 45 to 47.5 mg; e.g., about 41 mg, about 42 mg, about 43 mg, about 43.5 about 44 mg, about 44.5 mg, about 44.8 mg, about 44.9 mg, about 45 mg, about 45.1 mg, about 45.2 mg, about 45.3 mg, about 45.5 mg, about 46 mg, about 46.5 mg, about 47 mg, about 48 mg, about 49 mg, or about 50 mg). In some aspects, each additional dose of mosunetuzumab is about 45 mg. In some aspects, each additional dose of mosunetuzumab is 45 mg.

In some aspects, each additional dose of mosunetuzumab is between 25 mg and 35 mg (e.g., between 26 mg and 35 mg, between 27 mg and 35 mg, between 28 mg and 35 mg, between 29 mg and 35 mg, between 26 mg and 34 mg, between 26 mg and 33 mg, between 26 mg and 32 mg, between 26 mg and 31 mg, between 27.5 mg and 32.5 mg, between 28 mg and 32 mg, between 29 mg and 31 mg, between 25 mg and 30 mg, or between 30 mg and 35 mg; e.g., about 25 mg, about 26 mg, about 27 mg, about 28 mg, about 28.5 mg, about 29 mg, about 29.5 mg, about 29.8 mg, about 29.9 mg, about 30 mg, about 30.1 mg, about 30.2 mg, about 30.5 mg, about 31 mg, about 31.5 mg, about 32 mg, about 33 mg, about 34 mg, or about 35 mg). In some aspects, each additional dose of mosunetuzumab is about 30 mg. In some aspects, each additional dose of mosunetuzumab is 30 mg.

In some aspects, each additional dose of mosunetuzumab is administered on Day 1 of each respective additional dosing cycle.

In some aspects, each additional dosing cycle comprises administration of an additional dose of tiragolumab.

In some aspects, each additional dose of tiragolumab is about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg). In some aspects, each additional dose of tiragolumab is about 600 mg. In some aspects, each additional dose of tiragolumab is 600 mg.

In some aspects, each additional dose of tiragolumab is administered on Day 1 of each respective additional dosing cycle.

In some aspects, each additional dose of tiragolumab is administered after administration of each additional dose of mosunetuzumab. In some aspects, each additional dose of tiragolumab is administered simultaneously with the administration of each additional dose of mosunetuzumab.

In some aspects, each additional dosing cycle comprises administration of an additional dose of atezolizumab.

In some aspects, the each additional dose of atezolizumab is between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200). In some aspects, each additional dose of atezolizumab is about 1200 mg. In some aspects, each additional dose of atezolizumab is 1200 mg.

In some aspects, each additional dose of atezolizumab is administered on Day 1 of each respective additional dosing cycle.

In some aspects, each additional dose of atezolizumab is administered after administration of each additional dose of tiragolumab.

In some aspects, tiragolumab is administered intravenously to the subject.

In some aspects, mosunetuzumab is administered subcutaneously to the subject. In some aspects, mosunetuzumab is administered intravenously to the subject. In some aspects, each dose of mosunetuzumab is administered subcutaneously to the subject. In some aspects, each dose of mosunetuzumab is administered intravenously to the subject.

In some aspects, atezolizumab is administered intravenously to the subject.

In some aspects, the method further comprises administering to the subject one or more (e.g., one, two, three, four, five, six, seven, eight, nine, ten, or more) additional therapeutic agents.

In some aspects, the one or more additional therapeutic agents is an IL-6R antagonist or a corticosteroid. In some aspects, the one or more additional therapeutic agents is an IL-6R antagonist. In some aspects, the one or more additional therapeutic agents is a corticosteroid.

In some aspects, the IL-6R antagonist is tocilizumab.

In some aspects, the corticosteroid is methylprednisolone, dexamethasone, or prednisone. In some aspects, the corticosteroid is methylprednisolone. In some aspects, the corticosteroid is dexamethasone. In some aspects, the corticosteroid is prednisone.

In some aspects, the subject is human.

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 45 mg (e.g., 45 mg) and the C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 45 mg (e.g., 45 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 45 mg (e.g., 45 mg), the C2D1 of tiragolumab is about 600 mg (e.g., 600 mg), and the C2D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 45 mg (e.g., 45 mg) and the C2D1 of atezolizumab is about 1200 mg (e.g., 1200 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 45 mg (e.g., 45 mg), and wherein each single dose C2D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 45 mg (e.g., 45 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 45 mg (e.g., 45 mg), each single dose C2D1-C8D1 of tiragolumab is about 600 mg (e.g., 600 mg), and each single dose C2D1-C8D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 45 mg (e.g., 45 mg) and each single dose C2D1-C8D1 of atezolizumab is about 1200 mg (e.g., 1200 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 45 mg (e.g., 45 mg) and wherein each single dose C2D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 45 mg (e.g., 45 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 45 mg (e.g., 45 mg), each single dose C2D1-C17D1 of tiragolumab is about 600 mg (e.g., 600 mg), and each single dose C2D1-C17D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 45 mg (e.g., 45 mg), and the C1D3 of mosunetuzumab is about 45 mg (e.g., 45 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 45 mg (e.g., 45 mg) and each single dose C2D1-C17D1 of atezolizumab is about 1200 mg (e.g., 1200 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 45 mg (e.g., 45 mg) and the C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 45 mg (e.g., 45 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 45 mg (e.g., 45 mg), the C2D1 of tiragolumab is about 600 mg (e.g., 600 mg), and the C2D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 45 mg (e.g., 45 mg) and the C2D1 of atezolizumab is about 1200 mg (e.g., 1200 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 45 mg (e.g., 45 mg), and wherein each single dose C2D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 45 mg (e.g., 45 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 45 mg (e.g., 45 mg), each single dose C2D1-C8D1 of tiragolumab is about 600 mg (e.g., 600 mg), and each single dose C2D1-C8D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 45 mg (e.g., 45 mg) and each single dose C2D1-C8D1 of atezolizumab is about 1200 mg (e.g., 1200 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 45 mg (e.g., 45 mg) and wherein each single dose C2D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and subcutaneously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 45 mg (e.g., 45 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 45 mg (e.g., 45 mg), each single dose C2D1-C17D1 of tiragolumab is about 600 mg (e.g., 600 mg), and each single dose C2D1-C17D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and subcutaneously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 5 mg (e.g., 5 mg), the C1D2 of mosunetuzumab is about 15 mg (e.g., 15 mg), and the C1D3 of mosunetuzumab is about 45 mg (e.g., 45 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 45 mg (e.g., 45 mg) and each single dose C2D1-C17D1 of atezolizumab is about 1200 mg (e.g., 1200 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 30 mg (e.g., 30 mg) and the C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 30 mg (e.g., 30 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 30 mg (e.g., 30 mg), the C2D1 of tiragolumab is about 600 mg (e.g., 600 mg), and the C2D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 30 mg (e.g., 30 mg) and the C2D1 of atezolizumab is about 1200 mg (e.g., 1200 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 30 mg (e.g., 30 mg), and wherein each single dose C2D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein each single dose C2D1-C8D1 of mosunetuzumab is about 30 mg (e.g., 30 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 30 mg (e.g., 30 mg), each single dose C2D1-C8D1 of tiragolumab is about 600 mg (e.g., 600 mg), and each single dose C2D1-C8D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein each single dose C2D1-C8D1 of mosunetuzumab is about 30 mg (e.g., 30 mg) and each single dose C2D1-C8D1 of atezolizumab is about 1200 mg (e.g., 1200 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 30 mg (e.g., 30 mg) and wherein each single dose C2D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein each single dose C2D1-C17D1 of mosunetuzumab is about 30 mg (e.g., 30 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 30 mg (e.g., 30 mg), each single dose C2D1-C17D1 of tiragolumab is about 600 mg (e.g., 600 mg), and each single dose C2D1-C17D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 30 mg (e.g., 30 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein each single dose C2D1-C17D1 of mosunetuzumab is about 30 mg (e.g., 30 mg) and each single dose C2D1-C17D1 of atezolizumab is about 1200 mg (e.g., 1200 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 60 mg (e.g., 60 mg) and the C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second dosing cycle comprises a single dose (C2D1) of mosunetuzumab and a single dose (C2D1) of tiragolumab administered on Day 1 of the second dosing cycle, wherein the C2D1 of mosunetuzumab is about 60 mg (e.g., 60 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 60 mg (e.g., 60 mg), the C2D1 of tiragolumab is about 600 mg (e.g., 600 mg), and the C2D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising at least a first dosing cycle and a second dosing cycle, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second dosing cycle comprises a single dose (C2D1) of mosunetuzumab, a single dose (C2D1) of tiragolumab, and a single dose (C2D1) of atezolizumab administered on Day 1 of the second dosing cycle, wherein the C2D1 of mosunetuzumab is about 60 mg (e.g., 60 mg) and the C2D1 of atezolizumab is about 1200 mg (e.g., 1200 mg), and wherein each single dose C1D1 and C2D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein the C2D1 is about 60 mg (e.g., 60 mg), wherein each single dose C3D1-C8D1 of mosunetuzumab is about 30 mg (e.g., 30 mg), and wherein each single dose C2D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to eighth dosing cycles each comprises a single dose (C2D1-C8D1) of mosunetuzumab and a single dose (C2D1-C8D1) of tiragolumab administered on Day 1 of each respective dosing cycle, wherein the C2D1 is about 60 mg (e.g., 60 mg), wherein each single dose C3D1-C8D1 of mosunetuzumab is about 30 mg (e.g., 30 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising eight dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein the C2D1 is about 60 mg (e.g., 60 mg), wherein each single dose C3D1-C8D1 of mosunetuzumab is about 30 mg (e.g., 30 mg), each single dose C2D1-C8D1 of tiragolumab is about 600 mg (e.g., 600 mg), and each single dose C2D1-C8D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising eight dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to eighth dosing cycles each comprises a single dose (C2D1-C8D1) of mosunetuzumab, a single dose (C2D1-C8D1) of tiragolumab, and a single dose (C2D1-C8D1) of atezolizumab administered on Day 1 of each respective dosing cycle, wherein the C2D1 is about 60 mg (e.g., 60 mg), wherein each single dose C3D1-C8D1 of mosunetuzumab is about 30 mg (e.g., 30 mg) and each single dose C2D1-C8D1 of atezolizumab is about 1200 mg (e.g., 1200 mg), and wherein each single dose C1D1-C8D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein the C2D1 is about 60 mg (e.g., 60 mg), wherein each single dose C3D1-C17D1 of mosunetuzumab is about 30 mg (e.g., 30 mg) and wherein each single dose C2D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab and intravenously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to seventeenth dosing cycles each comprises a single dose (C2D1-C17D1) of mosunetuzumab and a single dose (C2D1-C17D1) of tiragolumab administered on Day 1 of each respective dosing cycle, wherein the C2D1 is about 60 mg (e.g., 60 mg), wherein each single dose C3D1-C17D1 of mosunetuzumab is about 30 mg (e.g., 30 mg), and wherein each single dose C1D1-C17D1 of tiragolumab is about 600 mg (e.g., 600 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising seventeen dosing cycles, wherein: (a) the first dosing cycle comprises a first dose (C1D1) of mosunetuzumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein the C2D1 is about 60 mg (e.g., 60 mg), wherein each single dose C3D1-C17D1 of mosunetuzumab is about 30 mg (e.g., 30 mg), each single dose C2D1-C17D1 of tiragolumab is about 600 mg (e.g., 600 mg), and each single dose C2D1-C17D1 of atezolizumab is about 1200 mg (e.g., 1200 mg).

In another aspect, the invention features a method of treating a population of subjects having a relapsed or refractory (R/R) non-Hodgkin's lymphoma (NHL) (e.g., an aggressive NHL or an R/R NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)), the method comprising intravenously administering to the population of subjects tiragolumab, intravenously administering to the population of subjects atezolizumab, and intravenously administering to the population of subjects mosunetuzumab in a dosing regimen comprising seventeen dosing cycles, wherein: (a) the first dosing cycle comprises a first dose (C1D1) of mosunetuzumab and a single dose (C1D1) of tiragolumab administered on Day 1 of the first dosing cycle, a second dose (C1D2) of mosunetuzumab administered on Day 8 (±1 day) of the first dosing cycle, and a third dose (C1D3) of mosunetuzumab administered on Day 15 (±1 day) of the first dosing cycle, wherein the C1D1 of mosunetuzumab is about 1 mg (e.g., 1 mg), the C1D2 of mosunetuzumab is about 2 mg (e.g., 2 mg), and the C1D3 of mosunetuzumab is about 60 mg (e.g., 60 mg); and (b) the second to seventeenth dosing cycles each comprises a single dose (C2D1-C17D1) of mosunetuzumab, a single dose (C2D1-C17D1) of tiragolumab, and a single dose (C2D1-C17D1) of atezolizumab administered on Day 1 of each respective dosing cycle, wherein the C2D1 is about 60 mg (e.g., 60 mg), wherein each single dose C3D1-C17D1 of mosunetuzumab is about 30 mg (e.g., 30 mg) and each single dose C2D1-C17D1 of atezolizumab is about 1200 mg (e.g., 1200 mg), and wherein each single dose C1D1-C17D1 of tiragolumab is about 600 mg (e.g., 600 mg).

In some aspects, the complete response rate in the population of subjects is higher than a reference complete response rate in a reference population of subjects treated with a monotherapy comprising mosunetuzumab.

In some aspects, the objective response rate in the population of subjects is higher than a reference objective response rate in a reference population of subjects treated with a monotherapy comprising mosunetuzumab.

In some aspects, the rate of adverse events in the population of subjects is substantially the same as a reference rate of adverse events in a reference population of subjects treated with a monotherapy comprising mosunetuzumab.

In some aspects, the rate of cytokine release syndrome (CRS) events in the population of subjects is substantially the same as a reference rate of CRS events in a reference population of subjects treated with a monotherapy comprising mosunetuzumab.

In some aspects, the rate of CRS events having a grade of 3 or higher as defined by the American Society of Transplantation and Cellular Therapy (ASTCT) Consensus Grading for Cytokine-Release Syndrome (“ASTCT CRS grading”) in the population of subjects is substantially the same as a reference rate of CRS events having a grade of 3 or higher as defined by ASCT CRS grading in a reference population of subjects treated with a monotherapy comprising mosunetuzumab.

In some aspects, the complete response rate in the population of subjects is higher than a reference complete response rate in a reference population of subjects treated with a monotherapy comprising subcutaneous administration of mosunetuzumab.

In some aspects, the objective response rate in the population of subjects is higher than a reference objective response rate in a reference population of subjects treated with a monotherapy comprising subcutaneous administration of mosunetuzumab.

In some aspects, the rate of adverse events in the population of subjects is substantially the same as a reference rate of adverse events in a reference population of subjects treated with a monotherapy comprising subcutaneous administration of mosunetuzumab.

In some aspects, the rate of CRS events in the population of subjects is substantially the same as a reference rate of CRS events in a reference population of subjects treated with a monotherapy comprising subcutaneous administration of mosunetuzumab.

In some aspects, the rate of CRS events having a grade of 3 or higher as defined by ASTCT CRS grading in the population of subjects is substantially the same as a reference rate of CRS events having a grade of 3 or higher as defined by ASCT CRS grading in a reference population of subjects treated with a monotherapy comprising subcutaneous administration of mosunetuzumab.

In some aspects, the subjects are human.

Any of the methods described herein may involve monitoring a subject for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. CRS grading and strategies for management of CRS events is discussed in detail below in Section XIII.

### B. Mosunetuzumab

The invention provides mosunetuzumab, a bispecific antibody that binds to CD20 and CD3, useful for treating relapsed and/or refractory (R/R) follicular lymphoma (FL). The FL may be of Grades 1, 2, or 3a, but not Grade 3b.

In some instances, the invention provides mosunetuzumab that includes (1) an anti-CD20 arm having a first binding domain comprising at least one, two, three, four, five, or six HVRs selected from (a) an HVR-H1 comprising the amino acid sequence of GYTFTSYNMH (SEQ ID NO: 74); (b) an HVR-H2 comprising the amino acid sequence of AIYPGNGDTSYNQKFKG (SEQ ID NO: 75); (c) an HVR-H3 comprising the amino acid sequence of VVYYSNSYWYFDV (SEQ ID NO: 76); (d) an HVR-L1 comprising the amino acid sequence of RASSSVSYMH (SEQ ID NO: 77); (e) an HVR-L2 comprising the amino acid sequence of APSNLAS (SEQ ID NO: 78); and (f) an HVR-L3 comprising the amino acid sequence of QQWSFNPPT (SEQ ID NO: 79); and (2) an anti-CD3 arm having a second binding domain comprising at least one, two, three, four, five, or six HVRs selected from (a) an HVR-H1 comprising the amino acid sequence of NYYIH (SEQ ID NO: 90); (b) an HVR-H2 comprising the amino acid sequence of WIYPGDGNTKYNEKFKG (SEQ ID NO: 91); (c) an HVR-H3 comprising the amino acid sequence of DSYSNYYFDY (SEQ ID NO: 92); (d) an HVR-L1 comprising the amino acid sequence of KSSQSLLNSRTRKNYLA (SEQ ID NO: 93); (e) an HVR-L2 comprising the amino acid sequence of WASTRES (SEQ ID NO: 94); and (f) an HVR-L3 comprising the amino acid sequence of TQSFILRT (SEQ ID NO: 95). In some instances, mosunetuzumab comprises (1) at least one (e.g., 1, 2, 3, or 4) of heavy chain framework regions FR-H1, FR-H2, FR-H3, and FR-H4 comprising the sequences of SEQ ID NOs: 82-85, respectively, and/or at least one (e.g., 1, 2, 3, or 4) of the light chain framework regions FR-L1, FR-L2, FR-L3, and FR-L4 comprising the sequences of SEQ ID NOs: 86-89, respectively, and (2) at least one (e.g., 1, 2, 3, or 4) of heavy chain framework regions FR-H1, FR-H2, FR-H3, and FR-H4 comprising the sequences of SEQ ID NOs: 98-101, respectively, and/or at least one (e.g., 1, 2, 3, or 4) of the light chain framework regions FR-L1, FR-L2, FR-L3, and FR-L4 comprising the sequences of SEQ ID NOs: 102-105, respectively.

In some instances, mosunetuzumab comprises (1) an anti-CD20 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 80; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 81; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 96; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 97; or (c) a VH domain as in (a) and a VL domain as in (b). In some instances, mosunetuzurab comprises (1) a first binding domain comprising a VH domain comprising an amino acid sequence of SEQ ID NO: 80 and a VL domain comprising an amino acid sequence of SEQ ID NO: 81 and (2) a second binding domain comprising a VH domain comprising an amino acid sequence of SEQ ID NO: 96 and a VL domain comprising an amino acid sequence of SEQ ID NO: 97.

In some instances, mosunetuzumab has the International Nonproprietary Names for Pharmaceutical Substances (INN) List 117 (WHO Drug Information, Vol. 31, No. 2, 2017, p. 303), or CAS Registry No. 1905409-39-3, and having (1) an anti-CD20 arm comprising the heavy chain and light chain sequences of SEQ ID NOs: 106 and 107, respectively; and (2) an anti-CD3 arm comprising the heavy chain and light chain sequences of SEQ ID NOs: 108 and 109, respectively. In some instances, mosunetuzumab comprises (1) an anti-CD20 arm comprising a first binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 106; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 107; or (c) a heavy chain as in (a) and a light chain as in (b), and (2) an anti-CD3 arm comprising a second binding domain comprising (a) a heavy chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 108; (b) a light chain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 109; or (c) a heavy chain as in (a) and a light chain as in (b). In some instances, mosunetuzumab comprises (1) an anti-CD20 arm comprising a first binding domain comprising a heavy chain comprising an amino acid sequence of SEQ ID NO: 106 and a light chain comprising an amino acid sequence of SEQ ID NO: 107 and (2) an anti-CD3 arm comprising a second binding domain comprising a heavy chain comprising an amino acid sequence of SEQ ID NO: 108 and a light chain comprising an amino acid sequence of SEQ ID NO: 109.

Amino acid sequences of mosunetuzumab are summarized in Table 1 below.

TABLE 1
Sequence IDs for Mosunetuzumab

| CD3 Arm | | CD20 Arm | |
|---|---|---|---|
| SEQ ID NO: | Description | SEQ ID NO: | Description |
| 90 | CD3 HVR-H1 | 74 | CD20 HVR-H1 |
| 91 | CD3 HVR-H2 | 75 | CD20 HVR-H2 |
| 92 | CD3 HVR-H3 | 76 | CD20 HVR-H3 |
| 93 | CD3 HVR-L1 | 77 | CD20 HVR-L1 |
| 94 | CD3 HVR-L2 | 78 | CD20 HVR-L2 |
| 95 | CD3 HVR-L3 | 79 | CD20 HVR-L3 |
| 96 | CD3 VH | 80 | CD20 VH |
| 97 | CD3 VL | 81 | CD20 VL |
| 108 | CD3 heavy chain | 106 | CD20 heavy chain |
| 109 | CD3 light chain | 107 | CD20 light chain |

Mosunetuzumab may be produced using recombinant methods and compositions, for example, as described in U.S. Pat. No. 4,816,567.

### C. Additional Therapeutic Agents

In some instances, the methods described herein include administering the bispecific anti-CD20/anti-CD3 antibody (e.g., mosunetuzumab) and anti-TIGIT antagonist antibody (e.g., tiragolumab) in combination with one or more additional therapeutic agents

In some instances, the additional therapeutic agent is a PD-1 axis binding antagonist described herein. In some instances, the additional therapeutic agent is atezolizumab. In some instances, atezolizumab is administered to a subject in combination with mosunetuzumab and tiragolumab according to a dosing regimen described herein.

In some instances, the one or more additional therapeutic agents may reduce the rate or the severity of cytokine release syndrome (CRS). In some instances, the one or more additional therapeutic agents may prevent symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #50-42-8), or methylprednisolone (CAS #: 83-43-2)) or an IL-6R antagonist (e.g., tocilizumab, sarilumab, vobarilizumab (ALX-0061), satralizumab (SA-237), and variants thereof). In some instances, the additional therapeutic agent is tocilizumab. In some instances, the additional therapeutic agent is a corticosteroid. In some instances, a corticosteroid is administered prior to administration of mosunetuzumab. In some instances, the corticosteroid is administered 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of mosunetuzumab. In some instances, the corticosteroid is administered intravenously. In some instances, the corticosteroid is dexamethasone. In some instances, 10 mg of dexamethasone is administered to a subject 5 minutes, 10 minutes, 15 minutes, 20 minutes, 30 minutes, 40 minutes, 50 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours before administration of mosunetuzumab to the subject. In some instances, the corticosteroid is methylprednisolone. In some instances, the corticosteroid is prednisone.

The methods described herein may result in an improved benefit-risk profile for subjects having a relapsed or refractory non-Hodgkin's lymphoma (NHL) ((e.g., a B cell proliferative disorder, e.g., an NHL (e.g., an aggressive NHL or an R/R NHL; e.g., an R/R DLBCL, an R/R FL (e.g., an R/R Grade 3b FL), or an R/R high grade B cell lymphoma (HGBL) or an R/R transformed FL (trFL)) being treated with mosunetuzumab. In some instances, treatment using the methods described herein that result in subcutaneously administering mosunetuzumab and the anti-TIGIT antagonist antibody (e.g., tiragolumab) in the context of a fractionated, dose-escalation dosing regimen results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or hepatotoxicities, following treatment with mosunetuzumab using the fractionated, dose-escalation dosing regimen of the invention relative to treatment with mosunetuzumab using an non-fractioned dosing regimen.

For all the methods described herein, mosunetuzumab is formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. Mosunetuzumab need not be, but is optionally formulated with, one or more agents currently used to prevent or treat the disorder in question. The effective amount of such other agents depends on the amount of mosunetuzumab present in the formulation, the type of disorder or treatment, and other factors discussed above. Mosunetuzumab may be suitably administered to the subject over a series of treatments. In some aspects, mosunetuzumab is administered subcutaneously. In some aspects, mosunetuzumab is administered intravenously.

In some instances, additional therapeutic agents useful in the present invention include therapeutic antibodies, such as alemtuzumab (CAMPATH®), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (BEXXAR®, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, tafasitamab, talizumab, tefibazumab, tocilizumab, toralizumab, tucotuzumab celmoleukin, tucusituzumab, umavizumab, urtoxazumab, ustekinumab, visilizumab, and briakinumab.

### V. Therapeutic Methods and Compositions for Colorectal Cancer

### A. Therapeutic Methods for Dosing of Tiragolumab and Atezolizumab

In one aspect, the disclosure provides a method for treating a subject having a colorectal cancer (CRC) (e.g., a metastatic CRC (e.g., a microsatellite instability-high (MSI-H) metastatic CRC)) comprising administering to the subject tiragolumab and atezolizumab.

In some aspects, the metastatic CRC (e.g., the MSI-H metastatic CRC) is an adenocarcinoma.

In some aspects, the subject has experienced disease progression on previous checkpoint-inhibitor-based therapy (e.g., at least one, at least two, at least three, at least four, at least five, at least six, at least seven, at least eight, at least nine, or at least ten previous therapies; e.g., one, two, three, four, five, six, seven, eight, nine, ten, or more than ten previous therapies).

In some aspects, the tiragolumab and atezolizumab are administered to the subject in a dosing regimen that comprises one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more dosing cycles). In some aspects, the dosing regimen comprises at least 16 dosing cycles (e.g., 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles).

In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days. In some aspects, the length of each of the one or more dosing cycles is 21 days. In some aspects, the tiragolumab and atezolizumab are administered to the subject on about Day 1 (e.g., on Day 1) of each of the one or more dosing cycles.

In some aspects, the tiragolumab is administered to the subject at a dose (e.g. a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the tiragolumab is administered to the subject at a dose (e.g., a fixed dose) of between about 30 mg to about 600 mg (e.g., between about 50 mg to about 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the tiragolumab is administered to the subject at a dose (e.g., a fixed dose) of about 600 mg every three weeks.

In some aspects, the tiragolumab is administered to the subject at a dose (e.g. a fixed dose) of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the tiragolumab is administered to the subject at a dose (e.g., a fixed dose) of between 30 mg to 600 mg (e.g., between 50 mg to 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg) every three weeks. In some aspects, the tiragolumab is administered to the subject at a dose (e.g., a fixed dose) of 600 mg every three weeks.

In some aspects, the atezolizumab is administered to the subject at a dose (e.g., a fixed dose) of between about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some aspects, the atezolizumab is administered to the subject at a dose (e.g., a fixed dose) of about 1200 mg every three weeks. In some aspects, the atezolizumab is administered to the subject at a dose (e.g., a fixed dose) of between 80 mg to 2000 mg (e.g., between 100 mg to 1600 mg, e.g., between 200 mg to 1600 mg, e.g., between 300 mg to 1600 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) every three weeks.

In some aspects, the tiragolumab is administered to the subject at a dose of about 600 mg (e.g., a fixed dose of 600 mg) every three weeks and the atezolizumab is administered at a dose of about 1200 mg (e.g., a fixed dose of 1200 mg) every three weeks.

In some aspects, the atezolizumab is administered to the subject before the tiragolumab. In some aspects, the tiragolumab is administered to the subject before the atezolizumab.

In another aspect, the invention features a method of treating a subject having a metastatic CRC (e.g., a MSI-H metastatic CRC), the method comprising administering to the subject a dosing regimen comprising one or more 21-day dosing cycles of tiragolumab at a dose of about 600 mg (e.g., a fixed dose of 600 mg) on Day 1 of each dosing cycle and atezolizumab at a dose of about 1200 mg (e.g., a fixed dose of 1200 mg) on Day 1 of each dosing cycle.

In some aspects, the tiragolumab is administered intravenously. In some aspects, the atezolizumab is administered intravenously.

In some aspects, the MSI-H status is determined by next-generation sequencing, polymerase chain reaction (PCR), immunohistochemistry (IHC), FOUNDATIONONE® Liquid CDx testing, or a combination thereof.

In some aspects, the subject is a human.

### B. Therapeutic Method for Dosing of Tiragolumab, Atezolizumab, and Bevacizumab

In one aspect, the disclosure provides a method for treating a subject having a colorectal cancer (CRC) (e.g., a metastatic CRC (e.g., a microsatellite instability-high (MSI-H) metastatic CRC)) comprising administering to the subject tiragolumab, atezolizumab, and bevacizumab.

In some aspects, the metastatic CRC (e.g., the MSI-H metastatic CRC) is an adenocarcinoma.

In some aspects, the subject has experienced disease progression on previous checkpoint-inhibitor-based therapy (e.g., at least one, at least two, at least three, at least four, at least five, at least six, at least seven, at least eight, at least nine, or at least ten previous therapies; e.g., one, two, three, four, five, six, seven, eight, nine, ten, or more previous therapies).

In some aspects, the tiragolumab, atezolizumab, and bevacizumab are administered to the subject in a dosing regimen that comprises one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more dosing cycles). In some aspects, the dosing regimen comprises at least 16 dosing cycles (e.g., 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles).

In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days. In some aspects, the length of each of the one or more dosing cycles is 21 days. In some aspects, the tiragolumab, atezolizumab, and bevacizumab are administered to the subject on about Day 1 (e.g., on Day 1) of each of the one or more dosing cycles.

In some aspects, the tiragolumab is administered to the subject at a dose (e.g. a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the tiragolumab is administered to the subject at a dose (e.g., a fixed dose) of between about 30 mg to about 600 mg (e.g., between about 50 mg to about 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the tiragolumab is administered to the subject at a dose (e.g., a fixed dose) of about 600 mg every three weeks.

In some aspects, the tiragolumab is administered to the subject at a dose (e.g. a fixed dose) of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the tiragolumab is administered to the subject at a dose (e.g., a fixed dose) of between 30 mg to 600 mg (e.g., between 50 mg to 600 mg, e.g., between 60 mg to 600 mg, e.g., between 100 mg to 600 mg, e.g., between 200 mg to 600 mg, e.g., between 200 mg to 550 mg, e.g., between 250 mg to 500 mg, e.g., between 300 mg to 450 mg, e.g., between 350 mg to 400 mg, e.g., 375 mg) every three weeks. In some aspects, the tiragolumab is administered to the subject at a dose (e.g., a fixed dose) of 600 mg every three weeks.

In some aspects, the atezolizumab is administered to the subject at a dose (e.g., a fixed dose) of between about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks. In some aspects, the atezolizumab is administered to the subject at a dose (e.g., a fixed dose) of about 1200 mg every three weeks. In some aspects, the atezolizumab is administered to the subject at a dose (e.g., a fixed dose) of between 80 mg to 2000 mg (e.g., between 100 mg to 1600 mg, e.g., between 200 mg to 1600 mg, e.g., between 300 mg to 1600 mg, e.g., between 400 mg to 1600 mg, e.g., between 500 mg to 1600 mg, e.g., between 600 mg to 1600 mg, e.g., between 700 mg to 1600 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg, e.g., 1200 mg±5 mg, e.g., 1200±2.5 mg, e.g., 1200±1.0 mg, e.g., 1200±0.5 mg, e.g., 1200 mg) every three weeks.

In some aspects, the bevacizumab is administered to the subject at a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some aspects, the bevacizumab is administered to the subject at a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some aspects, the bevacizumab is administered to the subject at a dose of about 15 mg/kg administered every three weeks.

In some aspects, the bevacizumab is administered to the subject at a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some aspects, the bevacizumab is administered to the subject at a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15±0.5 mg/kg, e.g., 15±0.2 mg/kg, e.g., 15±0.1 mg/kg, e.g., 15 mg/kg) every three weeks. In some aspects, the bevacizumab is administered to the subject at a dose of 15 mg/kg administered every three weeks.

In some aspects, the tiragolumab is administered to the subject at a dose of about 600 mg (e.g., a fixed dose of 600 mg) every three weeks, the atezolizumab is administered at a dose of about 1200 mg (e.g., a fixed dose of 1200 mg) every three weeks, and the bevacizumab is administered at a dose of about 15 mg/kg (e.g., 15 mg/kg) every three weeks.

In some aspects, the atezolizumab is administered to the subject before the tiragolumab. In some aspects, the tiragolumab is administered to the subject before the atezolizumab. In some aspects, the atezolizumab is administered before the bevacizumab and the bevacizumab is administered before the tiragolumab. In some aspects in which the tiragolumab, atezolizumab, and bevacizumab are administered on the same day, in some aspects, the tiragolumab is administered first, the bevacizumab is administered second, and the atezolizumab is administered third. In some aspects, the tiragolumab is administered first, the atezolizumab is administered second, and the bevacizumab is administered third. In some aspects, the atezolizumab is administered first, the tiragolumab is administered second, and the bevacizumab is administered third. In some aspects, the bevacizumab is administered first, the atezolizumab is administered second, and the tiragolumab is administered third. In some aspects, the bevacizumab is administered first, the tiragolumab is administered second, and the atezolizumab is administered third. In some aspects, the tiragolumab and the atezolizumab are administered simultaneously. In some aspects, the tiragolumab and the atezolizumab are combined in an IV bag prior to administration.

In another aspect, the invention features a method of treating a subject having a metastatic CRC (e.g., a MSI-H metastatic CRC), the method comprising administering to the subject a dosing regimen comprising one or more 21-day dosing cycles of tiragolumab at a dose of about 600 mg (e.g., a fixed dose of 600 mg) on Day 1 of each dosing cycle, atezolizumab at a dose of about 1200 mg (e.g., a fixed dose of 1200 mg) on Day 1 of each dosing cycle, and bevacizumab at a dose of about 15 mg/kg (e.g., 15 mg/kg) on Day 1 of each dosing cycle.

In some aspects, the tiragolumab is administered intravenously. In some aspects, the atezolizumab is administered intravenously. In some aspects, the bevacizumab is administered intravenously.

In some aspects, the MSI-H status is determined by next-generation sequencing, polymerase chain reaction (PCR), immunohistochemistry (IHC), FOUNDATIONONE® Liquid CDx testing, or a combination thereof.

In some aspects, the subject is a human.

### VI. Combination Dosing of Anti-TIGIT Antagonist Antibodies and PD-1 Axis Binding Antagonists

In some instances, a dose of an effective amount of an anti-TIGIT antagonist antibody (e.g., an anti-TIGIT antagonist antibody as disclosed herein, e.g., tiragolumab) is administered with a dose of an effective amount of a PD-1 axis binding antagonist (e.g., anti-PD-L1 antagonist antibody (e.g., atezolizumab)) in a combination therapy (e.g., a combination treatment of an anti-TIGIT antagonist antibody (e.g., an anti-TIGIT antagonist antibody as disclosed herein, e.g., tiragolumab) with a PD-1 axis binding antagonist (e.g., anti-PD-L1 antagonist antibody (e.g., atezolizumab)) for treatment of a subject having a cancer (e.g., esophageal cancer (e.g., metastatic esophageal cancer)).

In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from about 0.01 mg/kg to about 50 mg/kg (e.g., about 15 mg/kg) up to 1200 mg, e.g., every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from 0.01 mg/kg to 50 mg/kg (e.g., 15 mg/kg) up to 1200 mg, e.g., every three weeks. Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biosimulation studies based on extrapolations of pharmacokinetic parameters estimated from adult data. In some instances, the dose (e.g., about 600 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW) >40 kg: 600 mg, BW >15 kg and ≤40 kg: 400 mg, and BW ≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW) >40 kg: 600 mg, BW >15 kg and ≤40 kg: 400 mg, and BW ≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body surface area (e.g., BSA >1.25 m²: 600 mg, BSA >0.75 m² and ≤1.25 m²: 450 mg, BSA >0.5 m² and S 0.75 m²: 350 mg, and BSA ≤0.5 m²: 300 mg) every three weeks. In some embodiments, the PD-1 axis binding antagonist (e.g., anti-PD-L1 antagonist antibody (e.g., atezolizumab)) is administered at a maximum dose of 1200 mg every three weeks.

i. Dosing of Anti-TIGIT Antagonist Antibodies

As a general proposition, the therapeutically effective amount of an anti-TIGIT antagonist antibody (e.g., an anti-TIGIT antagonist antibody as disclosed herein, e.g., tiragolumab) administered to a subject having a cancer (e.g., esophageal cancer (e.g., metastatic esophageal cancer)) will be in the range of about 0.01 to about 50 mg/kg of subject body weight, whether by one or more administrations. In some embodiments, the therapeutically effective amount of an anti-TIGIT antagonist antibody (e.g., an anti-TIGIT antagonist antibody as disclosed herein, e.g., tiragolumab) administered to a subject is in the range of 0.01 to 50 mg/kg of subject body weight, whether by one or more administrations.

In some exemplary embodiments, the anti-TIGIT antagonist antibody (e.g., an anti-TIGIT antagonist antibody as disclosed herein, e.g., tiragolumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In exemplary embodiments, the anti-TIGIT antagonist antibody (e.g., an anti-TIGIT antagonist antibody as disclosed herein, e.g., tiragolumab) is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the anti-TIGIT antagonist antibody (e.g., an anti-TIGIT antagonist antibody as disclosed herein, e.g., tiragolumab) is administered on about Day 1 (e.g., Day −3, Day −2, Day −1, Day 1, Day 2, or Day 3) of a dosing cycle.

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W). In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab)) may be reduced as compared to a standard dose of the anti-TIGIT antagonist antibody administered as a monotherapy.

In some instances, the effective amount of the anti-TIGIT antagonist antibody (e.g., an anti-TIGIT antagonist antibody as disclosed herein, e.g., tiragolumab) is a fixed dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a fixed dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks).

In some instances, the effective amount of the anti-TIGIT antagonist antibody (e.g., an anti-TIGIT antagonist antibody as disclosed herein, e.g., tiragolumab) is a fixed dose of between about 200 mg to about 2000 mg (e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every four weeks (Q4W). In some instances, the effective amount of anti-TIGIT antagonist antibody (e.g., an anti-TIGIT antagonist antibody as disclosed herein, e.g., tiragolumab) is a fixed dose of about 840 mg every four weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every four weeks).

In some instances, the dose of the anti-TIGIT antagonist antibody (e.g., an anti-TIGIT antagonist antibody as disclosed herein, e.g., tiragolumab) is a tiered dose based on a subject's body weight (e.g., body weight (BW) >40 kg: 600 mg, BW >15 kg and ≤40 kg: 400 mg, and BW ≤15 kg: 300 mg).

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)) may be reduced as compared to a standard dose of the anti-TIGIT antagonist antibody administered as a monotherapy.

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, tiragolumab is administered to the subject intravenously at a dose of about 420 mg every 2 weeks, about 600 mg every 3 weeks, or about 840 mg of every 4 weeks. In some instances, tiragolumab is administered to the subject intravenously at a dose of 420 mg every 2 weeks, 600 mg every 3 weeks, or 840 mg of every 4 weeks.

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody administered as a monotherapy. In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel or nab-paclitaxel), and/or a topoisomerase II inhibitor (e.g., doxorubicin))) and/or G-CSF or GM-CSF, may be reduced as compared to a standard dose of the anti-TIGIT antagonist antibody administered as a monotherapy.

In some instances, a subject is administered a total of 1 to 60 doses of an anti-TIGIT antagonist antibody (e.g., an anti-TIGIT antagonist antibody as disclosed herein, e.g., tiragolumab), e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 doses. In some instances, a subject is administered a total of 1 to 60 doses of an anti-TIGIT antagonist antibody (e.g., an anti-TIGIT antagonist antibody as disclosed herein, e.g., tiragolumab), e.g., 1 to 60 doses, 1 to 55 doses, 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 60 doses, 2 to 55 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 60 doses, 3 to 55 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 60 doses, 4 to 55 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 60 doses, 5 to 55 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 60 doses, 10 to 55 doses, 10 to 50 doses, 10 to 45 doses, 10 to 40 doses, 10 to 35 doses, 10 to 30 doses, 10 to 25 doses, 10 to 20 doses, 10 to 15 doses, 15 to 60 doses, 15 to 55 doses, 15 to 50 doses, 15 to 45 doses, 15 to 40 doses, 15 to 35 doses, 15 to 30 doses, 15 to 25 doses, 15 to 20 doses, 20 to 60 doses, 20 to 55 doses, 20 to 50 doses, 20 to 45 doses, 20 to 40 doses, 20 to 35 doses, 20 to 30 doses, 20 to 25 doses, 25 to 60 doses, 25 to 55 doses, 25 to 50 doses, 25 to 45 doses, 25 to 40 doses, 25 to 35 doses, 25 to 30 doses, 30 to 60 doses, 30 to 55 doses, 30 to 50 doses, 30 to 45 doses, 30 to 40 doses, 30 to 35 doses, 35 to 60 doses, 35 to 55 doses, 35 to 50 doses, 35 to 45 doses, 35 to 40 doses, 40 to 60 doses, 40 to 55 doses, 40 to 50 doses, 40 to 45 doses, 45 to 50 doses, 50 to 60 doses, or 55 to 60 doses. In particular instances, the doses may be administered intravenously.

The PD-1 axis binding antagonist and anti-TIGIT antagonist antibody may be administered in any suitable manner known in the art. For example, the PD-1 axis binding antagonist and anti-TIGIT antagonist antibody may be administered sequentially (on different days) or concurrently (on the same day or during the same treatment cycle). In some instances, the anti-TIGIT antagonist antibody and/or the PD-1 axis binding antagonist are administered on about Day 1 (e.g., Day −3, Day −2, Day −1, Day 1, Day 2, or Day 3) of a dosing cycle. In some instances, the PD-1 axis binding antagonist and anti-TIGIT antagonist antibody may be administered on the same day. In some instances, the PD-1 axis binding antagonist is administered before the anti-TIGIT antagonist antibody. In some instances, the PD-1 axis binding antagonist is administered after the anti-TIGIT antagonist antibody. In some instances, the PD-1 axis binding antagonist is administered simultaneously with the anti-TIGIT antagonist antibody. In some instances, the PD-1 axis binding antagonist may be administered prior to an anti-TIGIT antagonist antibody that is administered on the same day. In some instances, the PD-1 axis binding antagonist may be administered after to an anti-TIGIT antagonist antibody that is administered on the same day. In yet other instances, the PD-1 axis binding antagonist is administered at the same time as the anti-TIGIT antagonist antibody. In some instances, the PD-1 axis binding antagonist is in a separate composition as the anti-TIGIT antagonist antibody. In some instances, the PD-1 axis binding antagonist is in the same composition as the anti-TIGIT antagonist antibody. In some instances, the PD-1 axis binding antagonist is administered through a separate intravenous line from any other therapeutic agent administered to the subject on the same day. The PD-1 axis binding antagonist and anti-TIGIT antagonist antibody may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the PD-1 axis binding antagonist is administered intravenously. In some instances, the anti-TIGIT antagonist antibody is administered intravenously, intramuscularly, subcutaneously, topically, orally, transdermally, intraperitoneally, intraorbitally, by implantation, by inhalation, intrathecally, intraventricularly, or intranasally. In some instances, the anti-TIGIT antagonist antibody is administered intravenously. In some instances, there is a first observation period following administration of the PD-1 axis binding antagonist. In some instances, there is a second observation period following administration of the PD-1 axis binding antagonist. In some instances, there is a first observation period following administration of the anti-TIGIT antagonist antibody. In some instances, there is a second observation period following administration of the anti-TIGIT antagonist antibody. In some instances, the observation period is between about 30 minutes to about 60 minutes in length. In some instances, the anti-TIGIT antagonist antibody and/or PD-1 axis binding antagonist are administered intravenously or subcutaneously.

In some instances, the intravenous infusion is over 30±10 minutes and/or over 60±10 minutes. In one example, atezolizumab may be administered intravenously over 60 minutes; if the first infusion is tolerated, all subsequent infusions may be delivered over 30 minutes. In one example, tiragolumab may be administered intravenously over 60 minutes; if the first infusion is tolerated, all subsequent infusions may be delivered over 30 minutes.

In some examples, the PD-1 axis binding antagonist is not administered as an intravenous push or bolus. In some examples, the anti-TIGIT antagonist antibody is not administered as an intravenous push or bolus.

In any of the preceding examples, each dosing cycle may have any suitable length, e.g., about 7 days (about 5, 6, 7, 8, or 9 days), about 14 days (e.g., about 12, 13, 14, 15, or 16 days), about 21 days (e.g., about 18, 19, 20, 21, 22, 23, or 24 days), about 28 days (about 25, 26, 27, 28, 29, 30, or 31 days), or longer. In some instances, each dosing cycle is about 21 days.

ii. Dosing of PD-1 Axis Binding Antagonists

As a general proposition, the therapeutically effective amount of a PD-1 axis binding antagonist (e.g., atezolizumab) administered to a subject having a cancer (e.g., esophageal cancer (e.g., metastatic esophageal cancer)) will be in the range of about 0.01 to about 50 mg/kg of subject body weight, whether by one or more administrations.

In some exemplary embodiments, the PD-1 axis binding antagonist (e.g., atezolizumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In some instances, the dose of the PD-1 axis binding antagonist (e.g., anti-PD-L1 antagonist antibody (e.g., atezolizumab)) is a dose based on a subject's body weight (e.g., 15 mg/kg). In some instances, the dose of the PD-1 axis binding antagonist (e.g., anti-PD-L1 antagonist antibody (e.g., atezolizumab)) is a dose based on a subject's body surface area (e.g., body surface area (BSA) >1.25 m²: 600 mg, BSA >0.75 m² and ≤1.25 m²: 450 mg, BSA >0.5 m² and ≤0.75 m²: 350 mg, and BSA ≤0.5 m²: 300 mg).

In some instances, the effective amount of the PD-1 axis binding antagonist (e.g., an anti-PD-L1 antagonist antibody (e.g., atezolizumab)) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some embodiments, the effective amount of the PD-1 axis binding antagonist is atezolizumab at a fixed dose of about 1200 mg every three weeks. In some embodiments, the effective amount of the PD-1 axis binding antagonist is pembrolizumab at a fixed dose of about 200 mg every three weeks or, alternatively, pembrolizumab at a fixed dose of about 400 mg every six weeks.

In some instances, the fixed dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., an anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., an anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of PD-1 axis binding antagonist (e.g., an anti-PD-L1 antagonist antibody (e.g., atezolizumab)) is a dose of about 15 mg/kg administered every three weeks. In some instances, the dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., anti-PD-L1 antagonist antibody (e.g., atezolizumab)) is a fixed dose of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist is atezolizumab at a fixed dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some embodiments, the effective amount of the PD-1 axis binding antagonist is avelumab at a fixed dose of about 800 mg every two weeks. In some embodiments, the effective amount of the PD-1 axis binding antagonist is nivolumab at a fixed dose of about 240 mg every two weeks.

In some instances, the effective amount of the PD-1 axis binding antagonist (e.g., anti-PD-L1 antagonist antibody (e.g., atezolizumab)) is a fixed dose of between about 500 mg to about 3000 mg (e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab)) is a fixed dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680±0.5 mg, e.g., 1680 mg every four weeks). In some embodiments, the effective amount of the PD-1 axis binding antagonist is nivolumab at a fixed dose of about 480 mg every four weeks.

In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein (e.g., tiragolumab) may be reduced as compared to a standard dose of the anti-PD-L1 antagonist antibody administered as a monotherapy. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF may be reduced as compared to a standard dose of the PD-1 axis binding antagonist administered as a monotherapy.

In some instances, a subject is administered a total of 1 to 60 doses of a PD-1 axis binding antagonist (e.g., atezolizumab), e.g., 1 to 60 doses, 1 to 55 doses, 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 60 doses, 2 to 55 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 60 doses, 3 to 55 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 60 doses, 4 to 55 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 60 doses, 5 to 55 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 60 doses, 10 to 55 doses, 10 to 50 doses, 10 to 45 doses, 10 to 40 doses, 10 to 35 doses, 10 to 30 doses, 10 to 25 doses, 10 to 20 doses, 10 to 15 doses, 15 to 60 doses, 15 to 55 doses, 15 to 50 doses, 15 to 45 doses, 15 to 40 doses, 15 to 35 doses, 15 to 30 doses, 15 to 25 doses, 15 to 20 doses, 20 to 60 doses, 20 to 55 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 60 doses, 30 to 55 doses, 30 to 50 doses, 30 to 45 doses, 30 to 40 doses, 30 to 35 doses, 35 to 60 doses, 35 to 55 doses, 35 to 50 doses, 35 to 45 doses, 35 to 40 doses, 40 to 60 doses, 40 to 55 doses, 40 to 50 doses, 40 to 45 doses, 45 to 50 doses, 50 to 60 doses, or 55 to 60 doses. In particular instances, the doses may be administered intravenously.

In some instances, atezolizumab is administered to the subject intravenously at a dose of about 840 mg every 2 weeks, about 1200 mg every 3 weeks, or about 1680 mg of every 4 weeks. For example, in some aspects, atezolizumab is administered to the subject intravenously at a dose of 1200 mg every 3 weeks. In some aspects, atezolizumab is administered to the subject intravenously at a dose of 840 mg every 2 weeks. In some aspects, atezolizumab is administered to the subject intravenously at a dose of 1680 mg every 4 weeks.

The PD-1 axis binding antagonist and/or any additional therapeutic agent(s) may be administered in any suitable manner known in the art. For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the subject on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating esophageal cancer in a subject comprising administering to the subject a treatment regimen comprising effective amounts of a PD-1 axis binding antagonist (e.g., atezolizumab) and an anti-TIGIT antagonist antibody (e.g., tiragolumab) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

iii. Dosing Cycles for Anti-TIGIT Antagonist Antibodies and PD-1 Axis Binding Antagonists

In any of the methods and uses of the invention, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) may be administered to the subject having a cancer (e.g., esophageal cancer (e.g., metastatic esophageal cancer)) in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more dosing cycles). In some aspects, the one or more dosing cycles comprise administration of one or more doses of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) as described in Sections IX and X, respectively, to the subject having a cancer (e.g., esophageal cancer (e.g., metastatic esophageal cancer)). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) continue until there is a loss of clinical benefit (e.g., confirmed disease progression, drug resistance, death, or unacceptable toxicity). In some instances, the length of each dosing cycle is about 7 to 42 days (e.g., 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 41 days, 42 days). In some instances, the length of each dosing cycle is about 14 days. In some instances, the length of each dosing cycle is about 21 days. In some instances, the length of each dosing cycle is about 28 days. In some instances, the length of each dosing cycle is about 42 days. In some instances, the length of each dosing cycle is about 7 days.

In some instances, the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are administered on about Day 1 (e.g., Day 1±3 days) of each dosing cycle. In some instances, the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) are administered on about Day 15 (e.g., Day 15±3 days) of each dosing cycle.

In some instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered on about Day 1 (e.g., Day 1±3 days) of each dosing cycle.

In some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks) and the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks) and the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In some instances, the anti-TIGIT antagonist antibody (e.g., an anti-TIGIT antagonist antibody as disclosed herein, e.g., tiragolumab) is administered intravenously at a dose of about 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of about 840 mg on Day 1 of each 14-day cycle (i.e., at a dose of about 840 mg every two weeks).

In some instances, the anti-TIGIT antagonist antibody (e.g., an anti-TIGIT antagonist antibody as disclosed herein, e.g., tiragolumab) is administered intravenously at a dose of 840 mg on Day 1 of each 28-day cycle (i.e., at a dose of 840 mg every four weeks) and the PD-1 axis binding antagonist (e.g., anti-PD-L1 antagonist antibody (e.g., atezolizumab)) is administered intravenously at a dose of 1680 mg on Day 1 of each 28-day cycle (i.e., at a dose of 1680 mg every four weeks).

iv. Intravenous Infusion and Subcutaneous Administration of Anti-TIGIT Antagonist Antibodies and PD-1 Axis Binding Antagonists

In some instances, the anti-TIGIT antagonist antibody (e.g., an anti-TIGIT antagonist antibody as disclosed herein, e.g., tiragolumab) is administered intravenously to the subject having a cancer (e.g., esophageal cancer (e.g., metastatic esophageal cancer)). Alternatively, in some embodiments, the anti-TIGIT antagonist antibody (e.g., an anti-TIGIT antagonist antibody as disclosed herein, e.g., tiragolumab) is administered subcutaneously. In some instances, the PD-1 axis binding antagonist (e.g., anti-PD-L1 antagonist antibody (e.g., atezolizumab)) is administered intravenously. Alternatively, in some embodiments, the PD-1 axis binding antagonist (e.g., anti-PD-L1 antagonist antibody (e.g., atezolizumab)) is administered subcutaneously.

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes). In some instances, the anti-TIGIT antagonist antibody (e.g., an anti-TIGIT antagonist antibody as disclosed herein, e.g., tiragolumab) is administered to the subject or population of subjects by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab) is administered to the subject by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes). In some instances, the PD-1 axis binding antagonist (e.g., anti-PD-L1 antagonist antibody (e.g., atezolizumab)) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

v. Administration Order and Observation Periods

In some instances in which both an anti-TIGIT antagonist antibody and PD-1 axis binding antagonist are administered to a subject or population of subjects having cancer (e.g., esophageal cancer (e.g., metastatic esophageal cancer)), the anti-TIGIT antagonist antibody (e.g., an anti-TIGIT antagonist antibody as disclosed herein, e.g., tiragolumab) is administered to the subject before the PD-1 axis binding antagonist (e.g., anti-PD-L1 antagonist antibody (e.g., atezolizumab)).

In some instances, for example, following administration of the anti-TIGIT antagonist antibody and before administration of the PD-1 axis binding antagonist the method includes an intervening first observation period. In some instances, for example, following administration of the anti-TIGIT antagonist antibody, the PD-1 axis binding antagonist (e.g., anti-PD-L1 antagonist antibody (e.g., atezolizumab)) is administered to the subject. In some instances, the anti-TIGIT antagonist antibody (e.g., an anti-TIGIT antagonist antibody as disclosed herein, e.g., tiragolumab) is first administered to the subject and the PD-1 axis binding antagonist (e.g., anti-PD-L1 antagonist antibody (e.g., atezolizumab)) is administered to the subject following administration of the anti-TIGIT antagonist antibody (e.g., an anti-TIGIT antagonist antibody as disclosed herein, e.g., tiragolumab).

In some instances, the method further includes a second observation period following administration of the PD-1 axis binding antagonist (e.g., anti-PD-L1 antagonist antibody (e.g., atezolizumab)).

In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab)). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody, the PD-1 axis binding antagonist (e.g., anti-PD-L1 antagonist antibody (e.g., atezolizumab)) during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody or the PD-1 axis binding antagonist (e.g., anti-PD-L1 antagonist antibody (e.g., atezolizumab)) during the first or second.

In some instances, the PD-1 axis binding antagonist (e.g., anti-PD-L1 antagonist antibody (e.g., atezolizumab)) is administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab)) and before administration of the anti-TIGIT antagonist antibody (e.g., an anti-TIGIT antagonist antibody as disclosed herein, e.g., tiragolumab), the method includes an intervening first observation period.

In some instances, the method further includes a second observation period following administration of the anti-TIGIT antagonist antibody (e.g., an anti-TIGIT antagonist antibody as disclosed herein, e.g., tiragolumab).

In some instances, the method includes both a first observation period following administration of the PD-1 axis binding antagonist (e.g., anti-PD-L1 antagonist antibody (e.g., atezolizumab)) and second observation period following administration of the anti-TIGIT antagonist antibody (e.g., an anti-TIGIT antagonist antibody as disclosed herein, e.g., tiragolumab). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab)) or the anti-TIGIT antagonist antibody (e.g., an anti-TIGIT antagonist antibody as disclosed herein, e.g., tiragolumab), during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), during the first or second observation periods.

### VII. Assessment of PD-L1 Expression

The expression of PD-L1 may be assessed in a subject treated according to any of the methods and compositions for use described herein. The methods and compositions for use may include determining the expression level of PD-L1 in a biological sample (e.g., a tumor sample) obtained from the subject having a cancer (e.g., esophageal cancer (e.g., metastatic esophageal cancer)). In other examples, the expression level of PD-L1 in a biological sample (e.g., a tumor sample) obtained from the subject has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1 L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the subject has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the subject has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some aspects, the esophageal cancer of a subject treated according to any of the methods provided herein has a PD-L1-positive tumor cell (TC) fraction or tumor-infiltrating immune cell (IC) fraction of <5%. In some aspects, the esophageal cancer has a PD-L1-positive TC fraction of <1%. In other aspects, the esophageal cancer of a subject treated according to any of the methods provided herein has a PD-L1-positive TC fraction or IC fraction of ≥5%. In some aspects, PD-L1 is detected using a Ventana SP142 IHC assay, a Ventana SP263 IHC assay, a pharmDx 22C3 IHC assay, or a pharmDx 28-8 IHC assay.

In some examples, tumor samples may be scored for PD-L1 positivity in tumor-infiltrating immune cells and/or in tumor cells according to the criteria for diagnostic assessment shown in Table 2 and/or Table 3, respectively.

TABLE 2
Tumor-infiltrating immune cell (IC) IHC diagnostic criteria

| PD-L1 Diagnostic Assessment | IC Score |
|---|---|
| Absence of any discernible PD-L1 staining OR | IC0 |
| Presence of discernible PD-L1 staining of any | |
| intensity in tumor-infiltrating immune cells | |
| covering <1% of tumor area occupied by tumor | |
| cells, associated intratumoral stroma, and | |
| contiguous peri-tumoral desmoplastic stroma | |
| Presence of discernible PD-L1 staining of any | IC1 |
| intensity in tumor-infiltrating immune cells | |
| covering ≥1% to <5% of tumor area occupied by | |
| tumor cells, associated intratumoral stroma, and | |
| contiguous peri-tumoral desmoplastic stroma | |
| Presence of discernible PD-L1 staining of any | IC2 |
| intensity in tumor-infiltrating immune cells | |
| covering ≥5% to <10% of tumor area occupied by | |
| tumor cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor | |
| cells, associated intratumoral stroma, and | |
| contiguous peri-tumoral desmoplastic stroma | |

TABLE 3
Tumor cell (TC) IHC diagnostic criteria

| PD-L1 Diagnostic Assessment | TC Score |
|---|---|
| Absence of any discernible PD-L1 staining OR | TC0 |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### VIII. Assessment of TIGIT Expression

The expression level of TIGIT may be assessed in a subject having a cancer (e.g., esophageal cancer (e.g., metastatic esophageal cancer)) who has been treated according to any of the methods, uses, and compositions for use described herein. The methods, uses, and compositions for use may include determining the expression level of TIGIT in a biological sample (e.g., a tumor sample) obtained from the subject. In other examples, the expression level of TIGIT in a biological sample (e.g., a tumor sample) obtained from the subject has been determined prior to initiation of treatment or after initiation of treatment. TIGIT expression may be determined using any suitable approach. Any suitable tumor sample may be used, e.g., a formalin-fixed and paraffin-embedded (FFPE) tumor sample, an archival tumor sample, a fresh tumor sample, or a frozen tumor sample.

For example, TIGIT expression may be determined in terms of the percentage of a tumor sample comprised by tumor-infiltrating immune cells expressing a detectable expression level of TIGIT, as the percentage of tumor-infiltrating immune cells in a tumor sample expressing a detectable expression level of TIGIT, and/or as the percentage of tumor cells in a tumor sample expressing a detectable expression level of TIGIT. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-TIGIT antagonist antibody. Any suitable anti-TIGIT antagonist antibody may be used. In some examples, the anti-TIGIT antagonist antibody is 10A7 (WO 2009/126688A3; U.S. Pat. No. 9,499,596).

### IX. Anti-TIGIT Antagonist Antibodies

The invention provides anti-TIGIT antagonist antibodies useful for treating cancer in a subject (e.g., a human) having a cancer (e.g., esophageal cancer (e.g., metastatic esophageal cancer)).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14), (e) an HVR-L2 comprising the amino acid sequence of WASTRES (SEQ ID NO: 15); and/or (f) an HVR-L3 comprising the amino acid sequence of QQYYSTPFT (SEQ ID NO: 16), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 11-16.

In some instances, anti-TIGIT antagonist antibodies may include (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14); (e) an HVR-L2 comprising the amino acid sequence of WASTRES (SEQ ID NO: 15); and (f) an HVR-L3 comprising the amino acid sequence of QQYYSTPFT (SEQ ID NO: 16). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 27) or an amino acid sequence having at least 90% sequence identity (e.g., at least 91%, 92%, 93%, 94%, 95%, 96%, 97%, 98%, or 99% sequence identity) to, or the sequence of, QVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 28); and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIK (SEQ ID NO: 29). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 27 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 29. In some instances, the anti-TIGIT antagonist antibody has a VH domain comprising the amino acid sequence of SEQ ID NO: 27 and a VL domain comprising the amino acid sequence of SEQ ID NO: 29. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 28 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 29. In some instances, the anti-TIGIT antagonist antibody has a VH domain comprising the amino acid sequence of SEQ ID NO: 28 and a VL domain comprising the amino acid sequence of SEQ ID NO: 29.

In some instances, the anti-TIGIT antagonist antibody includes a heavy chain and a light chain sequence, wherein: (a) the heavy chain comprises the amino acid sequence: EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSSASTKGPSVFPLA PSSKSTSGGTAALGCLVKDYFPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTPPVLDSDGSFFLY SKLTVDKSRWQQGNVFSCSVMHEALHNHYTQKSLSLSPGK (SEQ ID NO: 33); and (b) the light chain comprises the amino acid sequence: DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIKRTVAAPSVFIFPPSDEQLKSGTASVVC LLNNFYPREAKVQWKVDNALQSGNSQESVTEQDSKDSTYSLSSTLTLSKADYEKHKVYACEVTHQGLSS PVTKSFNRGEC (SEQ ID NO: 34).

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 17); an FR-L2 comprising the amino acid sequence of WYQQKPGQPPNLLIY (SEQ ID NO: 18); an FR-L3 comprising the amino acid sequence of GVPDRFSGSGSGTDFTLTISSLQAEDVAVYYC (SEQ ID NO: 19); and/or an FR-L4 comprising the amino acid sequence of FGPGTKVEIK (SEQ ID NO: 20), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 17-20. In some instances, for example, the antibody further comprises an FR-L1 comprising the amino acid sequence of DIVMTQSPDSLAVSLGERATINC (SEQ ID NO: 17); an FR-L2 comprising the amino acid sequence of WYQQKPGQPPNLLIY (SEQ ID NO: 18); an FR-L3 comprising the amino acid sequence of GVPDRFSGSGSGTDFTLTISSLQAEDVAVYYC (SEQ ID NO: 19); and an FR-L4 comprising the amino acid sequence of FGPGTKVEIK (SEQ ID NO: 20).

In some instances, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 21), wherein X₁ is E or Q; an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 21-24. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 25); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 22-25. In some instances, the anti-TIGIT antagonist antibody includes an FR-H1 comprising the amino acid sequence of EVQLQQSGPGLVKPSQTLSLTCAISGDSVS (SEQ ID NO: 25); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and an FR-H4 comprising the amino acid sequence of WGQGTLVTVSS (SEQ ID NO: 24). In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 26); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and/or an FR-H4 comprising the amino acid sequence of WGQGTLVTVSS (SEQ ID NO: 24), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 22-24 and 26. In some instances, the anti-TIGIT antagonist antibody includes an FR-H1 comprising the amino acid sequence of QVQLQQSGPGLVKPSQTLSLTCAISGDSVS (SEQ ID NO: 26); an FR-H2 comprising the amino acid sequence of WIRQSPSRGLEWLG (SEQ ID NO: 22); an FR-H3 comprising the amino acid sequence of RITINPDTSKNQFSLQLNSVTPEDTAVFYCTR (SEQ ID NO: 23); and an FR-H4 comprising the amino acid sequence of WGQGTLVTVSS (SEQ ID NO: 24).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a KD of about 10 nM or lower and cyno TIGIT with a KD of about 10 nM or lower (e.g., binds human TIGIT with a KD of about 0.1 nM to about 1 nM and cyno TIGIT with a KD of about 0.5 nM to about 1 nM, e.g., binds human TIGIT with a KD of about 0.1 nM or lower and cyno TIGIT with a KD of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization.

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 11); (b) an HVR-H2 comprising the amino acid sequence of KTYYRFKWYSDYAVSVKG (SEQ ID NO: 12); (c) an HVR-H3 comprising the amino acid sequence of ESTTYDLLAGPFDY (SEQ ID NO: 13); (d) an HVR-L1 comprising the amino acid sequence of KSSQTVLYSSNNKKYLA (SEQ ID NO: 14), (e) an HVR-L2 comprising the amino acid sequence of WASTRES (SEQ ID NO: 15); and (f) an HVR-L3 comprising the amino acid sequence of QQYYSTPFT (SEQ ID NO: 16). The methods described herein may also include administering an isolated anti-TIGIT antagonist antibody that binds to the same epitope as an anti-TIGIT antagonist antibody described above.

In some aspects, the anti-TIGIT antagonist antibody exhibits Fc-mediated effector function, e.g., participates in antibody-dependent cellular cytotoxicity (ADCC). In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG class antibody. In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308. In some aspects, the antibody is a human monoclonal full-length IgG1 class antibody comprising an Fc region.

In some aspects, the anti-TIGIT antagonist antibody is a human, monoclonal full-length IgG1 subclass antibody comprising a human IgG1 Fc region, a heavy chain variable region (VH) comprising the amino acid sequence of SEQ ID NO: 27, and a light chain variable region (VL) comprising the amino acid sequence of SEQ ID NO: 29.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or RO7092284), vibostolimab (MK-7684), ASP8374 (PTZ-201), EOS884448 (EOS-448), SEA-TGT (SGN-TGT)), BGB-A1217, BMS-986207 (ONO-4686), COM902 (CGEN-15137), IB1939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or RO7092284), vibostolimab (MK-7684), ASP8374 (PTZ-201), EOS-448, and SEA-TGT (SGN-TGT). The anti-TIGIT antibody may be tiragolumab (MTIG7192A, RG6058 or RO7092284).

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, IB1939, EOS884448 (EOS-448), domvanalimab (AB154), vibostolimab (MK-7684), and SEA-TGT (SGN-TGT).

### X. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used for treating a subject having a cancer (e.g., esophageal cancer (e.g., metastatic esophageal cancer)).

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region (VH) comprising the amino acid sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and
(b) the light chain variable region (VL) comprising the amino acid sequence:

(SEQ ID NO: 10)
DIQMTQSPSSLSASVGDRVTITCRASQDVSTAVAWY
QQKPGKAPKLLIYSASFLYSGVPSRFSGSGSGTDFT
LTISSLOPEDFATYYCQQYLYHPATFGQGTKVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYASTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPG,
and
(b) the light chain amino acid sequence:
(SEQ ID NO: 2)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen-binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### XI. Bispecific Antibodies Targeting PD-1 and LAG3

A. Exemplary Bispecific Antibodies that Bind to PD-1 and LAG3

In one aspect, the invention provides a bispecific antibody comprising a first antigen-binding domain that specifically binds to PD-1 and a second antigen-binding domain that specifically binds to LAG3, wherein said first antigen-binding domain specifically binding to PD-1 comprises
a VH domain comprising
(i) HVR-H1 comprising the amino acid sequence of GFSFSSY (SEQ ID NO: 35),
(ii) HVR-H2 comprising the amino acid sequence GGR, and
(iii) HVR-H3 comprising an amino acid sequence of TGRVYFALD (SEQ ID NO: 37); and
a VL domain comprising
(i) HVR-L1 comprising the amino acid sequence of SESVDTSDNSF (SEQ ID NO: 38);
(ii) HVR-L2 comprising the amino acid sequence RSS, and
(iii) HVR-L3 comprising the amino acid sequence of NYDVPW (SEQ ID NO: 40).

In one aspect, the bispecific antibody comprises a Fc domain that is an IgG, particularly an IgG1 Fc domain or an IgG4 Fc domain and wherein the Fc domain has reduced or even abolished effector function. In particular, the Fc domain comprises one or more amino acid substitution that reduces binding to an Fc receptor, in particular towards Fcγ receptor.

In a further aspect, provided is a bispecific antibody comprising a first antigen-binding domain that specifically binds to PD-1 and a second antigen-binding domain that specifically binds to LAG3, wherein the bispecific antibody comprises a Fc domain that is an IgG, particularly an IgG1 Fc domain or an IgG4 Fc domain and wherein the Fc domain comprises one or more amino acid substitution that reduces binding to an Fc receptor, in particular towards Fcγ receptor.

In another aspect, provided is a bispecific antibody comprising a first antigen-binding domain that specifically binds to PD-1 and a second antigen-binding domain that specifically binds to LAG3, wherein the second antigen-binding domain that specifically binds to LAG3 comprises
a VH domain comprising
   (i) HVR-H1 comprising the amino acid sequence of DYTMN (SEQ ID NO: 43),
   (ii) HVR-H2 comprising the amino acid sequence of VISWDGGGTYYTDSVKG (SEQ ID NO: 44), and
   (iii) HVR-H3 comprising an amino acid sequence of GLTDTTLYGSDY (SEQ ID NO: 45); and
   a VL domain comprising
   (i) HVR-L1 comprising the amino acid sequence of RASQSISSYLN (SEQ ID NO:46),
   (ii) HVR-L2 comprising the amino acid sequence of AASTLQS (SEQ ID NO:47), and
   (iii) HVR-L3 comprising the amino acid sequence of QQTYSSPLT (SEQ ID NO:48).

In a further aspect, provided is a bispecific antibody comprising a first antigen-binding domain that specifically binds to PD-1 and a second antigen-binding domain that specifically binds to LAG3, wherein the first antigen-binding domain specifically binding to PD-1 comprises
a VH domain comprising the amino acid sequence of EVQLLESGGGLVQPGGSLRLSCAASGFSFSSYTMSWVRQAPGKGLEWVATISGGGRDIYYPDSV KGRFTISRDNSKNTLYLQMNSLRAEDTAVYYCVLLTGRVYFALDSWGQGTLVTVSS (SEQ ID NO: 41) and a VL domain comprising the amino acid sequence of DIVMTQSPDSLAVSLGERATINCKASESVDTSDNSFIHWYQQKPGQSPKLLIYRSSTLESGVPDRF SGSGSGTDFTLTISSLQAEDVAVYYCQQNYDVPWTFGQGTKVEIK (SEQ ID NO: 42).

In another aspect, provided is a bispecific antibody comprising a first antigen-binding domain that specifically binds to PD-1 and a second antigen-binding domain that specifically binds to LAG3, wherein the second antigen-binding domain specifically binding to LAG3 comprises
a VH domain comprising the amino acid sequence of EVQLLESGGGLVQPGGSLRL SCAASGFIFDDYTMNWVRQAPGKGLEWVAVISWDGGGTYYTDSVKGRFTISRDDFKNTLY LQMNSLRAEDTAVYYCAKGLTDTTLYGSDYWGQGTLVTVSS (SEQ ID NO: 49) and a VL domain comprising the amino acid sequence of DIQMTQSPSSLSASVGDRVTITCRASQSISSYLNWYQQKPGKAPKLLIYAASTLQSGVPSRFSGSG SGTDFTLTISSLQPEDFATYYCQQ TYSSPLTFGGGTKVEIK (SEQ ID NO: 50).

In a particular aspect, provided is a bispecific antibody comprising a first antigen-binding domain that specifically binds to PD-1 and a second antigen-binding domain that specifically binds to LAG3, wherein
the first antigen-binding domain specifically binding to PD-1 comprises a VH domain comprising the amino acid sequence of SEQ ID NO: 41 and a VL domain comprising the amino acid sequence of SEQ ID NO: 42,
and the second antigen-binding domain specifically binding to LAG3 comprises a VH domain comprising the amino acid sequence of SEQ ID NO: 49 and a VL domain comprising the amino acid sequence of SEQ ID NO: 50.

In a further aspect, the bispecific antibody comprising a first antigen-binding domain that specifically binds to PD-1 and a second antigen-binding domain that specifically binds to LAG3 is a human, humanized or chimeric antibody. In particular, it is a humanized or chimeric antibody.

In one aspect, the bispecific antibody comprising a first antigen-binding domain that specifically binds to PD-1 and a second antigen-binding domain that specifically binds to LAG3 is bivalent. This means that the bispecific antibody comprises one antigen-binding domain that specifically binds to PD-1 and one antigen-binding domain that specifically binds to LAG3 (1+1 format).

In one aspect, provided is a bispecific antibody comprising a first antigen-binding domain that specifically binds to PD-1 and a second antigen-binding domain that specifically binds to LAG3, wherein the bispecific antibody comprises an Fc domain, a first Fab fragment comprising the antigen-binding domain that specifically binds to PD-1 and a second Fab fragment comprising the antigen-binding domain that specifically binds to LAG3. In a particular aspect, in one of the Fab fragments the variable domains VL and VH are replaced by each other so that the VH domain is part of the light chain and the VL domain is part of the heavy chain. In a particular aspect, in the first Fab fragment comprising the antigen-binding domain that specifically binds to PD-1 the variable domains VL and VH are replaced by each other.

In a particular aspect, provided is a bispecific antibody comprising a first antigen-binding domain that specifically binds to PD-1 and a second antigen-binding domain that specifically binds to LAG3, wherein the bispecific antibody comprises
a first heavy chain comprising an amino acid sequence with at least 95% sequence identity to the sequence of SEQ ID NO: 51, a first light chain comprising an amino acid sequence with at least 95% sequence identity to the sequence of SEQ ID NO: 52,
   a second heavy chain comprising an amino acid sequence with at least 95% sequence identity to the sequence of SEQ ID NO: 53, and a second light chain comprising an amino acid sequence with at least 95% sequence identity to the sequence of SEQ ID NO:54.

More particularly, the bispecific antibody comprises a first heavy chain comprising an amino acid sequence of SEQ ID NO: 96, a first light chain comprising an amino acid sequence of SEQ ID NO: 98, a second heavy chain comprising an amino acid sequence of SEQ ID NO: 100, and a second light chain comprising an amino acid sequence of SEQ ID NO:101 (RO7247699).

In a further aspect, provided is a bispecific antibody comprising a first antigen-binding domain that specifically binds to PD-1 and a second antigen-binding domain that specifically binds to LAG3, wherein the bispecific antibody comprises an Fc domain, a first Fab fragment comprising the antigen-binding domain that specifically binds to PD-1 and a second Fab fragment comprising the antigen-binding domain that specifically binds to LAG3 that is fused to the C-terminus of the Fc domain. Particularly, the Fab fragment comprising the antigen-binding domain that specifically binds to LAG3 is fused to the C-terminus of the Fc domain via its VH domain (trans 1+1 format).

In a particular aspect, the bispecific antibody comprises a first heavy chain comprising an amino acid sequence with at least 95% sequence identity to the sequence of SEQ ID NO: 51, a first light chain comprising an amino acid sequence with at least 95% sequence identity to the sequence of SEQ ID NO: 52, a second heavy chain comprising an amino acid sequence with at least 95% sequence identity to the sequence of SEQ ID NO: 73, and a second light chain comprising an amino acid sequence with at least 95% sequence identity to the sequence of SEQ ID NO: 54. More particularly, the bispecific antibody comprises a first heavy chain comprising an amino acid sequence of SEQ ID NO: 51, a first light chain comprising an amino acid sequence of SEQ ID NO:52, a second heavy chain comprising an amino acid sequence of SEQ ID NO: 73, and a second light chain comprising an amino acid sequence of SEQ ID NO: 54.

i. Fc Domain Modifications Reducing Fc Receptor Binding and/or Effector Function

In certain aspects, provided is a bispecific antibody comprising a first antigen-binding domain that specifically binds to PD-1 and a second antigen-binding domain that specifically binds to LAG3, wherein the bispecific antibody comprises a Fc domain comprising one or more amino acid modifications that reduce binding to an Fc receptor, in particular towards Fcγ receptor, and reduce or abolish effector function.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

The following section describes preferred aspects of the bispecific antigen binding molecules of the invention comprising Fc domain modifications reducing Fc receptor binding and/or effector function. In one aspect, the invention relates to a bispecific antibody comprising a first antigen-binding domain that specifically binds to PD-1 and a second antigen-binding domain that specifically binds to LAG3, wherein the Fc domain comprises one or more amino acid substitution that reduces binding to an Fc receptor, in particular towards Fcγ receptor. In particular, the Fc domain is of human IgG1 subclass with the amino acid mutations L234A, L235A and P329G (numbering according to Kabat EU index).

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 FC domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain domain exhibits substantially similar binding affinity to neonatal Fc receptor (FcRn), as compared to a native IgG1 Fc domain. Substantially similar binding to FcRn is achieved when the Fc domain (or the the bispecific antigen binding molecule of the invention comprising said Fc domain) exhibits greater than about 70%, particularly greater than about 80%, more particularly greater than about 90% of the binding affinity of a native IgG1 Fc domain (or the the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331 S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such antibody is an IgG1 with mutations L234A and L235A or with mutations L234A, L235A and P329G (numbering according to EU index of Kabat et al., Sequences of Proteins of Immunological Interest, 5th Ed. Public Health Service, National Institutes of Health, Bethesda, M D, 1991).

In one aspect, the bispecific antibody of the invention comprises (all positions according to EU index of Kabat) (i) a homodimeric Fc-region of the human IgG1 subclass optionally with the mutations P329G, L234A and L235A, or (ii) a homodimeric Fc-region of the human IgG4 subclass optionally with the mutations P329G, S228P and L235E, or (iii) a homodimeric Fc-region of the human IgG1 subclass optionally with the mutations P329G, L234A, L235A, I253A, H310A, and H435A, or optionally with the mutations P329G, L234A, L235A, H310A, H433A, and Y436A, or (iv) a heterodimeric Fc-region wherein one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C, or (v) a heterodimeric Fc-region of the human IgG1 subclass wherein both Fc-region polypeptides comprise the mutations P329G, L234A and L235A and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)). Thus, in one aspect, provided is a bispecific antibody, comprising (all positions according to EU index of Kabat) a heterodimeric Fc-region of the human IgG4 subclass wherein both Fc-region polypeptides comprise the mutations P329G, S228P and L235E and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined, e.g., by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

The following section describes preferred aspects of the bispecific antibodies of the invention comprising Fc domain modifications reducing Fc receptor binding and/or effector function. In one aspect, the invention relates to the bispecific comprising a first antigen-binding domain that specifically binds PD-1 and a second antigen-binding domain that specifically binds to LAG3, wherein the Fc domain comprises one or more amino acid substitution that reduces the binding affinity of the antibody to an Fc receptor, in particular towards Fcγ receptor. In another aspect, the invention relates to the bispecific antibody comprising a first antigen-binding domain that specifically binds to PD-1 and a second antigen-binding domain that specifically binds to LAG3, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

ii. Fc Domain Modifications Promoting Heterodimerization

The bispecific antigen binding molecules of the invention comprise different antigen-binding domains, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules of the invention a modification promoting the association of the desired polypeptides.

Accordingly, in particular aspects the invention relates to a bispecific antibody comprising a first antigen-binding domain that specifically binds to PD-1 and a second antigen-binding domain that specifically binds to LAG3, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to a bispecific antibody comprising a first antigen-binding domain that specifically binds to PD-1 and a second antigen-binding site that specifically binds to LAG3, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g., by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues leads to the formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

But also other knobs-in-holes technologies as described by EP 1 870 459, can be used alternatively or additionally. In one embodiment the multispecific antibody comprises the mutations R409D and K370E in the CH3 domain of the “knobs chain” and the mutations D399K and E357K in the CH3 domain of the “hole-chain” (numbering according to Kabat EU index).

In one aspect, the bispecific antibody comprises a T366W mutation in the CH3 domain of the “knobs chain” and the mutations T366S, L368A and Y407V in the CH3 domain of the “hole chain” and additionally the mutations R409D and K370E in the CH3 domain of the “knobs chain” and the mutations D399K and E357K in the CH3 domain of the “hole chain” (numbering according to the Kabat EU index).

In one aspect, the bispecific antibody comprises the mutations Y349C and T366W in one of the two CH3 domains and the mutations S354C, T366S, L368A and Y407V in the other of the two CH3 domains, or the multispecific antibody comprises the mutations Y349C and T366W in one of the two CH3 domains and the mutations S354C, T366S, L368A and Y407V in the other of the two CH3 domains and additionally the mutations R409D and K370E in the CH3 domain of the “knobs chain” and the mutations D399K and E357K in the CH3 domain of the “hole chain” (numbering according to the Kabat EU index).

In an alternative aspect, a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Apart from the “knob-into-hole technology” other techniques for modifying the CH3 domains of the heavy chains of a multispecific antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with a bispecific antibody.

In one aspect, in the bispecific antibody the approach described in EP 1870459 is used to support heterodimerization of the first heavy chain and the second heavy chain of the multispecific antibody. This approach is based on the introduction of charged amino acids with opposite charges at specific amino acid positions in the CH3/CH3-domain-interface between both, the first and the second heavy chain.

Accordingly, in this aspect in the tertiary structure of the multispecific antibody the CH3 domain of the first heavy chain and the CH3 domain of the second heavy chain form an interface that is located between the respective antibody CH3 domains, wherein the respective amino acid sequences of the CH3 domain of the first heavy chain and the amino acid sequence of the CH3 domain of the second heavy chain each comprise a set of amino acids that is located within said interface in the tertiary structure of the antibody, wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The bispecific antibody according to this aspect is herein also referred to as “CH3(+/−)-engineered bispecific antibody” (wherein the abbreviation “+/−” stands for the oppositely charged amino acids that were introduced in the respective CH3 domains).

In one aspect, in the CH3(+/−)-engineered bispecific antibody the positively charged amino acid is selected from K, R and H, and the negatively charged amino acid is selected from E or D.

In one aspect, in the CH3(+/−)-engineered bispecific antibody the positively charged amino acid is selected from K and R, and the negatively charged amino acid is selected from E or D.

In one aspect, in the CH3(+/−)-engineered bispecific antibody the positively charged amino acid is K, and the negatively charged amino acid is E.

In one aspect, in the CH3(+/−)-engineered bispecific antibody in the CH3 domain of one heavy chain the amino acid R at position 409 is substituted by D and the amino acid K at position is substituted by E, and in the CH3 domain of the other heavy chain the amino acid D at position 399 is substituted by K and the amino acid E at position 357 is substituted by K (numbering according to Kabat EU index).

In one aspect, the approach described in WO 2013/157953 is used to support heterodimerization of the first heavy chain and the second heavy chain of the multispecific antibody. In one embodiment in the CH3 domain of one heavy chain the amino acid T at position 366 is substituted by K, and in the CH3 domain of the other heavy chain the amino acid L at position 351 is substituted by D (numbering according to Kabat EU index). In another embodiment in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index).

In another aspect, in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index). Additionally at least one of the following substitutions is comprised in the CH3 domain of the other heavy chain: the amino acid Y at position 349 is substituted by E, the amino acid Y at position 349 is substituted by D and the amino acid L at position 368 is substituted by E (numbering according to Kabat EU index). In one embodiment the amino acid L at position 368 is substituted by E (numbering according to Kabat EU index).

In one aspect, the approach described in WO 2012/058768 is used to support heterodimerization of the first heavy chain and the second heavy chain of the multispecific antibody. In one aspect, in the CH3 domain of one heavy chain the amino acid L at position 351 is substituted by Y and the amino acid Y at position 407 is substituted by A, and in the CH3 domain of the other heavy chain the amino acid T at position 366 is substituted by A and the amino acid K at position 409 is substituted by F (numbering according to Kabat EU index). In another embodiment, in addition to the aforementioned substitutions, in the CH3 domain of the other heavy chain at least one of the amino acids at positions 411 (originally T), 399 (originally D), 400 (originally S), 405 (originally F), 390 (originally N) and 392 (originally K) is substituted (numbering according to Kabat EU index). Preferred substitutions are:
substituting the amino acid T at position 411 by an amino acid selected from N, R, Q, K, D, E and W (numbering according to Kabat EU index),
substituting the amino acid D at position 399 by an amino acid selected from R, W, Y, and K (numbering according to Kabat EU index),
substituting the amino acid S at position 400 by an amino acid selected from E, D, R and K (numbering according to Kabat EU index),
substituting the amino acid F at position 405 by an amino acid selected from I, M, T, S, V and W (numbering according to Kabat EU index;
substituting the amino acid N at position 390 by an amino acid selected from R, K and D (numbering according to Kabat EU index; and
substituting the amino acid K at position 392 by an amino acid selected from V, M, R, L, F and E (numbering according to Kabat EU index).

In another aspect, the bispecific antibody is engineered according to WO 2012/058768), i.e. in the CH3 domain of one heavy chain the amino acid L at position 351 is substituted by Y and the amino acid Y at position 407 is substituted by A, and in the CH3 domain of the other heavy chain the amino acid T at position 366 is substituted by V and the amino acid K at position 409 is substituted by F (numbering according to Kabat EU index). In another embodiment of the multispecific antibody, in the CH3 domain of one heavy chain the amino acid Y at position 407 is substituted by A, and in the CH3 domain of the other heavy chain the amino acid T at position 366 is substituted by A and the amino acid K at position 409 is substituted by F (numbering according to Kabat EU index). In the last aforementioned embodiment, in the CH3 domain of the other heavy chain the amino acid K at position 392 is substituted by E, the amino acid T at position 411 is substituted by E, the amino acid D at position 399 is substituted by R and the amino acid S at position 400 is substituted by R (numbering according to Kabat EU index).

In one aspect, the approach described in WO 2011/143545 is used to support heterodimerization of the first heavy chain and the second heavy chain of the multispecific antibody. In one aspect, amino acid modifications in the CH3 domains of both heavy chains are introduced at positions 368 and/or 409 (numbering according to Kabat EU index).

In one aspect, the approach described in WO 2011/090762 is used to support heterodimerization of the first heavy chain and the second heavy chain of the bispecific antibody. WO 2011/090762 relates to amino acid modifications according to the “knob-into-hole” (KiH) technology. In one embodiment in the CH3 domain of one heavy chain the amino acid T at position 366 is substituted by W, and in the CH3 domain of the other heavy chain the amino acid Y at position 407 is substituted by A (numbering according to Kabat EU index). In another embodiment in the CH3 domain of one heavy chain the amino acid T at position 366 is substituted by Y, and in the CH3 domain of the other heavy chain the amino acid Y at position 407 is substituted by T (numbering according to Kabat EU index).

In one aspect, the approach described in WO 2009/089004 is used to support heterodimerization of the first heavy chain and the second heavy chain of the bispecific antibody. In one embodiment in the CH3 domain of one heavy chain the amino acid K or N at position 392 is substituted by a negatively charged amino acid (in one embodiment by E or D, in one preferred embodiment by D), and in the CH3 domain of the other heavy chain the amino acid D at position 399 the amino acid E or D at position 356 or the amino acid E at position 357 is substituted by a positively charged amino acid (in one embodiment K or R, in one preferred embodiment by K, in one preferred embodiment the amino acids at positions 399 or 356 are substituted by K) (numbering according to Kabat EU index). In one further embodiment, in addition to the aforementioned substitutions, in the CH3 domain of the one heavy chain the amino acid K or R at position 409 is substituted by a negatively charged amino acid (in one embodiment by E or D, in one preferred embodiment by D) (numbering according to Kabat EU index). In one even further aspect, in addition to or alternatively to the aforementioned substitutions, in the CH3 domain of the one heavy chain the amino acid K at position 439 and/or the amino acid K at position 370 is substituted independently from each other by a negatively charged amino acid (in one embodiment by E or D, in one preferred embodiment by D) (numbering according to Kabat EU index).

In one aspect, the approach described in WO 2007/147901 is used to support heterodimerization of the first heavy chain and the second heavy chain of the multispecific antibody. In one embodiment in the CH3 domain of one heavy chain the amino acid K at position 253 is substituted by E, the amino acid D at position 282 is substituted by K and the amino acid K at position 322 is substituted by D, and in the CH3 domain of the other heavy chain the amino acid D at position 239 is substituted by K, the amino acid E at position 240 is substituted by K and the amino acid K at position 292 is substituted by D (numbering according to Kabat EU index).

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG.

In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

iii. Modifications in the Fab Domains

In one aspect, the invention relates to a bispecific antibody comprising a first Fab fragment that specifically binds to PD-1 and a second Fab fragment that specifically binds to LAG3, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252, WO2009/080253 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In a particular aspect, the invention relates to a bispecific antibody comprising a first Fab fragment that specifically binds to PD-1 and a second Fab fragment that specifically binds to LAG3, wherein in one of the Fab fragments the variable domains VL and VH are replaced by each other so that the VH domain is part of the light chain and the VL domain is part of the heavy chain. In a particular aspect, the bispecific antibody is one, wherein in the first Fab fragment comprising the antigen-binding domain that specifically binds to PD-1 the variable domains VL and VH are replaced by each other.

In another aspect, and to further improve correct pairing, the bispecific antibody comprising a first Fab fragment that specifically binds to PD-1 and a second Fab fragment that specifically binds to LAG3, can contain different charged amino acid substitutions (so-called “charged residues”). These modifications are introduced in the crossed or non-crossed CH1 and CL domains. Such modifications are described e.g., in WO2015/150447, WO2016/020309 and PCT/EP2016/073408.

In a particular aspect, the invention is concerned with a bispecific antibody comprising a first Fab fragment that specifically binds to PD-1 and a second Fab fragment that specifically binds to LAG3, wherein in one of the Fab fragments in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index). In a particular aspect, the bispecific antibody is one, wherein in the second Fab fragment comprising the antigen-binding domain that specifically binds to TIM3 the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In a particular aspect, the invention relates to a bispecific antibody comprising a first Fab fragment that specifically binds to PD-1 and a second Fab fragment that specifically binds to LAG3, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In a particular aspect, the bispecific antibody is one, wherein in the second Fab fragment comprising the antigen-binding domain that specifically binds to LAG3 the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

In a further aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

The antibody under a) does not contain a modification as reported under b) and the heavy chain and the light chain under a) are isolated chains.

In the antibody under b) within the light chain the variable light chain domain VL is replaced by the variable heavy chain domain VH of said antibody, and within the heavy chain the variable heavy chain domain VH is replaced by the variable light chain domain VL of said antibody.

In one aspect, (i) in the constant domain CL of the first light chain under a) the amino acid at position 124 (numbering according to Kabat) is substituted by a positively charged amino acid, and wherein in the constant domain CH1 of the first heavy chain under a) the amino acid at position 147 or the amino acid at position 213 (numbering according to Kabat EU index) is substituted by a negatively charged amino acid, or (ii) in the constant domain CL of the second light chain under b) the amino acid at position 124 (numbering according to Kabat) is substituted by a positively charged amino acid, and wherein in the constant domain CH1 of the second heavy chain under b) the amino acid at position 147 or the amino acid at position 213 (numbering according to Kabat EU index) is substituted by a negatively charged amino acid.

In another aspect, (i) in the constant domain CL of the first light chain under a) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the first heavy chain under a) the amino acid at position 147 or the amino acid at position 213 is substituted independently by glutamic acid (E) or aspartic acid (D) (numbering according to Kabat EU index), or (ii) in the constant domain CL of the second light chain under b) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the second heavy chain under b) the amino acid at position 147 or the amino acid at position 213 is substituted independently by glutamic acid (E) or aspartic acid (D) (numbering according to Kabat EU index).

In one aspect, in the constant domain CL of the second heavy chain the amino acids at position 124 and 123 are substituted by K (numbering according to Kabat EU index).

In one aspect, in the constant domain CL of the second heavy chain the amino acid at position 123 is substituted by R and the amino acid as position 124 is substituted by K (numbering according to Kabat EU index).

In one aspect, in the constant domain CH1 of the second light chain the amino acids at position 147 and 213 are substituted by E (numbering according to EU index of Kabat).

In one aspect, in the constant domain CL of the first light chain the amino acids at position 124 and 123 are substituted by K, and in the constant domain CH1 of the first heavy chain the amino acids at position 147 and 213 are substituted by E (numbering according to Kabat EU index).

In one aspect, in the constant domain CL of the first light chain the amino acid at position 123 is substituted by R and the amino acid at position 124 is substituted by K, and in the constant domain CH1 of the first heavy chain the amino acids at position 147 and 213 are both substituted by E (numbering according to Kabat EU index).

In one aspect, in the constant domain CL of the second heavy chain the amino acids at position 124 and 123 are substituted by K, and wherein in the constant domain CH1 of the second light chain the amino acids at position 147 and 213 are substituted by E, and in the variable domain VL of the first light chain the amino acid at position 38 is substituted by K, in the variable domain VH of the first heavy chain the amino acid at position 39 is substituted by E, in the variable domain VL of the second heavy chain the amino acid at position 38 is substituted by K, and in the variable domain VH of the second light chain the amino acid at position 39 is substituted by E (numbering according to Kabat EU index).

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other.

The antibody under a) does not contain a modification as reported under b) and the heavy chain and the light chain und a) are isolated chains. In the antibody under b) within the light chain the variable light chain domain VL is replaced by the variable heavy chain domain VH of said antibody, and the constant light chain domain CL is replaced by the constant heavy chain domain CH1 of said antibody; and within the heavy chain the variable heavy chain domain VH is replaced by the variable light chain domain VL of said antibody, and the constant heavy chain domain CH1 is replaced by the constant light chain domain CL of said antibody.

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

The antibody under a) does not contain a modification as reported under b) and the heavy chain and the light chain under a) are isolated chains. In the antibody under b) within the light chain the constant light chain domain CL is replaced by the constant heavy chain domain CH1 of said antibody; and within the heavy chain the constant heavy chain domain CH1 is replaced by the constant light chain domain CL of said antibody.

In one aspect, the bispecific antibody is a bispecific antibody comprising
a) a full-length antibody specifically binding to a first antigen and consisting of two antibody heavy chains and two antibody light chains, and
b) one, two, three or four single chain Fab fragments specifically binding to a second antigen,
wherein said single chain Fab fragments under b) are fused to said full-length antibody under a) via a peptide linker at the C- or N-terminus of the heavy or light chain of said full length antibody.

In one aspect, one or two identical single chain Fab fragments binding to a second antigen are fused to the full-length antibody via a peptide linker at the C terminus of the heavy or light chains of said full-length antibody.

In one aspect, one or two identical single chain Fab (scFab) fragments binding to a second antigen are fused to the full-length antibody via a peptide linker at the C terminus of the heavy chains of said full-length antibody.

In one aspect, one or two identical single chain Fab (scFab) fragments binding to a second antigen are fused to the full-length antibody via a peptide linker at the C terminus of the light chains of said full-length antibody.

In one aspect, two identical single chain Fab (scFab) fragments binding to a second antigen are fused to the full-length antibody via a peptide linker at the C-terminus of each heavy or light chain of said full-length antibody.

In one aspect, two identical single chain Fab (scFab) fragments binding to a second antigen are fused to the full-length antibody via a peptide linker at the C-terminus of each heavy chain of said full-length antibody.

In one aspect, two identical single chain Fab (scFab) fragments binding to a second antigen are fused to the full-length antibody via a peptide linker at the C-terminus of each light chain of said full-length antibody.

In one aspect, the bispecific antibody is a trivalent antibody comprising
a) a full-length antibody specifically binding to a first antigen and consisting of two antibody heavy chains and two antibody light chains,
b) a first polypeptide consisting of
   ba) an antibody heavy chain variable domain (VH), or
   bb) an antibody heavy chain variable domain (VH) and an antibody constant domain 1 (CH1),
wherein said first polypeptide is fused with the N-terminus of its VH domain via a peptidic linker to the C-terminus of one of the two heavy chains of said full-length antibody,
c) a second polypeptide consisting of
   ca) an antibody light chain variable domain (VL), or
   cb) an antibody light chain variable domain (VL) and an antibody light chain constant domain (CL),
wherein said second polypeptide is fused with the N-terminus of the VL domain via a peptide linker to the C-terminus of the other of the two heavy chains of said full-length antibody, and
wherein the antibody heavy chain variable domain (VH) of the first polypeptide and the antibody light chain variable domain (VL) of the second polypeptide together form an antigen-binding domain specifically binding to a second antigen.

In one aspect, the antibody heavy chain variable domain (VH) of the polypeptide under b) and the antibody light chain variable domain (VL) of the polypeptide under c) are linked and stabilized via an interchain disulfide bridge by introduction of a disulfide bond between the following positions:
(i) heavy chain variable domain position 44 to light chain variable domain position 100, or
(ii) heavy chain variable domain position 105 to light chain variable domain position 43, or
(iii) heavy chain variable domain position 101 to light chain variable domain position 100 (numbering always according to Kabat EU index).

Techniques to introduce unnatural disulfide bridges for stabilization are described e.g. in WO 94/029350, Rajagopal, V., et al., Prot. Eng. (1997) 1453-1459; Kobayashi, H., et al., Nucl. Med. Biol. 25 (1998) 387-393; and Schmidt, M., et al., Oncogene 18 (1999) 1711-1721. In one embodiment the optional disulfide bond between the variable domains of the polypeptides under b) and c) is between heavy chain variable domain position 44 and light chain variable domain position 100. In one embodiment the optional disulfide bond between the variable domains of the polypeptides under b) and c) is between heavy chain variable domain position 105 and light chain variable domain position 43 (numbering always according to Kabat). In one embodiment a trivalent, bispecific antibody without said optional disulfide stabilization between the variable domains VH and VL of the single chain Fab fragments is preferred.

In one aspect, the bispecific antibody is a trispecific or tetraspecific antibody, comprising
a) a first light chain and a first heavy chain of a full-length antibody which specifically binds to a first antigen, and
b) a second (modified) light chain and a second (modified) heavy chain of a full-length antibody which specifically binds to a second antigen, wherein the variable domains VL and VH are replaced by each other, and/or wherein the constant domains CL and CH1 are replaced by each other, and
c) wherein one to four antigen-binding domains which specifically bind to one or two further antigens (i.e. to a third and/or fourth antigen) are fused via a peptide linker to the C- or N-terminus of the light chains or heavy chains of a) and/or b).

The antibody under a) does not contain a modification as reported under b) and the heavy chain and the light chain und a) are isolated chains.

In one aspect, the trispecific or tetraspecific antibody comprises under c) one or two antigen-binding domains which specifically bind to one or two further antigens.

In one aspect, the antigen-binding domains are selected from the group of a scFv fragment and a scFab fragment.

In one aspect, the antigen-binding domains are scFv fragments.

In one aspect, the antigen-binding domains are scFab fragments.

In one aspect, the antigen-binding domains are fused to the C-terminus of the heavy chains of a) and/or b).

In one aspect, the trispecific or tetraspecific antibody comprises under c) one or two antigen-binding domains which specifically bind to one further antigen.

In one aspect, the trispecific or tetraspecific antibody comprises under c) two identical antigen-binding domains which specifically bind to a third antigen. In one preferred embodiment such two identical antigen-binding domains are fused both via the same peptidic linker to the C-terminus of the heavy chains of a) and b). In one preferred embodiment the two identical antigen-binding domains are either a scFv fragment or a scFab fragment.

In one aspect, the trispecific or tetraspecific antibody comprises under c) two antigen-binding domains which specifically bind to a third and a fourth antigen. In one embodiment said two antigen-binding domains are fused both via the same peptide connector to the C-terminus of the heavy chains of a) and b). In one preferred embodiment said two antigen-binding domains are either a scFv fragment or a scFab fragment.

In one aspect, the bispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a), and
wherein in the Fab fragments the following modifications were performed
(i) in both Fab fragments of a), or in both Fab fragments of b), the variable domains VL and VH are replaced by each other, and/or the constant domains CL and CH1 are replaced by each other, or
(ii) in both Fab fragments of a) the variable domains VL and VH are replaced by each other, and the constant domains CL and CH1 are replaced by each other, and in both Fab fragments of b) the variable domains VL and VH are replaced by each other, or the constant domains CL and CH1 are replaced by each other, or
(iii) in both Fab fragments of a) the variable domains VL and VH are replaced by each other, or the constant domains CL and CH1 are replaced by each other, and in both Fab fragments of b) the variable domains VL and VH are replaced by each other, and the constant domains CL and CH1 are replaced by each other, or
(iv) in both Fab fragments of a) the variable domains VL and VH are replaced by each other, and in both Fab fragments of b) the constant domains CL and CH1 are replaced by each other, or
(v) in both Fab fragments of a) the constant domains CL and CH1 are replaced by each other, and in both Fab fragments of b) the variable domains VL and VH are replaced by each other.

In one aspect, said additional Fab fragments are fused both via a peptidic linker either to the C-termini of the heavy chains of a), or to the N-termini of the heavy chains of a).

In one aspect, said additional Fab fragments are fused both via a peptidic linker either to the C-termini of the heavy chains of a).

In one aspect, said additional Fab fragments are fused both via a peptide linker to the N-termini of the heavy chains of a).

In one aspect, in the Fab fragments the following modifications are performed: in both Fab fragments of a), or in both Fab fragments of b), the variable domains VL and VH are replaced by each other, and/or the constant domains CL and CH1 are replaced by each other.

In one aspect, the bispecific antibody is a tetravalent antibody comprising:
a) a (modified) heavy chain of a first antibody, which specifically binds to a first antigen and comprises a first VH-CH1 domain pair, wherein to the C terminus of said heavy chain the N-terminus of a second VH-CH1 domain pair of said first antibody is fused via a peptide linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptide linker, and
d) two (modified) light chains of said second antibody of c), each comprising a CL-CH1 domain pair.

In one aspect, the bispecific antibody comprises
a) the heavy chain and the light chain of a first full-length antibody that specifically binds to a first antigen, and
b) the heavy chain and the light chain of a second full-length antibody that specifically binds to a second antigen, wherein the N-terminus of the heavy chain is connected to the C-terminus of the light chain via a peptide linker.

The antibody under a) does not contain a modification as reported under b) and the heavy chain and the light chain are isolated chains.

In one aspect, the bispecific antibody comprises
a) a full-length antibody specifically binding to a first antigen and consisting of two antibody heavy chains and two antibody light chains, and
b) an Fv fragment specifically binding to a second antigen comprising a VH2 domain and a VL2 domain, wherein both domains are connected to each other via a disulfide bridge,
wherein only either the VH2 domain or the VL2 domain is fused via a peptide linker to the heavy or light chain of the full-length antibody specifically binding to a first antigen.

In the bispecific antibody the heavy chains and the light chains under a) are isolated chains.

In one aspect, the other of the VH2 domain or the VL2 domain is not fused via a peptide linker to the heavy or light chain of the full-length antibody specifically binding to a first antigen.

In all aspects as reported herein the first light chain comprises a VL domain and a CL domain and the first heavy chain comprises a VH domain, a CH1 domain, a hinge region, a CH2 domain and a CH3 domain.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domains of the two Fab fragments are connected to the N-terminus of the heavy chain Fc-region polypeptides, and wherein the C-terminus of the CL domain of the CrossFab fragment is connected to the N-terminus of the VH domain of one of the Fab fragments.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domain of the first Fab fragment is connected to the N-terminus of one of the heavy chain Fc-region polypeptides and the C-terminus of the CL-domain of the CrossFab fragment is connected to the N-terminus of the other heavy chain Fc-region polypeptide, and wherein the C-terminus of the CH1 domain of the second Fab fragment is connected to the N-terminus of the VH domain of the first Fab fragment or to the N-terminus of the VH domain of the CrossFab fragment.

In one aspect, the bispecific antibody comprises
a) a full-length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody
wherein the heavy chain Fab fragment is inserted between the CH1 domain of one of the heavy chains of the full-length antibody and the respective Fc-region of the full-length antibody, and the N-terminus of the light chain Fab fragment is conjugated to the C-terminus of the light chain of the full-length antibody that is paired with the heavy chain of the full-length antibody into which the heavy chain Fab fragment has been inserted.

In one aspect, the bispecific antibody comprises
a) a full-length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody and wherein the C-terminus of the heavy chain fragment of the Fab fragment is conjugated to the N-terminus of one of the heavy chains of the full-length antibody and the C-terminus of the light chain fragment of the Fab fragment is conjugated to the N-terminus of the light chain of the full-length antibody that pairs with the heavy chain of the full-length antibody to which the heavy chain fragment of the Fab fragment is conjugated.
   B. Dosing of Bispecific Antibodies that Bind to PD-1 and LAG3

For the prevention or treatment of disease, the appropriate dosage of a bispecific antibodies comprising a first antigen-binding domain that specifically binds to PD-1 and a second antigen-binding domain that specifically binds to LAG3 of the invention (when used alone or in combination with one or more other additional therapeutic agents) will depend on the type of disease to be treated, the route of administration, the body weight of the subject, the type of fusion protein, the severity and course of the disease, whether the bispecific antibody is administered for preventive or therapeutic purposes, previous or concurrent therapeutic interventions, the subject's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The bispecific antibody comprising a first antigen-binding domain that specifically binds to PD-1 and a second antigen-binding domain that specifically binds to LAG3 as defined herein is suitably administered to the subject at one time or over a series of treatments. Depending on the type and severity of the disease, about 1 μg/kg to 15 mg/kg (e.g., 0.1 mg/kg-10 mg/kg) of the bispecific antibody can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antibody would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

### XII. VEGF Antagonists

Provided herein are methods for treating CRC (e.g., metastatic CRC, e.g., MSI-H metastatic CRC) in a subject comprising administering to the subject a treatment regimen comprising an anti-TIGIT antagonist antibody (e.g., tiragolumab), a PD-1 axis binding antagonist (e.g., atezolizumab), and a VEGF antagonist (e.g., bevacizumab). Also provided are related compositions (e.g., pharmaceutical compositions) for use, kits, and articles of manufacture. Any of the methods, compositions for use, kits, or articles of manufacture described herein may include or involve any of the agents described below.

VEGF antagonists include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879, issued Feb. 26, 2005, the entire disclosure of which is expressly incorporated herein by reference.

Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinib, cediranib, motesanib, sulfatinib, apatinib, foretinib, famitinib, or tivozanib). In some examples, the VEGF antagonist may be a tyrosine kinase inhibitor, including a receptor tyrosine kinase inhibitors (e.g., a multi-targeted receptor tyrosine kinase inhibitor such as sunitinib or axitinib).

### XIII. Tocilizumab for Treatment of Cytokine Release Syndrome (CRS)

### A. Background

Bispecific antibody therapeutics involving T-cell activation have been associated with cytokine release syndrome (CRS). CRS is a potentially life-threatening symptom complex caused by the excessive release of cytokines by immune effector or target cells during an exaggerated and sustained immune response.

CRS is associated with high IL-6 levels (Panelli et al., J Transl Med, 2: 17, 2004; Lee et al., Blood, 124:188-195, 2014; Doessegger and Banholzer, Clin Transl Immunology, 4: e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434:1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Consequently, tocilizumab premedication may also reduce the frequency or lower the severity of CRS associated with bispecific antibody therapy. Other anti-IL-6R antibodies that could be used in combination with tocilizumab include sarilumab, vobarilizumab (ALX-0061), SA-237, and variants thereof.

### B. CRS Symptoms and Grading

The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biology of Blood and Marrow Transplantation. 25(4): 625-638, 2019). The ASTCT criteria is intended to be objective, easy to apply, and more accurately categorize the severity of CRS. This revised CRS grading system is shown below in Table 4. In addition to diagnostic criteria, recommendations on management of CRS based on its severity, including early intervention with corticosteroids and/or anti-cytokine therapy, are provided and referenced in Table 4.

TABLE 4
CRS Grading System

| CRS | | | | |
|---|---|---|---|---|
| Parameter | Grade 1 | Grade 2 | Grade 3 | Grade 4 |
| Fever | Temperature ≥38° C. | Temperature ≥38° C. | Temperature ≥38° C. | Temperature ≥38° C. |
| | | with | | |
| Hypotension | None | Not requiring | Requiring a | Requiring multiple |
| | | vasopressors | vasopressor with or | vasopressors |
| | | | without vasopressin | (excluding vasopressin) |
| | | and/or | | |
| Hypoxia | None | Requiring low-glow | Requiring high-flow | Requiring positive |
| | | nasal cannula or | nasal cannula, facemask, | pressure (e.g., CPAP, |
| | | blow-by | nonrebreather mask | BiPAP, intubation and |
| | | | or Venturi mask | mechanical ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events. Grade 5 = death.
ASTCT consensus grading: Lee et al., Biol Blood Marrow Transplant, 25(4): 625-638, 2019.

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS).

### C. Tocilizumab as a Premedication

In some aspects, an effective amount of tocilizumab is administered as a premedication, e.g., is administered to the subject prior to the administration of the bispecific antibody. Administration of tocilizumab as a premedication may reduce the frequency or severity of CRS. In some aspects, tocilizumab is administered as a premedication in Cycle 1, e.g., is administered prior to a first dose (C1D1), a second dose (C1D2), and/or a third dose (C1D3) of the bispecific antibody. In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

For example, in one aspect, the bispecific antibody is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In some aspects, the disclosure features the use of a bispecific antibody of the invention in the manufacture of a medicament for the treatment of a subject having a relapsed or refractory non-Hodgkin's lymphoma (NHL) ((e.g., a B cell proliferative disorder, e.g., an NHL (e.g., an aggressive NHL or an R/R NHL; e.g., an R/R DLBCL, an R/R FL (e.g., an R/R Grade 3b FL), or an R/R high grade B cell lymphoma (HGBL) or an R/R transformed FL (trFL)) in combination with one or more additional therapeutic agents (e.g., tocilizumab).

In some aspects, the bispecific antibody and the one or more additional therapeutic agents are formulated separately. In some aspects, the bispecific antibody is to be administered to the subject prior to the one or more additional therapeutic agents. In other aspects, the bispecific antibody is to be administered to the subject subsequent to the one or more additional therapeutic agents, e.g., administered to the subject subsequent to administration of an effective amount of tocilizumab.

In some aspects, the bispecific antibody and the one or more additional therapeutic agents are formulated together.

In some aspects, the disclosure features a bispecific antibody of the invention for use in treating a subject having a relapsed or refractory non-Hodgkin's lymphoma (NHL) ((e.g., a B cell proliferative disorder, e.g., an NHL (e.g., an aggressive NHL or an R/R NHL; e.g., an R/R DLBCL, an R/R FL (e.g., an R/R Grade 3b FL), or an R/R high grade B cell lymphoma (HGBL) or an R/R transformed FL (trFL)) in combination with one or more additional therapeutic agents.

In some aspects, the bispecific antibody and the one or more additional therapeutic agents are formulated separately. In some aspects, the bispecific antibody is to be administered to the subject prior to the one or more additional therapeutic agents. In other aspects, the bispecific antibody is to be administered to the subject subsequent to the one or more additional therapeutic agents, e.g., administered to the subject subsequent to administration of an effective amount of tocilizumab.

In some aspects, the bispecific antibody and the one or more additional therapeutic agents are formulated together.

### D. Tocilizumab to Treat CRS

In some aspects, the subject experiences a CRS event during treatment with the therapeutic bispecific antibody and an effective amount of tocilizumab is administered to manage the CRS event.

In some aspects, the subject has a CRS event (e.g., has a CRS event following treatment with the bispecific antibody, e.g., has a CRS event following a first dose or a subsequent dose of the bispecific antibody), and the method further includes treating the symptoms of the CRS event while suspending treatment with the bispecific antibody.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment with the bispecific antibody. In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In some aspects, the corticosteroid is methylprednisone (methylprednisolone). In some instances, the methylprednisone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Tables 4-6. Tables 6 and 7 provide details about tocilizumab treatment of severe or life-threatening CRS.

In some aspects, the disclosure features the use of tocilizumab in the manufacture of a medicament for the treatment of a subject having a CRS event, wherein the CRS event arises during treatment of the subject with the bispecific antibody of the invention.

In some aspects, the medicament is to be administered to the subject while treatment with the bispecific antibody of the invention is suspended.

In some aspects, the medicament is formulated for intravenous administration of tocilizumab as a single dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the CRS event does not resolve or worsens within 24 hours of treating the symptoms of the CRS event and one or more additional doses of tocilizumab are to be administered to the subject. The one or more additional doses of tocilizumab may be to be administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the medicament is for use in combination with an effective amount of a corticosteroid to treat the CRS event. Tocilizumab and the corticosteroid may be formulated separately.

In some aspects, the corticosteroid is to be administered intravenously to the subject. In some aspects, the corticosteroid is methylprednisolone, e.g., methylprednisolone is to be administered to the subject at a dose of about 1 mg/kg per day to about 5 mg/kg per day, e.g., about 2 mg/kg per day. In some aspects, the corticosteroid is dexamethasone, e.g., dexamethasone is to be administered to the subject at a dose of about 10 mg or at a dose of about 0.5 mg/kg per day.

In some aspects, the disclosure features tocilizumab for use in treating a subject having a CRS event, wherein the CRS event arises during treatment of the subject with a bispecific antibody of the invention.

In some aspects, tocilizumab is to be administered to the subject while treatment with the bispecific antibody of the invention is suspended.

In some aspects, tocilizumab is formulated for intravenous administration as a single dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, the CRS event does not resolve or worsens within 24 hours of treating the symptoms of the CRS event and one or more additional doses of tocilizumab are to be administered to the subject. The one or more additional doses of tocilizumab may be to be administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

In some aspects, tocilizumab is for use in combination with an effective amount of a corticosteroid to treat the CRS event. Tocilizumab and the corticosteroid may be formulated separately.

In some aspects, the corticosteroid is to be administered intravenously to the subject. In some aspects, the corticosteroid is methylprednisolone, e.g., methylprednisolone is to be administered to the subject at a dose of about 1 mg/kg per day to about 5 mg/kg per day, e.g., about 2 mg/kg per day. In some aspects, the corticosteroid is dexamethasone, e.g., dexamethasone is to be administered to the subject at a dose of about 10 mg or at a dose of about 0.5 mg/kg per day.

### XIV. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. Further provided herein are pharmaceutical compositions and formulations comprising an anti-TIGIT antagonist antibody (e.g., tiragolumab) and, optionally, a pharmaceutically acceptable carrier. Further provided herein are pharmaceutical compositions and formulations comprising a bispecific antibody targeting PD-1 and LAG3 and, optionally, a pharmaceutically acceptable carrier. The disclosure also provides: (i) pharmaceutical compositions and formulations comprising a PD-1 axis binding antagonist (e.g., atezolizumab) and an anti-TIGIT antagonist antibody, and optionally, a pharmaceutically acceptable carrier; (ii) pharmaceutical compositions and formulations comprising a PD-1 axis binding antagonist (e.g., atezolizumab), an anti-TIGIT antagonist antibody (e.g., tiragolumab), and an anti-VEGF antibody (e.g., bevacizumab), and optionally, a pharmaceutically acceptable carrier; and (iii) pharmaceutical compositions and formulations comprising an anti-TIGIT antagonist antibody and a bispecific antibody targeting PD-1 and LAG3 and optionally, a pharmaceutically acceptable carrier. The disclosure also provides pharmaceutical compositions and formulations comprising mosunetuzumab, an anti-TIGIT antagonist antibody (e.g., tiragolumab), and/or a PD-1 axis binding antagonist (e.g., atezolizumab). Pharmaceutical compositions and formulations of mosunetuzumab, tiragolumab, and atezolizumab, and/or other agents describe herein (e.g., dexamethasone) can be prepared by mixing one, two, three, or all four agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, mosunetuzumab is formulated for administration subcutaneously. In some embodiments, mosunetuzumab is formulated for administration intravenously.

Pharmaceutical compositions and formulations as described herein can be prepared by mixing the active ingredients (e.g., a PD-1 axis binding antagonist, an anti-TIGIT antagonist antibody, and/or a bispecific antibody targeting PD-1 and LAG3; a PD-1 axis binding antagonist, an anti-TIGIT antagonist antibody, and an anti-VEGF antibody; or mosunetuzumab, an anti-TIGIT antagonist antibody, and/or a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary tiragolumab formulation comprises a histidine solution containing polysorbate 20, sucrose, L-methionine, and WFI. Tiragolumab may be provided in a 15-mL vial containing 10 mL of tiragolumab drug product at an approximate concentration of tiragolumab antibody of 60 mg/mL.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### XV. Articles of Manufacture or Kits

### A. Kits Comprising a PD-1 Axis Binding Antagonist and an Anti-TIGIT Antagonist Antibody

In another aspect, provided herein is an article of manufacture or a kit comprising a PD-1 axis binding antagonist (e.g., atezolizumab) and an anti-TIGIT antagonist antibody (e.g., tiragolumab). In some instances, the article of manufacture or kit further comprises a package insert comprising instructions for using the anti-TIGIT antagonist antibody in combination with the PD-1 axis binding antagonist to treat or delay progression of cancer (e.g., esophageal cancer (e.g., metastatic esophageal cancer) or colorectal cancer (CRC) (e.g., metastatic CRC (e.g., microsatellite instability-high (MSI-H) metastatic CRC))) or melanoma in a subject. In some instances, the article of manufacture or kit further comprises package insert comprising instructions for using the anti-TIGIT antagonist antibody and the PD-1 axis binding antagonist to treat or delay progression of cancer (e.g., esophageal cancer (e.g., metastatic esophageal cancer) or CRC (e.g., metastatic CRC (e.g., MSI-H metastatic CRC))) or melanoma in a subject. Any of the PD-1 axis binding antagonists and/or anti-TIGIT antagonist antibodies described herein may be included in the article of manufacture or kits.

In another embodiment of the invention, a kit is provided comprising a PD-1 axis binding antagonist for use in combination with an anti-TIGIT antagonist antibody for treating a subject having a cancer according to any of the methods described herein. In some instances, the kit further comprises the anti-TIGIT antagonist antibody. In some instances, the article of manufacture or kit further comprises a package insert comprising instructions for using the PD-1 axis binding antagonist in combination with anti-TIGIT antagonist antibody (e.g., tiragolumab) to treat or delay progression of a cancer in a subject.

In another embodiment, a kit comprises tiragolumab for use in combination with atezolizumab for treating a subject having a cancer according to any of the methods described herein. In some embodiments, the kit further comprises atezolizumab. In some instances, the article of manufacture or kit further comprises package insert comprising instructions for using tiragolumab in combination with atezolizumab to treat or delay progression of a cancer in a subject.

In another embodiment, a kit comprises atezolizumab for use in combination with tiragolumab for treating a subject having a cancer according to any of the methods described herein. In some embodiments, the kit further comprises tiragolumab. In some instances, the article of manufacture or kit further comprises package insert comprising instructions for using atezolizumab in combination with tiragolumab to treat or delay progression of cancer in a subject.

In some instances, the PD-1 axis binding antagonist and the anti-TIGIT antagonist antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the PD-1 axis binding antagonists and/or anti-TIGIT antagonist antibodies described herein may be included in the article of manufacture or kits. Any of the articles of manufacture or kits may include instructions to administer a PD-1 axis binding antagonist and/or an anti-TIGIT antagonist antibody to a subject in accordance with any of the methods described herein, e.g., any of the methods set forth in Section II, III, or V above.

### B. Kits Comprising a Bispecific Antibody Targeting PD-1 and LAG and an Anti-TIGIT Antagonist Antibody

In another aspect, provided herein is an article of manufacture or a kit comprising a PD-1 axis binding antagonist (e.g., atezolizumab) and a bispecific antibody targeting PD-1 and LAG3. In some instances, the article of manufacture or kit further comprises a package insert comprising instructions for using the anti-TIGIT antagonist antibody in combination with the bispecific antibody targeting PD-1 and LAG3 to treat or delay progression of cancer (e.g., esophageal cancer (e.g., metastatic esophageal cancer)) or melanoma in a subject. In some instances, the article of manufacture or kit further comprises package insert comprising instructions for using the anti-TIGIT antagonist antibody and the bispecific antibody targeting PD-1 and LAG3 to treat or delay progression of cancer (e.g., esophageal cancer (e.g., metastatic esophageal cancer)) or melanoma in a subject. Any of the bispecific antibody targeting PD-1 and LAG3 and/or anti-TIGIT antagonist antibodies described herein may be included in the article of manufacture or kits.

In another embodiment of the invention, a kit is provided comprising a bispecific antibody targeting PD-1 and LAG3 for use in combination with an anti-TIGIT antagonist antibody for treating a subject having a cancer according to any of the methods described herein. In some instances, the kit further comprises the anti-TIGIT antagonist antibody. In some instances, the article of manufacture or kit further comprises a package insert comprising instructions for using the bispecific antibody targeting PD-1 and LAG3 in combination with anti-TIGIT antagonist antibody (e.g., tiragolumab) to treat or delay progression of a cancer in a subject.

In some instances, the bispecific antibody targeting PD-1 and LAG3 and the anti-TIGIT antagonist antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the bispecific antibodies targeting PD-1 and LAG3 and/or anti-TIGIT antagonist antibodies described herein may be included in the article of manufacture or kits. Any of the articles of manufacture or kits may include instructions to administer a bispecific antibody targeting PD-1 and LAG3 and/or an anti-TIGIT antagonist antibody to a subject in accordance with any of the methods described herein, e.g., any of the methods set forth in Section III above.

C. Kits Comprising Mosunetuzumab and an Anti-TIGIT Antagonist Antibody, and/or a PD-1 Axis Binding Antagonist

In another aspect, provided herein is an article of manufacture or a kit comprising mosunetuzumab, an anti-TIGIT antagonist antibody (e.g., tiragolumab), and optionally a PD-1 axis binding antagonist (e.g., atezolizumab). In some instances, the article of manufacture or kit further comprises a package insert comprising instructions for using mosunetuzumab in combination with the anti-TIGIT antagonist antibody (e.g., tiragolumab), and optionally in combination with the PD-1 axis binding antagonist (e.g., atezolizumab) to treat or delay progression of cancer a CD20-positive cell proliferative disorder (e.g., a B cell proliferative disorder, e.g., an NHL (e.g., an aggressive NHL or a relapsed or refractory (R/R) NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)) in a subject. In some instances, the article of manufacture or kit further comprises package insert comprising instructions for using mosunetuzumab, the anti-TIGIT antagonist antibody (e.g., tiragolumab), and optionally with the PD-1 axis binding antagonist (e.g., atezolizumab) to treat or delay progression of cancer a CD20-positive cell proliferative disorder (e.g., a B cell proliferative disorder, e.g., an NHL (e.g., an aggressive NHL or a relapsed or refractory (R/R) NHL; e.g., an R/R diffuse large B cell lymphoma (DLBCL), an R/R follicular lymphoma (FL) (e.g., an R/R Grade 3b FL), or an R/R high grade B cell lymphoma (HGBL) or an R/R transformed FL (trFL)) in a subject. Any of mosunetuzumab, the anti-TIGIT antagonist antibodies (tiragolumab), and/or the PD-1 axis binding antagonists (atezolizumab) described herein may be included in the article of manufacture or kits.

In another embodiment, a kit comprises mosunetuzumab for use in combination with tiragolumab, and optionally in combination with atezolizumab, for treating a subject having a cancer according to any of the methods described herein. In some embodiments, the kit further comprises mosunetuzumab. In some instances, the article of manufacture or kit further comprises package insert comprising instructions for using mosunetuzumab in combination with tiragolumab, and optionally in combination with atezolizumab, to treat or delay progression of a cancer in a subject.

In another embodiment, a kit comprises tiragolumab for use in combination with mosuentuzumab, and optionally in combination with atezolizumab, for treating a subject having a cancer according to any of the methods described herein. In some embodiments, the kit further comprises tiragolumab. In some instances, the article of manufacture or kit further comprises package insert comprising instructions for using tiragolumab in combination with mosunetuzumab, and optionally in combination with atezolizumab, to treat or delay progression of cancer in a subject.

In some instances, mosunetuzumab, the anti-TIGIT antagonist antibody (e.g., tiragolumab), and/or the PD-1 axis binding antagonist (e.g., atezolizumab) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags, and syringes.

Any of mosunetuzumab, the anti-TIGIT antagonist antibodies (tiragolumab), and/or the PD-1 axis binding antagonists (atezolizumab) described herein may be included in the article of manufacture or kits. Any of the articles of manufacture or kits may include instructions to administer mosunetuzumab, an anti-TIGIT antagonist antibody (tiragolumab), and/or a PD-1 axis binding antagonist (atezolizumab) to a subject in accordance with any of the methods described herein, e.g., any of the methods set forth in Section IV above.

### D. Kits Comprising a PD-1 Axis Binding Antagonist, an Anti-TIGIT Antagonist Antibody, and an Anti-VEGF Antibody

In another aspect, provided herein is an article of manufacture or a kit comprising a PD-1 axis binding antagonist (e.g., atezolizumab), an anti-TIGIT antagonist antibody (e.g., tiragolumab), and an anti-VEGF antibody (e.g., bevacizumab). In some instances, the article of manufacture or kit further comprises a package insert comprising instructions for using the anti-TIGIT antagonist antibody, the PD-1 axis binding antagonist, and the anti-VEGF antibody to treat or delay progression of cancer (e.g., colorectal cancer (CRC) (e.g., metastatic CRC (e.g., microsatellite instability (MSI) high (MSI-H) metastatic CRC))) in a subject. In some instances, the article of manufacture or kit further comprises package insert comprising instructions for using the anti-TIGIT antagonist antibody, the PD-1 axis binding antagonist, and the anti-VEGF antibody to treat or delay progression of cancer (e.g., metastatic CRC (e.g., MSI-H metastatic CRC)) in a subject. Any of the PD-1 axis binding antagonists, anti-TIGIT antagonist antibodies, and VEGF antagonists (e.g., anti-VEGF antibodies) described herein may be included in the article of manufacture or kits.

In another embodiment of the invention, a kit is provided comprising a PD-1 axis binding antagonist for use in combination with an anti-TIGIT antagonist antibody and an anti-VEGF antibody for treating a subject having a cancer according to any of the methods described herein. In some instances, the kit further comprises the anti-TIGIT antagonist antibody. In some instances, the kit further comprises the anti-VEGF antibody. In some instances, the kit further comprises the anti-TIGIT antagonist antibody and the anti-VEGF antibody. In some instances, the article of manufacture or kit further comprises a package insert comprising instructions for using the PD-1 axis binding antagonist in combination with the anti-TIGIT antagonist antibody (e.g., tiragolumab) and the anti-VEGF antibody (e.g., bevacizumab) to treat or delay progression of a cancer in a subject.

In another embodiment, a kit comprises tiragolumab for use in combination with atezolizumab and bevacizumab for treating a subject having a cancer according to any of the methods described herein. In some embodiments, the kit further comprises atezolizumab. In some embodiments, the kit further comprises bevacizumab. In some embodiments, the kit further comprises atezolizumab and bevacizumab. In some instances, the article of manufacture or kit further comprises package insert comprising instructions for using tiragolumab in combination with atezolizumab and bevacizumab to treat or delay progression of a cancer in a subject.

In another embodiment, a kit comprises atezolizumab for use in combination with tiragolumab for treating a subject having a cancer according to any of the methods described herein. In some embodiments, the kit further comprises tiragolumab. In some embodiments, the kit further comprises bevacizumab. In some embodiments, the kit further comprises tiragolumab and bevacizumab. In some instances, the article of manufacture or kit further comprises package insert comprising instructions for using atezolizumab in combination with tiragolumab and bevacizumab to treat or delay progression of cancer in a subject.

In another embodiment, a kit comprises bevacizumab for use in combination with atezolizumab for treating a subject having a cancer according to any of the methods described herein. In some embodiments, the kit further comprises atezolizumab. In some embodiments, the kit further comprises tiragolumab. In some embodiments, the kit further comprises atezolizumab and tiragolumab. In some instances, the article of manufacture or kit further comprises package insert comprising instructions for using bevacizumab in combination with atezolizumab and tiragolumab to treat or delay progression of cancer in a subject.

In some instances, the PD-1 axis binding antagonist, the anti-TIGIT antagonist antibody, and the anti-VEGF antibody are in the same container or separate containers. Suitable containers include, for example, bottles, vials, bags, and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the PD-1 axis binding antagonists, anti-TIGIT antagonist antibodies, and VEGF antagonists (e.g., anti-VEGF antibodies) described herein may be included in the article of manufacture or kits. Any of the articles of manufacture or kits may include instructions to administer a PD-1 axis binding antagonist, an anti-TIGIT antagonist antibody, and a VEGF antagonist (e.g., an anti-VEGF antibody) to a subject in accordance with any of the methods described herein, e.g., any of the methods set forth in Section II, III, or V above.

### EXAMPLES

### Example 1: A Phase Ib, Dose-Expansion Study of the Anti-TIGIT Antibody Tiragolumab in Combination with Atezolizumab in Patients with Metastatic Esophageal Cancer

The safety, pharmacodynamics, and anti-tumor activity of tiragolumab administered in combination with atezolizumab in patients with metastatic esophageal cancer were evaluated in a Phase Ib expansion cohort study.

This study was an indication-specific expansion cohort of G030103, a Phase Ia/Ib open-label, multicenter, dose-escalation and dose expansion study designed to evaluate the safety, tolerability, and PK of tiragolumab in combination with atezolizumab in patients with locally advanced, recurrent, or metastatic incurable tumors. The G030103 study showed that tiragolumab was well tolerated at the recommended Phase II dose of 600 mg IV Q3W with atezolizumab 1200 mg IV Q3W, and activity was seen in a Phase Ib expansion cohort of patients with PD-L1-positive non-small cell lung cancer, with a confirmed overall response rate (ORR) of 46% (Bendell et al., Cancer Res: 80 (16 Supplement), 779, 2020).

The present study provides safety and anti-tumor activity results for tiragolumab in combination with atezolizumab in patients with metastatic esophageal cancer who have not been previously treated with cancer immunotherapy.

### A. Objectives and Endpoints

The objective of the study was to determine the preliminary safety, tolerability, and efficacy of tiragolumab (600 mg, IV, Q3W) in combination with atezolizumab (1200 mg, IV, Q3W) in patients with metastatic esophageal cancer. Specific objectives and corresponding endpoints for the study are outlined in Table 5.

TABLE 5
Objectives and Endpoints

| | |
|---|---|
| Objectives | Corresponding Endpoints |

Safety Objective:

| | |
|---|---|
| To evaluate the safety | Incidence and nature of DLTs |
| and tolerability of | Incidence, nature, and severity of adverse events |
| tiragolumab in | graded according to NCI CTCAE v4.0 |
| combination with | Change from baseline in targeted vital signs |
| atezolizumab. | Change from baseline in targeted clinical |
| | laboratory test results, including ECGs |
| | Number of cycles received and dose intensity |
| | Incidence of anti-tiragolumab antibodies and/or |
| | anti-atezolizumab antibodies, and the potential |
| | correlation with PK, pharmacodynamic, safety, |
| | and preliminary efficacy parameters |

Activity Objective:

| | |
|---|---|
| To make a preliminary | Objective response, defined as a complete |
| assessment of the anti- | response (CR) or partial response (PR) per |
| tumor activity of | Response Evaluation Criteria in Solid Tumors |
| tiragolumab in | (RECIST) v.1.1, as determined by the investigator |
| combination with | and confirmed by repeat assessment ≥4 weeks |
| atezolizumab in | after initial documentation. |
| patients with locally | Duration of objective response (DOR), defined as |
| advanced or metastatic | the time from the first occurrence of a |
| tumors | documented objective response to the time of the |
| | first documented disease progression or death |
| | from any cause, whichever occurs first, per |
| | RECIST v1.1 as determined by the investigator |
| | PFS, defined as the time from the first study |
| | treatment to the first occurrence of progression or |
| | death from any cause, whichever occurs first, per |
| | RECIST v.1.1 as determined by the investigator |

### B. Study Design

This study was designed to enable evaluation of safety, tolerability, and efficacy of tiragolumab when administered in combination with atezolizumab in patients with metastatic esophageal cancer (e.g., patients having metastatic esophageal cancer who have not received prior immunotherapy).

### C. Study Treatment

### Dosing and Administration

Tiragolumab at 600 mg was administered every 3 weeks by IV infusion (Q3W).

The dose of atezolizumab administered in combination with tiragolumab was 1200 mg IV every 3 weeks. Atezolizumab was administered after the tiragolumab infusion and subsequent observation period.

For details on dose preparation and administration instructions for tiragolumab and atezolizumab, refer to the respective Investigator's Brochures and to the GO30103 Pharmacy Manual.

### D. Concomitant Therapy

Concomitant therapy included any medication (e.g., prescription drugs, over-the-counter drugs, herbal or homeopathic remedies, and/or nutritional supplements) used by a patient from 7 days prior to screening to the treatment discontinuation visit.

### E. Inclusion Criteria

### Esophageal Cancer-Specific Inclusion Criteria

The eligibility criteria are summarized below.
Metastatic esophageal cancer of any histology (including, e.g., squamous cell carcinoma and adenocarcinoma).
No limit on prior lines of therapy.
No prior treatment with immunotherapy.
Any PD-L1 status.

### Additional Cancer-Specific Inclusion Criteria

Patients with histologic documentation of locally advanced, recurrent, or metastatic incurable malignancy that had progressed after at least one available standard therapy; or for whom standard therapy had proven to be ineffective or intolerable, or was considered inappropriate; or for whom a clinical trial of an investigational agent was a recognized standard of care.
Patients with histologic documentation of locally advanced, recurrent, or metastatic incurable malignancy for whom a clinical trial of an investigational agent in combination with an anti-PD-L1 antibody with and without chemotherapy is considered an acceptable treatment option were also eligible.
Patients with measurable disease per RECIST v1.1
   Previously irradiated lesions were counted as target lesions.
   Lesions that were intended to be biopsied were not counted as target lesions.

### Additional Inclusion Criteria for Patients in Expansion Cohorts

Enrollment in this expansion cohort was limited to patients whose tumors were PD-L1 and/or TIGIT selected. Therefore, archival tumor tissue was required to be submitted and evaluated for PD-L1 and/or TIGIT expression prior to enrollment. Patients whose tumor tissue was not evaluable for PD-L1 and/or TIGIT expression were not eligible. PD-L1 and/or TIGIT expression may have been evaluated in either immune infiltrating cells or tumor cells. If multiple tumor specimens were submitted (e.g., an archival specimen and tissue from relapsed disease), patients may have been eligible if at least one specimen was evaluable for PD-L1 and/or TIGIT expression. The PD-L1 and/or TIGIT score of the patient was the maximum PD-L1 and/or TIGIT score among the samples, respectively.
Enrollment was managed such that approximately up to half of the accrued patients in the expansion cohort were those who consented to undergo optional biopsies.

### General Criteria

Additional inclusion criteria are described below. These patient criteria were required for study entry.
Age ≥18 years
Able to comply with the study protocol, in the investigator's judgment
Eastern Cooperative Oncology Group (ECOG) performance status of 0 or 1
Life expectancy ≥12 weeks
   Adequate hematologic and end organ function, defined by the following laboratory results obtained within 14 days prior to the first study treatment (Cycle 1, Day 1): absolute neutrophil count (ANC) ≥1,500 cells/μL; white blood cell (WBC) count ≥2,500/μL; lymphocyte count ≥500/μL; platelet count ≥100,000/μL (without transfusion within 14 days prior to Cycle 1, Day 1); hemoglobin ≥9 g/dL (Patients may have been transfused or may have received erythropoietic treatment as per local standard of care); total bilirubin ≤1.5×the upper limit of normal (ULN) (patients receiving paclitaxel (Cohort B) were required to have total bilirubin ≤1.25×ULN); AST and ALT≤3×ULN; alkaline phosphatase ≤2.5×ULN (patients with documented liver or bone metastases may have had alkaline phosphatase ≤5×ULN); serum albumin ≥2.5 g/dL; PT and activated partial thromboplastin time (aPTT) ≤1.5×ULN (applies only to patients who were not receiving therapeutic anticoagulation. Patients receiving therapeutic anticoagulation were on a stable dose).
Measured or calculated creatinine clearance 50 mL/min on the basis of the Cockcroft-Gault glomerular filtration rate estimation:

$$\begin{matrix}( 14 \; 0 - age ) \times ( weight in kilograms ) \times ( 0.85 if female ) \\ 72 \times ( serum creatinine in mg / dL )\end{matrix}$$

Patients receiving cisplatin (Cohort A or Cohort C) must have measured or calculated creatinine clearance; 60 mL/min on the basis of the Cockcroft-Gault GFR estimation
Serum pregnancy test for women of childbearing potential (including women who have had a tubal ligation) was required to be performed and documented as negative within 14 days prior to Cycle 1, Day 1.
For women of childbearing potential: agreement to remain abstinent (refrain from heterosexual intercourse) or use contraception, and agreement to refrain from donating eggs.
For men: agreement to remain abstinent (refrain from heterosexual intercourse) or use contraceptive measures, and agreement to refrain from donating sperm.

### F. Exclusion Criteria

### Cancer-Specific Exclusion Criteria

Any anti-cancer therapy, whether investigational or approved, including chemotherapy, hormonal therapy, and/or radiotherapy, within 3 weeks prior to initiation of study treatment, with the following exceptions:
   Hormonal therapy with gonadotropin-releasing hormone (GnRH) agonists or antagonists for prostate cancer
   Hormone-replacement therapy or oral contraceptives
   Tyrosine kinase inhibitor(s) (TKIs) approved by local regulatory authorities for treatment of cancer that have been discontinued >7 days prior to Cycle 1, Day 1; baseline scans were required to be obtained after discontinuation of prior TKIs, and criteria pertaining to adverse events attributed to prior cancer therapies must have been met
   Herbal therapy >1 week before Cycle 1, Day 1
   Palliative radiotherapy for painful metastases or metastases in potentially sensitive locations (e.g., epidural space) >2 weeks prior to Cycle 1, Day 1
Prior anti-TIGIT agents were not allowed
Prior treatment with cancer vaccines and/or cytokines was allowed provided that at least 6 weeks or 5 half-lives of the drug, whichever is shorter, had elapsed between the last dose and the proposed Cycle 1, Day 1.
Minimum washout was 3 weeks for any prior systemic cancer therapy.
Patients with the pulmonary lymphoepithelioma-like carcinoma subtype of NSCLC
Primary CNS malignancy, untreated CNS metastases, or active CNS metastases (progressing or requiring corticosteroids for symptomatic control)
   Patients with a history of treated CNS metastases were eligible, provided they met all of the following criteria: Measurable disease outside the CNS; no ongoing requirement for corticosteroids as therapy for CNS metastases, with corticosteroids discontinued for 2 weeks prior to enrollment with no ongoing symptoms attributable to CNS metastases (anticonvulsants at a stable dose were allowed); radiographic demonstration of improvement upon the completion of CNS-directed therapy and no evidence of interim progression between the completion of CNS-directed therapy and the screening radiographic study; screening CNS radiographic study was 4 weeks since completion of radiotherapy.
Leptomeningeal disease
Uncontrolled tumor-related pain
Uncontrolled pleural effusion, pericardial effusion, or ascites requiring recurrent drainage procedures (once monthly or more frequently). Patients with indwelling catheters (e.g., PleurX catheters) were allowed.
Malignancies other than disease under study within 5 years prior to Cycle 1, Day 1, with the exception of those with a negligible risk of metastasis or death (such as adequately treated carcinoma in situ of the cervix, basal or squamous cell skin cancer, localized prostate cancer, or ductal carcinoma in situ)
Uncontrolled hypercalcemia (>1.5 mmol/L ionized calcium or Ca >12 mg/dL or corrected serum calcium ≥ULN) or symptomatic hypercalcemia requiring continued use of bisphosphonate therapy or denosumab.
Patients with spinal cord compression not definitively treated with surgery and/or radiation or previously diagnosed and treated spinal cord compression without evidence that disease had been clinically stable for ≥2 weeks prior to screening.

### Treatment-Specific Exclusion Criteria

History of autoimmune disease, including but not limited to systemic lupus erythematosus, rheumatoid arthritis, inflammatory bowel disease, vascular thrombosis associated with antiphospholipid syndrome, Wegener's granulomatosis, Sjögren's syndrome, Bell's palsy (of autoimmune etiology only), Guillain-Barré syndrome, multiple sclerosis, vasculitis, or glomerulonephritis, with the following caveats:

Patients with a history of autoimmune hypothyroidism on a stable dose of thyroid replacement hormone may have been eligible.

Patients with eczema, psoriasis, lichen simplex chronicus, or vitiligo with dermatologic manifestations only (e.g., no psoriatic arthritis) may have been eligible provided that they met the following conditions: rash was required to cover less than 10% of the body surface area; disease is well controlled at baseline and only requires low potency topical steroids; no acute exacerbations of underlying condition within the last 12 months.
Treatment with systemic immunosuppressive medications (including but not limited to prednisone >10 mg/day, cyclophosphamide, azathioprine, methotrexate, thalidomide, and tumor necrosis factor-α [TNF-α] antagonists) within 2 weeks prior to Cycle 1, Day 1. Patients who have received acute, low-dose, systemic immunosuppressant medications (e.g., a one-time dose of dexamethasone for nausea) may have been enrolled in the study. The use of inhaled corticosteroids (e.g., fluticasone for chronic obstructive pulmonary disease), oral mineralocorticoids (e.g., fludrocortisone for patients with orthostatic hypotension), and physiologic doses of corticosteroids for adrenal insufficiency were allowed.
History of idiopathic pulmonary fibrosis, pneumonitis (including drug-induced), organizing pneumonia (i.e., bronchiolitis obliterans, cryptogenic organizing pneumonia, etc.), or evidence of active pneumonitis on screening chest CT scan

History of radiation pneumonitis in the radiation field (fibrosis) was permitted.
Positive test for HIV infection
Active hepatitis B (defined as having a positive hepatitis B surface antigen [HBsAg] test at screening); active hepatitis C; active EBV infection and known or suspected chronic active EBV infection at screening; or active tuberculosis. Severe infections within 4 weeks prior to Cycle 1, Day 1, including but not limited to hospitalization for complications of infection, bacteremia, or severe pneumonia
Recent infections not meeting the above criteria for severe infections, including the following:
   Signs or symptoms of infection within 2 weeks prior to Cycle 1, Day 1
   Received oral or IV antibiotics within 2 weeks prior to Cycle 1, Day 1
   Patients receiving prophylactic antibiotics (e.g., for prevention of a urinary tract infection or chronic obstructive pulmonary disease) were eligible.
Prior allogeneic bone marrow transplantation or prior solid organ transplantation
Administration of a live, attenuated vaccine within 4 weeks before Cycle 1, Day 1 or anticipation that such a live attenuated vaccine was required during the study
   Influenza vaccination was given during influenza season only. Patients were required to not receive live, attenuated influenza vaccine (e.g., FluMist®) within 4 weeks prior to Cycle 1, Day 1 or at any time during the study, and for 5 months following the last study treatment.
History of severe allergic, anaphylactic, or other hypersensitivity reactions to chimeric or humanized antibodies or fusion proteins
Known hypersensitivity to CHO-cell products
Allergy or hypersensitivity to components of the atezolizumab formulation

### G. Analysis

### Activity Analyses

The analyses described below are based on the definitions of objective response according to RECIST v1.1.

### Objective Response Rate

The analysis of ORR included patients who received any amount of the study treatment and had measurable disease at baseline. Objective response was defined as a CR or PR, as determined by investigator assessment and confirmed by repeat assessment ≥4 weeks after initial documentation. Patients with missing baseline or no response assessments were classified as non-responders.

### Duration of Response

Among patients with an objective response, duration of objective response was defined as the time from the initial complete or partial response to the time of disease progression or death, whichever occurred first. For patients who did not die or experience disease progression before the end of the study or who were lost to follow-up, duration of objective response was censored at the day of the last tumor assessment.

### Progression-Free Survival

The analyses of PFS included patients who had received any amount of study treatment. PFS was defined as the time from the first day of study treatment until documented disease progression or death, whichever occurred first. For patients who did not have documented PD or death before the end of the study or who were lost to follow-up, PFS was censored at the day of the last tumor assessment. For patients without any post-baseline tumor assessments, PFS was censored at the first day of study treatment.

### Safety Analyses

Safety was assessed through summaries of DLTs, adverse events, changes in laboratory test results, changes in vital signs and ECGs, and exposure to any study treatment (tiragolumab or atezolizumab). All patients who received any amount of study treatment were included in the safety analyses.

### H. Patient Characteristics

The baseline characteristics and disposition of patients are shown in Table 6. Twenty-one patients with metastatic esophageal cancers were treated (histopathological subtypes: squamous (13 patients), adenocarcinoma (7 patients) and neuroendocrine (1 patient)). Heavily pre-treated populations were included, with 15 patients (71.4%) having received ≥2 prior therapies. The median age was 62 years. Five patients (23.8%) had ECOG PS 0, and 16 patients (76.2%) had ECOG PS 1.

TABLE 6
Baseline characteristics and patient disposition

| | | Tiragolumab + | |
|---|---|---|---|
| | | Atezolizumab (n = 21) | |
| Characteristic, n (%) | | | |
| Age in years, median (range) | | 62 | (50-77) |
| Male | | 18 | (86%) |
| ECOG Performance Status 1 | | 16 | (76%) |
| Race | White | 9 | (43%) |
| | Asian | 7 | (33%) |
| | Other | 5 | (24%) |
| Prior cancer | 1 | 6 | (29%) |
| therapies^{a} | 2 | 8 | (38%) |
| | ≥3 | 7 | (33%) |
| Histopathologic | Squamous | 13 | (62%) |
| subtype | Adenocarcinoma | 8 | (38%) |
| PD-L1 status | TC or IC ≥5% | 5 | (24%) |

(VENTANA PD-L1
SP142 assay)

| Disposition, n (%) | | |
|---|---|---|
| Ongoing | 1 | (5%) |
| Discontinued study | 20 | (95%) |
| Progression/death | 15 | (71%) |
| Withdrawal | 3 | (14%) |
| Lost to follow-up | 2 | (10%) |

^{a}Includes neoadjuvant and/or adjuvant therapy
ECOG; Eastern Cooperative Oncology Group;
IC, immune cell;
TC, tumor cell

### I. Adverse Events

A safety summary of adverse events is shown in Table 7. Treatment-related adverse events (TRAEs), as assessed by investigators, occurred in 14 patients (66.7%), with a Grade 3 TRAE seen in 1 patient. Immune-mediated AEs (imAEs) occurred in 12 patients (57.1%). No Grade 4 or 5 TRAEs or imAEs were observed.

TABLE 7
Safety summary of adverse events

| | Tiragolumab + | |
|---|---|---|
| Patients with ≥1 adverse event (AE), n (%) | Atezolizumab (n = 21) | |
| Any-cause AE | 21 | (100%) |
| Grade 3-4 AEs (all cause) | 14 | (67%) |
| Treatment-related Grade 3-4 AEs^{a} | 1 | (5%) |
| Serious AE | 15 | (71%) |
| AE leading to any treatment interruption | 8 | (38%) |
| AE leading to any treatment discontinuation^{b} | 1 | (5%) |
| All treatment-related AEs | 14 | (67%) |
| Grade 1 | 7 | (33%) |
| Grade 2 | 6 | (29%) |
| Grade 3^{a} | 1 | (5%) |
| All immune-mediated AEs | 12 | (57%) |
| Grade 1 | 5 | (24%) |
| Grade 2 | 4 | (19%) |
| Grade 3^{c} | 3 | (14%) |

^{a}One patient had a related Grade 3 AE of lymphocyte count decreased.
^{b}One patient discontinued treatment for Grade 5 upper airway obstruction not related to study drugs.
^{c}Grade 3 immune-mediated AEs included amylase increased (n = 2) and transaminase increased (n = 1).

The most common AEs and all immune-mediated AEs are shown in Table 8. The most common AEs reported (in ≥15% of patients) were malignant neoplasm progression (28.6%), anemia (23.8%), decreased appetite, cough, aspartate aminotransferase increase, and amylase increase (all 19.0%). TRAEs, including immune-mediated AEs (mainly rash and laboratory abnormalities), were primarily Grade 1-2. Overall, tiragolumab and atezolizumab was well tolerated in patients with metastatic esophageal cancer and had an acceptable safety profile.

TABLE 8
Most common AEs and all immune-mediated AEs

| | Tiragolumab + | |
|---|---|---|
| | Atezolizumab (n = 21) | |
| All adverse events (AE) ≥10% | | |
| Anemia | 5 | (24%) |
| Decreased appetite | 4 | (19%) |
| Cough | 4 | (19%) |
| Aspartate aminotransferase increase | 4 | (19%) |
| Amylase increase | 4 | (19%) |
| Dysphagia | 3 | (14%) |
| Pyrexia | 3 | (14%) |
| Pruritus | 3 | (14%) |
| Rash | 3 | (14%) |
| Alanine aminotransferase increased | 3 | (14%) |
| All immune-mediated AEs (imAEs)^{a} | | |
| Rash | 8 | (38%) |
| Hepatitis (laboratory abnormalities)^{b} | 5 | (24%) |
| Pancreatitis (laboratory abnormalities)^{b} | 4 | (19%) |
| Diabetes | 1 | (5%) |
| Hyperthyroidism | 1 | (5%) |
| Hypophysitis | 1 | (5%) |
| Hypothyroidism | 1 | (5%) |

^{a}imAEs reported by medical concepts;
^{b}All cases only laboratory abnormalities and not confirmed diagnosis (no clinical symptoms).

### J. Efficacy

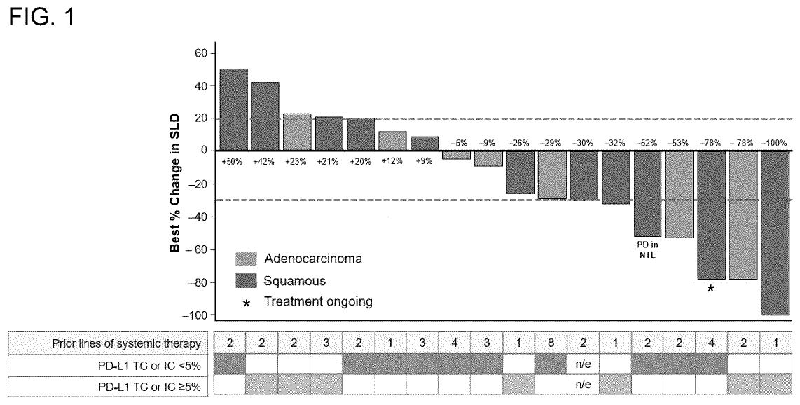

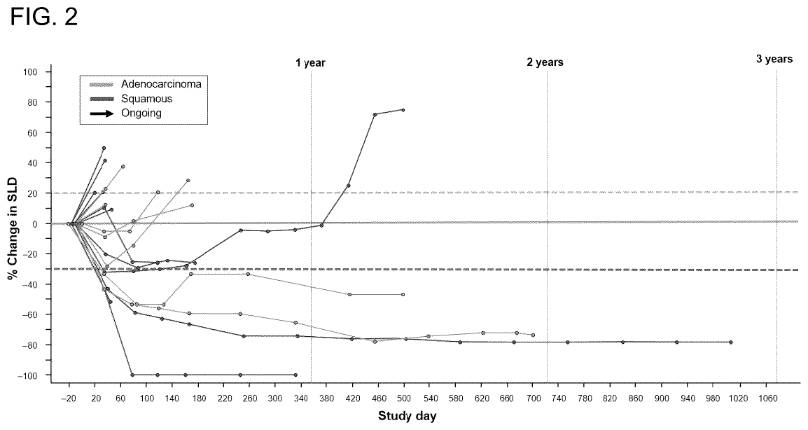

An efficacy analysis was performed. Of 18 evaluable patients with at least one tumor assessment, there were 5 partial responses (PR) (confirmed objective response rate (ORR) of 27.8% (5/18 patients)) (FIG. 1). The disease control rate (DCR) was 50% (9/18 patients). The median progression-free survival was 3.5 months (95% confidence interval (CI): 1.2-5.6) (Table 9). One patient was still in the study at over 2 years (Treatment ongoing; FIG. 1), demonstrating durable response (FIG. 2). The size of tumors was reduced in patients with PD-L1 TC or IC that was <5% or ≥5% (3/5 of the responders had PD-L1 TC or IC<5%) (FIG. 1), indicating anti-tumor activity regardless of PD-L1 status.

TABLE 9
Treatment Response

| | Tiragolumab + Atezolizumab (n = 18) | |
|---|---|---|
| Median PFS, months (95% CI) | 3.5 | (1.2-5.6) |
| Median DOR, months (95% CI) | 15.3 | (7.0-NR) |

CI, confidence interval; DOR, duration of response; NR, not reached; PFS, progression-free survival.

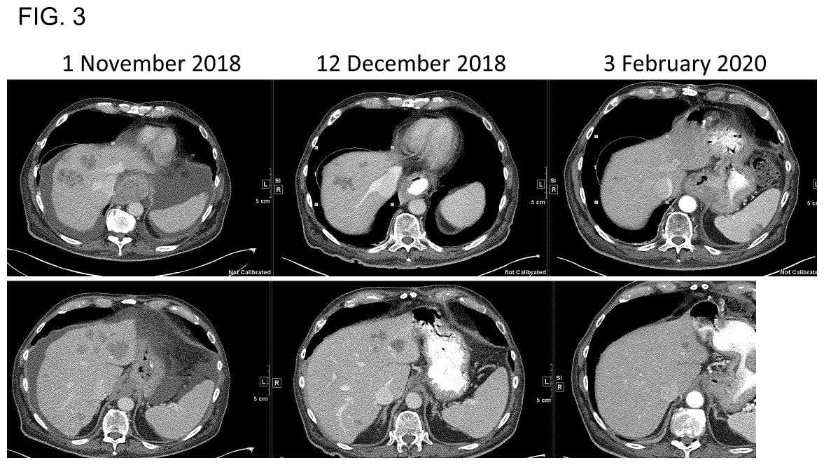

A 78-year-old Caucasian male with metastatic esophageal adenocarcinoma was enrolled in the study in November 2018 and showed a partial response (PR) at the first tumor assessment, which was maintained for 2 years (FIG. 3). The PD-L1 status of this patient was PD-L1 TC<1%, PD-L1 IC 6% according to the VENTANA SP142 assay. The patient had received multiple prior lines of therapy, including first-line modified 5-fluorouracil, leucovorin, and oxaliplatin (mFOLFOX6) with stable disease (SD) as the best response to this therapy before progression of disease (PD) (March 2018 to June 2018) and second-line paclitaxel and ramucirumab with progression of disease (PD) as best response (June 2018 to September 2018). This case demonstrates durability of clinical response in a heavily pre-treated patient.

In summary, tiragolumab in combination with atezolizumab showed promising preliminary anti-tumor activity in patients with heavily pre-treated metastatic esophageal cancer who had not received prior immunotherapy. Durable responses occurred in patients with tumors independent of PD-L1 status or histology.

### Example 2: A Phase Ib/II, Open-Label, Multicenter, Randomized Umbrella Study Evaluating the Efficacy and Safety of Multiple Treatment Combinations in Patients with Melanoma

Melanoma is a potentially deadly form of skin cancer and is one of the fastest-growing malignancies (Algazi et al. Cancer Manag Res. 2:197-211, 2010; Finn et al. BMC Med. 10: 23, 2012). More than 300,000 people worldwide are currently diagnosed with melanoma each year, and 57,000 people die of the disease. The clinical outcomes of patients with melanoma are highly dependent on the stage at presentation. Most people who present with more advanced melanoma have a poor prognosis (Finn et al. BMC Med. 10: 23, 2012). Patients with lymph-node involvement (Stage III) have a high risk of local and distant relapse after surgery, and the 5-year survival rate is 32%-93% in this patient group (Gershenwald et al. CA Cancer J Clin. 67: 472-492, 2017). Few patients have metastatic disease (Stage IV) at presentation, but some develop metastases after their initial definitive treatment. Immunotherapy and targeted therapies have improved the outcomes of those patients, and the 5-year survival rate is around 50% (Larkin et al. N Engl J Med. 373: 23-34, 2015; Wolchok et al. N Engl J Med. 377: 1345-1356, 2017; Larkin et al. N Engl J Med. 381: 1535-1546, 2019; Robert et al. Lancet Oncol. 20: 1239-1251, 2019; Long et al. J Clin Oncol. 38(Suppl 15): 10013, 2020). Despite recent therapeutic advances, melanoma continues to be a serious health issue, with a high medical need and a steadily increasing incidence over the past 30 years (Bataille. Expert Rev Dermatol. 4: 533-539, 2009).

BO43328 is a Phase Ib/II, open-label, multicenter, randomized, umbrella study in patients with resectable Stage III (Cohort 1) or Stage IV (Cohort 2) melanoma. The study is designed with the flexibility to open new treatment arms as new treatments become available, close existing treatment arms that demonstrate minimal clinical activity or unacceptable toxicity, modify the patient population (e.g., with regard to prior anti-cancer treatment or biomarker status), or introduce additional cohorts of patients with other types of melanoma.

### A. Overview of Study Design

This study evaluates the efficacy, safety, and pharmacokinetics of treatment combinations in cancer immunotherapy (CIT)-naive patients with resectable Stage III melanoma (Cohort 1) and in patients with Stage IV melanoma (Cohort 2). Specific objectives and corresponding endpoints for the study are outlined below for Cohort 1 (see Table 10) and Cohort 2 (see Table 11).

TABLE 10
Objectives and Corresponding Endpoints for Cohort 1

| | Corresponding Endpoints |
|---|---|
| Primary Efficacy Objective | |
| To evaluate the efficacy of | pRR, (defined as the proportion of patients with pCR, pnCR, and |
| treatment | pPR) at time of surgery, as determined by independent pathologic |
| | review. |
| Secondary Efficacy Objective | |
| To evaluate the efficacy of | pRR (defined as the proportion of patients with pCR, pnCR, and |
| treatment | pPR) at time of surgery, as determined by local pathologic |
| | assessment. |
| | EFS, defined as the time from randomization to any of the following |
| | events (whichever occurs first): Disease progression that precludes |
| | surgery, as assessed by the investigator according to RECIST v1.1; |
| | local, regional or distant disease recurrence; or death from any |
| | cause. |
| | RFS, defined as the time from surgery to the first documented |
| | recurrence of disease or death from any cause. |
| | OS, defined as the time from randomization to death from any |
| | cause. |
| | ORR, defined as the proportion of patients with a CR or PR as |
| | determined by the investigator according to RECIST v1.1, prior to |
| | surgery. |
| | Responses are assessed and determined according to RECIST |
| | v1.1 but are not required to be confirmed by later imaging studies. |
| Exploratory Efficacy Objective | |
| To evaluate the efficacy of | Landmark EFS, defined as the time from randomization to any of the |
| treatment | following events (whichever occurs first): Disease progression that |
| | precludes surgery, as assessed by the investigator according to |
| | RECIST v1.1; local, regional or distant disease recurrence; or death |
| | from any cause at specific timepoints (1, 2, 3, and 5 years). |
| | Landmark RFS, defined as the time from surgery to the first |
| | documented recurrence of disease or death from any cause at |
| | specific timepoints (1, 2, 3, and 5 years). |
| | Landmark OS, defined as the time from randomization to death from |
| | any cause at specific timepoints (1, 2, 3, and 5 years). |
| Safety Objective | |
| To evaluate the safety of | Incidence, nature, and severity of adverse events and laboratory |
| treatment | abnormalities, with severity determined according to NCI CTCAE |
| | v5.0. |
| | CRS severity is also determined according to the ASTCT CRS |
| | Consensus Grading Scale. |
| | Incidence and nature of immune-related adverse events Grade ≥3 |
| | during the first 12 weeks. |
| | Rate and duration of delayed surgery due to treatment-related |
| | adverse events. |
| | Surgical complication rates according to Clavien-Dindo surgical |
| | classification after CLND. |
| Exploratory Pharmacokinetic | |
| Objectives | |
| To characterize the PK | Plasma or serum concentrations of each drug (as appropriate) at |
| profile of drugs that are | specified timepoints |
| administered as part of | |
| treatment | |
| To evaluate potential | Relationship between plasma or serum concentration or PK |
| relationships between drug | parameters for each drug (as appropriate, on the basis of available |
| exposure and the efficacy | data) and efficacy endpoints. |
| and safety of treatment | Relationship between plasma or serum concentration or PK |
| | parameters for each drug (as appropriate, on the basis of available |
| | data) and safety endpoints. |
| Exploratory Immunogenicity | |
| Objectives | |
| To evaluate the immune | For drugs for which ADA formation is measured: Presence of ADAs |
| response to drugs that are | during the study relative to the presence of ADAs at baseline. |
| administered | |
| To evaluate potential effects | For drugs for which ADA formation is measured: Relationship |
| of ADAS | between ADA status and efficacy, safety, or PK endpoints. |
| Exploratory Biomarker Objective | Corresponding Endpoint |
| To identify biomarkers | Relationship between biomarkers in blood and tumor tissue and |
| biology that are predictive of | efficacy, safety, PK, immunogenicity, or other biomarker endpoints. |
| response to study treatment | |
| (i.e., predictive biomarkers), | |
| are associated with | |
| progression to a more | |
| severe disease state (i.e., | |
| prognostic biomarkers), are | |
| associated with resistance to | |
| study treatment, are | |
| associated with susceptibility | |
| to developing adverse | |
| events (i.e., safety | |
| biomarkers), can provide | |
| evidence of study treatment | |
| activity (i.e., | |
| pharmacodynamic | |
| biomarkers), or can increase | |
| the knowledge and | |
| understanding of disease | |
| biology. | |

ADA = anti-drug antibody; ASTCT = American Society for Transplantation and Cellular Therapy; CLND = completion lymph node dissection; CR = complete response; CRS = cytokine-release syndrome; EFS = event-free survival; NCI CTCAE v5.0 = National Cancer Institute Common Terminology Criteria for Adverse Events, Version 5.0; ORR = objective response rate; OS = overall survival; pCR = pathologic complete response; PK = pharmacokinetic; pnCR = pathologic near complete response; pPR = pathologic partial response; PR = partial response; pRR = pathologic response rate; RECIST v1.1 = Response Evaluation Criteria in Solid Tumors, Version 1.1; RFS = relapse-free survival.

TABLE 11
Objectives and Corresponding Endpoints for Cohort 2

| Primary Efficacy Objective | Corresponding Endpoint |
|---|---|
| To evaluate the efficacy of | ORR, defined as the proportion of patients with a CR or PR on two |
| treatment | consecutive occasions ≥4 weeks apart, as determined by the |
| | investigator according to RECIST v1.1. |
| | Corresponding Endpoints |
| Secondary Efficacy Objective | |
| To evaluate the efficacy of | PFS after randomization/enrollment, defined as the time from |
| treatment | randomization/enrollment to the first occurrence of disease |
| | progression or death from any cause (whichever occurs first), as |
| | determined by the investigator according to RECIST v1.1. |
| | OS after randomization/enrollment, defined as the time from |
| | randomization/enrollment to death from any cause. |
| | OS at specific timepoints (e.g., 6 months). |
| | DOR, defined as the time from the first occurrence of a documented |
| | objective response to disease progression or death from any cause |
| | (whichever occurs first), as determined by the investigator according |
| | to RECIST v1.1. |
| | Disease control, defined as stable disease for ≥12 weeks or a CR or |
| | PR, as determined by the investigator according to RECIST v1.1. |
| Exploratory Efficacy Objective | |
| To evaluate the efficacy of | ORR, PFS, DOR, and disease control as determined by the |
| treatment | investigator according to iRECIST. |

| Safety Objective | Corresponding Endpoint |
|---|---|
| To evaluate the safety of | Incidence, nature, and severity of adverse events and laboratory |
| treatment | abnormalities, with severity determined according to NCI CTCAE |
| | v5.0. |
| | CRS severity will also be determined according to the ASTCT |
| | CRS Consensus Grading Scale. |
| | Corresponding Endpoints |
| Exploratory Pharmacokinetic | |
| Objective | |
| To characterize the PK | Plasma or serum concentration of each drug (as appropriate) at |
| profile of drugs that are | specified timepoints. |
| administered as part of | |
| treatment | |
| To evaluate potential | Relationship between plasma or serum concentration or PK |
| relationships between drug | parameters for each drug (as appropriate on the basis of available |
| exposure and the efficacy | data) and efficacy endpoints. |
| and safety of treatment. | Relationship between plasma or serum concentration or PK |
| | parameters for each drug (as appropriate on the basis of available |
| | data) and safety endpoints. |
| Exploratory Immunogenicity | |
| Objectives | |
| To evaluate the immune | For drugs for which ADA formation is measured: Presence of ADAs |
| response to drugs that are | during the study relative to the presence of ADAs at baseline. |
| administered. | |
| To evaluate potential effects | For drugs for which ADA formation is measured: Relationship |
| of ADAs. | between ADA status and efficacy, safety, or PK endpoints. |
| Exploratory Biomarker | |
| Objective | Corresponding Endpoint |
| To identify biomarkers that | Relationship between biomarkers in blood and tumor tissue and |
| are predictive of response to | efficacy, safety, PK, immunogenicity, or other biomarker endpoints. |
| study treatment (i.e., | |
| predictive biomarkers), are | |
| associated with progression | |
| to a more severe disease | |
| state (i.e., prognostic | |
| biomarkers), are associated | |
| with resistance to study | |
| treatment, are associated | |
| with susceptibility to | |
| developing adverse events | |
| (i.e., safety biomarkers), can | |
| provide evidence of study | |
| treatment activity (i.e., | |
| pharmacodynamic | |
| biomarkers), or can increase | |
| the knowledge and | |
| understanding of disease | |
| biology | |

ADA = anti-drug antibody; ASTCT = American Society for Transplantation and Cellular Therapy; CR = complete response; CRS = cytokine-release syndrome; DOR = duration of response; iRECIST = modified RECIST v1.1 for immune-based therapeutics; NCI CTCAE v5.0 = National Cancer Institute Common Terminology Criteria for Adverse Events, Version 5.0; ORR = objective response rate; OS = overall survival; PFS = progression-free survival; PK = pharmacokinetic; PR = partial response; RECIST v1.1 = Response Evaluation Criteria in Solid Tumors, Version 1.1.
Note:
Overall response at a single timepoint is assessed by the investigator using RECIST v1.1.

Two cohorts are enrolled in parallel in this study. Cohort 1 enrolls patients with resectable Stage III melanoma with measurable lymph node metastases according to Response Evaluation Criteria in Solid Tumors, Version 1.1 (RECIST v1.1) that can be biopsied, who have no history of in-transit metastases within the last 6 months, and who have not received systemic CIT for their disease, e.g., PD-1/PD-L1 and/or CTLA-4 blocking agents or other agents.

Cohort 2 enrolls patients with Stage IV melanoma who experienced disease progression during or after at least one but not more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy) or may have received targeted therapy and checkpoint inhibition therapy concurrently as one combination treatment.

### Treatment Assignment

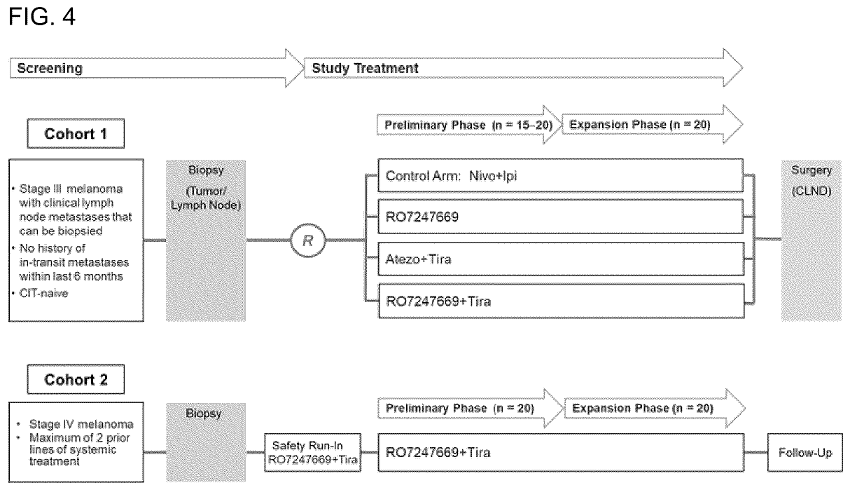

In Cohort 1, patients are randomly assigned to a control arm (nivolumab plus ipilimumab (Nivo+Ipi)) or an experimental arm consisting of RO7247669 (a bispecific antibody that binds to PD-1 and LAG3), atezolizumab in combination with tiragolumab (Atezo+Tira), or RO7247669 in combination with tiragolumab (RO7247669+Tira). Patients are stratified by geographic region (Australia vs. Rest of the World) and baseline LDH (≤the upper limit of normal (ULN) vs. >ULN). Details on the treatment regimens are provided in Table 12 and FIG. 4.

In Cohort 2, patients are enrolled into an experimental arm consisting of RO7247669 in combination with tiragolumab (RO7247669+Tira). Enrollment begins with a 6-patient safety run-in phase.

Approximately 61-191 patients are enrolled during the study, including approximately 6 patients who are enrolled in the safety run-in phase of Cohort 2. Enrollment within the experimental arms takes place in two phases: a preliminary phase, followed by an expansion phase. Approximately 15-20 patients are enrolled in each treatment arm during the preliminary phase. If clinical activity (pathologic response in Cohort 1) is observed in an experimental arm during the preliminary phase, approximately 20 additional patients may be enrolled in that arm during the expansion phase.

The Sponsor may decide to delay or suspend enrollment within a given treatment arm. Experimental arms with insufficient clinical activity or unacceptable toxicity are not expanded. Additional patients may be enrolled to ensure balance among treatment arms with respect to demographic and baseline characteristics, including potential predictive biomarkers, in order to enable further subgroup analyses.

The randomization ratio depends on the number of experimental arms that are available (e.g., if an arm is added or enrollment in an arm is suspended, pending analysis of results from the preliminary phase), with the stipulation that the likelihood of being allocated to the control arm is no more than 35%. Randomization takes into account arm-specific exclusion criteria. Patients are ineligible for a specific arm if they meet any of the exclusion criteria outlined for that arm.

Details on the treatment regimens are provided in Table 12.

TABLE 12
Treatment Regimens

| | | | Number of Patients | |
|---|---|---|---|---|
| | | Number of Patients | (Random Assignment) ^{c} | |
| | | (Sponsor Assignment) ^{b} | Preliminary | Expansion |
| Cohort | Study Treatment ^{a} | Safety Run-in Phase | Phase | Phase ^{d} |
| 1 | Control arm: nivolumab + | N/A | Variable ^{c} | |
| | ipilimumab | | | |
| 1 | RO7247669 | N/A | 20 ^{e} | 20 |
| 1 | Atezolizumab + tiragolumab | N/A | 20 ^{e} | 20 |
| 1 | RO7247669 + tiragolumab ^{f} | N/A | 20 ^{e} | 20 |
| 2 | RO7247669 + tiragolumab | ~6 | 20 | 20 |

^{a} The Sponsor may decide to delay or suspend enrollment within a given treatment arm. Thus, all experimental arms may not open for enrollment at the same time.
^{b} During the safety run-in phase, patients are assigned to available treatment arms. The treatment assignment ratio depends on the number of experimental arms that are open for enrollment.
^{c} The randomization ratio depends on the number of experimental arms that are open for randomization (e.g., if an arm is added or randomization into an arm is suspended pending analysis of results from the preliminary phase), with the stipulation that the likelihood of being allocated to the control arm is no more than 35%.
^{d} If clinical activity is observed in an experimental arm during the preliminary phase, approximately 20 additional patients are enrolled in that arm during the expansion phase. Experimental arms with minimal clinical activity or unacceptable toxicity do not undergo expansion.
^{e} Enrollment is suspended in the Cohort 1 RO7247669, Atezo + Tira, and RO7247669 + Tira arms to allow for a safety evaluation in a minimum of 6 patients.
^{f} Enrollment in the RO7247669 + Tira arm opens in Cohort 1 after safety assessment of the treatment combination in Cohort 2.

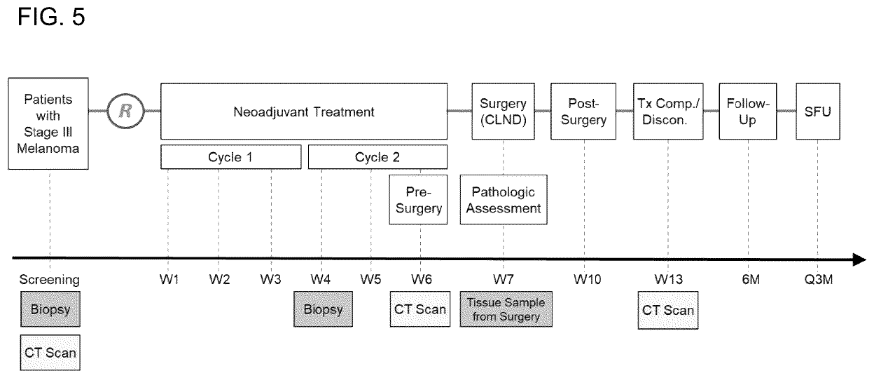

In Cohort 1, patients in the control arm and the experimental arms receive neoadjuvant treatment during a 6-week period. After completion of neoadjuvant treatment, or in case of discontinuation due to toxicity and in absence of disease progression, patients undergo surgery (completion lymph node dissection (CLND)) in Week 7. At the discretion of the investigator, outside this study, patients subsequently start either adjuvant therapy or observation commencing in Week 13 (FIG. 5).

Because of the possibility of an initial increase in the size of metastatic lymph nodes caused by immune-cell infiltration in the context of a T-cell response (termed pseudoprogression) with CITs, suspected clinical or radiographic progression per RECIST v1.1 may not be indicative of true disease progression. In the absence of unacceptable toxicity, patients who meet the criteria for disease progression per RECIST v1.1 while receiving treatment with a CIT drug are permitted to continue study treatment until surgery. Before discontinuation of study treatment and/or cancellation of surgery, progression is confirmed by biopsy or repeated radiographic assessment by an additional expert reviewer. All patients are expected to proceed with surgery, provided that there are no distant metastases and the surgeon considers the disease to be completely resectable.

In Cohort 2, patients continue to receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune-cell infiltration in the setting of a T-cell response (termed pseudoprogression) with atezolizumab and other CITs, radiographic progression per RECIST v1.1 may not be indicative of true disease progression. In the absence of unacceptable toxicity, patients who meet criteria for disease progression per RECIST v1.1 while receiving treatment with a CIT combination are permitted to continue treatment if they meet all of the following criteria:
Evidence of clinical benefit, as determined by the investigator following a review of all available data.
Absence of symptoms and signs (including laboratory values, such as new or worsening hypercalcemia) indicating unequivocal progression of disease.
Absence of decline in Eastern Cooperative Oncology Group (ECOG) Performance Status that can be attributed to disease progression.
Absence of tumor progression at critical anatomical sites (e.g., leptomeningeal disease) that cannot be managed by protocol-allowed medical interventions.

Patients eligible for treatment beyond progression are informed by the investigator that they may be foregoing other treatment options known to confer clinical benefit while continuing to receive the study treatment. Patients have the right to voluntarily withdraw from the study at any time for any reason. In addition, the investigator has the right to withdraw a patient from the study for medical conditions that the investigator or Sponsor determines may jeopardize the patient's safety if he/she continues in the study.

If at a subsequent tumor assessment, pseudoprogression is ruled out and progression of the disease is confirmed, the patient is discontinued from study treatment.

### Safety Evaluation Phase (Cohort 1)

To evaluate the toxicities of the experimental treatments in the neoadjuvant setting, enrollment is suspended after approximately 6 patients have been enrolled to allow for a safety evaluation. The safety evaluation is based on safety data from a minimum of 6 patients who have received at least one dose of treatment (i.e., one dose of each agent for a given combination) and who have completed safety follow-up assessments until surgery. Notably, timely conduct of surgery (CLND) is an indicator of treatment tolerability. During the 6-patient safety evaluation, or at any time following the safety evaluation, if ≥30% of patients experience one or more of the following events that is considered to be at least possibly related to study treatment, enrollment for that combination is put on hold while the Sponsor evaluates the benefit-risk profile of that treatment:
A treatment-related Grade ≥3 adverse event that does not improve (with or without treatment) to Grade 2 or better within 2 weeks.
A treatment-related adverse event causing >2-week delay in surgery.
A treatment-related serious adverse event.
A treatment-related adverse event that requires permanent discontinuation of study drug.
Death, except those that are incontrovertibly related to disease progression or extraneous causes such as accidents.

If no new safety signals are detected, enrollment resumes in that arm.

### Safety Run-In Phase (Cohort 2)

To assess the safety and tolerability of novel combinations that are tested clinically for the first time, an initial safety run-in phase is implemented in Cohort 2. Approximately 6 patients with metastatic disease are treated with the novel combination (i.e., RO7247669+Tira) and assessed for safety and tolerability for a minimum of 28 days.

A minimum of 6 patients in Cohort 2 must complete the initial safety run-in phase. If the RO7247669+Tira combination is determined to be tolerable, enrollment for the preliminary phase may be opened, and the RO7247669+Tira arm in Cohort 1 can be opened for enrollment. Patients in the safety run-in phase are enrolled and treated in a sequential manner, with at least one week between the first patient and the remaining patients.

The assessment is based on safety data from a minimum of 6 patients who have received at least one dose of treatment (i.e., one dose of each agent) and who have completed safety follow-up assessments for at least 28 days. If ≥30% of patients experience one or more of the following events that is considered to be at least possibly related to study treatment, enrollment for that combination is put on hold while the Sponsor evaluates the benefit-risk profile of that treatment:
A treatment-related Grade ≥3 adverse event that does not improve (with or without treatment) to Grade 2 or better within 2 weeks.
A treatment-related serious adverse event.
A treatment-related adverse event that requires permanent discontinuation of study drug.
Death, except those that are incontrovertibly related to disease progression or extraneous causes such as accidents.

If no new safety signals are detected, the combination is also initiated in Cohort 1.

### B. End of Study and Length of Study

The end of this study is defined as the date when the last patient completes the last visit, including survival follow-up visits conducted by telephone or in the clinic. The total length of the study, from screening of the first patient to the end of the study, is expected to be approximately 5 years.

### C. Rationale for Study Design

### Rationale for Patient Population

Cohort 1 enrolls patients with resectable Stage III melanoma with measurable lymph node metastases (according to RECIST v1.1) that can be biopsied, and who have no history of in-transit metastases within the last 6 months. Enrolled patients must not have received prior immunotherapy for their disease.

This same patient population is enrolled in the OpACIN and OpACIN-neo studies, including the PRADO extension cohort. These studies evaluated the neoadjuvant (and adjuvant) combination of nivolumab and ipilimumab in patients with resectable melanoma. Neoadjuvant therapy was found to have a statistically significant and clinically meaningful benefit as compared with adjuvant therapy (Rozeman et al. Lancet Oncol. 20: 948-960, 2019; Blank et al. J Clin Oncol. 38: 15S, 2020; Rozeman et al. Nat Med. 27: 256-263, 2021). In addition, the safety profile of the treatment was found to be tolerable in an optimized treatment schedule.

Despite the recently demonstrated benefit of checkpoint inhibition therapy, there is a continuing need for treatment regimens that are more efficacious (i.e., broader and deeper pathologic response in the surgical specimen) and better tolerated for patients with resectable melanoma. The multiple treatment options in this study are expected to stimulate the immune system through a variety of mechanisms. The aim is to extend the benefit of CIT beyond that of current checkpoint inhibition to a larger population with resectable melanoma.

Cohort 2 enrolls patients with Stage IV melanoma who experienced disease progression during or after at least one but not more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy) or may have received targeted therapy and checkpoint inhibition therapy concurrently as one combination treatment.

Novel combinations of compounds with a clinical and/or biological rationale for anti-melanoma activity that have not yet been tested clinically are investigated in Cohort 2. Importantly, for individual compounds considered for novel combinations, safety and tolerability have been already established in other studies, and a safe dose and schedule are available. The Cohort 2 safety run-in phase assesses the safety of the novel combination with regards to potential overlapping toxicities.

### D. Inclusion Criteria

### Shared Inclusion Criteria for Cohort 1 and Cohort 2

Patients must meet all of the following criteria to qualify for Cohort 1 and Cohort 2:
Age ≥18 years at the time of signing Informed Consent Form.
Availability of a representative tumor specimen that is suitable for determination of PD-L1 and/or additional biomarker status via central testing.
   Baseline tumor tissue samples are collected from all patients (except patients in the Cohort 2 safety run-in phase) by biopsy of a metastatic lymph node (Cohort 1) or other metastatic lesion (Cohort 2) at screening.
   In addition, archival primary tumor tissue is submitted from all patients if available. In case no archival primary tissue is available (e.g., for patients with unknown primary tumor), enrollment is permitted. For archival tissue, a formalin-fixed, paraffin-embedded (FFPE) tumor specimen in a paraffin block (preferred) with sufficient size and tumor content representation, preferably including the invasive margin, or at least 16 slides containing unstained, freshly cut, serial sections must be submitted along with an associated pathology report. If only 10-15 slides are available, the patient is still eligible for the study.
Adequate hematologic and end-organ function, defined by the following laboratory test results, obtained within 14 days prior to initiation of study treatment: Absolute neutrophil count (ANC) ≥1.5×10⁹/L (1500/μL); lymphocyte count ≥0.5×10⁹ cells/L (500/μL) (borderline machine lymphocyte counts may be confirmed by a manual count); platelet count ≥100×10⁹/L (100,000/μL); hemoglobin ≥90 g/L (9 g/dL); AST, ALT, and ALP ≤2.5×ULN (participants with documented liver metastases may have AST and ALT s 5×ULN; participants with documented liver or bone metastasis may have ALP ≤5×ULN); total bilirubin ≤1.5×ULN (patients with known Gilbert disease may have bilirubin level ≤3×ULN); creatinine s 1.5×ULN or creatinine clearance ≥30 mL/min (calculated using the Cockcroft-Gault formula); serum albumin ≥25 g/L (2.5 g/dL). Patients not receiving therapeutic anticoagulation may have INR and aPTT ≤1.5×ULN.
For patients receiving therapeutic anticoagulation: stable anticoagulant regimen (i.e., no new thrombosis, thromboembolic event, or bleeding episode within 3 months prior to study treatment start).
Negative HIV test at screening. Patients without a prior positive HIV test result undergo an HIV test at screening, unless not permitted per local regulations.
Negative hepatitis B surface antibody (HBsAb), and negative total hepatitis B core antibody (HBcAb) test at screening. If a patient has a negative hepatitis B surface antigen (HBsAg) test and a positive total HBcAb test at screening, an hepatitis B virus (HBV) DNA test must also be performed to rule out active HBV.
Negative hepatitis C virus (HCV) antibody test at screening, or positive HCV antibody test followed by a negative HCV RNA test at screening. The HCV RNA test is performed only for patients who have a positive HCV antibody test.
For women of childbearing potential: agreement to remain abstinent (refrain from heterosexual intercourse) or use contraceptive measures.
For men: agreement to remain abstinent (refrain from heterosexual intercourse) or use contraceptive measures, and agreement to refrain from donating sperm, as outlined for each specific treatment arm.

### Inclusion Criteria for Cohort 1

Patients must meet all of the following criteria to qualify for Cohort 1:
ECOG performance status (PS) of 0 or 1.
Histologically confirmed resectable Stage III melanoma (T: T0, Tx or T1-4; N: cN1-3, pN1b/2b/3b; M: M0 according to AJCC-8 (Gershenwald et al. CA Cancer J Clin. 67: 472-492, 2017)) and no history of in-transit metastases within the last 6 months.
   Patients may present with primary melanoma with concurrent regional nodal metastasis, or a history of primary melanoma or unknown primary melanoma with clinically detected regional nodal recurrence, and may belong to any of the following groups: Primary cutaneous melanoma with concurrent clinically/radiologically apparent regional lymph node metastases; clinically/radiologically detected recurrent melanoma at the proximal regional lymph node(s) basin; or clinically/radiologically detected nodal melanoma (if single site) arising from an unknown primary.
Fit and planned for CLND (as assessed by surgeon prior to randomization according to local guidelines).
Measurable disease (at least one target lesion) according to RECIST v1.1. At least one macroscopic lymph node metastasis (measurable according to RECIST v1.1) to be biopsied.

### Inclusion Criteria for Cohort 2

Patients must meet all of the following criteria to qualify for Cohort 2:
ECOG PS of 0, 1, or 2.
Life expectancy ≥3 months, as determined by the investigator.
Histologically confirmed Stage IV (metastatic) melanoma according to AJCC-8 (Gershenwald et al. CA Cancer J Clin. 67: 472-492, 2017).
Disease progression during or following at least one but no more than two lines of treatment for metastatic disease. Up to two lines of checkpoint inhibition therapy (monotherapy or combination therapy) are allowed. Patients with BRAF-mutant disease may have received an additional line of targeted therapy (either before, intermittent with, or after the checkpoint inhibition therapy), or may have received targeted therapy and checkpoint inhibition therapy concurrently as one combination treatment.
Patients who relapse or systemically progress during or within 6 months of completion of adjuvant therapy for localized melanoma may be eligible.
Measurable disease (at least one target lesion) according to RECIST v1.1.

At least one metastasis (measurable according to RECIST v1.1).

### E. Exclusion Criteria

Patients are excluded from enrollment in specific arms if they meet any of the applicable criteria outlined in subsequent sections, as specified by treatment arm below:

### Exclusion Criteria for Cohort 1 and Cohort 2

Patients who meet any of the following criteria are excluded from study entry:
Mucosal and uveal melanoma.
   Acral lentiginous melanoma is excluded for Cohort 1.
   For Cohort 2, acral lentiginous melanoma is permitted; however, the proportion of patients should not exceed 20% of response-evaluable patients.
Treatment with investigational therapy within 28 days prior to initiation of study treatment.
Treatment with systemic immunostimulatory agents (including, but not limited to, IFN and IL-2) within 4 weeks or 5 drug-elimination half-lives (whichever is longer) prior to initiation of study treatment.
Prior allogeneic stem cell or solid organ transplantation.
Known immunodeficiency or conditions requiring treatment with systemic immunosuppressive medication (including, but not limited to, cyclophosphamide, azathioprine, methotrexate, thalidomide, and anti-tumor necrosis factor-α agents), or anticipation of need for systemic immunosuppressant medication during study treatment, with the following exceptions: patients on replacement doses of corticosteroids to manage hypopituitary or adrenal insufficiency are eligible for the study; patients who received acute, low-dose, systemic immunosuppressant medications, or a one-time pulse dose of systemic immunosuppressant medication (e.g., 48 hours of corticosteroids for a contrast allergy) are eligible for the study after discussion with Medical Monitor; patients who received mineralocorticoids (e.g., fludrocortisone), corticosteroids for chronic obstructive pulmonary disease or asthma, or low-dose corticosteroids for orthostatic hypotension or adrenal insufficiency are eligible for the study.
Treatment with a live, attenuated vaccine within 4 weeks prior to initiation of study treatment, or anticipation of need for such a vaccine during study treatment or within 5 months after the final dose of study treatment.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
   Patients with a history of autoimmune-related hypothyroidism who are on thyroid-replacement hormone are eligible for the study.
   Patients with controlled Type 1 diabetes mellitus who are on a stable insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met: rash must cover <10% of body surface area; disease is well controlled at baseline and requires only low-potency topical corticosteroids; there is no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan. Patients with a history of CIT-related pneumonitis Grade <2 may be eligible after discussion with the Medical Monitor.
History of malignancy other than malignant melanoma within 2 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.
Active tuberculosis (TB).
Severe infection within 4 weeks prior to initiation of study treatment, including, but not limited to, hospitalization for complications of infection, bacteremia, or severe pneumonia, or any active infection that, in the opinion of the investigator, could impact patient safety.
Treatment with therapeutic or prophylactic oral or IV antibiotics within 2 weeks prior to initiation of study treatment.
Significant cardiovascular disease such as New York Heart Association cardiac disease (Class II or greater), myocardial infarction or cerebrovascular accident within 3 months prior to initiation of study treatment, unstable arrhythmia, or unstable angina.
Uncontrolled hypertension (defined as resting systolic blood pressure >150 mmHg and/or diastolic blood pressure >100 mmHg in two or more serial measurements).
Major surgical procedure, other than for diagnosis, within 4 weeks prior to initiation of study treatment, or anticipation of need for a major surgical procedure other than CLND, during the study.
   Placement of central venous access catheter (e.g., port or similar) is not considered a major surgical procedure and is therefore permitted.
Any other disease, metabolic dysfunction, physical examination finding, or clinical laboratory finding that contraindicates the use of an investigational drug, may affect the interpretation of the results, impair the ability of the patient to participate in the study, or may render the patient at high risk from treatment complications.
History of severe allergic reactions to chimeric or humanized antibodies or fusion proteins.
Known hypersensitivity to Chinese hamster ovary cell products or recombinant human antibodies.
Known allergy or hypersensitivity to any of the study drugs or their excipients.
Known intolerance to any of the drugs required for premedication (acetaminophen, ranitidine, diphenhydramine, and methylprednisolone).
Pregnancy or breastfeeding, or intention of becoming pregnant during the study.
   Women of childbearing potential must have a negative serum pregnancy test result within 14 days prior to initiation of study treatment.
Eligible only for the control arm.

### Exclusion Criteria for Cohort 1

Patients who meet any of the following criteria are excluded from Cohort 1:
Distantly metastasized melanoma
History of in-transit metastases within the last 6 months
Prior radiotherapy
Prior immunotherapy, including anti-CTLA-4, anti-PD-1, and anti-PD-L1 therapeutic antibodies, and other systemic therapy for melanoma

### Exclusion Criteria for Cohort 2

Patients who meet any of the following criteria are excluded from Cohort 2:
Symptomatic, untreated, or progressing CNS metastases.
   Asymptomatic patients with treated CNS lesions are eligible, provided that all of the following criteria are met: Measurable disease, per RECIST v1.1, must be present outside the CNS; the patient has no history of intracranial hemorrhage or spinal cord hemorrhage; CNS metastases are stable for ≥4 weeks prior to initiation of study, or neurosurgical resection occurred ≥28 days prior to initiation of study treatment; the patient has no requirement for corticosteroids as therapy for CNS disease for at least 14 days prior to initiation of study treatment; anti-convulsant therapy at a stable dose is permitted.
Active or history of carcinomatous meningitis/leptomeningeal disease.
Uncontrolled tumor-related pain. Patients requiring pain medication must be on a stable regimen at screening. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for loco-regional therapy, if appropriate, prior to enrollment.
Uncontrolled pleural effusion, pericardial effusion, or ascites requiring recurrent drainage procedures (once monthly or more frequently). Patients with indwelling catheters (e.g., PLEURX®) are allowed.
Uncontrolled or symptomatic hypercalcemia (ionized calcium >1.5 mmol/L, calcium >12 mg/dL, or corrected calcium >ULN).
Any history of an immune-mediated Grade 4 adverse event attributed to prior CIT (other than endocrinopathy managed with replacement therapy or asymptomatic elevation of serum amylase or lipase) that resulted in permanent discontinuation of the prior immunotherapeutic agent.
All immune-mediated adverse events related to prior immunomodulatory therapy (other than endocrinopathy managed with replacement therapy or stable vitiligo) that have not resolved completely to baseline. Patients treated with corticosteroids for immune-mediated adverse events, except for corticosteroids replacement therapy for adrenal insufficiency (provided that the patient receives ≤10 mg prednisone/day or equivalent), must not have related symptoms or signs for ≥4 weeks following discontinuation of corticosteroids.
Adverse events related to any prior radiotherapy, chemotherapy, targeted therapy, CPI therapy or surgical procedure must have resolved to Grade 1 or better, except alopecia (any grade), Grade 2 peripheral neuropathy, and hypothyroidism and/or hypopituitarism on a stable dosage of hormone replacement therapy (e.g., thyroxine, hydrocortisone, prednisolone, others).

### Exclusion Criteria for RO7247669-Containing Arms (Cohort 1 and Cohort 2)

Patients who meet any of the following criteria are excluded from the RO7247669-containing arm:
Prior treatment with an anti-LAG-3 agent.
History of myocarditis (regardless of etiology).
Left ventricular ejection fraction (LVEF)<50% assessed by either transthoracic echocardiogram (TTE) or multiple-gated acquisition (MUGA) scan (TTE preferred test) within 6 months prior to initiation of study treatment.
Troponin T (TnT) or troponin I (TnI) >institutional ULN. Patients with TnT or TnI levels between >1 and <2×ULN are eligible if repeat levels within 24 hours are ≤1×ULN. If repeat levels within 24 hours are between >1 and <2×ULN, patients need to undergo a cardiac evaluation and may be considered for treatment if there are no clinically significant findings.

### Exclusion Criteria for Tiragolumab-Containing Arms (Cohort 1 and Cohort 2)

Patients who meet any of the following criteria are excluded from the tiragolumab-containing arm:
Prior treatment with an anti-TIGIT agent.
Active Epstein-Barr virus (EBV) infection or known or suspected chronic active EBV infection at screening. Patients with a positive EBV viral capsid antigen (VCA) IgM test at screening are excluded from the tiragolumab-containing arms. An EBV PCR test should be performed as clinically indicated to screen for active infection or suspected chronic active infection. Patients with a positive EBV PCR test are excluded from the tiragolumab-containing arms.

### F. Study Treatment

The investigational medicinal products for this study are atezolizumab, tiragolumab, RO7247669, nivolumab, and ipilimumab.

### Control Arm (Nivolumab+Ipilimumab)

Patients in the nivolumab plus ipilimumab (Nivo+Ipi) control arm receive treatment for 2 cycles (6 weeks) as outlined in Table 13 until surgery, or until unacceptable toxicity or loss of clinical benefit, whichever occurs first. It is recommended that treatment be initiated no later than 7 days after randomization.

TABLE 13
Treatment Regimen for Nivolumab + Ipilimumab Arm

| | Dose, Route, and Regimen |
|---|---|
| Cycle Length | (drugs listed in order of administration) |
| 21 days | Nivolumab 3 mg/kg IV on Day 1 of each cycle |
| | Ipilimumab 1 mg/kg IV on Day 1 of each cycle |

Nivolumab is administered by IV infusion at a dose of 3 mg/kg on Day 1 of each 21-day cycle (Q3W). Ipilimumab is administered by IV infusion at a dose of 1 mg/kg on Day 1 of each 21-day cycle (Q3W).

### RO7247669 Arm

Patients in the RO7247669 arm receive treatment for 2 cycles (6 weeks) as outlined in Table 14 until surgery, or until unacceptable toxicity or loss of clinical benefit, whichever occurs first. It is recommended that treatment be initiated no later than 7 days after randomization.

TABLE 14
Treatment Regimen for RO7247669 Arm

| Cycle | Dose, Route, and Regimen |
|---|---|
| Length | (drugs listed in order of administration) |
| 21 days | RO7247669 2100 mg by IV infusion on Day 1 of each cycle |

RO7247669 is administered at a fixed dose of 2100 mg Q3W (2100 mg on Day 1 of each 21-day cycle). Administration of RO7247669 is performed in a monitored setting where there is immediate access to trained personnel and adequate equipment and medicine to manage potentially serious reactions. RO7247669 infusions are administered per the instructions outlined in Table 15.

TABLE 15
Administration of First and Second RO7247669 Infusions

| First Infusion | Second Infusion |
|---|---|
| No premedication is permitted prior to the | If the patient experienced an IRR with the first |
| first RO7247669 infusion. | infusion, premedication with antihistamines, |
| Vital signs (pulse rate, respiratory rate, | antipyretics, and/or analgesics may be |
| blood pressure, pulse oximetry, and | administered for subsequent doses at the |
| temperature) should be measured within 60 | discretion of the investigator. |
| minutes prior to the infusion. | Vital signs should be measured within 60 |
| RO7247669 should be infused over 60 (±10) | minutes prior to the infusion. |
| minutes. | RO77247669 should be infused over 30 (±10) |
| After the infusion of RO7247669, the patient | minutes if the first infusion was tolerated without |
| begins a 60-minute observation period. | an IRR, followed by a 30-minute observation |
| If clinically indicated, vital signs should be | period. |
| measured every 15 (±5) minutes during the | If the patient experienced an IRR with the |
| infusion and at 30 (±10) minutes after the | previous infusion or if clinically indicated, vital |
| infusion. | signs should be measured every 15 (±5) |
| Patients should be informed about the | minutes during the infusion and at 30 (±10) |
| possibility of delayed post-infusion | minutes after the infusion. |
| symptoms and instructed to contact their | |
| study physician if they develop such | |
| symptoms. | |

IRR = infusion-related reaction.

For patients who experience a Grade 2 infusion-related reaction (IRR), premedication with paracetamol 500-1000 mg orally (PO) or IV and diphenhydramine 25-50 mg PO or IV (or an alternative histamine H_{1/2} antagonist at an adequate dose) is required prior to subsequent infusions. In case of Grade 3 or 4 IRRs related to study treatment, the patient should be permanently discontinued from the study treatment.

No dose modification for RO7247669 is allowed.

### Atezolizumab 120 Tiragolumab

Patients in the atezolizumab plus tiragolumab (Atezo+Tira) arm will receive treatment for 2 cycles (6 weeks) as outlined in Table 16 until surgery, or until unacceptable toxicity or loss of clinical benefit, whichever occurs first. It is recommended that treatment be initiated no later than 7 days after randomization.

TABLE 16
Treatment Regimen for Atezolizumab + Tiragolumab Arm

| | Dose, Route, and Regimen |
|---|---|
| Cycle Length | (drugs listed in order of administration) |
| 21 days | Atezolizumab 1200 mg IV on Day 1 of each cycle |
| | Tiragolumab 600 mg IV on Day 1 of each cycle |

Atezolizumab is administered at a fixed dose of 1200 mg every 3 weeks (Q3W) (1200 mg on Day 1 of each 21-day cycle). Administration of atezolizumab is performed in a monitored setting where there is immediate access to trained personnel and adequate equipment and medicine to manage potentially serious reactions. Atezolizumab infusions are administered per the instructions outlined in Table 17.

No dose modification for atezolizumab is allowed.

TABLE 17
Administration of First and Second Atezolizumab Infusions

| First Infusion | Second Infusion |
|---|---|
| No premedication is permitted | If the patient experienced an |
| prior to the atezolizumab | IRR with the first infusion, |
| infusion. | premedication with antihistamines, |
| Vital signs (pulse rate, | anti-pyretics, and/or analgesics |
| respiratory rate, pulse | may be administered for subsequent |
| oximetry, blood pressure, | doses at the discretion of the |
| and temperature) should be | investigator. |
| measured within 60 minutes | Vital signs should be measured |
| prior to the infusion. | within 60 minutes prior to the |
| Atezolizumab should be | infusion. |
| infused over 60 (±15) minutes. | Atezolizumab should be infused |
| After the infusion of | over 30 (±10) minutes if the |
| atezolizumab, the patient | previous infusion was tolerated |
| begins a 30-minute observation | without an IRR, or |
| period. | 60 (±15) minutes if the patient |
| If clinically indicated, vital | experienced an IRR with the |
| signs should be measured every | previous infusion. |
| 15 (±5) minutes during the | If the patient experienced an |
| infusion and 30 (±10) minutes | IRR with the previous infusion |
| after the infusion. | or if clinically indicated, vital |
| Patients should be informed | signs should be measured during |
| about the possibility of delayed | the infusion and at 30 (±10) |
| post-infusion symptoms and | minutes after the infusion. |
| instructed to contact their | |
| study physician if they develop | |
| such symptoms. | |

IRR = infusion-related reaction.

Tiragolumab is administered at a fixed dose of 600 mg IV Q3W (600 mg on Day 1 of each 21-day cycle). Administration of tiragolumab is performed in a monitored setting where there is immediate access to trained personnel and adequate equipment and medicine to manage potentially serious reactions. Tiragolumab infusions are administered per the instructions outlined in Table 18.

TABLE 18
Administration of First and Subsequent Tiragolumab Infusions

| First Infusion | Second and Susequent Infusions |
|---|---|
| No premedication is permitted | If the patient experienced |
| prior to the tiragolumab | an IRR with the first infusion, |
| infusion. | premedication with antihistamines, |
| Vital signs (pulse rate, | antipyretics, and/or analgesics |
| respiratory rate, pulse | may be administered for subsequent |
| oximetry, blood pressure, | doses at the discretion of the |
| and temperature) should be | investigator. |
| measured within 60 minutes | Vital signs should be measured |
| prior to the infusion. | within 60 minutes prior to the |
| Tiragolumab should be infused | infusion. |
| over 60 (±15) minutes. | Tiragolumab should be infused |
| After the infusion of tiragolumab, | over 30 (±10) minutes if the |
| the patient begins a 60-minute | previous infusion was tolerated |
| observation period. | without an IRR, or 60 (±15) |
| Record vital signs every 15 (±5) | minutes if the patient experienced |
| minutes during the infusion and | an infusion-related reaction with |
| at 30 (±10) minutes after the | the previous infusion. |
| infusion. | Patients should be observed for |
| Patients are informed about the | 30 minutes after completion of |
| possibility of delayed postinfusion | the tiragolumab infusion if the |
| symptoms and are instructed to | previous infusion was tolerated |
| contact their study physician if | without an IRR, or for 60 minutes |
| they develop such symptoms. | after completion of the tiragolumab |
| | infusion if the patient experienced |
| | an IRR with the previous infusion. |
| | If clinically indicated, vital |
| | signs should be recorded every 15 |
| | (±5) minutes during the infusion |
| | and at 30 (±10) minutes after the |
| | infusion. |

IRR = infusion-related reaction.

Atezolizumab and tiragolumab treatment may be temporarily suspended in patients experiencing toxicity considered to be related to study treatment. If corticosteroids are initiated for treatment of the toxicity, they must be tapered over ≥1 month to the equivalent of ≤10 mg/day oral prednisone before study treatment can be resumed, if warranted. In the neoadjuvant setting, the study treatment is limited to a pre-surgery window of 6 weeks. Treatment during this period should not be interrupted, unless a patient experiences toxicity. If toxicity meets criteria for interrupting/withholding atezolizumab and/or tiragolumab, atezolizumab and/or tiragolumab should be interrupted/withheld. After resolution of the toxicity, subsequent treatment cycles should only be considered if the benefit/risk profile is acceptable and if the surgery can be conducted within 2 weeks of the planned date. Otherwise, subsequent treatment cycles should be omitted to allow the patient to proceed directly to surgery without further delay.

On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, adverse events should generally be attributed to both agents, and dose interruptions or treatment discontinuation in response to adverse events should be applied to both tiragolumab and atezolizumab. If atezolizumab is withheld or discontinued, tiragolumab should also be withheld or discontinued. If tiragolumab is withheld or discontinued, atezolizumab should also be withheld or discontinued.

### RO7247669+Tiragolumab (Cohorts 1 and 2)

Patients in the RO7247669 plus tiragolumab (RO7247669+Tira) arm receive treatment as outlined in Table 19.

Patients in Cohort 1 receive treatment for 2 cycles (6 weeks) until surgery, or until unacceptable toxicity or loss of clinical benefit, whichever occurs first.

Patients in Cohort 2 receive treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease).

It is recommended that treatment be initiated no later than 7 days after randomization (Cohort 1) or enrollment (Cohort 2).

TABLE 19
Treatment Regimen for RO7247669 + Tiragolumab Arm

| | Dose, Route, and Regimen |
|---|---|
| | (drugs listed in order |
| Cycle Length | of administration) |
| 21 days | RO7247669 2100 mg IV on |
| | Day 1 of each cycle ^{a} |
| | Tiragolumab 600 mg IV on |
| | Day 1 of each cycle |

^{a} After the safety run-in has been completed, the Sponsor may decide to explore lower doses (e.g., 1200 mg and 600 mg).

RO7247669 is administered by IV infusion at a fixed dose of 2100 mg on Day 1 of each 21-day cycle. Tiragolumab is administered by IV infusion at a fixed dose of 600 mg on Day 1 of each 21-day cycle with a post-infusion observation period as described in Table 18.

Administration of RO7247669 is performed in a monitored setting where there is immediate access to trained personnel and adequate equipment and medicine to manage potentially serious reactions. RO7247669 infusions are administered per the instructions outlined in Table 20.

TABLE 20
Administration of First, Second, and
Subsequent RO7247669 Infusions

| First and Second Infusion | Subsequent Infusions |
|---|---|
| No premedication is permitted | If the patient experienced |
| prior to the first RO7247669 | an IRR with any previous |
| infusion. | infusion, premedication |
| Vital signs (respiratory rate, | with antihistamines, |
| pulse rate, blood pressure, | antipyretics, and/or |
| pulse oximetry, and temperature) | analgesics may be |
| should be measured within 60 | administered for subsequent |
| minutes prior to the infusion. | doses at the discretion |
| For the first infusion, | of the investigator. |
| RO7247669 should be infused | Vital signs should be |
| over 60 (±10) minutes. | measured within 60 minutes |
| After the infusion of | prior to the infusion. |
| RO7247669, the patient begins | RO77247669 should be infused |
| a 60-minute observation | over 30 (±10) minutes |
| period. | if the previous infusion |
| For the second infusion, | was tolerated without an |
| RO7247669 should be infused | IRR. If the patient |
| over 30 (±10) minutes | tolerated the previous |
| if the first infusion was | infusion of RO77247669 |
| tolerated without an IRR. | well without infusion- |
| If clinically indicated, | associated adverse events, |
| vital signs should be measured | the observation period may |
| every 15 (±5) minutes | be reduced to 30 minutes. |
| during the infusion and at | If the patient experienced |
| 30 (±10) minutes after | an IRR with the previous |
| the infusion. | infusion or if clinically |
| Patients should be informed | indicated, vital signs |
| about the possibility of | should be measured every |
| delayed post-infusion symptoms | 15 (±5) minutes during |
| and instructed to contact | the infusion and at 30 |
| their study physician if | (±10) minutes after |
| they develop such symptoms. | the infusion. |

IRR = infusion-related reaction.

For patients who experience a Grade 2 infusion-related reaction (IRR), premedication with paracetamol 500-1000 mg orally (PO) or IV and diphenhydramine 25-50 mg PO or IV (or an alternative histamine H_{1/2} antagonist at an adequate dose) is required prior to subsequent infusions. In case of Grade 3 or 4 IRRs related to study treatment, the patient should be permanently discontinued from the study treatment.

No dose modification for RO7247669 is allowed. However, based on emerging safety and efficacy data, the Sponsor may explore lower doses (e.g., 1200 mg and 600 mg). RO7247669 treatment may be interrupted for reasons other than toxicity (e.g., surgical procedures).

The investigator and the Medical Monitor will determine the acceptable length of treatment interruption.

Treatment with RO7247669 and tiragolumab may be temporarily suspended in patients experiencing toxicity considered to be related to study treatment. If corticosteroids are initiated for treatment of the toxicity, they must be tapered over ≥1 month to the equivalent of ≤10 mg/day oral prednisone before study treatment can be resumed, if warranted.

For Cohort 1, in the neoadjuvant setting the study treatment is limited to a pre-surgery window of 6 weeks. Treatment during this period should not be interrupted unless a patient experiences toxicity. If toxicity meets criteria for interrupting/withholding RO7247669 and tiragolumab, RO7247669 and tiragolumab should be interrupted/withheld. After resolution of the toxicity, subsequent treatment cycles should only be considered if the benefit/risk profile is acceptable and if the surgery can be conducted within 2 weeks of the planned date. Otherwise, subsequent treatment cycles should be omitted to allow the patient to proceed directly to surgery without further delay.

For Cohort 2, if RO7247669 and tiragolumab are withheld for 12 weeks or longer due to toxicity, the patient should be discontinued from RO7247669 and tiragolumab. However, RO7247669 and tiragolumab may be withheld for more than 12 weeks to allow for patients to taper off corticosteroids prior to resuming treatment. RO7247669 and tiragolumab may be resumed after being withheld for more than 12 weeks if the Medical Monitor agrees that the patient is likely to derive clinical benefit. RO7247669 and tiragolumab treatment may be suspended for reasons other than toxicity (e.g., surgical procedures). The acceptable length of the extended period of time must be agreed upon by the investigator and the Medical Monitor.

On the basis of the available characterization of mechanism-of-action, tiragolumab may cause adverse events similar to, but independent of, RO7247669. Tiragolumab may also exacerbate the frequency or severity of RO7247669-related adverse events or may have non-overlapping toxicities with RO7247669. Because these scenarios may not be distinguishable from each other in the clinical setting, adverse events should generally be attributed to both agents, and dose interruptions or treatment discontinuation in response to adverse events should be applied to both tiragolumab and RO7247669. If RO7247669 is withheld or discontinued, tiragolumab should also be withheld or discontinued. If tiragolumab is withheld or discontinued, RO7247669 should also be withheld or discontinued.

### G. Concomitant Therapy

Concomitant therapy consists of any medication (e.g., prescription drugs, over-the-counter drugs, vaccines, herbal or homeopathic remedies, nutritional supplements) used by a patient in addition to protocol-mandated study treatment from 7 days prior to initiation of study treatment to the treatment discontinuation visit.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and P2-adrenergic agonists).

### Permitted Therapy for RO7247669, Atezo+Tira, and RO7247669+Tira Arms

Patients are permitted to use the following therapies during the study:
Oral contraceptives with a failure rate of <1% per year.
Hormone-replacement therapy.
Prophylactic or therapeutic anticoagulation therapy (such as warfarin at a stable dose or low-molecular-weight heparin).
Inactivated vaccinations (e.g., influenza).
Megestrol acetate administered as an appetite stimulant
Mineralocorticoids (e.g., fludrocortisone)
Corticosteroids administered for chronic obstructive pulmonary disease (COPD) or asthma.
Low-dose corticosteroids administered for orthostatic hypotension or adrenocortical insufficiency.
Local therapy (e.g., surgery (other than complete lymph node dissection (CLND) and not melanoma specific).

For the RO7247669 arm, premedication with antihistamines, antipyretics, and/or analgesics may be administered for the second RO7247669 infusion only, at the discretion of the investigator.

For the atezolizumab plus tiragolumab arm, premedication with antihistamines, antipyretics, and/or analgesics may be administered for the second atezolizumab and tiragolumab infusions only, at the discretion of the investigator.

### Additional Permitted Therapy for RO7247669+Tira Arm in Cohort 2

Patients are permitted to use the following therapies during the study:
Palliative radiotherapy (e.g., treatment of known bony metastases or symptomatic relief of pain) as outlined: Palliative radiotherapy is permitted, provided it does not interfere with the assessment of tumor target lesions (e.g., the lesion to be irradiated must not be the only site of measurable disease). Treatment with tiragolumab may be continued during palliative radiotherapy. Treatment with RO7247669 may be continued during palliative radiotherapy with one exception: palliative radiotherapy is not permitted on days when RO7247669 is administered.
Local therapy (e.g., surgery, stereotactic radiosurgery, radiotherapy, radiofrequency ablation) as outlined: Patients experiencing a mixed response requiring local therapy for control of three or fewer lesions may still be eligible to continue study treatment after Medical Monitor approval has been obtained. Patients who receive local therapy directed at a target lesion will no longer be evaluable for radiographic response but will remain evaluable for progression.

Premedication with antihistamines, antipyretics, and/or analgesics may be administered for the second and subsequent RO7247669 and tiragolumab infusions only, at the discretion of the investigator.

### H. Assessments

All patients are closely monitored for adverse events throughout the study. Adverse events are graded according to the National Cancer Institute Common Terminology Criteria for Adverse Events, Version 5.0 (NCI CTCAE v5.0). Cytokine-release syndrome (CRS) severity is also graded according to the American Society for Transplantation and Cellular Therapy (ASTCT) CRS Consensus Grading Scale.

Patients in Cohort 1 receive neoadjuvant treatment for 2 cycles (6 weeks) and undergo surgery (CLND) in Week 7. All patients are expected to proceed with surgery, provided that there are no distant metastases and the surgeon considers the disease to be completely resectable. Pathologic response is assessed locally and by independent pathologic review.

Patients who discontinue treatment due to unacceptable toxicity and continue to have no evidence of metastatic disease are still eligible for surgery and proceed with CLND after the adverse event has resolved and re-staging confirms Stage III disease. If patients have confirmed disease progression, patient management and treatment selection are at the discretion of the treating physician. These patients remain in the study for follow-up.

Patients undergo radiological tumor assessments in Week 6 (from Day 1 of Cycle 1) prior to surgery (CLND). Response is assessed and determined by the investigator in accordance with RECIST v1.1, but confirmation by later imaging studies is not required.

Patients in Cohort 2 undergo tumor assessments every 9 weeks (from Day 1 of Cycle 1) for the first 54 weeks and then every 12 weeks thereafter. Response is assessed by the investigator using RECIST v1.1. Response per modified RECIST v1.1 for immune-based therapeutics (iRECIST) is determined programmatically by the Sponsor on the basis of investigator-assessed individual lesion data.

For Cohort 1 and Cohort 2, if clinical activity is demonstrated in an experimental arm, the Sponsor may request that tumor assessment scans for that arm be submitted for evaluation by an independent review facility.

### Tumor and Response Evaluations

All measurable and evaluable lesions should be assessed and documented at screening. Tumor assessments performed as standard of care prior to obtaining informed consent and within 14 days prior to randomization/enrollment do not have to be repeated at screening.

All measurable and/or evaluable lesions identified at baseline should be re-assessed at subsequent tumor evaluations for Cohorts 1 and 2. The same radiographic procedures used to assess disease sites at screening should be used for subsequent tumor assessments (e.g., the same contrast protocol for CT scans).

### Cohort 1 Tumor and Response Evaluations

Patients in Cohort 1 are assessed for pathologic and radiologic response to treatment. Patients undergo pathological tumor assessments at baseline, and after 6 weeks of treatment at surgery (CLND). The complete resection of Stage III lymph nodes (CLND) in Week 7 must be performed in compliance with the criteria for adequate surgical procedures for therapeutic lymph node dissection. CLND should be performed as planned if the patient is receiving corticosteroids or other anti-inflammatory drugs for the management of immune-mediated adverse events, provided these are being given at a stable or tapering dose and the severity of adverse events is Grade 2 or better. CLND may be delayed for up to 2 weeks if study treatment-related adverse events have not improved sufficiently at the time of planned surgery. Pathological response is determined by local and independent pathologic review according to INMC guidelines (Tetzlaff et al. Ann Oncol. 29: 1861-1868, 2018).

Surgical complications are scored according to Clavien-Dindo classification. Complication rates for every grade are reported and scored for patients that underwent CLND.

Patients undergo radiographic tumor assessments at baseline, after 6 weeks of treatment before surgery (CLND), and at treatment completion/discontinuation at Week 13 according to RECIST v1.1.

Overall response at a single timepoint is assessed by the investigator using RECIST v1.1.

### Disease Follow-Up and Confirmation of Disease Progression or Recurrence

During the neoadjuvant treatment, diagnosis of disease progression should be confirmed by clinical, laboratory, radiological, and/or histological findings. After surgery, prior to commencing adjuvant treatment or observation, a tumor assessment is performed in Week 13 to conclude the neoadjuvant therapy-surgery intervention window.

Thereafter, outside the study during the adjuvant treatment/observation phase (i.e., commencing in Week 13), all patients must be followed to assess disease recurrence and survival as outlined for each arm.

Patients who complete the treatment period have their first survival follow-up visit 3 months after surgery. Patients who discontinue study drug prematurely have their first survival follow-up visit 3 months after the final dose of study treatment. The designation of disease recurrence, whether local, regional, or distant, can be made only when clinical, laboratory, radiological and/or histological findings confirm the diagnosis.

During the post-surgery period, disease status should be clinically evaluated and documented as per institutional guidelines (e.g., every 3 months for the first 2 years; every 6 months in the third year; once a year in the fourth and following years). In addition, liver function tests, bone scans, chest X-ray/diagnostic CT scan, liver imaging, and/or other radiographic modalities may be considered when clinically indicated to exclude metastatic disease.

The diagnosis of a progression or recurrence should be confirmed histologically whenever clinically possible. The earliest date of diagnosis of disease progression or recurrent disease should be used and recorded. This date should be based on objective clinical, radiological, histological, or cytological evidence. Recurrent disease includes local, regional, or distant recurrence.

The definitions of and procedures for confirming disease recurrence, death, and other noteworthy events on follow-up are provided below. Documentation of recurrence requires specification of all sites involved to establish the pattern of recurrence. The following criteria of treatment failure constitute the only acceptable evidence of disease recurrence:
Lung: Positive cytology or biopsy in the presence of a single new lesion or the appearance of multiple lesions consistent with metastatic disease
Liver: Positive cytology or biopsy in the presence of a single new lesion or the appearance of multiple lesions consistent with metastatic disease
Central nervous system: A positive brain CT or MRI scan or CSF cytology
Cutaneous, subcutaneous, and lymph node recurrence: Positive cytology or biopsy in the presence of a single new lesion or the appearance of multiple lesions consistent with metastatic disease
Bone and other organs: Positive cytology or biopsy in the presence of a single new lesion or the appearance of multiple lesions consistent with metastatic disease identified on two different radiologic studies (i.e., positive nuclear bone scan or PET scan and contrast GI series or ultrasound, X-ray or CT of abdomen for abdominal disease).

### Cohort 2 Tumor and Response Evaluations

Patients in Cohort 2 undergo tumor assessments every 9 (±1) weeks (from Day 1 of Cycle 1) for the first 54 weeks and then every 12 (±2) weeks thereafter, regardless of dose delays. The exception is patients who continue treatment after radiographic disease progression. Such patients undergo tumor assessments every 9 weeks until loss of clinical benefit as determined by the investigator. Thus, tumor assessments continue according to schedule in patients who discontinue treatment for reasons other than loss of clinical benefit, even if they start new, non-protocol-specified anti-cancer therapy. At the investigator's discretion, tumor assessments may be repeated at any time if progressive disease is suspected.

Brain metastases treated with radiotherapy or surgery are not considered measurable or evaluable but are documented at screening as a site of metastatic disease. Brain metastases identified at baseline that have been treated with radiotherapy or surgery are not considered to be measurable or evaluable unless there is suspected disease progression in the brain (i.e., the patient becomes symptomatic). Thus, subsequent head scans are not required unless clinically indicated.

To facilitate evaluation of response per iRECIST in Cohort 2, tumor assessments must be continued after disease progression per RECIST v1.1 for patients who receive treatment beyond progression. This includes continued measurement of target lesions, evaluation of non-target lesions (including monitoring for further worsening of any non-target lesions that have shown unequivocal progression), and evaluation of any newly identified lesions (including measurements, if lesions are measurable) at all subsequent assessments.

Overall response at a single timepoint is assessed by the investigator using RECIST v1.1.

### Biomarker Assessments

Baseline tumor tissue samples are collected from all patients (except patients in the Cohort 2 safety run-in phase) by biopsy of a metastatic lymph node (Cohort 1) or other metastatic lesion (Cohort 2) at screening. For patients in Cohort 1, on-treatment tissue samples are collected by biopsy on Day 1 of Cycle 2, and at surgery (CLND). For patients enrolled in Cohort 2, on-treatment tissue samples are collected by biopsy on Day 8 of Cycle 2.

Exploratory biomarker analyses are performed in an effort to understand the association of biomarkers with response to study drugs, taking into account efficacy and safety endpoints. Exploratory biomarker research may include, but is not limited to, analysis of genes or gene signatures associated with tumor immunobiology, PD-L1, lymphocyte subpopulations, T-cell receptor repertoire, or cytokines associated with T-cell activation. Research may involve DNA or RNA extraction, analysis of somatic mutations, and use of next-generation sequencing (NGS) (including whole exome sequencing (WES)). Research may involve extraction of DNA, cell-free DNA, or RNA; analysis of mutations, single nucleotide polymorphisms, and other genomic variants; and genomic profiling through use of NGS of a comprehensive panel of genes. DNA extracted from blood may be compared with DNA extracted from tissue to identify somatic variants by distinguishing germline variants from somatic variants.

NGS methods may include whole genome sequencing (WGS) or WES of tissue and blood samples. At participating sites, blood samples are collected for DNA extraction to enable WGS or WES to identify variants that are predictive of response to study drug, are associated with progression to a more severe disease state, are associated with acquired resistance to study drug, are associated with susceptibility to develop adverse events, can lead to improved adverse event monitoring or investigation, or can increase the knowledge and understanding of disease biology and drug safety. DNA extracted from blood may be compared with DNA extracted from tissue to identify somatic variants by distinguishing germline variants from somatic variants.

### I. Analysis

The final study analysis is based on patient data collected through study discontinuation. If not otherwise specified, efficacy analyses are based on the efficacy-evaluable population, defined as all patients who receive at least one dose of each drug for their assigned treatment regimen, and safety analyses are based on the safety-evaluable population, defined as all patients who receive any amount of study treatment.

Enrollment is summarized by region, country, and investigator by treatment arm. Patient disposition is summarized by treatment arm. Major protocol deviations, including major deviations with regard to the inclusion and exclusion criteria, are summarized by treatment arm.

For safety-evaluable patients, study drug administration data are tabulated or listed by treatment arm, and any dose modifications are flagged. Means and standard deviations are used to summarize the total dose and dose intensity for each study drug. Reasons for discontinuation of study drugs are tabulated.

Demographic and baseline characteristics (including age, sex, race/ethnicity, weight, malignancy duration, metastatic disease site (if applicable), and baseline ECOG PS) are summarized overall and by treatment arm.

### Determination of Sample Size

This study is not designed to make explicit power and type I error considerations for a hypothesis test. Instead, this study is designed to obtain preliminary efficacy, safety, and PK data on treatments or treatment combinations when administered to patients with melanoma. Cohort 1 consists of patients with resectable Stage III melanoma who have not received prior systemic therapy for their disease. Cohort 2 consists of patients with Stage IV melanoma who experienced disease progression during or after at least one but not more than two lines of treatment for metastatic disease.

In Cohort 1, approximately 55-145 patients are randomly allocated to the control and experimental arms during the study. In Cohort 2, approximately 6-46 patients are assigned to an experimental arm.

### Efficacy Analyses

### Primary Efficacy Endpoint in Cohort 1

The primary efficacy endpoint in Cohort 1 is pRR at time of surgery. pRR is assessed after completion of neoadjuvant treatment (Week 7) at time of CLND. The pRR is defined as the proportion of patients who achieve pCR (a complete absence of viable tumor in the treated tumor bed), pathologic near complete response (pnCR; <10% of viable tumor in the treated tumor bed); and pathologic partial response (pPR; <50% of the treated tumor bed is occupied by viable tumor cells) as determined by independent pathologic review. pRR is calculated for each arm along with 90% CIs. The difference in the pRR between the experimental arms and the control arm is also calculated along with 90% CIs. Confidence intervals are estimated by the exact method or the Wald method, depending on the sample size.

### Secondary Efficacy Endpoints in Cohort 1

The secondary efficacy endpoints in Cohort 1 are pRR at time of surgery as determined by local pathologic assessment, event-free survival (EFS), RFS, OS, and ORR prior to surgery. pRR is defined in Table 10.

EFS is defined as the time from randomization to any of the following events (whichever occurs first): disease progression that precludes surgery, as assessed by the investigator according to RECIST v1.1; local, regional, or distant disease recurrence; or death from any cause. Patients who have not experienced such events are censored at the time of their last post-tumor tumor assessment.

RFS is defined as the time from surgery to the first documented recurrence of disease or death from any cause. For patients who do not have documented recurrence of disease or death, RFS is censored at the day of the last tumor assessment.

OS is defined as the time from randomization to death from any cause. Patients who are still alive at the time of OS analysis are censored at the last date they were known to be alive.

The Kaplan-Meier method is used to estimate the median for RFS, EFS, and OS, 90% CIs are constructed using the Brookmeyer and Crowley method. The RFS, EFS, and OS rate at specific timepoints are also estimated using the Kaplan-Meier method, with 90% CIs calculated on the basis of Greenwood's estimate for the variance.

The ORR according to RECIST v1.1 is assessed after completion of neoadjuvant treatment (Week 7) and is defined as the proportion of patients with a CR or PR, as determined by the investigator according to RECIST v1.1. Patients with missing or no response assessments are classified as non-responders. Note that ORR is determined using unconfirmed pre-operative radiologic responses. Although RECIST v1.1 requires confirmatory imaging assessments to be completed at least 4 weeks after the initial response, due to the timing of CLND, these responses cannot be confirmed with subsequent imaging.

ORR is calculated for each arm, along with 90% CIs, using the Clopper-Pearson method. The difference in ORR between the experimental arms and the control arm is also calculated, along with 90% CIs. CIs are estimated by the exact method or the Wald method, depending on the sample size.

### Exploratory Efficacy Endpoints in Cohort 1

The exploratory efficacy endpoints in Cohort 1 are landmark EFS, landmark RFS, and landmark OS at specific timepoints (1, 2, 3, and 5 years).

Landmark EFS rates, landmark RFS rates, and landmark OS rates are estimated for each study arm using the Kaplan-Meier method, with 90% CIs calculated through use of Greenwood's formula.

### Primary Efficacy Endpoint in Cohort 2

The primary efficacy endpoint in Cohort 2 is ORR, as defined in Table 11. ORR is determined by the investigator according to RECIST v1.1. Patients with missing or no response assessments are classified as non-responders.

ORR, the proportion of patients with a complete or partial response, is calculated for each arm, along with 90% CIs (Clopper-Pearson method). CIs are estimated by the exact method or the Wald method, depending on the sample size.

### Secondary Efficacy Endpoints in Cohort 2

The secondary efficacy endpoints in Cohort 2 are PFS, OS, OS at specific timepoints (e.g., 6 months), duration of response (DOR), and disease control, as defined in Table 11. PFS, DOR, and disease control are determined by the investigator according to RECIST v1.1.

DOR is derived for efficacy-evaluable patients with a CR or PR.

For patients who do not have documented disease progression or death, PFS and DOR is censored at the day of the last tumor assessment.

Patients who are still alive at the time of OS analysis are censored at the last date they were known to be alive.

The Kaplan-Meier method is used to estimate the median for PFS, OS, and DOR, with 90% CIs constructed through use of the Brookmeyer and Crowley method. OS rate at specific timepoints is also estimated using the Kaplan-Meier method, with 90% CIs calculated on the basis of Greenwood's estimate for the variance.

Disease control rate (the proportion of patients with SD for ≥12 weeks), a PR, or a CR, will be calculated for each treatment arm, with 90% CIs estimated through use of Clopper-Pearson's exact method.

### Exploratory Efficacy Endpoints in Cohort 2

The exploratory efficacy endpoints are ORR, PFS, DOR, and disease control as determined by the investigator according to iRECIST.

ORR, PFS, DOR, and disease control are analyzed through use of the same methods described in the above sections, “Primary Efficacy Endpoint in Cohort 2” and “Secondary Efficacy Endpoints in Cohort 2.” DOR is derived for efficacy-evaluable patients with a complete or partial response.

### Safety Analyses

Verbatim adverse event terms are mapped to Medical Dictionary for Regulatory Activities thesaurus terms, and adverse event severity is graded according to NCI CTCAE v5.0 and also according to the ASTCT CRS Consensus Grading Scale for CRS.

Safety is assessed through summaries of adverse events, changes in laboratory test results, changes in vital signs and ECGs, and exposure to study drugs. Exposure to combination treatment and length of safety follow-up is summarized by treatment arm.

All verbatim adverse event terms are mapped to Medical Dictionary for Regulatory Activities thesaurus terms. Adverse event severity is graded according to NCI CTCAE v5.0, and severity of CRS will also be graded by the investigator according to the ASTCT Consensus Grading (Lee et al. Biol Blood Marrow Transplant. 25: 625-638, 2019). All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and causes of death are summarized.

Relevant laboratory, vital sign (pulse rate, respiratory rate, blood pressure, pulse oximetry, and temperature), and ECG data will be displayed by time, with grades identified where appropriate. Additionally, a shift table of selected laboratory test results is used to summarize the baseline and maximum postbaseline severity grade. Changes in vital signs and ECGs are summarized.

Additionally, in Cohort 1, the incidence, nature of immune-related adverse events Grade ≥3 during the first 12 weeks, and the rate and duration of delayed surgery due to treatment-related adverse events will be summarized by treatment arm. CLND may be delayed for up to 2 weeks if study treatment-related adverse events have not improved sufficiently at the time of planned surgery.

Additionally, surgical complications are scored according to Clavien-Dindo classification. Complication rates for every grade are reported and scored for patients that underwent CLND.

### Immunogenicity Analyses

Immunogenicity may be assessed for atezolizumab and other study treatments as appropriate. The immunogenicity analyses include all patients with at least one anti-drug antibody (ADA) assessment. Patients are grouped according to treatment received or, if no treatment is received prior to study discontinuation, according to treatment assigned.

For atezolizumab, the numbers and proportions of ADA-positive patients and ADA-negative patients at baseline (baseline prevalence) and after drug administration (postbaseline incidence) are summarized by treatment group. When determining postbaseline incidence, patients are considered to be ADA positive if they are ADA negative or have missing data at baseline but develop an ADA response following study drug exposure (treatment-induced ADA response), or if they are ADA positive at baseline and the titer of one or more postbaseline samples is at least 0.60-titer unit greater than the titer of the baseline sample (treatment-enhanced ADA response). Patients are considered to be ADA negative if they are ADA negative or have missing data at baseline and all postbaseline samples are negative, or if they are ADA positive at baseline but do not have any postbaseline samples with a titer that is at least 0.60-titer unit greater than the titer of the baseline sample (treatment unaffected).

For other study treatments where ADA is tested, positivity is determined according to standard methods established in previous studies of that drug.

The relationship between ADA status and safety, efficacy, PK, and biomarker endpoints may be analyzed and reported via descriptive statistics.

### Interim Analyses

Given the exploratory nature of this study, it is anticipated that interim analyses are conducted during the study, with the earliest interim analysis taking place when at least one experimental arm has completed enrollment in the preliminary phase, and patients have completed their pathologic response assessment (Cohort 1), or when at least one experimental arm has completed enrollment in the preliminary phase and patients have been followed for a minimum of 9 weeks for the primary endpoint analysis (ORR) (Cohort 2). Further interim analyses may be conducted as deemed appropriate by the Sponsor. In Cohort 1, a posterior probability may be used to guide further enrollment based on the interim analysis of clinical activity in the experimental arm compared with the control arm. If the interim analysis suggests that the activity in an experimental arm is higher than that in the control arm, there may be further enrollment of 20 additional patients in the experimental arm (expansion phase).

In Cohort 2, a posterior probability may be used to guide further enrollment in a treatment arm based on an interim analysis of clinical activity in the experimental arm compared with a predefined ORR threshold, defined as an improvement over standard of care. For example, if available data suggest a standard of care ORR of 10%, and an ORR improvement of 10% is considered to be a clinical meaningful change, this would lead to an ORR threshold of 20% in the calculation of the posterior probability.

The ORR for standard of care treatment is based on emerging internal and external data for in-class immune-modulating investigational and other compounds for the patient population in Cohort 2 who have received at least two lines of prior treatment at the time of the analysis.

The Sponsor may make a decision to expand enrollment in an arm based on the totality of available data including, but not limited to, duration of the observed responses, PFS, and potentially early OS data. Safety and biomarker data (available at the time of making this decision) are also taken into consideration from the perspective of an adequate benefit-risk assessment.

### Example 3: A Phase Ib/II, Open-Label, Multicenter, Study Evaluating the Safety, Efficacy, and Pharmacokinetics of Mosunetuzumab in Combination with Tiragolumab with or without Atezolizumab in Patients with Relapsed or Refractory B-Cell Non-Hodgkin's Lymphoma

The purpose of this study is to assess the safety, efficacy and pharmacokinetics of mosunetuzumab, a bispecific antibody targeting CD20 and CD3, in combination with tiragolumab, an anti-TIGIT (T cell immunoreceptor with Ig and ITIM domains) antibody, with or without additional combination of atezolizumab, an antibody that targets PD-L1, in patients with relapsed or refractory (R/R) follicular lymphoma (FL) or diffuse large B cell lymphoma (DLBCL). A proportion of patients with these diseases are refractory to or eventually relapse after the standard first-line chemoimmunotherapy, and relapses are characterized by increasing refractoriness and decreasing duration of response to subsequent lines of therapy. This underscores the need for novel treatments in subsequent lines of therapy, which result in longer progression-free survival (PFS) and overall survival (OS) and have an improved benefit-risk profile.

### A. Objectives and Endpoints

This study evaluates the safety, efficacy, and pharmacokinetics of mosunetuzumab in combination with tiragolumab, with or without atezolizumab, in participants with R/R DLBCL or FL who have received at least two previous lines of systemic therapy. Specific objectives and endpoints for the study are outlined below in Table 21.

TABLE 21
Objectives and Corresponding Endpoints for Study
Phase Ib-Specific Objectives

| Primary Safety Objective | Corresponding Endpoint |
|---|---|
| To evaluate the safety | Incidence and severity |
| of mosunetuzumab SC | of adverse events, |
| in combination with | including DLTs, with |
| tiragolumab IV, | severity determined |
| including evaluation | according to NCI CTCAE |
| of the tolerability | v5.0; for CRS, severity |
| of the dosing | determined according to |
| schedule and dose, | ASTCT CRS consensus |
| and characterization | grading criteria. |
| of DLTs in Cohort | |
| A and Cohort B | |
| To evaluate the safety | |
| of mosunetuzumab SC | |
| with tiragolumab IV | |
| and atezolizumab IV, | |
| including evaluation | |
| of the tolerability | |
| of the dosing schedule | |
| and dose, and | |
| characterization of | |
| DLTs in Cohort E | |
| Secondary | |
| Efficacy Objective | Corresponding Endpoint |
| To evaluate the preliminary | Best ORR (CR or PR at |
| efficacy of mosunetuzumab | any time) on study as |
| SC in combination with | determined by the |
| tiragolumab IV in Cohorts | investigator using Lugano |
| A and B | 2014 criteria (Cheson BD, |
| To evaluate the preliminary | et al. J Clin Oncol 2014; |
| efficacy of mosunetuzumab | 32: 1-9) |
| SC with tiragolumab IV | Best CR rate, defined as |
| and atezolizumab IV in | the proportion of |
| Cohort E | participants whose best |
| | overall response is a |
| | complete response during the |
| | study, as determined by |
| | the investigator using |
| | Lugano 2014 criteria |
| | DOR, defined as the time |
| | from the first occurrence |
| | of a documented objective |
| | response (CR or PR) to |
| | disease progression or |
| | relapse, as determined |
| | by the investigator using |
| | Lugano 2014 criteria, or |
| | death from any cause, |
| | whichever occurs first |

Phase II-Specific Objectives

| Primary Efficacy Objective | Corresponding Endpoint |
|---|---|
| To evaluate the efficacy | Best ORR, defined as the |
| of mosunetuzumab SC in | proportion of participants |
| combination with | whose best overall |
| tiragolumab IV in | response is a PR or a |
| participants with R/R | CR during the study, as |
| FL (Cohort C) | determined by the |
| To evaluate the efficacy | investigator using Lugano |
| of mosunetuzumab SC | 2014 criteria |
| in combination with | |
| tiragolumab IV in | |
| participants with | |
| R/R DLBCL (Cohort D) | |
| To evaluate the efficacy | |
| of mosunetuzumab SC in | |
| combination with | |
| tiragolumab IV and | |
| atezolizumab IV in | |
| participants with | |
| FL (Cohort F) | |
| Secondary | |
| Efficacy Objective | Corresponding Endpoint |
| To evaluate the efficacy | Best CR rate on study, |
| of mosunetuzumab SC in | as determined by the |
| combination with | investigator using Lugano |
| tiragolumab IV in | 2014 criteria DOR, |
| participants with R/R | defined as the time from |
| FL (Cohort C) | the first occurrence of |
| To evaluate the efficacy | a documented objective |
| of mosunetuzumab SC in | response (CR or PR) to |
| combination with | disease progression or |
| tiragolumab IV in | relapse, as determined |
| participants with R/R | by the investigator using |
| DLBCL (Cohort D) | Lugano 2014 criteria, or |
| To evaluate the efficacy | death from any cause, |
| of mosunetuzumab SC | whichever occurs first |
| in combination with | PFS, defined as the time |
| | from the first study |
| | treatment to the first |
| | occurrence of disease |
| tiragolumab IV and | progression or relapse, |
| atezolizumab IV in | as determined by the |
| participants with R/R | investigator using Lugano |
| FL (Cohort F) | 2014 criteria, or death |
| | from any cause, whichever |
| | occurs first |
| | EFS, defined as the time |
| | from the first study |
| | treatment to the first |
| | occurrence of disease |
| | progression or relapse, |
| | as determined by the |
| | investigator using |
| | Lugano 2014 criteria, |
| | initiation of NALT, or |
| | death from any cause, |
| | whichever occurs first. |
| | OS, defined as the time |
| | from the first study |
| | treatment to death |
| | from any cause |
| Secondary | |
| Safety Objectives | Corresponding Endpoint |
| To evaluate the safety | Occurrence and severity |
| of mosunetuzumab in | of adverse events, with |
| combination with | severity determined |
| tiragolumab in | according to NCI CTCAE |
| participants with | v5.0. For CRS, severity |
| R/R FL (Cohort C) | determined according to |
| To evaluate the | the ASTCT CRS Consensus |
| safety of | Grading criteria |
| mosunetuzumab in | |
| combination | |
| with tiragolumab | |
| in participants | |
| with R/R DLBCL | |
| (Cohort D) | |
| To evaluate the | |
| safety of | |
| mosunetuzumab | |
| with tiragolumab | |
| and atezolizumab | |
| in participants | |
| with R/R FL | |
| (Cohort F) | |

Objectives for Both Phase Ib and Phase II

| Pharmacokinetic Objectives | Corresponding Endpoint |
|---|---|
| To characterize the | Cₘₐₓ |
| PK profile of | Cₘᵢₙ |
| mosunetuzumab SC in | Total exposure (AUC), |
| combination with | CL, and volume of |
| tiragolumab IV (Cohort | distribution, as |
| A, Cohort B, Cohort | estimated by population |
| C, and Cohort D) | PK modeling, as |
| To characterize the | appropriate, and |
| PK profile of | supported by data |
| mosunetuzumab SC in | |
| combination with | |
| tiragolumab IV and | |
| atezolizumab IV | |
| (Cohort E and Cohort F) | |
| Exploratory | |
| Efficacy Objective | Corresponding Endpoint |
| To evaluate the efficacy | Best ORR, defined as the |
| of mosunetuzumab SC in | proportion of participants |
| combination with | whose best overall response |
| tiragolumab IV in | is a PR or a CR during |
| participants with R/R | the study, as determined |
| FL (Cohort C) | by automated response |
| To evaluate the efficacy | using Lugano 2014 criteria |
| of mosunetuzumab SC | (aLugano) |
| in combination with | |
| tiragolumab IV in | |
| participants with R/R | |
| DLBCL (Cohort D) | |
| To evaluate the efficacy | |
| of mosunetuzumab SC | |
| in combination with | |
| tiragolumab IV an | |
| atezolizumab IV in | |
| participants with R/R | |
| FL (Cohort F) | |
| Exploratory | |
| Pharmacokinetic Objective | Corresponding Endpoint |
| To characterize the | Cₘₐₓ |
| pharmacokinetics of | Cₘᵢₙ |
| tiragolumab IV when | Total exposure (AUC), |
| administered in | CL, and volume of |
| combination with | distribution, as |
| mosunetuzumab SC | estimated by population |
| (Cohort A, Cohort B, | PK modeling, as |
| Cohort C, and Cohort | appropriate, and |
| D) and when in | supported by data |
| combination with | |
| mosunetuzumab SC | |
| and atezolizumab IV | |
| (Cohort E and Cohort F) | |
| To characterize the | |
| pharmacokinetics of | |
| atezolizumab IV when | |
| administered in | |
| combination with | |
| mosunetuzumab SC | |
| and tiragolumab IV | |
| (Cohort E and Cohort F) | |
| To assess potential | PK parameters for mosunetuzumab |
| PK interactions between | given in combination with |
| mosunetuzumab SC and | tiragolumab compared with |
| tiragolumab IV | mosunetuzumab given alone |
| To assess potential PK | based on historical data |
| interactions between | PK parameters for tiragolumab |
| mosunetuzumab SC, | given in combination with |
| tiragolumab IV and | mosunetuzumab compared with |
| atezolizumab IV | tiragolumab given alone |
| | based on historical data |
| | PK parameters for mosunetuzumab |
| | in combination with tiragolumab |
| | and atezolizumab compared with |
| | mosunetuzumab given alone |
| | based on historical data |
| | PK parameters for tiragolumab |
| | given in combination with |
| | mosunetuzumab and atezolizumab |
| | compared with tiragolumab given |
| | alone based on historical data |
| | PK parameters for atezolizumab |
| | given in combination with |
| | mosunetuzumab and tiragolumab |
| | compared with atezolizumab given |
| | alone based on historical data |
| To evaluate potential | Relationship between PK and |
| relationships between | safety, biomarkers, or efficacy |
| drug exposure and the | endpoints, as appropriate |
| efficacy, biomarkers | |
| and safety | |
| Exploratory | |
| Immunogenicity Objective | Corresponding Endpoint |
| To assess the ADA incidence | Prevalence of ADAs at |
| to mosunetuzumab | baseline and incidence |
| To assess the ADA incidence | of ADAs during the study |
| to tiragolumab | |
| To assess the ADA incidence | |
| to atezolizumab | |
| Exploratory | |
| Biomarker Objective | Corresponding Endpoint |
| To identify biomarkers that | Association between |
| are predictive of response | prognostic subtypes, |
| to mosunetuzumab SC in | exploratory biomarkers, |
| combination with only | and safety and efficacy |
| tiragolumab IV (Cohorts | endpoints |
| A-D), and in combination | |
| with tiragolumab IV and | |
| atezolizumab IV (Cohorts | |
| E and F) (i.e., predictive | |
| biomarkers), are associated | |
| with progression to a more | |
| severe disease state (i.e., | |
| prognostic biomarkers), | |
| are associated with acquired | |
| resistance to mosunetuzumab | |
| plus tiragolumab (with or | |
| without atezolizumab), | |
| are associated with | |
| susceptibility to | |
| developing adverse | |
| events, can provide | |
| evidence of | |
| mosunetuzumab plus | |
| tiragolumab (with or | |
| without atezolizumab) | |
| activity, or can | |
| increase the knowledge | |
| and understanding of | |
| disease biology | |
| To make a preliminary | |
| assessment of response | |
| following mosunetuzumab | |
| SC in combination with | |
| only tiragolumab IV | |
| (Cohorts A-D), and in | |
| combination with | |
| tiragolumab IV and | |
| atezolizumab IV (Cohorts | |
| E and F) in different | |
| clinical and biologic | |
| prognostic subgroups of NHL | |

ADA = anti-drug antibody; ASTCT = American Society for Transplantation and Cellular Therapy; AUC = area under the concentration-time curve; Cₘₐₓ = maximum concentration observed; Cₘᵢₙ = minimum concentration under steadystate; CR = complete response; CRS = cytokine release syndrome; CT = computed tomography; DLT = dose-limiting toxicity; DLBCL = diffuse large B cell lymphoma; DOR = duration of response; FL = follicular lymphoma; NALT = new antilymphoma treatment; NCI CTCAE = National Cancer Institute Common Terminology Criteria for Adverse Events; ORR = objective response rate; PR = partial response; R/R = relapsed or refractory.
Lugano 2014 criteria: Cheson BD, et al. J Clin Oncol 2014; 32: 1-9

### B. Study Design

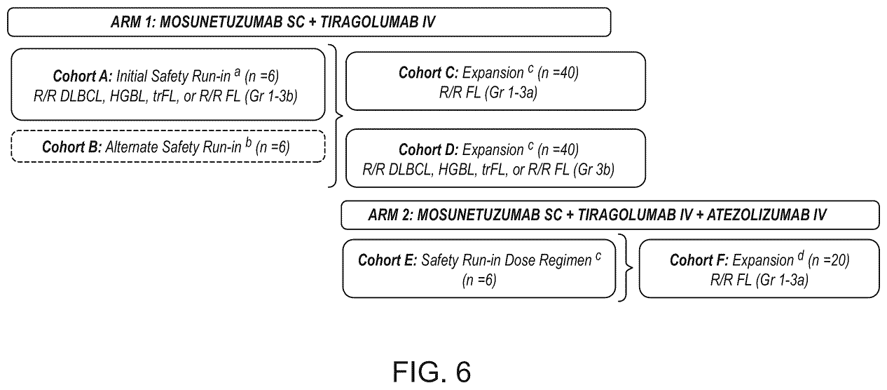

This study is designed to evaluate the safety, tolerability, pharmacokinetics, and preliminary efficacy of mosunetuzumab SC in combination with tiragolumab IV with or without atezolizumab IV in participants with R/R B-cell NHL, specifically participants with DLBCL, HGBL, trFL, or FL (Grades 1-3b) who have received at least two prior lines of systemic therapy. A schematic of the present study design is presented in FIG. 6.

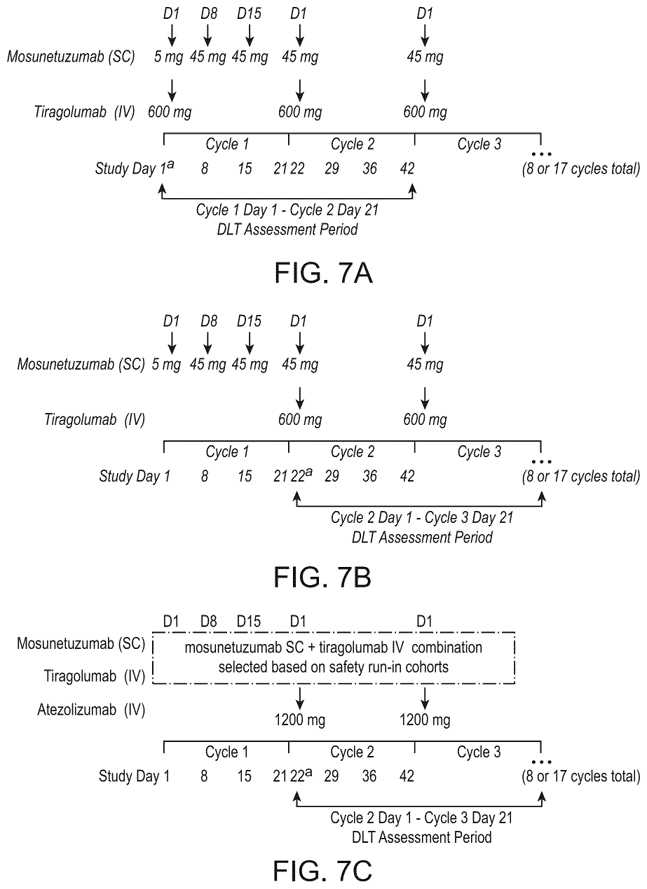

Participants receive 8 cycles of study treatment, including all cycles in which at least one study drug is administered. Participants who achieve PR or SD at the time of primary response assessment (PRA) continues treatment for a total of 17 cycles in the absence of disease progression.
Arm 1: mosunetuzumab SC in combination with tiragolumab IV: Safety Run-In Cohort A (initial; see FIG. 7A) and Cohort B (alternate; see FIG. 7B) enrolls approximately 6 participants with R/R DLBCL, HGBL, trFL, or R/R FL (Grade 1-3b) in each cohort. Following IMC recommendations on dosing, Expansion Cohort C enrolls approximately 40 participants with R/R FL (Grades 1-3a) and Expansion Cohort D enrolls approximately 40 participants with R/R DLBCL, HGBL, trFL, or R/R FL (Grade 3b)
Arm 2: mosunetuzumab SC in combination with tiragolumab IV and atezolizumab IV: Safety Run-In Cohort E (see FIG. 7C) enrolls approximately 6 participants with R/R DLBCL, HGBL, trFL, or R/R FL (Grade1-3b). Following IMC recommendations on dosing, Expansion Cohort F enrolls approximately 20 participants with R/R FL (Grades 1-3a).

Individuals who do not meet the criteria for participation in this study (screen failure) may qualify for 2 re-screening opportunities (for a total of 3 screenings per individual) at the investigator's discretion. For participants who are re-screened, all eligibility criteria must be re-evaluated and screening assessments are repeated as applicable to meet the eligibility criteria.

All participants are closely monitored for adverse events throughout the study and for at least 90 days after the final dose of study treatment. Adverse events is graded according to NCI CTCAE 5.0, with CRS graded according to the ASTCT 2019 CRS Consensus Grading (Lee et al. 2019, see Table 4). Participants are assessed for tumor response by positron emission tomography (PET)/computed tomography (CT) and CT at the interim response assessment (IRA; Cycle 4, Day 15-21) and PRA (Cycle 8, Day 15-21) and at regular intervals during the study treatment and follow-up periods. Tumor response is assessed using the 2014 Lugano Response Criteria (Cheson B D, et al. J Clin Oncol 2014; 32:1-9). To characterize the PK profile and immune response towards study treatment, blood samples are taken at various timepoints before and after dosing. Consenting participants in Expansion Cohorts C, D, or F may undergo optional paired tumor biopsies at baseline and between Day 15 of Cycle 1 and Day 1 of Cycle 2 or Day 15 of Cycle 2 and Day 1 of Cycle 3 (dependent on the regimen selected from the Safety Run-In Cohorts). Participants may also undergo additional on-treatment biopsies at any other time at the investigator's discretion (if deemed clinically feasible by the investigator).

### Number of Participants

Overall, approximately 6-118 participants are enrolled in this study at approximately 30 investigative sites globally.

### Study Treatment

The investigational medicinal products (IMP) for this study are mosunetuzumab SC, tiragolumab IV, atezolizumab IV, and tocilizumab IV. See Table 22 for specific dosing information.

TABLE 22
Study Treatment Description

| Primary | | | | |
|---|---|---|---|---|
| Safety Objective | Mosunetuzumab | Tiragolumab | Atezolizumab | Tocilizumab |
| Use | Experimental | Experimental | Experimental | Other |
| Type of medicinal | Investigational | Investigational | Investigational | Investi-gational |
| product | medicinal product | medicinal product | medicinal product | medicinal product |
| Dosage level(s) | C1D1: 5 mg | 600 mg Q3W | 1200 mg Q3W | See Section XIII |
| | C1D2: 45 mg | | | |
| | C1D3: 45 mg | | | |
| | C2 + D1: 45 mg | | | |
| Route of | Subcutaneous | Intravenous | Intravenous | Intravenous |
| administration | | | | |
| Source | Sponsor | Sponsor | Sponsor | Sponsor or site |

C = cycle; D = dose; C1D1 dose administered on Day 1 of Cycle 1; C1D2 dose administered on Day 8 of Cycle 1; C1D3 dose administered on Day 15 of Cycle 1; C2 + D1 doses each administered on Day 1 of each respective dose on Cycles 2+.

### Duration of Participation

Treatment continues for a total of 8 treatment cycles if a CR is achieved at PRA, or for 17 cycles if response is assessed as PR or SD at PRA as determined by the Lugano classification (Cheson B D, et al. J Clin Oncol 2014; 32:1-9). Treatment is discontinued if there is confirmed disease progression or unacceptable toxicity. The total duration of study participation for each individual ranges from 1 day to more than 36 months.

### Phase IB: Safety Run-In Cohorts

The purpose of the Phase Ib Safety Run-In Cohorts is to determine the mosunetuzumab SC step-up dosing schedule and regimen for use in combination with tiragolumab IV with or without atezolizumab IV in patients with R/R NHL.

In order to assess for any severe and unexpected acute drug or injection- or infusion-related toxicities, enrollment into the Safety Run-In Cohorts A, B, and E is staggered. There must be at least 72 hours between each C1D1 dose for the first 3 participants in each cohort. There must be at least 24 hours between each C1D1 dose for subsequent participants in the same cohort. The Sponsor must receive confirmation on the status of the prior participant before the next participant receives study treatment. Additionally, all participants in the Safety Run-In Cohorts are hospitalized for 72 hours (based on the end of the last drug administered) after receiving a combination dose for the first time that has not been previously evaluated:
Participants in Cohort A are hospitalized for 72 hours (based on the end of the last drug administered) on C1D1
Participants in Cohort B and Cohort E are hospitalized for 72 hours (based on the end of the last drug administered) on C2D1

If a participant experiences a CRS Grade ≥2 following single-agent mosunetuzumab or combination administrations, hospitalization at subsequent administration(s) may be required. The investigator actively assess the need for hospitalization based on the participants medical factors such as frailty, risk factors for CRS and prior CRS events, as well as social factors including availability of caregivers at home and distance to the trial sites. All adverse events, including dose-limiting toxicities (DLTs), defined herein, are reported and graded according to NCI CTCAE v5.0 unless otherwise indicated. CRS events are graded according to the ASTCT CRS Consensus Grading criteria (Table 4).

Mosunetuzumab SC in Combination with Tiragolumab IV (Arm 1, Cohorts A and B)

Cohort A (FIG. 7A) is initiated at the recommended Phase II mosunetuzumab SC step-up dose and schedule of 5 mg on Day 1 of Cycle 1 (C1D1 dose), 45 mg on Day 8 of Cycle 1 (C1D2 dose) and 45 mg on Day 15 of Cycle 1 (C1D3 dose). Tiragolumab IV is administered at 600 mg Q3W on D1 of each cycle, starting in C1.

If no more than 1 out of 6 DLT-evaluable participants experiences a DLT, the expansion cohorts of Arm 1 and the Safety Run-In Cohort of Arm 2 can be opened for enrollment. Tiragolumab administration in these cohorts is based on the schedule in Cohort A, i.e., starting on C1D1.

However, if 2 or more participants experience DLT(s) during the Cohort A DLT Assessment Period, or if the totality of data supports a different dose regimen, Cohort B (with an alternate dose regimen) may be initiated per IMC recommendation (FIG. 7B). In Cohort B, mosunetuzumab SC is administered at the same dose and schedule as in Cohort A, but the first dose of tiragolumab is administered on Day 1 of Cycle 2 (C2D1 dose).

If no more than 1 out of 6 DLT-evaluable participants experiences a DLT in Cohort B, the expansion cohorts of Arm 1 and the Safety Run-In Cohort of Arm 2 can be opened for enrollment. Tiragolumab administration in these cohorts is based on the schedule in Cohort B, i.e., starting on C2D1.

However, if 2 or more participants experience a DLT in Cohort B, or the totality of data supports a lower dose, an additional dose de-escalation cohort of approximately 6 participants each may be initiated per IMC recommendation to assess a lower dose level of mosunetuzumab or tiragolumab in combination with the same dosing schedule and hospitalization requirement as in Cohort B (i.e., mosunetuzumab step-up dosing in Cycle 1 and start of tiragolumab administration in Cycle 2). The safety data for this lower dose group is evaluated after 6 participants in that cohort have completed 21 days of study treatment (Days 1-21 of Cycle 2).

If no more than 1 out of 6 DLT-evaluable participants experience a DLT in this dose de-escalation cohort, the Expansion Cohorts of Arm 1 and the Safety Run-In Cohort of Arm 2 may be opened for enrollment, with the start of tiragolumab administration on C2D1 and the doses evaluated in the dose de-escalation cohort.

Mosunetuzumab SC in Combination with Tiragolumab IV with Atezolizumab IV (Cohort E)

Cohort E may be opened for enrollment upon recommendation of the IMC based on review of the safety data for mosunetuzumab SC in combination with tiragolumab IV from the Safety Run-In Cohorts of Arm 1. Cohort E assesses the safety of mosunetuzumab SC in combination with tiragolumab IV and atezolizumab 1200 mg IV in participants with R/R DLBCL, HGBL, trFL, or R/R FL (Grades 1-3b) (FIG. 7C). If no more than 1 out of 6 DLT-evaluable participants experience a DLT in Cohort E, the expansion cohort of Arm 2 may be opened for enrollment.

### Phase II: Expansion Phase

The purposes of the Phase II Expansion Cohorts C, D and F are to further assess the safety and efficacy of mosunetuzumab SC and tiragolumab IV with or without atezolizumab IV at the dose and schedule determined by the Safety Run-In. The dose and schedule of study treatment to be assessed in the Expansion Cohorts are selected based on the Safety Run-In Cohorts and review of cumulative safety data by the IMC. Patients with R/R FL or R/R DLBCL are enrolled during the expansion phase and treated as described below:
Cohort C (mosunetuzumab SC and tiragolumab IV): R/R FL (Grade 1-3a); approximately 40 patients
Cohort D (mosunetuzumab SC and tiragolumab IV): R/R DLBCL, HGBL, trFL, or R/R FL (Grade 3b); approximately 40 patients
Cohort F (mosunetuzumab SC, tiragolumab IV, and atezolizumab IV): R/R FL grade (Grade 1-3a); approximately 20 patients

### Mosunetuzumab SC, Tiragolumab IV, and Atezolizumab IV Re-Treatment

Participants who achieve a complete response during initial treatment and experience disease relapse after completion of study treatment are eligible for re-treatment with mosunetuzumab and tiragolumab (Cohort A, Cohort B, Cohort C, and Cohort D) or mosunetuzumab, tiragolumab, and atezolizumab (Cohort E and Cohort F) as described below. The study re-treatment dose and schedule is one that has been previously demonstrated to be safe in the Safety Run-In Cohorts, provided the following criteria are met:
Pertinent eligibility criteria are met at the time treatment is re-initiated with the following exceptions:
   Previous treatment with mosunetuzumab is allowed
   Serology tests to demonstrate human immunodeficiency virus (HIV), hepatitis C virus (HCV), and hepatitis B virus (HBV) status do not need to be repeated unless clinically indicated. Epstein-Barr virus (EBV) and cytomegalovirus (CMV) quantitative PCR must be repeated
   Manageable and reversible immune-related adverse events of Grades 1-3 with initial study treatment are allowed and do not constitute an exclusionary history of autoimmune disease
   Endocrinopathy of any grade managed with replacement therapy or asymptomatic elevation of serum amylase or lipase of any grade are allowed.
Patient must not have experienced Grade 4 non-hematologic adverse events that were not considered to be attributable to another clearly identifiable cause during initial study treatment, with TLS and CRS as possible exceptions.
Patients who experienced Grade 2 or 3 adverse events that were not considered to be attributable to another clearly identifiable cause during initial treatment must have resolved to s Grade 1. Laboratory values must meet the requirements specified in the inclusion criteria with the respective exceptions.
No intervening systemic anti-cancer therapy was administered between the completion of initial study treatment and re-initiation of study treatment.
Written informed consent is provided to acknowledge deferring any standard treatment options that may exist in favor of reinitiating study treatment and to undergo a biopsy of recurrent or progressing tumor if clinically feasible.

For patients proceeding to re-treatment following disease progression, a repeat tumor biopsy is strongly suggested if a lesion is amenable for biopsy at disease progression, in order to assess the status of the tumor (e.g., CD20, TIGIT and PD-1 expression status), and changes/status of the immune microenvironment. The dose and schedule of study treatment to be administered for patients receiving re-treatment is determined by the Medical Monitor and is a previously tested dose and schedule for which all patients of the respective Safety Run-In Cohort cleared the DLT observation period. The hospitalization requirements for the respective dose and schedule apply based on the recommendation of the IMC. Patients may require hospitalization following the first re-treatment administration.

### Duration of Participation

Treatment will continue for a total of 8 treatment cycles if a CR is achieved at primary response assessment (PRA), or for 17 cycles if response is assessed as partial response (PR) or stable disease (SD) at PRA as determined by the Lugano classification (Cheson B D, et al. J Clin Oncol 2014; 32:1-9). Treatment will be discontinued if there is confirmed disease progression or unacceptable toxicity. The total duration of study participation for each individual is expected to range from 1 day to more than 36 months.

### C. Inclusion Criteria

Participants are eligible to be included in the study only if all of the following criteria apply:
Participants who are capable of giving signed informed consent, which includes compliance with the requirements and restrictions listed in the Informed Consent Form and in this protocol
Participants who are age ≥18 years at the time of signing the Informed Consent Form required hospitalizations, in the investigator's judgment
Participants who have an Eastern Cooperative Oncology Group (ECOG) Performance Status of 0, 1, or 2
Participants who have a life expectancy of at least 12 weeks
Participants who have histologically documented FL or DLBCL
   That has relapsed or failed to respond to at least two prior systemic treatment regimens and for which no suitable therapy of curative intent or higher priority exists (e.g., standard chemotherapy, ASCT, CAR T cells), including at least one line of therapy containing a CD20-directed therapy, at least one prior regimen containing anthracycline for participants with DLBCL, HGBL, trFL and FL Grade 3b, at least one prior regimen containing an alkylating agent for participants with FL. Myeloablative high dose chemotherapy followed by consolidative autologous HSCT should be counted as one line of therapy, separate from the prior salvage chemotherapy. Bridging therapy followed by CAR T-cell therapy will be counted as one line of therapy. Local therapies (e.g., radiotherapy or intrathecal therapy) will not be considered as lines of therapy
   That expresses CD20 as determined by the local laboratory
   That are included in the following list of diagnoses by 2016 WHO classification of lymphoid neoplasms: FL (including in situ follicular neoplasia and duodenal-type FL), Pediatric-type FL, DLBCL, NOS (including germinal center B-cell type and activated B-cell type), T-cell/histiocyte-rich large B-cell lymphoma, High grade B-cell lymphoma with MYC and BCL-2, BCL-6 rearrangements, High grade B-cell lymphoma, not otherwise specified (NOS), EBV+DLBCL, NOS, HHV8+DLBCL, NOS, and/or Anaplastic lymphoma kinase (ALK)+large B-cell lymphoma
   Transformed FL is an eligible diagnosis if the disease histology after transformation is DLBCL or HGBCL and participants must be R/R to standard therapies for transformed FL participants with Richter's transformation are not eligible for enrollment into the study participants with FL grade 3b are eligible for enrollment into only the Safety Run-In Cohorts A, B, and E and Expansion Cohort D if they are R/R to standard therapies for aggressive NHL.
Participants who have at least one bi-dimensionally measurable (>1.5 cm) nodal lesion, or at least one bi-dimensionally measurable (>1.0 cm) extranodal lesion
Participants who have confirmed availability of a tumor tissue (a newly collected tumor tissue sample obtained at baseline is preferred; if a fresh biopsy is unobtainable per investigator assessment, archival tissue is acceptable).
Participants with FL (including trFL) for whom a bone marrow biopsy and aspirate can be collected
Participants with adequate hematologic and organ function, defined by the following laboratory results obtained within 14 days prior to the first study treatment (C1D1): AST and ALT≤2.5×ULN; Total bilirubin ≤1.5×ULN; participants with a documented history of Gilbert syndrome and in whom total bilirubin elevations are accompanied by elevated indirect bilirubin are eligible; INR ≤1.5×ULN in the absence of therapeutic anticoagulation; PTT or aPTT ≤1.5×ULN in the absence of lupus anticoagulant and in the absence of therapeutic anticoagulation; Measured or estimated creatinine clearance ≥50 mL/min by institutional standard method; Platelet count ≥75,000/μL in the absence of platelet transfusion within 72 hours; Total hemoglobin ≥9 g/dL without transfusion within 21 days; Absolute neutrophil count (ANC) ≥1000/μL; and participants must not have received growth factor within the previous 7 days prior to the ANC used for eligibility. For participants who do not meet criteria for hematologic function because of extensive marrow involvement of NHL and/or disease-related cytopenias (e.g., immune thrombocytopenia) may be enrolled into the study if each of the below criteria are met: platelet count ≥50,000/μL without transfusion within 14 days, ANC ≥500/mm³, and any hemoglobin but without transfusion within 7 days
For women of childbearing potential: participants who agree to remain abstinent (refrain from heterosexual intercourse) or use contraception.
For men: participants who agree to remain abstinent (refrain from heterosexual intercourse) or use a condom, and agree to refrain from donating sperm.

### D. Exclusion Criteria

Participants who are pregnant or breastfeeding, or intending to become pregnant during the study or within 3 months after the final dose of mosunetuzumab, 3 months after the final dose of tiragolumab, 5 months after the last dose of atezolizumab (if applicable) and 3 months after the last dose of tocilizumab (if applicable), whichever is longer.
Women of childbearing potential must have a negative serum pregnancy test result within 14 days prior to initiation of study treatment.
Participants who have received any of the following treatments prior to study entry: treatment with mosunetuzumab or other CD20/CD3-directed bispecific antibodies; treatment with tiragolumab or other anti-TIGIT agent; Allogeneic SCT; and/or solid organ transplantation
Participants who received any of the following treatments, whether investigational or approved, within the respective time periods prior to initiation of study treatment: radiotherapy within 2 weeks prior to the first dose of study treatment If participants have received radiotherapy within 4 weeks prior to the first study treatment administration, participants must have at least one measurable lesion outside of the radiation field; autologous SCT within 100 days prior to first study treatment; CAR T-cell therapy within 30 days before first study treatment use of monoclonal antibodies or antibody-drug conjugates within 4 weeks prior to first study treatment use of radioimmunoconjugates within 12 weeks prior to first study treatment; systemic immunosuppressive medications (including, but not limited to, cyclophosphamide, azathioprine, methotrexate, thalidomide, and anti-tumor necrosis factor agents) within 2 weeks prior to first dose of study treatment; systemic corticosteroid treatment s 10 mg/day prednisone or equivalent and inhaled corticosteroids are permitted; administration of acute, low-dose, systemic immunosuppressant medications (e.g., single dose of dexamethasone for nausea or B symptoms) is permitted; use of mineralocorticoids for management of orthostatic hypotension and corticosteroids for management of adrenal insufficiency is permitted; and/or any other anti-cancer therapy, whether investigational or approved, including but not limited to chemotherapy, within 4 weeks or 5 half-lives of the drug, whichever is shorter, prior to initiation of study treatment.
Participants who received a live, attenuated vaccine within 4 weeks before first dose of study treatment, or in whom it is anticipated that such a live attenuated vaccine will be required during the study period or within 5 months after the final dose of study treatment
Participants with aggressive NHL who are currently eligible for autologous SCT
Participants with current or past history of CNS lymphoma or leptomeningeal infiltration
Participants with a history of severe allergic or anaphylactic reactions to humanized or murine monoclonal antibody therapy (or recombinant antibody-related fusion proteins)
Participants with a contraindication to atezolizumab (specific to Arm 2 of the study) or tocilizumab
Participants in whom clinically significant toxicities from prior treatment have not resolved to Grade ≤1 (per NCI CTCAE v5.0) prior to the first study drug administration with the following exceptions: Grade 2 peripheral sensory or motor neuropathy; any grade alopecia or vitiligo; and/or endocrinopathy managed with replacement therapy
Participants with treatment-emergent immune-mediated adverse events associated with prior immunotherapeutic agents as follows: history of a Grade ≥3 immune-mediated adverse event attributed to prior immune checkpoint-inhibitor (ICI) therapy; other than endocrinopathy managed with replacement therapy or asymptomatic elevation of serum amylase or lipase); and/or all immune-mediated adverse events related to prior cancer immunotherapy (other than endocrinopathy managed with replacement therapy, stable vitiligo and stable cytopenias that meet the inclusion criteria) must have resolved to baseline
Participants treated with corticosteroids for immune-mediated adverse events must demonstrate absence of related symptoms or signs for ≥4 weeks following discontinuation of corticosteroids.
Participants with evidence of any significant, concomitant disease that could affect compliance with the protocol or interpretation of results, including, but not limited to:
   A history of other malignancy that could affect compliance with the protocol or interpretation of results, with the exception of the following: any of the following malignancies previously curatively treated: carcinoma in situ of the cervix, good-prognosis ductal carcinoma in situ of the breast, basal, or squamous cell skin cancer; prostate cancer with no evidence of metastatic disease and not on active therapy except for anti-androgen therapy; and/or participants with any other malignancy appropriately treated with curative intent and in remission without treatment for ≥2 years prior to enrollment are eligible
   Significant cardiovascular disease (e.g., New York Heart Association Class III or IV cardiac disease, myocardial infarction within the previous 6 months, unstable arrhythmia, or unstable angina)
   Significant pulmonary disease (such as obstructive pulmonary disease or history of bronchospasm)
   Clinically significant history of liver disease, including viral or other hepatitis, or cirrhosis
   Current or past history of CNS disease, such as stroke, epilepsy, CNS vasculitis, or neurodegenerative disease
   Participants with a history of stroke who have not experienced a stroke or transient ischemic attack in the past 1 year and have no residual neurologic deficits as judged by the investigator are allowed.
   Participants with a history of epilepsy who have had no seizures in the past 2 years with or without anti-epileptic medications can be eligible only in the Expansion Cohorts C, D and F.
   History of confirmed progressive multifocal leukoencephalopathy (PML)
   Known active bacterial, viral (including SARS-CoV-2), fungal, mycobacterial, parasitic, or other infection (excluding fungal infections of nail beds) at study enrollment, or any major episode of infection requiring treatment with IV antibiotics or hospitalization within 4 weeks (relating to the completion of the course of antibiotics) prior to first study treatment administration
   Positive serologic HIV test at screening
   Positive test results for chronic hepatitis B infection (defined as positive hepatitis B surface antigen [HBsAg] serology)
   Participants with occult or prior hepatitis B infection (defined as positive total hepatitis B core antibody and negative HBsAg) may be included if HBV DNA is undetectable at the time of screening. These participants must be willing to undergo monthly DNA testing and appropriate antiviral therapy as indicated.
   Acute or chronic HCV infection
   Participants who are positive for HCV antibody must be negative for HCV by PCR to be eligible for study participation.
   Known or suspected chronic active EBV infection
   Known or suspected history of HLH
   History of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis
   Participants with a history of autoimmune-related hypothyroidism on a stable dose of thyroid replacement hormone may be eligible.
   Participants with controlled Type 1 diabetes mellitus who are on an insulin regimen are eligible for the study.
   Participants with a history of disease-related immune thrombocytopenic purpura or autoimmune hemolytic anemia may be eligible
   Patients with eczema, psoriasis, lichen simplex chronicus, or vitiligo with dermatologic manifestations only (e.g., patients with psoriatic arthritis are excluded) are eligible for the study provided all of following conditions are met: rash must cover 10% of body surface area; disease is well controlled at baseline and requires only low-potency topical corticosteroids; and no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
Participants who underwent recent major surgery within 4 weeks prior to first study treatment administration, with the exception of protocol-mandated procedures (e.g., tumor biopsies and bone marrow biopsies)

### E. Study Treatments and Concomitant Therapy

The investigational medicinal products (IMP) for this study are mosunetuzumab, tiragolumab, atezolizumab, and tocilizumab. Any pre-medications (e.g., corticosteroids) are considered non-investigational medicinal products.

Assigned study treatments of this study are described above in Table 22. Treatment regiments are summarized above in Section B and FIGS. 6 and 7A-7C.

On days when two or more of the IMPs are given, the order of the administration should be mosunetuzumab, tiragolumab with an intervening observation period, and atezolizumab (if applicable).

### Mosunetuzumab

Flat dosing independent of body weight is used for mosunetuzumab. The starting dose of mosunetuzumab in Cohort A is 5 mg/45 mg/45 mg (C1D1/C1D2/C1D3 doses administered on Days 1, 8, and 15 of Cycle 1, respectively). The dose on Day 1 of Cycle 2 and on Day 1 of subsequent cycles is the same as the C1D3 dose (45 mg).

When administered SC, mosunetuzumab is delivered by standard medical syringe with a final volume not to exceed 2.0 mL. Compatibility testing has shown that mosunetuzumab is stable in extension sets and polypropylene syringes.

Mosunetuzumab is administered to well-hydrated participants. Corticosteroid premedication with dexamethasone 20 mg should be administered prior the administration of each mosunetuzumab dose. The administration of corticosteroid premedication may be optional for Cycle 2 and beyond based on the investigator's assessment. The use of an alternative corticosteroid compound (e.g., due to unavailability of dexamethasone) is only permitted after discussion with the Medical Monitor at the time of enrollment. However, if the participant experiences CRS with prior administration of mosunetuzumab, premedication with steroids must be administered for subsequent doses until no additional CRS events are observed.

In addition, premedication with oral acetaminophen or paracetamol (e.g., 500-1000 mg) and/or 50-100 mg diphenhydramine may be administered per standard institutional practice prior to administration of mosunetuzumab.

Vital signs are assessed prior to mosunetuzumab injection (within 30 minutes prior to injection). Mosunetuzumab is administered by qualified staff over 30 seconds to 2 minutes. All participants must have an IV access in place prior to mosunetuzumab SC administration for at least the first 2 cycles. Following the first administration of mosunetuzumab, participants will be observed for at least 30 minutes for fever, chills, rigors, hypotension, nausea, or other signs and symptoms of CRS after the mosunetuzumab administration. Patients should also be observed for 30 minutes after injection for subsequent doses if a CRS event occurred with the previous dose. The observation time may be shortened to 15 minutes for subsequent doses if no CRS occurred with the previous dose. Vital signs are recorded every 15 (±10) minutes during this observation period (for 30 minutes following the first mosunetuzumab injection, and 15 minutes for subsequent doses if no CRS occurred with the previous dose). Thereafter, vital signs are monitored every 4 hours until discharge. If required by the infusion guidelines for the other study drugs, additional vital sign measurements are taken.

### Tiragolumab

Tiragolumab will be administered by IV infusion at a fixed dose of 600 mg on Day 1 of each 21-day cycle starting in Cycle 1 or Cycle 2 according to Table 23. No individual dose modification for tiragolumab is allowed.

TABLE 23
Administration of First and Subsequent Infusions of Tiragolumab

| | First Infusion | Subsequent Infusions |
|---|---|---|
| Before | Vital signs (pulse | If the participant |
| infusion of | rate, respiratory | experienced an IRR |
| tiragolumab | rate, blood pressure, | during any previous |
| | and temperature) | infusion of tiragolumab, |
| | should be recorded | premedication with an |
| | within 60 minutes | antihistamine and/or |
| | prior to the infusion. | antipyretic may be |
| | | administered for subsequent |
| | | doses, at the discretion |
| | | of the investigator. |
| | | Vital signs should be |
| | | recorded within 60 minutes |
| | | prior to the tiragolumab |
| | | infusion. |
| Infusion of | Tiragolumab should | Tiragolumab should be |
| tiragolumab | be infused over 60 | infused over 30 (±10) |
| | (±15) minutes. | minutes if the previous |
| | Vital signs should | infusion was tolerated |
| | be recorded every | without an infusion-related |
| | 15 (±5) minutes | reaction, or 60 (±15) |
| | during the infusion. | minutes if the participant |
| | | experienced an infusion- |
| | | related reaction with the |
| | | previous infusion. |
| | | Vital signs should be |
| | | recorded during the |
| | | infusion if clinically |
| | | indicated. |
| After infusion | After the infusion | If the participant |
| of tiragolumab | of tiragolumab, | tolerated the previous |
| | the participant | infusion of tiragolumab |
| | begins a 60-minute | well without infusion- |
| | observation period. | associated adverse events, |
| | Vital signs should | the observation period may |
| | be recorded at 30 | be reduced to 30 minutes. |
| | (±10) minutes | If the participant |
| | after the infusion | experienced an infusion- |
| | of tiragolumab. | associated adverse event |
| | Participants will be | in the previous infusion, |
| | informed about the | the observation period |
| | possibility of delayed | should be 60 minutes. |
| | post-infusion symptoms | If clinically indicated, |
| | and will be instructed | vital signs should be |
| | to contact their study | recorded at 30 (±10) |
| | physician if they develop | minutes after the infusion |
| | such symptoms. | of tiragolumab. |
| | | Participants will be |
| | | informed about the |
| | | possibility of delayed |
| | | post-infusion symptoms |
| | | and will be instructed |
| | | to contact their study |
| | | physician if they develop |
| | | such symptoms. |

### Atezolizumab

Atezolizumab will be administered by IV infusion at a fixed dose of 1200 mg on Day 1 of each 21-day cycle starting in Cycle 2 according to Table 24 in Cohorts E and F. On days when atezolizumab is given in combination with mosunetuzumab and tiragolumab, atezolizumab will be administered after the end of the observation period for tiragolumab. No individual dose modification for atezolizumab is allowed.

TABLE 24
Administration of First and Subsequent Infusions of Atezolizumab

| | First Infusion | Subsequent Infusions |
|---|---|---|
| Before | Vital signs (pulse rate, | If the participant |
| infusion of | respiratory rate, blood | experienced an IRR |
| atezolizumab | pressure, and temperature) | during any previous |
| | should be recorded within | infusion of atezolizumab, |
| | 60 minutes prior to the | premedication with |
| | infusion. | an antihistamine and/ |
| | | or antipyretic may be |
| | | administered for |
| | | subsequent doses, at |
| | | the discretion of the |
| | | investigator. |
| | | Vital signs should be |
| | | recorded within 60 |
| | | minutes prior to the |
| | | atezolizumab infusion. |
| Infusion of | Atezolizumab should be | Atezolizumab should be |
| atezolizumab | infused over 60 (±15) | infused over 30 (±10) |
| | minutes. | minutes if the previous |
| | Vital signs should be | infusion was tolerated |
| | recorded every 15 (±5) | without an infusion-related |
| | minutes during the infusion. | reaction, or 60 (±15) |
| | | minutes if the participant |
| | | experienced an infusion- |
| | | related reaction with the |
| | | previous infusion. |
| | | Vital signs should be |
| | | recorded during the |
| | | infusion if clinically |
| | | indicated. |
| After | After the infusion of | If the participant |
| infusion of | atezolizumab, the | tolerated the previous |
| atezolizumab | participant begins a | infusion of atezolizumab |
| | 60-minute observation | well without infusion- |
| | period. | associated adverse events, |
| | Vital signs should | the observation period may |
| | be recorded at 30 | be reduced to 30 minutes. |
| | (±10) minutes | If the participant |
| | after the infusion | experienced an infusion- |
| | of atezolizumab. | associated adverse event |
| | Participants will be | in the previous infusion, |
| | informed about the | the observation period |
| | possibility of delayed | should be 60 minutes. |
| | post-infusion symptoms | If clinically indicated, |
| | and will be instructed | vital signs should be |
| | to contact their study | recorded at 30 (±10) |
| | physician if they develop | minutes after the infusion |
| | such symptoms. | of atezolizumab. |

### Tocilizumab

Tocilizumab (ACTEMRA®/ROACTEMRA®) is a recombinant, humanized, anti-human monoclonal antibody directed against soluble and membrane-bound IL-6 receptor, which inhibits IL-6-mediated signaling. Tocilizumab will be administered as a rescue IMP when necessary, to participants who experience a CRS event (see Section XIII).

### Concomitant Therapy

Any medication or vaccine, including over-the-counter or prescription medicines, vitamins, and/or herbal supplements, used by a participant in addition to protocol-mandated treatment from 7 days prior to initiation of study treatment to the study completion/discontinuation visit must be recorded on the Concomitant Medications and associated eCRF(s) along with the following information:
Reason for use
Dates of administration, including start and end dates
Dosage information, including dose and frequency

### Permitted Therapy

In general, investigators may manage a participant's care (including preexisting conditions) through use of supportive therapies, as clinically indicated and per local standard practice, with the exception of prohibited therapies defined herein and taking into account cautionary therapies defined herein. Participants who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β₂-adrenergic agonists):

Use of the following concomitant therapies is permitted as described below:
Premedication with antihistamines, anti-pyretics, and/or analgesics may be administered at the discretion of the investigator.
Oral contraceptives with a failure rate of <1% per year
Hormone-replacement therapy
Treatment of CRS according to Section XIII
Treatment of HLH according to published recommendations and/or institutional practice
TLS prophylaxis/therapy according to published recommendations and/or institutional practice
Anti-emetic prophylaxis/therapy according to published recommendations and/or institutional practice
Prophylactic and therapeutic use of G-CSF (filgrastim, pegfilgrastim) is allowed in accordance with instructions provided in the package inserts, institutional practice, and/or published guidelines (Smith et al. 2015). Growth factor support should be started when ANC is below 500/mm³, unless medically contraindicated; if growth factor is contraindicated, this should be discussed with the Medical Monitor.
Non-live vaccinations
Concomitant use of other hematopoietic growth factors such as erythropoietin, granulocyte/macrophage colony-stimulating factor (sargramostim), or thrombopoietin (oprelvekin, eltrombopag) is allowed in accordance with instructions provided in the package inserts, institutional practice and/or published guidelines.

Anti-infective prophylaxis for viral, fungal, bacterial, or Pneumocystis infections is permitted and should be instituted per institutional practice or investigator preference based on individual patient risk factors.

### F. Efficacy Assessments

### Tumor and Response Evaluations

Participants undergo tumor assessments at screening, IRA at the end of Cycle 4 (between Days 15 and 21 of Cycle 4, prior to starting Cycle 5), and PRA at the end of Cycle 8 (between Days 15 and 21 of Cycle 8). Response is evaluated according to 2014 Lugano criteria (Cheson B D, et al. J Clin Oncol 2014; 32:1-9). After PRA, response continues to be evaluated every 3 months during the first year after treatment initiation, and then every 6 months until the participant develops progressive disease, study discontinuation, and/or the start of new lymphoma treatment, or at any time disease progression is suspected. At the investigator's discretion, tumor assessments may be repeated at any time if progressive disease is suspected.

All measurable and/or evaluable disease is documented at screening and re-assessed at each subsequent tumor evaluation. Response is assessed by the investigator on the basis of physical examinations, CT scans, fluorodeoxyglucose (FDG) PET-CT scans, and bone marrow examinations.

Diagnosis of disease progression based on clinical examination must be confirmed by imaging (e.g., CT scan, FDG PET-CT scan) as soon as feasible (within 30 days) and prior to initiation of non-protocol-specified anti-cancer therapy.

Tumor assessments performed as standard of care prior to obtaining informed consent and within 28 days prior to initiation of study treatment do not have to be repeated at screening, so long as they meet criteria outlined below and the participant has not received anti-cancer therapy since the assessment.

### Radiographic Assessments

Fluorodeoxyglucose PET-CT scans, in conjunction with diagnostic-quality CT scans, are required at screening, IRA, and PRA. Following the PRA, CT scans with or without PET should be performed.

The FDG PET-CT scans should extend from skull base to mid-thigh. Full-body FDG PET-CT scans should be performed when clinically appropriate.

CT scans with contrast (per institutional standard operating procedures) should include the chest, abdomen, and pelvis. CT or magnetic resonance imaging (MRI) scans of other disease sites should be performed as clinically indicated. If a CT scan with contrast is contraindicated (e.g., in participants with contrast allergy or impaired renal clearance), a non-contrast CT scan is permitted if it allows for consistent and precise measurement of target lesions during the study. The MRI scans may be used instead of CT scans in patients for whom they are contraindicated. The same radiographic procedures used to assess disease sites at screening should be used for subsequent tumor assessments (e.g., the same contrast protocol for CT scans). Diagnostic contrast enhanced CT scans obtained as part of a PET-CT scan may be used in lieu of dedicated CT scans.

### Bone Marrow Examinations

Participants with DLBCL may use screening PET/CT scans to assess bone marrow involvement; bone marrow examinations are not required unless clinically indicated (Cheson B D, et al. J Clin Oncol 2014; 32:1-9).

Participants with FL or trFL who had bone marrow infiltration at any time prior to study initiation are required to undergo bone marrow examinations at screening (within 90 days prior to initiation of study treatment) for staging purposes. Participants with FL are required to undergo repeat bone marrow examinations to confirm a radiologic assessment of CR if there was tumor-infiltrated bone marrow at screening and to confirm relapse in the bone marrow.

Bone marrow examinations should include a biopsy for morphology and an aspirate for local hematology (flow studies are optional). Unsuccessful attempts at bone marrow aspiration/biopsy will not be considered a protocol deviation.

### Response Evaluation

Objective response will be determined by the investigator at specified timepoints according to the Lugano Response Criteria (Cheson B D, et al. J Clin Oncol 2014; 32:1-9). Endpoints (e.g., ORR, CRR, PFS, EFS) will be calculated programmatically on the basis of investigator assessments of response at each specified timepoint.

### G. Safety Assessments

### Adverse Events of Special Interest

Adverse events of special interest for this study are as follows:
Cases of potential drug-induced liver injury that include an elevated ALT or AST in combination with either an elevated bilirubin or clinical jaundice, as defined by Hy's Law
Suspected transmission of an infectious agent by a study treatment, as defined below:
   Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a participant exposed to a medicinal product. This term applies only when a contamination of the study treatment is suspected.

### Adverse Events of Special Interest Specific to Mosunetuzumab

Adverse events of special interest specific to mosunetuzumab include: Grade ≥2 CRS; Grade ≥2 neurologic adverse event; Grade ≥2 injection-site reaction; any suspected HLH or MAS; TLS (minimum Grade 3 by definition); febrile neutropenia (minimum Grade 3 by definition); Grade ≥2 AST, ALT, or total bilirubin elevation; any Grade disseminated intravascular coagulation (minimum Grade 2 by definition); Grade ≥2 tumor flare (e.g., manifestation of signs/symptoms associated with an increase in size of known nodal or extranodal lesions by clinical or radiographic assessment, new onset or worsening of preexisting pleural effusions); and any Grade pneumonitis/Interstitial lung disease (ILD) (excluding pneumonia of infectious etiology)

### Adverse Events of Special Interest Specific to Atezolizumab and Tiragolumab

Adverse events of special interest specific to atezolizumab and/or tiragolumab include: pneumonitis; colitis; endocrinopathies: diabetes mellitus, pancreatitis, adrenal insufficiency, hyperthyroidism, and hypophysitis; gepatitis, including AST or ALT >10×ULN; systemic lupus erythematosus neurological disorders: Guillain-Barré syndrome, myasthenic syndrome or myasthenia gravis, and meningoencephalitis; events suggestive of hypersensitivity, infusion-related reactions, CRS, HLH and MAS; nephritis; ocular toxicities (e.g., uveitis, retinitis, optic neuritis); myositis; myopathies, including rhabdomyolysis; Grade ≥2 cardiac disorders (e.g., atrial fibrillation, myocarditis, pericarditis); vasculitis autoimmune hemolytic anemia; and severe cutaneous reactions (e.g., Stevens-Johnson syndrome, dermatitis bullous, toxic epidermal necrolysis)

### Pharmacokinetics

Samples will be used to evaluate the pharmacokinetics of mosunetuzumab, tiragolumab and atezolizumab. Samples collected for analyses of mosunetuzumab, tiragolumab and atezolizumab serum concentrations may also be used to evaluate safety or efficacy aspects related to concerns arising during or after the study. Also, these data will be used to understand the relationship of PK exposure to dose and support characterization of dose/exposure-response relationships in the combination setting. In addition, these data will be used to explore and characterize the potential PK interactions between tiragolumab, atezolizumab and mosunetuzumab.

### Biomarker Assessments

The following biomarker samples will be collected, as applicable, from participants at all sites:
Blood, PBMC and plasma samples for exploratory research on biomarkers and biomarker assay development
Newly collected (or archival) tumor tissue sample obtained at baseline for exploratory research on biomarkers and biomarker assay development

Exploratory biomarker research may include, but will not be limited to, RNA based expression analysis and DNA based NGS mutation profiling of genes such as MS4A1 (CD20) or gene signatures associated with tumor immunobiology, lymphocytes, activation status and phenotypes of immune cells, cytokines associated with T-cell activation, CRS and neurotoxicity.

Screening plasma, blood and tumor tissue samples, including those collected from individuals who do not enroll in the study, may be used for future research and/or development of disease-related tests or tools.

### H. Analysis

### Lugano Criteria

The primary efficacy endpoint is best ORR (CR or PR) on study, as determined by the investigator using Lugano 2014 criteria.

Response should be determined on the basis of radiographic and clinical evidence of disease.

Assessment of the positron emission tomography/computed tomography (PET/CT) scan should follow the criteria presented below (Cheson B D, et al. J Clin Oncol 2014; 32:1-9). Contrast-enhanced CT is preferred for more precise measurements of nodal masses and differentiation between normal anatomy and disease nodal infiltration of mediastinum and abdominal cavity.

### Target and Non-Target Lesions

Up to six of the largest target nodes, nodal masses, or other lymphomatous lesions that are measurable in two diameters should be identified from different body regions representative of the participant's overall disease burden and include mediastinal and retroperitoneal disease, if involved. At baseline, a measurable node must be >15 mm in the longest diameter (LDi). Measurable extranodal disease may be included in the six representative, measured lesions. At baseline, measurable extranodal lesions should be greater than 10 mm LDi. All other lesions (including nodal, extranodal, and assessable disease) should be followed as non-measured disease as non-target lesions (e.g., cutaneous, gastrointestinal, bone, spleen, liver, kidneys, pleural or pericardial effusions, ascites, bone, bone marrow).

### Spleen Involvement

Spleen may be of normal size or enlarged, homogeneous splenomegaly or diffuse infiltration with small lesions or large solid mass. A single measurement that correlate well with volume is preferable and the cut off >13 cm in the vertical length for splenomegaly is recommended.

### Liver Involvement

Measurement of liver size is not reliable by CT. Liver involvement is similar to the spleen involvement as diffuse increased or focal uptake on PET, with or without focal lesions.

### Bone Marrow Involvement

If applicable, bone marrow involvement uptake in PET is scored using the 5-point scale as nodal sites.

### Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease. The LDi and smallest diameter (SDi) are no longer needed to determine progression.

### Primary Endpoints

The primary endpoints are as follows for each study phase:

### Phase Ib

Incidence and severity of adverse events, including DLTs, with severity determined according to NCI CTCAE v5.0; for CRS, severity determined according to ASTCT CRS consensus grading criteria.

### Phase II

Best ORR, defined as the proportion of participants whose best overall response is a PR or a CR during the study, as determined by the investigator using Lugano 2014 criteria.

### Secondary Endpoints

The secondary endpoints are as follows for each study phase:

### Phase Ib

Best ORR (CR or PR at any time) on study as determined by the investigator using Lugano 2014 criteria.
Best CR rate, defined as the proportion of participants whose best overall response is a complete response during the study, as determined using Lugano 2014 criteria.
DOR, defined as the time from the first occurrence of a documented objective response (CR or PR) to disease progression or relapse, as determined using Lugano 2014 criteria, or death from any cause, whichever occurs first.

### Phase II

Best CR rate on study as determined by the investigator using Lugano 2014 criteria.
DOR, defined as the time from the first occurrence of a documented objective response (CR or PR) to disease progression or relapse, as determined using Lugano 2014 criteria, or death from any cause, whichever occurs first.
PFS, defined as the time from the first study treatment to the first occurrence of disease progression or relapse, as determined using Lugano 2014 criteria, or death from any cause, whichever occurs first
EFS, defined as the time from the first study treatment to the first occurrence of disease progression or relapse, as determined using Lugano 2014 criteria, initiation of NALT, or death from any cause, whichever occurs first.
OS, defined as the time from the first study treatment to death from any cause.
Occurrence and severity of adverse events, with severity determined according to NCI CTCAE v5.0. For CRS, severity determined according to the ASTCT CRS Consensus Grading criteria.

### Phase Ib and Phase II

Cₘₐₓ
Cₘᵢₙ
Total exposure (AUC), CL, and volume of distribution, as estimated by population PK modeling, as appropriate, and supported by data.

### Exploratory Endpoints

The exploratory efficacy endpoint is best ORR (CR or PR at any time) on study as determined by automated response using Lugano 2014 criteria (aLugano), a deep learning algorithm. Analysis of aLugano is performed on the PET/CT-evaluable populations, defined as all patients with available PET/CT scans at baseline and during treatment.

Automated total metabolic tumor volume (aTMTV), an exploratory imaging biomarker, is calculated using a deep learning algorithm on the evaluable population of patients with PET/CT scans available at baseline.

Additional analyses of aLugano, aTMTV, and other exploratory biomarkers may be performed.

### Example 4: A Phase Ib/II, Global, Multicenter, Open-Label, Multi-Cohort Umbrella Study in Patients with Metastatic Colorectal Cancer—Microsatellite Instability-High (MSI-H) Cohort

INTRINSIC (identifying and targeting subpopulations in colorectal cancer (CRC)) is a Phase I/Ib, global, multicenter, open-label, multi-cohort umbrella study. The study is designed to evaluate the safety and efficacy of targeted therapies or immunotherapy as single agents or in rational, specified combinations in patients with metastatic CRC whose tumors are biomarker positive as per cohort-specific definitions. This example describes the microsatellite instability-high (MSI-H) cohort of the INTRINSIC study.

### A. Overview of Study Design

The overarching structure of the INTRINSIC study is an umbrella interventional study in which eligible patients with metastatic CRC are enrolled in specific cohorts based on their biomarker assay results. Patients are assigned to cohorts based on the presence of genomic alterations identified by the blood-based FOUNDATIONONE® Liquid CDx Next-Generation Sequencing (NGS) assay during biomarker eligibility testing, screening, or by prior testing using a validated test as described in the inclusion criteria. Randomization within cohorts may be used to enable the quantitative evaluation of evidence relating to treatment effects. The study is designed with the flexibility to introduce new validated biomarker tests, to open new treatment arms and/or cohorts as tailored treatments for various identified somatic mutations or other biomarkers become available, to close existing treatment arms and/or cohorts that demonstrate minimal clinical activity or unacceptable toxicity, or to modify the patient population (e.g., with regard to prior anti-cancer treatment or biomarker status).

Patients meeting all general eligibility criteria and respective cohort-specific criteria are assigned to the appropriate treatment based on their genetic alteration(s) or respective biomarker.

The MSI-H cohort uses a randomized, open-label study design whereby patients are randomized to receive either atezolizumab, tiragolumab, and bevacizumab (Atezo+Tira+Bev) or atezolizumab and tiragolumab (Atezo+Tira). Enrollment in the MSI-H cohort is based on the confirmed presence of MSI-H by prior results of a validated NGS-, polymerase chain reaction- (PCR-), or immunohistochemistry- (IHC-) based assay as described in the inclusion criteria. Alternatively, patients may enroll in the cohort based on central FOUNDATIONONE® Liquid CDx testing provided by Foundation Medicine, Inc. (FMI) during biomarker eligibility testing or screening.

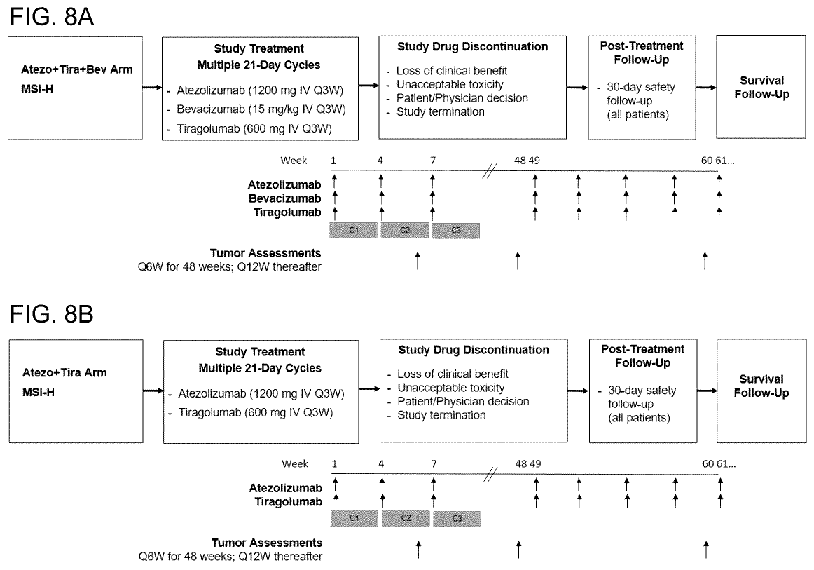

Summaries of the study design for the arms are shown in FIGS. 8A and 8B. Specific objectives and corresponding endpoints for the MSI-H cohort are outlined below (see Table 25).

TABLE 25
Objectives and Endpoints: Atezolizumab +
Tiragolumab + Bevacizumab and Atezolizumab +
Tiragolumab Treatment Arms of the MSI-H Cohort

| Primary Efficacy Objective | Corresponding Endpoints |
|---|---|
| To evaluate the efficacy of | Objective response rate (ORR), |
| atezolizumab in combination | defined as the proportion of |
| with tiragolumab, with or | patients with a complete response |
| without bevacizumab, in | or partial response on two |
| microsatellite instability- | consecutive occasions ≥4 weeks |
| high (MSI-H) CRC | apart, as determined by the |
| | investigator according to |
| | RECIST v1.1. |
| Secondary Efficacy Objective | Corresponding Endpoints |
| To evaluate the efficacy of | Duration of response (DOR), |
| atezolizumab in combination | defined as the time from the |
| with tiragolumab, with or | first occurrence of a documented |
| without bevacizumab, in MSI- | objective response to disease |
| H CRC. | progression or death from any |
| | cause (whichever occurs first), |
| | as determined by the investigator |
| | according to RECIST v1.1. |
| | Disease control rate (DCR), defined |
| | as the proportion of patients with |
| | stable disease for ≥12 weeks or a |
| | complete or partial response, as |
| | determined by the investigator |
| | according to RECIST v1.1. |
| Exploratory Efficacy Objectives | Corresponding Endpoints |
| To evaluate the efficacy | Overall survival (OS), defined |
| of atezolizumab in combination | as the time from start of treatment |
| with tiragolumab, with or | to death from any cause. |
| without bevacizumab, in MSI- | Progression-free survival (PFS) |
| H CRC. | after enrollment, defined as the |
| | time from enrollment to the first |
| | occurrence of disease progression |
| | or death from any cause (whichever |
| | occurs first), as determined by |
| | the investigator according to |
| | RECIST v1.1. |
| | OS at specific timepoints |
| | (e.g., 6 months). |
| | PFS at specific timepoints |
| | (e.g., 6 months). |
| Safety Objective | Corresponding Endpoints |
| To evaluate the safety and | Incidence, type, and severity |
| tolerability of atezolizumab | of adverse events (based on |
| in combination with tiragolumab, | National Cancer Institute Common |
| with or without bevacizumab. | Terminology Criteria for Adverse |
| | Events, Version 5.0 (NCI CTCAE |
| | v5.0)), including serious adverse |
| | events and adverse events of |
| | special interest. |
| | Severity for cytokine release |
| | syndrome (CRS) is also determined |
| | according to the American Society |
| | for Transplantation and Cellular |
| | Therapy (ASTCT) CRS Consensus |
| | Grading scale. |
| | Change from baseline in targeted |
| | clinical laboratory results. |
| Exploratory | |
| Pharmacokinetic Objective | Corresponding Endpoints |
| To characterize the | Serum concentrations of |
| pharmacokinetics (PK) | atezolizumab and |
| of atezolizumab and | tiragolumab at specified |
| tiragolumab when | timepoints. |
| administered in | |
| combination in | |
| this population. | |
| Exploratory | |
| Immunogenicity Objective | Corresponding Endpoints |
| To evaluate the immune | Prevalence of anti-drug |
| response to atezolizumab | antibodies (ADAs) to |
| and tiragolumab. | atezolizumab and tiragolumab |
| | at baseline and incidence |
| | of ADAs to atezolizumab |
| | and tiragolumab after |
| | initiation of study treatment. |
| Biomarker Objective | Corresponding Endpoint |
| To identify biomarkers | Relationship between biomarkers |
| that are predictive of | in blood and tumor tissue |
| response to treatment | (listed in Assessments section) |
| with atezolizumab in | and efficacy, safety, PK, |
| combination with tiragolumab, | or other biomarker endpoints. |
| with or without bevacizumab. | |
| Biomarkers are evaluated | |
| for their use as early | |
| surrogates of efficacy, | |
| association with | |
| progression to a more | |
| severe disease state (i.e., | |
| prognostic biomarkers), | |
| association with acquired | |
| resistance to treatment | |
| with atezolizumab in | |
| combination with tiragolumab, | |
| with or without bevacizumab, | |
| or that can increase the | |
| knowledge and understanding | |
| of disease biology and drug | |
| safety. | |

ADA = anti-drug antibody; ASTCT = American Society for Transplantation and Cellular Therapy; CRC = colorectal cancer; CRS = cytokine release syndrome; DCR = disease control rate; DOR = duration of response; MSI-H = microsatellite instability-high; NCI CTCAE v5.0 = National Cancer Institute Common Terminology Criteria for Adverse Events, Version 5.0; ORR = objective response rate; OS = overall survival; PFS = progression-free survival; PK = pharmacokinetic; RECIST v1.1 = Response Evaluation Criteria in Solid Tumors, Version 1.1.

The study has cohort-specific inclusion and exclusion criteria. Patients are assigned to cohorts on the basis of relevant oncogenotype, and, unless otherwise specified, continue until disease progression, as assessed by the investigator using Response Evaluation Criteria in Solid Tumors, Version 1.1 (RECIST v1.1; Eisenhauer et al. Eur J Cancer. 45: 228-247, 2009); loss of clinical benefit; unacceptable toxicity; patient or physician decision to discontinue; or death, whichever occurs first.

Following study treatment discontinuation, patients are followed for safety assessment for 30 days after final study treatment (30-day safety follow up, including a 30-day follow-up visit), or until the initiation of another anti-cancer therapy, whichever occurs first.

Follow-up data capture, including survival and subsequent anti-cancer therapies, continues for each patient until death, loss to follow-up, withdrawal of consent, or study or treatment arm and/or cohort closure, whichever occurs first. Information regarding the nature and duration of subsequent therapies is collected.

After prior consent, patients who discontinue study treatment due to disease progression, loss of clinical benefit, or toxicity may re-enter screening and could be eligible for treatment in a different cohort in INTRINSIC. In this scenario, patients who are biomarker-positive per cohort-specific definition, as determined by blood-based FOUNDATIONONE® Liquid CDx analysis on the blood sample obtained during the treatment discontinuation visit prior to starting a new anti-cancer therapy, and who meet and continue to meet all eligibility criteria for a different cohort currently open for enrollment, may be re-enrolled after prior cohort-specific informed consent. Cohort-specific restrictions still apply.

For re-enrollment in a different cohort, INTRINSIC patients can begin new study treatment as soon as possible while ensuring sufficient washout of the prior treatment regimen.

The umbrella structure of this study allows for the addition of new treatment arms and/or cohorts via future protocol amendments as new tailored treatments for various identified somatic mutations or other biomarkers become available. Any additional treatment arms and/or cohorts increase the total sample size and the number of patients screened.

### B. End of Study and Length of Study

The end of this study is defined as the date when the last patient, last visit (LPLV) or assessment occurs for the collection of the last data point for the last treatment arm. Any of the treatment arms and/or cohorts may close before the end of the entire study, when all pre-defined treatment, follow-up, and data collection are completed for that treatment arm. In addition, the Sponsor may decide to terminate a treatment arm, a cohort, or the study at any time.

Based on the longitudinal nature of this trial, the projected duration of this study from first patient enrolled to end of study, including survival follow-up visits, is expected to be approximately 36 months with the current treatment arms. However, the umbrella platform nature of this protocol may provide for an extension of the overall duration of this study to meet the objectives of additional treatment arms and/or cohorts added through protocol amendments, thus changing the assumptions and projections for LPLV.

### C. Rationale for Study Design

### Rationale for MSI-H Metastatic CRC Patient Population

Dysfunctional or absent mismatch repair (MMR) proteins are unable to properly correct primary single nucleotide insertion or deletion errors during DNA replication (Chung and Rustgi. Gastroenterology. 109: 1685-1699, 1995). These correction events during DNA replication often occur at DNA regions that contain repeating base pairs, otherwise known as microsatellites. When these MMR-deficient tumors harbor areas of DNA that are unable to be repaired, the errors cause a variation in the length of microsatellites present in the tumor and germline sequence, which is referred to as being MSI-H. MMR deficiency can occur sporadically or as a result of a germline mutation in one of several MMR genes, a condition known as Lynch syndrome. MSI-H CRC can also occur through sporadic mechanisms, such as hypermethylation of the promoter region of MLH1, which leads to epigenetic silencing of its gene product.

Historically, MSI-H status has had mainly prognostic implications in the advanced setting. However, recent discoveries show that targeting immune checkpoints, such as the PD-L1/PD-1 pathway, provides meaningful and durable responses in MSI-H CRC and improves survival. Pembrolizumab was the first U.S. Food and Drug Administration (FDA)-approved immune checkpoint inhibitor for MSI-H metastatic CRC. The first study to demonstrate the efficacy of pembrolizumab in this patient population was the KEYNOTE-016 study, which treated patients with MSI-H advanced cancers, demonstrating a response rate of greater than 50% across tumor types, with median PFS and OS not reached at the initial data cutoff date in December 2016 (Le et al. Science. 357: 409-413, 2017). Pooled analyses of patients with MSI-H CRC from a series of other multi-cohort trials, such as the KEYNOTE-028 study, demonstrated anti-tumor activity in this patient population, leading to FDA approval of pembrolizumab in the chemotherapy-refractory setting (Marcus et al. Clin Cancer Res. 25: 3753-3758, 2019).

Despite the aforementioned successes in treating patients with MSI-H metastatic CRC, a large proportion of patients do not benefit from the current approved immunotherapies, and some patients who initially respond to therapy later develop secondary resistance to therapy. To date, there have yet to be any novel, effective therapies developed for patients with MSI-H metastatic CRC that are refractory to standard checkpoint-inhibitor based therapies. The combination therapies in the MSI-H cohort of the study evaluates whether atezolizumab and tiragolumab, with or without bevacizumab, may be of benefit for patients with MSI-H metastatic CRC that is refractory to standard immune checkpoint inhibitors.

Rationale for Combination Treatment with Anti-PD-L1, Anti-TIGIT, and Anti-VEGF

Therapies targeting the mechanisms of resistance to anti-PD-L1/PD-1 therapies are needed to improve outcomes in patients who are refractory to these therapies. A strong scientific rationale and emerging clinical data suggest that combined PD-L1, VEGF, and TIGIT inhibition may be clinically beneficial in a number of tumor types.

Resistance to PD-L1/PD-1 blockade may result in the expression of multiple co-inhibitory receptors on the surface of effector T cells. Nonclinical tumor models have shown that TIGIT selectively suppresses the effector function of chronically stimulated CD8+ T cells, and that inhibiting both TIGIT and PD-L1/PD-1 results in superior efficacy compared with single-agent treatments (Johnston et al. Cancer Cell. 26: 923-937, 2014). Higher levels of several co-inhibitory receptors, including PD-1 and TIGIT, have also been observed in tumor tissue from patients with advanced CRC (Saleh et al. Cancer Immunol Immunother. 69: 1989-1999, 2020). Hence, targeting TIGIT and PD-L1 with tiragolumab and atezolizumab, respectively, may enhance the efficacy of PD-L1/PD-1 blockade across different cancer types, including MSI-H CRC.

Anti-VEGF agents promote the normalization of tumor vasculature, thereby increasing access of therapeutic agents (Jain. Nat Med. 7: 987-989, 2001). In addition, bevacizumab can restore and/or maintain the antigen-presentation capacity of DCs, leading to enhanced T-cell infiltration in tumors (Oelkrug and Ramage. Clin Exp Immunol. 178: 1-8, 2014; Wallin et al. Nat Commun. 7: 12624, 2016). Administration of anti-VEGF-A has been shown to attenuate tumor endothelial FasL expression and produce a significant increase in the influx of tumor-rejecting CD8+ T cells, leading to tumor growth suppression (Motz et al. Nat Med. 20: 607-615, 2014). Anti-VEGF therapies can also reduce the frequency of myeloid-derived suppressor cells, decrease production of suppressive cytokines, and lower expression of inhibitory checkpoints on CD8+ T cells in tumors (Roland et al. PloS One. 4: e7669, 2009; Voron et al. J Exp Med. 212: 139-148, 2015).

The immunomodulatory effects of bevacizumab are anticipated to increase CD8+ T-cell recruitment and relieve intratumoral immunosuppression, thereby boosting the effects of immunotherapy. In addition, VEGF recruits macrophages into the tumor microenvironment that have a M2 polarization state, which is typically involved in wound healing. These M2 tumor-associated macrophages ultimately help establish and maintain an immunosuppressive microenvironment (Chen and Mellman. Immunity. 39: 1-10, 2013). Indeed, clinical data have demonstrated a beneficial effect of anti-angiogenesis and immunomodulation within the context of atezolizumab treatment. The activity of combination treatment with atezolizumab and bevacizumab has been demonstrated in multiple, large, randomized Phase III clinical studies in patients with NSCLC, RCC, and HCC.

Based on the above-described data, it is hypothesized that combination treatment with atezolizumab, bevacizumab, and tiragolumab may augment the anti-tumor immune response, resulting in an improved and more durable clinical benefit in patients with checkpoint-inhibitor refractory MSI-H CRC.

### D. Inclusion Criteria

To qualify for enrollment, patients must meet the following general inclusion criteria. To be enrolled in the MSI-H cohort, patients must meet and continue to meet all general inclusion criteria, in addition to the treatment arm-specific criteria outlined below.

### General Inclusion Criteria

### Inclusion Criteria for Biomarker Eligibility Testing

Patients must meet all of the following criteria for biomarker eligibility testing:
Signed NGS Biomarker Eligibility Informed Consent Form.
Age ≥18 years at the time of signing Informed Consent Form.
Full report of prior testing results (if available) that does not meet screening criteria (see Biomarker Eligibility Testing and Screening section). Patients with a positive biomarker status test result that does meet screening criteria may directly enter screening.

### Inclusion Criteria for Screening

Patients must meet all of the following criteria for entry in the study:
Biomarker eligibility (per MSI-H cohort definition) as determined at a College of American Pathologists/Clinical Laboratory Improvement Amendments-certified or equivalently accredited diagnostic laboratory using a validated test based on:
   Prior test results and availability of a full report of the testing results (see Additional Inclusion Criteria for the MSI-H Cohort below).
      or
   Blood-based FOUNDATIONONE® Liquid CDx biomarker eligibility test result generated prior to or during screening or, in case of re-enrollment after treatment discontinuation, prior to starting a new anti-cancer therapy.
Age ≥18 years at the time of signing Informed Consent Form.
ECOG Performance Status ≤1.
Life expectancy ≥3 months, as determined by the investigator.
Histologically confirmed adenocarcinoma originating from the colon or rectum.
Metastatic disease (Stage IV American Joint Committee on Cancer, Version 7).
Ability to comply with the study protocol, in the investigator's judgment.
Measurable disease (at least one target lesion) according to RECIST v1.1. Previously irradiated lesions can be considered as measurable disease only if progressive disease has been unequivocally documented at that site since radiation.
Adequate hematologic and organ function within 14 days prior to initiation of study treatment, defined by the following: absolute neutrophil count (ANC) ≥1500/μL without granulocyte colony-stimulating factor; white blood cell (WBC) count ≥2.5×10⁹/L (2500/μL); lymphocyte count ≥0.5×10⁹/L (500/μL); platelet count ≥100,000/μL; hemoglobin ≥9 g/dL (patients must not have been transfused within 2 weeks prior to screening to meet this criterion); total bilirubin ≤1.5×upper limit of normal (ULN) (≤3×ULN if Gilbert syndrome); serum albumin ≥2.8 g/dL or 28 g/L; AST and ALT≤2.5×ULN (patients with documented liver metastases may have AST and/or ALT≤5.0×ULN); ALP≤2.5×ULN (patients with documented liver or bone metastases may have ALP ≤5.0×ULN); creatinine clearance ≥50 mL/min (calculated through use of the Cockcroft-Gault formula) or creatinine ≤1.5×ULN.
For patients not receiving therapeutic anticoagulation: INR ≤1.5×ULN and activated partial thromboplastin time (aPTT) ≤1.5×ULN
For patients receiving therapeutic anticoagulation: stable anticoagulant regimen
For women of childbearing potential: agreement to remain abstinent (refrain from heterosexual intercourse) or use contraceptive measures as outlined in each respective appendix
For men: agreement to remain abstinent (refrain from heterosexual intercourse) or use contraceptive measures, and agreement to refrain from donating sperm, as outlined in each respective appendix

### Additional Inclusion Criteria for the MSI-H Cohort

In addition to the inclusion criteria above, patients must meet the additional inclusion criteria listed below for entry into the MSI-H cohort (i.e., Atezo+Tira+Bev and Atezo+Tira treatment arms).
Biomarker eligibility as determined by prior results of a validated next-generation sequencing- (NGS-), PCR-, or immunohistochemistry- (IHC-) based assay.
   Patients may enroll based on central FOUNDATIONONE® Liquid CDx testing provided by Foundation Medicine during biomarker eligibility testing or screening.
Biomarker eligibility as per treatment arm-specific definition (to be read in conjunction with Additional Exclusion Criteria section below):
   MSI-H or high MSI designation (not microsatellite stable)
Disease progression on prior checkpoint-inhibitor-based therapy
   There is no limit to the number of prior treatment regimens.
Negative hepatitis B surface antigen (HBsAg) test at screening.
Positive hepatitis B surface antibody (HBsAb) test at screening, or negative HBsAb at screening accompanied by either of the following: Negative total hepatitis B core antibody (HBcAb) or positive total HBcAb test followed by quantitative hepatitis B virus (HBV) DNA <500 IU/mL.
Negative hepatitis C virus (HCV) antibody test at screening, or positive HCV antibody test followed by a negative HCV RNA test at screening.
For women of childbearing potential: agreement to remain abstinent (refrain from heterosexual intercourse) or use contraception.
For men: agreement to remain abstinent (refrain from heterosexual intercourse) or use a condom, and agreement to refrain from donating sperm.

### E. Exclusion Criteria

### General Exclusion Criteria

Patients who meet any of the following criteria are excluded from study entry in any cohort:
Current participation or enrollment in another interventional clinical trial.
Any systemic anti-cancer treatment within 2 weeks or 5 half-lives (whichever is shorter) prior to start of study treatment.
Treatment with investigational therapy within 28 days prior to initiation of study treatment. Note: For re-enrollment in a different cohort after study treatment discontinuation, the timepoint for initiation of the new study treatment (i.e., Day 1 of Cycle 1) may be sooner than 28 days since the final dose of the prior study treatment.
Pregnant or breastfeeding, or intending to become pregnant during the study.
History of or concurrent serious medical condition or abnormality in clinical laboratory tests that, in the investigator's judgment, precludes the patient's safe participation in and completion of the study or confounds the ability to interpret data from the study.
Severe infection within 4 weeks prior to initiation of study treatment, including, but not limited to, hospitalization for complications of infection, bacteremia, or severe pneumonia, or any active infection that, in the opinion of the investigator, could impact patient safety.
Incomplete recovery from any surgery prior to the start of study treatment that would interfere with the determination of safety or efficacy of study treatment.
Uncontrolled pleural effusion, pericardial effusion, or ascites requiring recurrent drainage procedures (once monthly or more frequently). Use of an in-dwelling catheter (e.g., PLEURX®) is allowed.
Uncontrolled tumor-related pain.
   Patients requiring pain medication must be on a stable regimen at study entry. Symptomatic lesions (e.g., bone metastases or metastases causing nerve impingement) amenable to palliative radiotherapy should be treated prior to enrollment. Patients should be recovered from the effects of radiation. There is no required minimum recovery period. Asymptomatic metastatic lesions that would likely cause functional deficits or intractable pain with further growth (e.g., epidural metastasis that is not presently associated with spinal cord compression) should be evaluated for loco-regional therapy, if appropriate, prior to enrollment.
Uncontrolled or symptomatic hypercalcemia (ionized calcium >1.5 mmol/L, calcium >12 mg/dL, or corrected serum calcium >ULN).
Clinically significant and active liver disease, including viral or other hepatitis, current alcohol abuse, or cirrhosis.
Known HIV infection.
Symptomatic, untreated, or actively progressing CNS metastases.
   Patients with a history of treated CNS metastases are eligible provided that all of the following criteria are met: Measurable disease, per RECIST v1.1, must be present outside the CNS; the patient has no history of intracranial hemorrhage or spinal cord hemorrhage; metastases are limited to the cerebellum or the supratentorial region (i.e., no metastases to the midbrain, pons, medulla, or spinal cord); there is no evidence of interim progression between completion of CNS-directed therapy and the screening brain scan; the patient has not undergone stereotactic radiotherapy within 7 days prior to initiation of study treatment or whole-brain radiotherapy within 14 days prior to initiation of study treatment; the patient has no ongoing requirement for corticosteroids as therapy for CNS disease; if the patient is receiving anti-convulsant therapy, the dose is considered stable; asymptomatic patients with CNS metastases newly detected at screening are eligible for the study after receiving radiotherapy or surgery, with no need to repeat the screening brain scan.
History of leptomeningeal disease or carcinomatous meningitis.
History of malignancy other than CRC within 2 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.

### Additional Exclusion Criteria for the MSI-H Cohort

Patients who meet any of the additional exclusion criteria listed below are excluded from entry into the Atezo+Tira+Bev and Atezo+Tira treatment arms:
History of or active inflammatory bowel disease (e.g., Crohn's disease or ulcerative colitis).
Any active bowel inflammation (including diverticulitis).
History of abdominal fistula, gastrointestinal (GI) perforation, intra-abdominal abscess, or active GI bleeding within 6 months prior to initiation of study treatment.
Clinical signs or symptoms of GI obstruction or requirement for routine parenteral hydration, parenteral nutrition, or tube feeding.
Evidence of abdominal free air not explained by paracentesis or recent surgical procedure.
Grade ≥2 proteinuria, as demonstrated by ≥2+protein on dipstick urinalysis and ≥1.0 g of protein in a 24-hour urine collection.
History of or active clinically significant cardiovascular dysfunction, including the following: History of stroke or transient ischemic attack within 6 months prior to first dose of study treatment; history of myocardial infarction within 6 months prior to first dose of study treatment; New York Heart Association Class III or IV cardiac disease; uncontrolled arrhythmias, history of or active ventricular arrhythmia requiring medication; coronary heart disease that is symptomatic or unstable angina.
Chronic corticosteroid therapy of 10 mg of prednisone per day or an equivalent dose of other anti-inflammatory corticosteroids or immunosuppressants for a chronic disease.
Allergy or hypersensitivity to components of the atezolizumab, tiragolumab, or bevacizumab formulation.
Treatment with a live, attenuated vaccine within 4 weeks prior to initiation of study treatment, or anticipation of need for a live, attenuated vaccine during atezolizumab and tiragolumab treatment, within 5 months after the final dose of atezolizumab or within 90 days after the final dose of tiragolumab.
Prior treatment with an anti-TIGIT agent.
Active Epstein-Barr virus (EBV) infection or known or suspected chronic active EBV infection at screening.
Major surgical procedure, or significant traumatic injury, within 28 days prior to Day 1 of Cycle 1 or anticipation of the need for major surgery during the course of study treatment.
Minor surgical procedures, excluding placement of a vascular access device, within 7 days prior to first dose of study treatment.
Core biopsy or other minor surgical procedure, excluding placement of a vascular access device, within 3 days prior to initiation of study treatment. Patients must have sufficiently recovered from any prior surgery, including adequate wound healing.
Serious, non-healing wound; active ulcer; or, untreated bone fracture.
Symptomatic, untreated, or actively progressing CNS metastases.
   Asymptomatic patients with treated CNS lesions are eligible, provided that all of the following criteria are met: Measurable disease, per RECIST v1.1, must be present outside the CNS; the patient has no history of intracranial hemorrhage or spinal cord hemorrhage; the patient has not undergone stereotactic radiotherapy within 7 days prior to initiation of study treatment, whole-brain radiotherapy within 14 days prior to initiation of study treatment, or neurosurgical resection within 28 days prior to initiation of study treatment; the patient has no ongoing requirement for corticosteroids as therapy for CNS disease (anti-convulsant therapy at a stable dose is permitted); there is no evidence of interim progression between completion of CNS-directed therapy and initiation of study treatment. Asymptomatic patients with CNS metastases newly detected at screening are eligible for the study after receiving radiotherapy or surgery, with no need to repeat the screening brain scan.
Prior allogenic stem cell or solid organ transplantation.
Inadequately controlled hypertension (defined as systolic blood pressure >150 mmHg and/or diastolic blood pressure >100 mmHg and based on an average of ≥3 blood pressure readings on ≥2 sessions. Anti-hypertensive therapy to achieve these parameters is allowed.
History of hypertensive crisis or hypertensive encephalopathy.
Significant vascular disease (e.g., aortic aneurysm requiring surgical repair or recent arterial thrombosis) within 6 months prior to initiation of study treatment.
History of Grade 4 venous thromboembolism.
History of Grade ≥2 hemoptysis (defined as ≥2.5 mL of bright red blood per episode) within 1 month prior to screening.
History or evidence of inherited bleeding diathesis or significant coagulopathy at risk of bleeding (i.e., in the absence of therapeutic anticoagulation).
Current or recent (<10 days prior to initiation of study treatment) use of aspirin (>325 mg/day), or clopidogrel (>75 mg/day). Note: The use of full-dose oral or parenteral anticoagulants for therapeutic purpose is permitted as long as the INR and aPTT is within therapeutic limits (according to institution standards) within 7 days prior to initiation of study treatment and the patient has been on a stable dose of anticoagulants for ≥2 weeks prior to initiation of study treatment. Prophylactic use of anticoagulants is allowed.

### F. Biomarker Eligibility Testing and Screening

This study seeks to enroll patients with a prior known positive biomarker result. For patients to directly enter screening, the following criteria must be met:
Consenting patients must be biomarker-positive per the cohort-specific definition for a cohort currently open for enrollment.
The prior positive biomarker result must be based on a validated test as described in the inclusion criteria (see Inclusion Criteria for Screening section), and a full report of the testing results must be available.
The prior biomarker result must meet the cohort-specific biomarker eligibility criteria for type of assay and timepoint of assay (see Table 26).
Consenting patients must agree to provide a predose blood sample on Day 1 of Cycle 1 for retrospective biomarker confirmation by FOUNDATIONONE® Liquid CDx testing.
In case of discordant results, treatment of the patient is not affected. However, the Sponsor may decide to replace patients to achieve the necessary sample size for efficacy-evaluable patients. The related (discordant) patient data are excluded from efficacy analysis but included in safety analyses (see Analysis section below).

TABLE 26
MSI-H Cohort Biomarker Eligibility
Criteria for Prior Biomarker Test

| | | Biomarker Eligibility Criteria | |
|---|---|---|---|
| Biomarker- | | to Be Met by Prior Test | |
| Based | | Type of | Timepoint |
| Cohort | Treatment Arm | Assay | of Test |
| MSI-H ^{a} | Atezo + Tira + Bev | NGS, PCR, IHC | No restrictions |
| | Atezo + Tira | NGS, PCR, IHC | No restrictions |

Atezo = atezolizumab; Bev = bevacizumab; IHC = immunohistochemistry; MSI-H = microsatellite instability-high; NGS = next-generation sequencing; Tira = tiragolumab.
Note:
The positive biomarker result must be based on a validated test as described in the inclusion criteria (see Inclusion Criteria for Screening section).
^{a} The prevalence of MSI-H among patients with metastatic CRC is 4% to 12% (Source: Foundation Medicine, Inc. FOUNDATIONCORE ® Database, Version November 2020. Available from: www.foundationmedicine.com/insights-and-trials/foundation-insights).

Consenting patients who do not meet the above criteria for screening may undergo central biomarker eligibility testing using FOUNDATIONONE® Liquid CDx. This is allowed only under the following conditions:
The prior biomarker test result does not meet the requirements outlined in Table 26, but there is sufficient confidence that the patient is biomarker-positive due to one or more of the following:
   There is a known previous positive biomarker result that cannot be located or verified.
   There is a known previous positive biomarker result, but the biomarker eligibility criteria for timepoint of assay are not met by prior test.
   There is a previous positive biomarker result that is based on a test that is not considered validated as described in the inclusion criteria (see Inclusion Criteria for Biomarker Eligibility Testing section), or does not meet the criteria for type of assay.
   There is a partially positive biomarker profile not fully meeting the cohort-specific definition.
In case of excessively slow enrollment, as identified by the Sponsor, biomarker eligibility testing may also be performed on consenting patients whose biomarker status is not known.
All patients, who undergo central biomarker eligibility testing, require a positive FOUNDATIONONE® Liquid CDx biomarker result before entering screening. If, at the discretion of the investigator, a streamlined process is required, the patient may enter screening and undergo the other screening assessments while waiting for the FOUNDATIONONE® Liquid CDx biomarker eligibility test result.

### G. Study Treatment

Patients in the atezolizumab+tiragolumab+bevacizumab (Atezo+Tira+Bev) and atezolizumab+tiragolumab (Atezo+Tira) treatment arms receive treatment as outlined in Tables 27 and 28, respectively, until unequivocal disease progression, unacceptable toxicity, patient or physician decision, or study/treatment arm termination. It is recommended that treatment be initiated no later than 7 days after arm enrollment; however, the first dose of study treatment should not occur within 3 days after a core biopsy or other surgical procedure.

Atezolizumab is administered at a fixed dose of 1200 mg Q3W (1200 mg on Day 1 of each 21-day cycle), and tiragolumab is administered at a fixed dose of 600 mg Q3W (600 mg on Day 1 of each 21-day cycle). For patients who are randomized into the triplet with bevacizumab, bevacizumab is administered at a dose of 15 mg/kg Q3W (15 mg/kg on Day 1 of each 21-day cycle).

TABLE 27
Treatment Regimen for Atezo + Tira + Bev Treatment Arm

| | Dose, Route, and Regimen |
|---|---|
| | (drugs listed in order |
| Cycle Length | of administration) |
| 21 days | Atezolizumab 1200 mg by |
| | IV infusion on Day 1 |
| | Bevacizumab 15 mg/kg IV |
| | infusion on Day 1 |
| | Tiragolumab 600 mg by IV |
| | infusion on Day 1 |

Atezo = atezolizumab; Bev = bevacizumab; Tira = tiragolumab.

TABLE 28
Treatment Regimen for Atezo + Tira Treatment Arm

| | Dose, Route, and Regimen |
|---|---|
| Cycle | (drugs listed in order |
| Length | of administration) |
| 21 days | Atezolizumab 1200 mg by IV |
| | infusion on Day 1 |
| | Tiragolumab 600 mg by IV |
| | infusion on Day 1 |

Atezo = atezolizumab; Tira = tiragolumab.

### Atezolizumab Administration

Atezolizumab is administered by IV infusion at a fixed dose of 1200 mg on Day 1 of each 21-day cycle. Administration of atezolizumab is performed in a monitored setting where there is immediate access to trained personnel and adequate equipment and medicine to manage potentially serious reactions. Atezolizumab infusions are administered per the instructions outlined in Table 29.

No dose modification for atezolizumab is allowed.

TABLE 29
Administration of First and Subsequent Atezolizumab Infusions

| First Infusion | Subsequent Infusions |
|---|---|
| No premedication is permitted | If the patient experienced |
| prior to the atezolizumab | an IRR with any previous |
| infusion. Vital signs | infusion, premedication |
| (pulse rate, respiratory | with antihistamines, anti- |
| rate, pulse oximetry, blood | pyretics, and/or analgesics |
| pressure, and temperature) | may be administered for |
| should be measured within | subsequent doses at the |
| 60 minutes prior to the | discretion of the investigator. |
| infusion. | |
| Atezolizumab should be | Vital signs should be measured |
| infused over 60 (±15) | within 60 minutes prior to the |
| minutes. | infusion. |
| If clinically indicated, | Atezolizumab should be infused |
| vital signs should be | over 30 (±10) minutes if the |
| measured every 15 (±5) | previous infusion was tolerated |
| minutes during the infusion | without an IRR, or 60 (±15) |
| and 30 (±10) minutes | minutes if the patient experienced |
| after the infusion. | an IRR with the previous infusion. |
| Patients should be informed | If the patient experienced an IRR |
| about the possibility | with the previous infusion or if |
| of delayed post-infusion | clinically indicated, vital signs |
| symptoms and instructed | should be measured during the |
| to contact their study | infusion and at 30 (±10) |
| physician if they develop | minutes after the infusion. |
| such symptoms. | |

IRR = infusion-related reaction.

### Bevacizumab Administration

Bevacizumab is administered at a dose of 15 mg/kg of body weight Q3W by IV infusion on Day 1 of each 21-day cycle for patients in the Atezo+Tira+Bev treatment arm. On Day 1 of each cycle, bevacizumab is administered at least 5 minutes after completion of the atezolizumab infusion. Body weight at baseline should be used to calculate the required dose of bevacizumab. If a weight change of >10% from baseline is observed, the treatment dosage should be modified accordingly (i.e., this becomes the new weight for dose calculation). A rounding up or down of the dose is acceptable to allow practical ease of administration. Rounding of the dose is optional, and if the treating physician decides to round the total dose of bevacizumab, it should be rounded to the nearest 5 mg.

Administration of bevacizumab is performed in a monitored setting where there is immediate access to trained personnel and adequate equipment and medicine to manage potentially serious reactions. Bevacizumab is administered per the instructions outlined in Table 30.

No dose modification for bevacizumab is allowed (with the exception of recalculating the dose if a >10% change in weight from baseline is noted).

TABLE 30
Administration of First and Subsequent Bevacizumab Infusions

| First Infusion | Subsequent Infusions |
|---|---|
| No premedication is permitted | If the patient experienced |
| prior to the bevacizumab | an IRR with any previous |
| infusion. | infusion, premedication with |
| Vital signs (pulse rate, | antihistamines, antipyretics, |
| respiratory rate, blood | and/or analgesics may be |
| pressure, pulse oximetry, | administered for subsequent |
| and temperature) should be | doses at the discretion of the |
| measured within 60 minutes | investigator. The infusion time |
| prior to the infusion. | may not be decreased for that |
| The initial infusion should | infusion. |
| be given over 90 (±10) | If the next infusion is well |
| minutes. | tolerated with premedication, |
| Vital signs should be measured at | the subsequent infusion time |
| the end of infusion and 2 (±1) | may then be decreased by 30 |
| hours after the infusion. | (±10) minutes as long as |
| Patients should be informed about | the patient continues to be |
| the possibility of delayed post- | premedicated. If a patient |
| infusion symptoms and instructed | experiences an infusion-associated |
| to contact their study physician | adverse event with the 60-minute |
| if they develop such symptoms. | infusion despite premedication, |
| | all subsequent doses should be |
| | delivered over 90 (±10) |
| | minutes. Similarly, if a patient |
| | experiences an infusion-associated |
| | adverse event with the 30-minute |
| | infusion despite premedication, |
| | all subsequent doses should |
| | be delivered over 60 (±10) |
| | minutes. |
| | Vital signs should be recorded |
| | within 60 minutes prior to the |
| | infusion. |
| | Bevacizumab should be infused |
| | over 60 (±10) minutes if |
| | the previous 90-minute infusion |
| | was tolerated without an IRR, |
| | or 90 (±15) minutes if |
| | the patient experienced an IRR |
| | with the previous infusion. If |
| | the 60-minute infusion was well |
| | tolerated, bevacizumab may be |
| | infused over 30 (±5) |
| | minutes thereafter. |
| | Vital signs should be measured |
| | within 30 minutes after completion |
| | of the infusion. |

IRR = infusion-related reaction.

### Tiragolumab Administration

Tiragolumab is administered by IV infusion at a fixed dose of 600 mg on Day 1 of each 21-day cycle with a post-infusion observation period as described in Table 31.

For patients in the Atezo+Tira+Bev treatment arm, on Day 1 of Cycle 1, tiragolumab is administered 60 minutes after completion of the bevacizumab infusion. The interval between subsequent infusions is 30 minutes if the previous bevacizumab infusion was given without premedication and tolerated without an IRR or 60 minutes if the patient experienced an IRR with the previous bevacizumab infusion.

For patients in the Atezo+Tira treatment arm, on Day 1 of Cycle 1, tiragolumab is administered 60 minutes after completion of the atezolizumab infusion. The interval between subsequent infusions is 30 minutes if the previous atezolizumab infusion was given without premedication and tolerated without an IRR or 60 minutes if the patient experienced an IRR with the previous atezolizumab infusion.

Administration of tiragolumab is performed in a monitored setting where there is immediate access to trained personnel and adequate equipment and medicine to manage potentially serious reactions.

No dose modification for tiragolumab is allowed.

TABLE 31
Administration of First and Subsequent Tiragolumab Infusions

| First Infusion | Subsequent Infusions |
|---|---|
| No premedication is permitted | If the patient experienced |
| prior to the tiragolumab | an IRR with any previous infusion, |
| infusion. | premedication with antihistamines, |
| Vital signs (pulse rate, | antipyretics, and/or analgesics |
| respiratory rate, pulse | may be administered for subsequent |
| oximetry, blood pressure, | doses at the discretion of the |
| and temperature) should | investigator. |
| be recorded within 60 minutes | Vital signs should be recorded |
| prior to the infusion. | within 60 minutes prior to the |
| Tiragolumab should be | infusion. |
| infused over 60 (±10) | Tiragolumab should be infused |
| minutes. | over 30 (±10) minutes if |
| Vital signs should be | the previous infusion was |
| recorded every 15 (±5) | tolerated without an IRR, or 60 |
| minutes during the infusion | (±10) minutes if the |
| and at 30 (±10) minutes | patient experienced an IRR with |
| after the infusion. | the previous infusion. |
| After the infusion of | Patients should be observed for |
| tiragolumab, the patient | 30 minutes after completion of |
| begins a 120-minute | the tiragolumab infusion if the |
| observation period. | previous infusion was tolerated |
| Patients are informed | without an IRR, or 60 minutes |
| about the possibility | after completion of the tiragolumab |
| of delayed post-infusion | infusion if the patient experienced |
| symptoms and instructed | an IRR with the previous infusion. |
| to contact their study | If the patient experienced an |
| physician if they | IRR with the previous infusion |
| develop such symptoms. | or if clinically indicated, |
| | vital signs should be recorded |
| | during the infusion and at 15 |
| | (±10) minutes after the |
| | infusion. |

IRR = infusion-related reaction.

### Treatment Interruption

Atezolizumab and/or tiragolumab treatment may be temporarily suspended in patients experiencing toxicity considered to be related to study treatment. If corticosteroids are initiated for treatment of the toxicity, they must be tapered over ≥1 month to ≤10 mg/day oral prednisone or equivalent before atezolizumab and tiragolumab can be resumed. However, the drug may be withheld for >12 weeks to allow for patients to taper off corticosteroids prior to resuming treatment. Atezolizumab or tiragolumab can be resumed after being withheld for >12 weeks if the patient is likely to derive clinical benefit. The decision to re-challenge patients with atezolizumab and tiragolumab should be based on investigator's assessment of benefit-risk and documented by the investigator.

On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, immune-mediated adverse events should generally be attributed to both agents, and dose interruptions or treatment discontinuation in response to immune-mediated adverse events should be applied to both tiragolumab and atezolizumab.

Bevacizumab treatment may be temporarily suspended in patients experiencing toxicity considered to be related to study treatment. If bevacizumab is withheld for >42 days, the patient is discontinued from bevacizumab. Bevacizumab can be resumed after being withheld for >42 days if the patient is likely to derive clinical benefit. The decision to re-challenge patients with bevacizumab should be based on investigator's assessment of benefit-risk and documented by the investigator.

Atezolizumab, tiragolumab, and bevacizumab treatment may be suspended for reasons other than toxicity (e.g., surgical procedures). The acceptable length of treatment interruption must be based on an assessment of benefit-risk by the investigator and in alignment with the protocol requirements for the duration of treatment and documented by the investigator.

If atezolizumab, bevacizumab, or tiragolumab are discontinued and there is not a clear contraindication to continuing the other drugs, the other drugs can be continued if the patient is likely to derive clinical benefit, as determined by the investigator per medical judgment.

Dosing of Study Treatment beyond Disease Progression

Patients in immunotherapy-containing treatment arms are treated until unacceptable toxicity or loss of clinical benefit defined as:
1. Confirmed disease progression in the tumor assessment following the finding of radiographic progression per RECIST v1.1; or
2. Lack of continued benefit as determined by the investigator after an integrated assessment of radiographic and biochemical data, local biopsy results (if available), and clinical status (e.g., symptomatic deterioration such as pain secondary to disease).

Because of the possibility of an initial increase in tumor burden caused by immune-cell infiltration in the setting of a T-cell response (termed pseudoprogression) with cancer immunotherapy (CIT) (such as atezolizumab), radiographic progression per RECIST v1.1 may not be indicative of true disease progression. After the first tumor assessment meeting the criteria for disease progression per RECIST v1.1, patients receiving treatment with a CIT drug are permitted to continue study treatment if they meet all of the following criteria:
Evidence of clinical benefit, as determined by the investigator following a review of all available data.
Absence of symptoms and signs (including laboratory values, such as new or worsening hypercalcemia) indicating unequivocal progression of disease.
Absence of decline in Eastern Cooperative Oncology Group (ECOG) Performance Status that can be attributed to disease progression.
Absence of tumor progression at critical anatomical sites (e.g., leptomeningeal disease) that cannot be managed by protocol-allowed medical interventions.

### H. Concomitant Therapy

Concomitant therapy consists of any medication (e.g., prescription drugs, over-the-counter drugs, vaccines, herbal or homeopathic remedies, nutritional supplements) used by a patient in addition to protocol-mandated study treatment from 7 days prior to initiation of study treatment to the treatment discontinuation visit.

If a patient experiences an infusion related reaction, premedication with antihistamines, antipyretics, and/or analgesics may be administered prior to subsequent infusions, at the discretion of the investigator.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H₂-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

Permitted Therapy Patients are permitted to use the following therapies during the study:
Oral contraceptives with a failure rate of <1% per year.
Hormone-replacement therapy.
For patients in the Atezo+Tira treatment arm: prophylactic or therapeutic anticoagulation therapy (such as warfarin at a stable dose or low-molecular-weight heparin (LMWH)).
For patients in the Atezo+Tira+Bev treatment arm: only prophylactic use of low-dose anticoagulation, unfractionated heparin, or LMWH. Adherence to the label-recommended dose for prophylactic use of anticoagulants is required. The preferred choice for anticoagulation treatment should be LMWH as per American Society of Clinical Oncology (ASCO) guidelines (Key et al. J Clin Oncol. 38: 496-520, 2020).
Vaccinations (such as influenza, COVID-19). Live, attenuated vaccines are not permitted.
For patients in the Atezo+Tira+Bev treatment arm: low-dose aspirin (<325 mg/day). Co-administration of proton pump inhibitors is strongly recommended to reduce potential GI damage.
Megestrol acetate administered as an appetite stimulant.
Mineralocorticoids (e.g., fludrocortisone).
Corticosteroids administered for chronic obstructive pulmonary disease or asthma.
Low-dose corticosteroids administered for orthostatic hypotension or adrenocortical insufficiency.
Palliative radiotherapy (e.g., treatment of known bony metastases or symptomatic relief of pain) as outlined: Palliative radiotherapy is permitted, provided it does not interfere with the assessment of tumor target lesions (e.g., the lesion to be irradiated must not be the only site of measurable disease). Treatment with atezolizumab and tiragolumab may be continued during palliative radiotherapy. Treatment with bevacizumab should be suspended during palliative radiotherapy. Upon completion of palliative radiotherapy, continuation of bevacizumab treatment may be continued when felt to be safe by the investigator.
Radiotherapy to the brain as outlined: patients whose extracranial tumor burden is stable or responding to study treatment and who are subsequently found to have three or fewer brain metastases may receive radiotherapy to the brain (either stereotactic radiosurgery or whole-brain radiation therapy) provided that all of the following criteria are met: the patient has no evidence of progression or hemorrhage after completion of CNS-directed therapy; the patient has no ongoing requirement for corticosteroids as therapy for CNS disease; and anti-convulsant therapy, if required, is at a stable dose. Note: Treatment with atezolizumab, bevacizumab, and tiragolumab should be withheld during CNS-directed radiation therapy.
Local therapy (e.g., surgery, stereotactic radiosurgery, radiotherapy, radiofrequency ablation) as outlined: Patients experiencing a mixed response requiring local therapy for control of three or fewer lesions may still be eligible to continue study treatment at the investigator's discretion. Patients who receive local therapy directed at a target lesion are no longer evaluable for radiographic response but remain evaluable for progression.

### I. Assessments

Patients are closely monitored for safety and tolerability throughout the study. Patients should be assessed for toxicity prior to each dose; dosing occurs only if the clinical assessment and local laboratory test values are acceptable.

### Biomarker Eligibility Testing

Patients who are biomarker eligible based on a prior test result who directly enter screening provide a predose blood sample on Day 1 of Cycle 1 and the prior biomarker test result is retrospectively confirmed by blood-based FOUNDATIONONE® Liquid CDx testing.

Patients who do not meet the biomarker eligibility criteria outlined in the Biomarker Eligibility Testing and Screening section to enter directly into screening may first undergo biomarker eligibility testing (or screening if a streamlined process is needed, at the discretion of the investigator) using FOUNDATIONONE® Liquid CDx.

### Tumor and Response Evaluations

Patients undergo tumor assessments at baseline, every 6 weeks (±1 week) for the first 48 weeks following initiation of treatment, and every 12 weeks (±2 weeks) thereafter, regardless of dose delays, until radiographic disease progression according to investigator-assessed RECIST v1.1, except in the case of patients in immunotherapy-containing cohorts who continue treatment after radiographic disease progression; such patients undergo tumor assessments every 6 weeks (±1 week) until loss of clinical benefit defined as (1) confirmed disease progression or (2) lack of continued benefit as determined by the investigator (see Study Treatment section for details). Thus, tumor assessments continue according to schedule in patients who discontinue treatment for reasons other than disease progression or loss of clinical benefit, even if they start new, non-protocol-specified anti-cancer therapy. At the investigator's discretion, tumor assessments may be repeated at any time if progressive disease is suspected.

Screening assessments must include computed tomography (CT) scans (with IV contrast; with or without oral contrast) or magnetic resonance imaging (MRI) scans (with IV contrast) of the chest, abdomen, pelvis, and head. A spiral CT scan of the chest may be obtained but is not a requirement. If a CT scan with contrast is contraindicated (i.e., in patients with contrast allergy or impaired renal clearance), a non-contrast CT scan of the chest may be performed and MRI scans (with IV contrast, if feasible) of the abdomen, pelvis, and head should be performed.

A CT or MRI scan of the head must be done with contrast at screening to evaluate CNS metastasis in all patients; if contrast is contraindicated, this assessment must be done by MRI. Bone scans and CT scans of the neck should also be performed if clinically indicated. At the investigator's discretion, other methods of assessment of measurable disease as per RECIST v1.1 may be used. If a CT scan for tumor assessment is performed in a positron emission tomography/CT scanner, the CT acquisition must be consistent with the standards for a full-contrast diagnostic CT scan.

All measurable and/or evaluable lesions identified at baseline should be re-assessed at subsequent tumor evaluations according to the schedule described above. Brain metastases identified at baseline that have been treated with radiotherapy or surgery are not considered measurable or evaluable unless there is suspected disease progression in the brain (i.e., the patient becomes symptomatic). Thus, subsequent head scans are not required unless clinically indicated. The same radiographic procedures used to assess disease sites at screening should be used for subsequent tumor assessments (e.g., the same contrast protocol for CT scans).

Overall response at a given timepoint is assessed by the investigator using RECIST v1.1.

### Biomarker Assessments

Blood samples are obtained for biomarker evaluation (including, but not limited to, biomarkers related to disease pathology or tumor immune biology) from all eligible patients. Blood samples are processed for the determination of changes in blood-based biomarkers and evaluated for cancer-related, immune-related, tumor type-related, and other exploratory biomarkers.

An archival tumor tissue sample for exploratory research on biomarkers must be submitted prior to study enrollment. Tumor samples are processed to obtain their derivatives (e.g., DNA, RNA) and evaluated for biomarker eligibility as well as cancer-related, immune-related, tumor type-related, and other exploratory biomarkers (e.g., alterations in gene expression or single-nucleotide polymorphisms).

Exploratory biomarker research may include, but is not limited to, cancer-related genomic alterations, analysis of ctDNA, genes or gene signatures associated with tumor biology and tumor immunobiology. Research may involve IHC extraction of DNA, cell-free DNA, or RNA; analysis of mutations, single-nucleotide polymorphisms, and other genomic variants; and genomic profiling through use of NGS of a comprehensive panel of genes. DNA extracted from blood may be compared with DNA extracted from tissue to identify somatic variants by distinguishing germline variants from somatic variants. NGS methods may include whole genome sequencing (WGS) or whole exome sequencing (WES) of tissue samples.

At participating sites, patients provide stool samples for exploratory biomarker research, including whole metagenomic sequencing and comprehensive analysis of the microbiome. Patients receive the collection device prior to baseline and the Cycle 3, Day 1 visit.

### J. Analysis

Efficacy analyses are based on the efficacy-evaluable population, defined as all patients who receive at least one dose of each drug for their assigned treatment regimen and who meet the MSI-H cohort-specific biomarker eligibility definition (see Table 26). Safety analyses are based on the safety-evaluable population, defined as all patients who receive any amount of study treatment. For efficacy analyses, the Sponsor may also conduct analyses on the per-protocol population, defined as efficacy evaluable population with at least one available tumor assessment (see Assessments section) that has received the target dose level.

### Determination of Sample Size

This Phase I/Ib exploratory study is not designed to make explicit power and type I error considerations for a hypothesis test. Instead, this study is designed to obtain preliminary efficacy, safety, and PK data on molecular guided treatment regimens including monotherapy as well as rational drug combinations when administered to patients with biomarker-positive metastatic CRC (as identified by blood-based FOUNDATIONONE® Liquid CDx testing during biomarker eligibility testing or screening or by prior testing using a validated test as described in the Inclusion Criteria section that is retrospectively confirmed by FOUNDATIONONE® Liquid CDx testing).

Approximately 15-80 patients are enrolled in each cohort. Enrollment is conducted at approximately 41 sites. Treatment arms where preliminary efficacy is shown after the preliminary stage of enrollment may pursue expansion and continued enrollment within the treatment arm. Numbers may increase due to additional enrollment of patients who prematurely discontinue study drug or study for reasons other than disease progression or treatment emergent toxicity and/or replacement of patients with discordant biomarker results if enrollment is based on a prior test result to achieve an adequate number of evaluable patients.

### Efficacy Analyses

Except where indicated, the following efficacy analyses are performed for all treatment arms. The analysis population for efficacy endpoints is the arm-specific efficacy-evaluable population. The Sponsor may also conduct analyses on the per-protocol population.

### Primary Efficacy Analyses

For each treatment arm, the primary efficacy endpoint is ORR, defined as the proportion of patients with an objective response per RECIST v1.1, as assessed by the investigator. An objective response is defined as a complete response (CR) or partial response (PR) per RECIST v1.1. Patients not meeting this criterion are considered non-responders.

For ORR, confirmation of objective response is required (confirmed ≥4 weeks apart in two separate tumor assessments). Unconfirmed response rate is also calculated.

An estimate of the ORR and its 90% confidence interval is calculated (estimated using the Clopper-Pearson method) for comparison with a set threshold for the given study treatment/cohort.

### Secondary Efficacy Analyses

The secondary efficacy endpoints include duration of response (DOR) and disease control rate (DCR), as determined by the investigator according to RECIST v1.1.

DOR is defined as the time from the date of the first occurrence of a confirmed CR or PR (whichever status is recorded first) to the date of the first documented disease progression or death due to any cause, whichever occurred first. DOR is assessed in patients who had a confirmed objective response during the study, as determined by the investigator according to RECIST v1.1. Patients who have neither progressed nor died at the time of analysis are censored at the last tumor assessment date. If no tumor assessments are performed after the date of the first occurrence of a CR or PR, DOR is censored at the date of the first occurrence of a CR or PR plus 1 day.

The Kaplan-Meier method is used to estimate the median DOR with 90% confidence intervals constructed through use of the Brookmeyer and Crowley method.

DCR, defined as the proportion of patients with stable disease for ≥12 weeks or a CR or PR, as determined by the investigator according to RECIST v1.1, is calculated for each treatment arm with 90% confidence intervals estimated using Clopper-Pearson's exact method.

### Exploratory Efficacy Analyses

The exploratory efficacy endpoints are PFS, OS, PFS at specific timepoints, and OS at specific timepoints.

PFS is defined as the time from the first date of treatment to the date of first documented disease progression or death, whichever occurs first. Disease progression is assessed by the investigator using RECIST v1.1. Patients who have neither experienced disease progression nor died at the time of analysis are censored at the last tumor assessment date. Patients with no post-baseline tumor assessment are censored at the first date of treatment plus 1 day.

OS is defined as the time from the first date of treatment to the date of death due to any cause. Patients who are not reported as dead at the time of the analysis are censored at the date when they were last known to be alive. If no post-baseline information is available, then OS is censored the date of the first treatment plus 1 day.

The Kaplan-Meier method is used to estimate the median for PFS and OS with 90% confidence intervals constructed through use of the Brookmeyer and Crowley method.

Landmark PFS rates and OS rates at specific timepoints are also estimated using the Kaplan-Meier method, with 90% confidence intervals calculated on the basis of Greenwood's estimate for the variance.

### Safety Analyses

The safety-evaluable population consists of all enrolled patients who received at least one dose of study treatment, with patients grouped according to treatment received.

Safety is assessed through summaries of exposure to study treatment, adverse events, and changes in laboratory test results.

Study treatment exposure (such as treatment duration, total dose received, and number of cycles and dose modifications) is summarized with descriptive statistics.

All verbatim adverse event terms are mapped to MedDRA thesaurus terms, and adverse event severity is graded according to the NCI CTCAE v5.0. All adverse events, serious adverse events, adverse events leading to death, adverse events of special interest, adverse events leading to dose reductions or interruptions, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and cause of death are summarized.

A shift table of selected laboratory tests is used to summarize the baseline and maximum post-baseline severity grade.

### Immunogenicity Analyses

Immunogenicity may be assessed for study treatments as appropriate. The immunogenicity analyses include all patients with at least one ADA assessment. Patients are grouped according to treatment received.

### Biomarker Analyses

Exploratory biomarker analyses are performed in an effort to understand the association of these biomarkers with response to study drugs, taking into account efficacy and safety endpoints.

### Interim Analyses

An interim analysis is conducted after approximately 20 efficacy-evaluable patients have been enrolled in each treatment arm during the preliminary stage. The Atezo+Tira+Bev treatment arm is compared with the Atezo+Tira treatment arm to demonstrate the contribution of bevacizumab. Both the Atezo+Tira+Bev and Atezo+Tira treatment arms are compared with the pre-specified standard-of-care benchmark. If a treatment arm shows a positive benefit-risk balance over the standard-of-care, approximately 20 additional patients are enrolled during the expansion stage, for a total of approximately 40 patients in each treatment arm. The treatment arm selected to undergo expansion depends on the contribution of bevacizumab.

Patients enrolled in the MSI-H cohort may consist of those who are primary refractory to CIT and those who are secondary refractory to CIT. As differential activity based on CIT refractory status cannot be ruled out, the Sponsor may decide to limit or stop enrollment of a patient population with a specific refractory status that demonstrates no advantage in benefit-risk assessment when expanding enrollment, while not excluding enrollment of patients with a specific refractory status during the preliminary stage.

A sample size of approximately 20 patients in each arm in the preliminary stage enables control of both the false positive rate and the false negative rate within the desired tolerance, ensuring an acceptable probability of a demonstration of the clinical effect of atezolizumab in combination with tiragolumab, with or without bevacizumab. The Sponsor may make a decision to expand enrollment based on the totality of available data including, but not limited to, ORR, duration of the observed responses, PFS, disease control rate, and—potentially—early OS data. Safety and biomarker data (available at the time of making this decision) are also taken into consideration from the perspective of an adequate benefit-risk assessment.

### OTHER EMBODIMENTS

Although the foregoing invention has been described in some detail by way of illustration and example for purposes of clarity of understanding, the descriptions and examples should not be construed as limiting the scope of the invention.

1. A method for treating a subject having a melanoma, the method comprising administering to the subject one or more dosing cycles of an anti-TIGIT antagonist antibody and a bispecific antibody targeting PD-1 and LAG3 comprising a first antigen-binding domain that specifically binds to programmed cell death protein 1 (PD-1) and a second antigen-binding domain that specifically binds to lymphocyte activation gene 3 (LAG3).

2. The method of claim 1, wherein the method comprises administering to the subject:
(a) the anti-TIGIT antagonist antibody at a fixed dose of 600 mg every three weeks; and
(b) the bispecific antibody at a fixed dose of 2100 mg every three weeks.

3. The method of claim 1, wherein the method comprises administering to the subject:
(a) the anti-TIGIT antagonist antibody at a fixed dose of 600 mg every three weeks; and
(b) the bispecific antibody at a fixed dose of 600 mg every three weeks.

4-6. (canceled)

7. The method of claim 1, wherein the one or more dosing cycles are administered as a neoadjuvant therapy.

8. A method for treating a subject having a melanoma, the method comprising administering to the subject one or more dosing cycles of a bispecific antibody targeting PD-1 and LAG3 comprising a first antigen-binding domain that specifically binds to PD-1 and a second antigen-binding domain that specifically binds to LAG3, wherein the one or more dosing cycles are administered as a neoadjuvant therapy.

9. The method of claim 8, wherein the method comprises administering to the subject the bispecific antibody at a fixed dose of 2100 mg every three weeks.

10. The method of claim 8, wherein the method comprises administering to the subject the bispecific antibody at a fixed dose of 600 mg every three weeks.

11-12. (canceled)

13. A method for treating a subject having a melanoma, the method comprising administering to the subject one or more dosing cycles of an anti-TIGIT antagonist antibody and a PD-1 axis binding antagonist, wherein the one or more dosing cycles are administered as a neoadjuvant therapy.

14-52. (canceled)

53. A method of achieving a clinical response in a subject having a metastatic esophageal cancer comprising administering to the subject a dosing regimen comprising one or more dosing cycles of tiragolumab and atezolizumab in an amount effective to achieve the clinical response.

54-55. (canceled)

56. The method of claim 53, wherein the method comprises:
(a) administering to the subject tiragolumab at a dose of about 600 mg every three weeks and atezolizumab at a dose of about 1200 mg every three weeks;
(b) administering to the subject tiragolumab at a dose of about 420 mg every two weeks and atezolizumab at a dose of about 840 mg every two weeks; or
(c) administering to the subject tiragolumab at a dose of about 840 mg every four weeks and atezolizumab at a dose of about 1680 mg every four weeks.

57-62. (canceled)

63. A method of treating a subject having a relapsed or refractory (R/R) non-Hodgkin's lymphoma (NHL) comprising administering to the subject tiragolumab and mosunetuzumab.

64-106. (canceled)

107. A method of treating a subject having a metastatic colorectal cancer (CRC) comprising administering to the subject tiragolumab and atezolizumab, wherein the metastatic CRC is a microsatellite instability-high (MSI-H) CRC.

108. A method of treating a subject having a metastatic CRC comprising administering to the subject tiragolumab, atezolizumab, and bevacizumab, wherein the metastatic CRC is a MSI-H CRC.

109-122. (canceled)
